(19) United States
(12) Patent Application Publication
(10) Pub. No.: US 2024/0270806 A1
(43) Pub. Date: Aug. 15, 2024

(54) PROTEASE-ACTIVATED CYTOKINE

(71) Applicant: CHUGAI SEIYAKU KABUSHIKI KAISHA, Tokyo (JP)

(72) Inventors: Vishnu Priyanka Reddy CHICHILI, Singapore (SG); Tomoyuki IGAWA, Tokyo (JP); Yohei YAMAMOTO, Kanagawa (JP); Naoka HIRONIWA, Singapore (SG); Takeru NAMBU, Kanagawa (JP); Tatsuya KAWA, Kanagawa (JP); Hitoshi KATADA, Kanagawa (JP); Tomoko YASUGI, Kanagawa (JP); Masahiro AZUMA, Kanagawa (JP)

(21) Appl. No.: 18/414,813

(22) Filed: Jan. 17, 2024

(30) Foreign Application Priority Data
Jan. 18, 2023 (EP) 23152286.3

(51) Int. Cl. C07K 14/54; A61K 38/00; A61K 39/00; C07K 16/24

(52) U.S. Cl. CPC C07K 14/5434; C07K 16/244; A61K 38/00; A61K 2039/505; C07K 2317/565; C07K 2319/30

(57) ABSTRACT

The present invention relates to fusion proteins or protein complexes comprising a ligand binding moiety fused to a ligand moiety, wherein the ligand binding moiety comprises an IL-12 binding domain and a protease cleavage site that, when activated by cleavage by a protease, restores biological activity of the ligand moiety, wherein the ligand moiety is an IL-12 fusion protein. The invention also relates to a polypeptide representing a mutated p40 subunit of IL-12, and to an IL-12 fusion protein or a protein complex comprising the mutated p40 subunit. The invention also relates to anti-IL-12 binding molecule or anti-IL-12 binding molecule complex defined by its variable light and heavy chains. The invention also relates to polynucleotides encoding the fusion proteins or fusion protein complexes or polypeptides or binding molecules, to methods of producing same, to their uses and pharmaceutical compositions comprising same.

### CROSS-REFERENCE TO RELATED APPLICATIONS

This application claims priority to European Patent Application No. 23152286.3, filed Jan. 18, 2023, the contents of which are incorporated herein by reference in their entirety.

### REFERENCE TO SEQUENCE LISTING SUBMITTED ELECTRONICALLY

The content of the electronically submitted sequence listing (Name: 6663_0286_Sequence_Listing.xml, Size: 1,197,040 bytes, and Date of Creation: Mar. 14, 2024) is herein incorporated by reference in its entirety.

### TECHNICAL FIELD

The present invention relates to protein complexes, polypeptides, proteins, fusion proteins or fusion protein complexes comprising an ligand binding moiety fused to a ligand moiety, wherein the ligand binding moiety comprises an IL-12 binding domain and a protease cleavage site that, when activated by cleavage by a protease, restores biological activity of the ligand moiety, wherein the ligand moiety is an IL-12 fusion protein. The invention also relates to a polypeptide representing a mutated p40 subunit of IL-12, and to an IL-12 fusion protein or a protein complex comprising the mutated p40 subunit. The invention also relates to an anti-IL-12 binding molecule or anti-IL-12 binding molecule complex defined by its variable light and variable heavy chains. The invention also relates to polynucleotides encoding the protein complexes, proteins, fusion proteins or fusion protein complexes or polypeptides or binding molecules, to methods of producing same, to their uses and pharmaceutical compositions comprising same.

### BACKGROUND ART

The innate ability of our immune system is characterized by its potency, specificity, and memory. Motivated by these features, immunotherapies are being developed in diverse areas, including infectious diseases, autoimmunity, allergies, transplant rejection, graft versus host diseases and cancer. Cytokines and chemokines which are small proteins well known in their roles in the body's immune response to inflammation and immune attack are the center stage of the development of immunotherapy.

However, to date, there remains a common concern for cytokine mediated immunotherapies regarding high systemic toxicity and low to negligible efficacy. Upon administration, cytokines are systemically exposed and therefore elicit toxicity by systemic action. Therefore, cytokines can often only be administered at very low doses to circumvent such toxicity. An appealing strategy to overcome this includes coupling cytokines to antibodies to locally increase the cytokine concentrations at tumor sites. The cytokine delivered to solid cancer by the immunocytokine activates immunity and thereby exerts an antitumor effect. Since cytokines including IL-2, IL-12, and TNF have strong toxicity, it is expected that the localized action of these cytokines on cancer may be strengthened when delivered by antibodies in a localized delivery while alleviating adverse reactions (NPL1-NPL3). However, it has been reported that such immunocytokines diffuse throughout the body and thus, can bind to any cells in the blood or tissues so long as there are specific, high-affinity cytokine receptors present, leading to unwarranted side effects. In a particular instance, it was reported that an IL-2 fused to an antibody binding a cancer antigen exhibited the same anti-tumor effect as an IL-2 fused to an antibody that does not bind to the cancer antigen, suggesting the IL-2 moiety directed its biodistribution not the antibody component (NPL4).

Other alternative approaches include having cytokines fused to their receptors via a protease cleavable linker. In an environment, such as a cancer environment, where protease expression is high, the linker is cleaved and the cytokine is released from its receptor. Immunocytokines comprised of such formats include TNF-alpha and TNF-receptor connected via a linker cleavable by urokinase-type plasminogen activator (uPA) (NPL5) and IL-2 and IL-2 receptor cleavable by matrix metalloproteinase-2 (MMP-2) (NPL6). However, the cytokines in these molecules are active even while fused to their receptors, and when activated upon protease cleavage, the improvement in activity is limited, i.e. approximately 10 times.

More recently, a variety of fusion polypeptides comprising cytokines that are released upon protease cleavage have been reported including for example, a single-chain fragment variable (scFv) fused to IL-2 and IL-12 cleavable by matrix metalloproteinases (MMPs) (NPL6, NPL7, PTL2, PTL5) and other fusion polypeptides comprising a protease cleavable region as reported in PTL1, PTL3, PTL4, PTL6, PTL7 and PTL8.

### CITATION LIST

### Patent Literature

[PTL 1] WO 2009/025846
[PTL 2] WO 2011/123683
[PTL 3] WO 2018/097307
[PTL 4] WO 2019/107380
[PTL 5] WO 2019/010219
[PTL 6] WO 2019/010224
[PTL 7] WO 2020/061526
[PTL 8] WO 2021/016640

### Non Patent Literature

[NPL 1] Cyclophosphamide and tucotuzumab (huKS-IL2) following first-line chemotherapy in responding patients with extensive-disease small-cell lung cancer.

### Gladkov O, Ramlau R, Serwatowski P, Milanowski J, Tomeczko J, Komarnitsky P B, Kramer D, Krzakowski M J. Anticancer Drugs. 2015 November; 26 (10): 1061-8.

[NPL 2] Defining the Pharmacodynamic Profile and Therapeutic Index of NHS-IL12 Immunocytokine in Dogs with Malignant Melanoma. Paoloni M, Mazcko C, Selting K, Lana S, Barber L, Phillips J, Skorupski K, Vail D, Wilson H, Biller B, Avery A, Kiupel M, LeBlanc A, Bernhardt A, Brunkhorst B, Tighe R, Khanna C. PLoS One. 2015 Jun. 19; 10 (6): e0129954.
[NPL 3] Isolated limb perfusion with the tumor-targeting human monoclonal antibodycytokine fusion protein L19-TNF plus melphalan and mild hyperthermia in patients with locally advanced extremity melanoma. Papadia F, Basso V, Patuzzo R, Maurichi A, Di Florio A, Zardi L, Ventura E, Gonzalez-Iglesias R, Lovato V, Giovannoni L, Tasciotti A, Neri D, Santinami M, Menssen H D, De Cian F. J Surg Oncol. 2013 February; 107 (2): 173-9.
[NPL 4] Antigen specificity can be irrelevant to immunocytokine efficacy and biodistribution. Tzeng A, Kwan B H, Opel C F, Navaratna T, Wittrup K D. Proc Natl Acad Sci USA. 2015 Mar. 17; 112 (11): 3320-5.
[NPL 5] Cancer Immunol Immunother. 2006 December; 55 (12): 1590-600. Epub 2006 April 25. Target-selective activation of a TNF prodrug by urokinase-type plasminogen activator (uPA) mediated proteolytic processing at the cell surface. Gerspach J1, Nemeth J, Munkel S, Wajant H, Pfizenmaier K.
[NPL 6] Immunology. 2011 June; 133 (2): 206-20. doi:10.1111/j.1365-2567.2011.03428.x. Epub 2011 March 23. Development of an attenuated interleukin-2 fusion protein that can be activated by tumor-expressed proteases.

### Puskas J1, Skrombolas D, Sedlacek A, Lord E, Sullivan M, Frelinger J.

[NPL 7] Development of an Interleukin-12 Fusion Protein That Is Activated by Cleavage with Matrix Metalloproteinase 9. Skrombolas D, Sullivan M, Frelinger J G. J Interferon Cytokine Res. 2019 April; 39(4):233-245

### SUMMARY OF INVENTION

### Technical Problem

It is well known that cytokines are key immune mediators residing in many lesion sites whose effects, when harnessed, can significantly improve immune responses. This also applies to IL-12. While many cytokine-mediated immune therapies have been developed, the issue of high toxicity and low efficacy remain of concern.

### Solution to the Problem

The present inventors have thought that the ability to deliver a site-specifically activated IL-12 cytokine at high dose would overcome systemic toxicity and low efficacy issue. Moreover, the inventors have thought to provide a site-specifically activated IL-12 cytokine with improved window between a non-cleaved molecule and the activated molecule. To achieve such site specific delivery with improved window between a non-cleaved molecule and the activated molecule, the present inventors have inter alia developed fusion proteins, protein complexes, polypeptides or proteins, that comprise an affinity maturated IL-12 binding moiety, a protease cleavable site and an improved IL-12 ligand. The IL-12 is bound to the C-terminal region of the constant region of the ligand-binding moiety by a non-cleavable peptide linker. In a first state, the IL-12 is also bound non-covalently to the affinity maturated IL-12 ligand-binding domain and its ability to bind a binding partner (IL-12 receptor) is attenuated. In a second state, after cleavage at the protease cleavable site, the ligand is not bound to the ligand-binding domain, because by said cleavage, the binding of the IL-12 to the ligand-binding domain has been released, but remains bound to the constant region by the non-cleavable peptide linker. By said cleavage, the ability of the IL-12 to bind to a binding partner is restored (IL-12 receptor) and it is able to exert its biological activity upon binding to said binding partner (see, for example, FIG. 1 and FIG. 3B).

Furthermore, in one nonexclusive aspect, the present inventors have provided affinity matured IL-12 binding molecules, which are characterized by their enhanced and optimal affinity to IL-12, which improves the window between non-cleaved and active molecule. Furthermore, in another nonexclusive aspect, the present inventors have provided protease cleavable linkers, which are characterized by their stability in the absence of a protease, and by effective cleavage in the presence of a protease. Furthermore, in another nonexclusive aspect, the present inventors have provided an improved IL-12 ligand as well as a p40 subunit thereof, which is characterized by its protease resistance, and its low potency. The low potent ligand provides the advantage of lower toxicity while maintaining efficacy. As explained above, these affinity matured IL-12 binding molecules, protease cleavable linkers and the improved IL-12 may be part of the fusion proteins, protein complexes, polypeptides, or proteins of the invention.

In some embodiments, the fusion proteins, polypeptides, protein complexes, or proteins of the invention are bivalent homodimers comprising two IL-12-binding moieties, each comprising an IL-12-binding domain with a protease cleavage site and one IL-12 ligand bound to the IL-12 ligand-binding domain. The improved activities of the fusion proteins, polypeptides, protein complexes, proteins or pharmaceutical compositions comprising the fusion protein, protein complex, polypeptide, or protein of the invention are also shown in the attached examples.

The fusion proteins, polypeptides, protein complexes, or proteins and pharmaceutical compositions comprising the fusion protein, polypeptide, protein complex, or protein are useful in the treatment of a disease where IL-12 is helpful. In one nonexclusive aspect, a method of administering said fusion proteins, polypeptides, protein complexes, proteins, or pharmaceutical compositions are also included. In another nonexclusive aspect, the invention further provides said fusion proteins, polypeptides, protein complexes, proteins or pharmaceutical compositions for use in a method of treatment of a disease where IL-12 is helpful. Without wishing to be bound by any theory, the present inventors have found that an activated form of the fusion protein, protein complex, polypeptide, or protein of the invention is capable of accumulating in high concentrations at the disease site and exhibit fast clearance from the site when compared to the natural ligand IL-12. This offers advantage of administering the fusion protein, polypeptide, protein complex, or protein in a higher dose with lesser side effects when compared to the natural ligand IL-12 and other molecular formats described in the prior art that delivers the natural ligand in their activated form.

### Exemplary Embodiments

The present invention is based on such findings, and specifically includes the exemplary aspects and embodiments described below.

[A-1] A protein complex comprising or consisting of two subunits associated with each other, wherein each subunit comprises a first polypeptide and a second polypeptide, wherein the first polypeptide comprises or consists of a light chain variable domain (VL) and a light chain constant domain (CL), and the second polypeptide is a fusion protein represented by the general formula (I), from the N- to the C-terminus:

[VH]-[Lx]-[Cx]-[Ly]-[ligand moiety] (I)
wherein:
   Lx represents a cleavable peptide linker comprising a protease cleavage site,
   Cx represents a constant domain associated with the CL of the first polypeptide, optionally, wherein Cx comprises or consists of, from the N- to the C-terminus, a CH1 domain, a second peptide linker, a CH2 domain, and a CH3 domain, and
   Ly represents a third peptide linker,
   VH represents a heavy chain variable domain (VH), wherein the VH is associated with the VL of the first polypeptide to form a ligand-binding domain, wherein the VH and the VL comprise any one of the following sequences:
      (a) a heavy chain variable domain (VH) comprising an amino acid sequence with at least 85%, 90%, 95%, 96%, 97%, 98%, or 99% sequence identity to the amino acid sequence of SEQ ID NO: 189, and a light chain variable domain (VL) comprising an amino acid sequence with at least 85%, 90%, 95%, 96%, 97%, 98%, or 99% sequence identity to the amino acid sequence of SEQ ID NO: 190; or
      (b) a heavy chain variable domain (VH) comprising an amino acid sequence with at least 85%, 90%, 95%, 96%, 97%, 98%, or 99% sequence identity to the amino acid sequence of SEQ ID NO: 189, and a light chain variable domain (VL) comprising an amino acid sequence with at least 85%, 90%, 95%, 96%, 97%, 98%, or 99% sequence identity to the amino acid sequence of SEQ ID NO: 342; or
      (c) a heavy chain variable domain (VH) comprising an amino acid sequence with at least 85%, 90%, 95%, 96%, 97%, 98%, or 99% sequence identity to the amino acid sequence of SEQ ID NO: 340, and a light chain variable domain (VL) comprising an amino acid sequence with at least 85%, 90%, 95%, 96%, 97%, 98%, or 99% sequence identity to the amino acid sequence of SEQ ID NO: 343; or
      (d) a heavy chain variable domain (VH) comprising an amino acid sequence with at least 85%, 90%, 95%, 96%, 97%, 98%, or 99% sequence identity to the amino acid sequence of SEQ ID NO: 340, and a light chain variable domain (VL) comprising an amino acid sequence with at least 85%, 90%, 95%, 96%, 97%, 98%, or 99% sequence identity to the amino acid sequence of SEQ ID NO: 344; or
      (e) a heavy chain variable domain (VH) comprising an amino acid sequence with at least 85%, 90%, 95%, 96%, 97%, 98%, or 99% sequence identity to the amino acid sequence of SEQ ID NO: 341, and a light chain variable domain (VL) comprising an amino acid sequence with at least 85%, 90%, 95%, 96%, 97%, 98%, or 99% sequence identity to the amino acid sequence of SEQ ID NO: 344; or
      (f) a heavy chain variable domain (VH) comprising an amino acid sequence with at least 85%, 90%, 95%, 96%, 97%, 98%, or 99% sequence identity to the amino acid sequence of SEQ ID NO: 189, and a light chain variable domain (VL) comprising an amino acid sequence with at least 85%, 90%, 95%, 96%, 97%, 98%, or 99% sequence identity to the amino acid sequence of SEQ ID NO: 191; or
      (g) a heavy chain variable domain (VH) comprising the amino acid sequence of SEQ ID NO: 189, and a light chain variable domain (VL) comprising the amino acid sequence of SEQ ID NO: 190; or
      (h) a heavy chain variable domain (VH) comprising the amino acid sequence of SEQ ID NO: 189, and a light chain variable domain (VL) comprising the amino acid sequence of SEQ ID NO: 342; or
      (i) a heavy chain variable domain (VH) comprising the amino acid sequence of SEQ ID NO: 340, and a light chain variable domain (VL) comprising the amino acid sequence of SEQ ID NO: 343; or
      (j) a heavy chain variable domain (VH) comprising the amino acid sequence of SEQ ID NO: 340, and a light chain variable domain (VL) comprising the amino acid sequence of SEQ ID NO: 344; or
      (k) a heavy chain variable domain (VH) comprising the amino acid sequence of SEQ ID NO: 341, and a light chain variable domain (VL) comprising the amino acid sequence of SEQ ID NO: 344; or
      (l) a heavy chain variable domain (VH) comprising the amino acid sequence of SEQ ID NO: 189, and a light chain variable domain (VL) comprising the amino acid sequence of SEQ ID NO: 191;
wherein Lx links VH and the CH1 domain and comprises any one of the following sequences:
   (i) an amino acid sequence of SEQ ID NO: 199; or
   (ii) an amino acid sequence of SEQ ID NO: 194,
wherein Cx comprises an amino acid sequence of SEQ ID NO: 75;
wherein Ly comprises an amino acid sequence of SEQ ID NO: 68;
wherein the ligand moiety is a protease resistant single-chain IL-12 fusion protein and comprises any one of the following sequences:
   (i) an amino acid sequence of SEQ ID NO: 317; or
   (ii) the amino acid sequence of SEQ ID NO: 317, wherein the tyrosine at position 16 of SEQ ID NO: 317 is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W); preferably proline (P); or
   (iii) an amino acid sequence of SEQ ID NO: 285; or
   (iv) the amino acid sequence of SEQ ID NO: 285, wherein the tyrosine at position 16 of SEQ ID NO: 285 is substituted with an amino acid selected from the group consisting of: phenylalanine (F), asparagine (N), glutamic acid (E), proline (P), glutamic acid (G), lysine (K), alanine (A), aspartic acid (D), histidine (H), isoleucine
   (I), leucine (L), methionine (M), glutamine (Q), arginine (R), serine (S), threonine (T), valine (V), and tryptophan (W); preferably proline (P); or
   (v) an amino acid sequence of SEQ ID NO: 325; or
   (vi) an amino acid sequence of SEQ ID NO: 335.
      [A-1a] A protein complex comprising or consisting of two subunits associated with each other, wherein each subunit comprises a first polypeptide and a second polypeptide, wherein the first polypeptide comprises or consists of a light chain variable domain (VL) and a light chain constant domain (CL), and the second polypeptide is a fusion protein represented by the general formula (I), from the N- to the C-terminus:

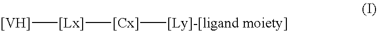

wherein:
   Lx represents a cleavable peptide linker comprising a protease cleavage site,
   Cx represents a constant domain associated with the CL of the first polypeptide, optionally, wherein Cx comprises or consists of, from the N- to the C-terminus, a CH1 domain, a second peptide linker, a CH2 domain, and a CH3 domain, and
   Ly represents a third peptide linker,
   VH represents a heavy chain variable domain (VH), wherein the VH is associated with the VL of the first polypeptide to form a ligand-binding domain, wherein the VH and the VL comprise any one of the following sequences:
      (a) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
      (b) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 354, and (iii) LCDR3 comprising at least 85%, 90%, 95%, 96%, 97%, 98%, or 99% sequence identity to the amino acid sequence of SEQ ID NO: 355; or
      (c) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 352, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
      (d) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
      (e) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 354, and (iii) LCDR3 comprising at least 85%, 90%, 95%, 96%, 97%, 98%, or 99% sequence identity to the amino acid sequence of SEQ ID NO: 355; or
      (f) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 352, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
      (g) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 351, (ii) LCDR2 comprising the amino acid sequence of SEQ ID NO: 353, and (iii) LCDR3 comprising the amino acid sequence of SEQ ID NO: 355; or
      (h) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 351, (ii) LCDR2 comprising the amino acid sequence of SEQ ID NO: 354, and (iii) LCDR3 comprising the amino acid sequence of SEQ ID NO: 355; or
      (i) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 352, (ii) LCDR2 comprising the amino acid sequence of SEQ ID NO: 353, and (iii) LCDR3 comprising the amino acid sequence of SEQ ID NO: 355; or
      (j) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 351, (ii) LCDR2 comprising the amino acid sequence of SEQ ID NO: 353, and (iii) LCDR3 comprising the amino acid sequence of SEQ ID NO: 355; or
      (k) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 351, (ii) LCDR2 comprising the amino acid sequence of SEQ ID NO: 354, and (iii) LCDR3 comprising the amino acid sequence of SEQ ID NO: 355; or
      (l) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 352, (ii) LCDR2 comprising the amino acid sequence of SEQ ID NO: 353, and (iii) LCDR3 comprising the amino acid sequence of SEQ ID NO: 355;
wherein Lx links VH and the CH1 domain and comprises any one of the following sequences:
   (i) an amino acid sequence of SEQ ID NO: 199; or
   (ii) an amino acid sequence of SEQ ID NO: 194,
wherein Cx comprises an amino acid sequence of SEQ ID NO: 75;
wherein Ly comprises an amino acid sequence of SEQ ID NO: 68;
wherein the ligand moiety is a protease resistant single-chain IL-12 fusion protein and comprises any one of the following sequences:
   (i) an amino acid sequence of SEQ ID NO: 317; or
   (ii) the amino acid sequence of SEQ ID NO: 317, wherein the tyrosine at position 16 of SEQ ID NO: 317 is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W); preferably proline (P); or
   (iii) an amino acid sequence of SEQ ID NO: 285; or
   (iv) the amino acid sequence of SEQ ID NO: 285, wherein the tyrosine at position 16 of SEQ ID NO: 285 is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W); preferably proline (P); or
   (v) an amino acid sequence of SEQ ID NO: 325; or
   (vi) an amino acid sequence of SEQ ID NO: 335.
      [A-1b] A protein complex comprising or consisting of two subunits associated with each other, wherein each subunit comprises a first polypeptide and a second polypeptide, wherein the first polypeptide comprises or consists of a light chain variable domain (VL) and a light chain constant domain (CL), and the second polypeptide is a fusion protein represented by the general formula (I), from the N- to the C-terminus:

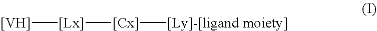

wherein:
   Lx represents a cleavable peptide linker comprising a protease cleavage site,
   Cx represents a constant domain associated with the CL of the first polypeptide, optionally, wherein Cx comprises or consists of, from the N- to the C-terminus, a CH1 domain, a second peptide linker, a CH2 domain, and a CH3 domain, and
   Ly represents a third peptide linker,
   VH represents a heavy chain variable domain (VH), wherein the VH is associated with the VL of the first polypeptide to form a ligand-binding domain, wherein the VH and the VL comprise any one of the following sequences:
      (a) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 190; or
      (b) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 342; or
      (c) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 340, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 343; or
      (d) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 340, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 344; or
      (e) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 341, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 344; or
      (f) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 191; or
      (g) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 190; or
      (h) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 342; or
      (i) a heavy chain variable domain (VH) comprising HCDR1, HCDR2, and HCDR3 that is identical to the amino acids of HCDR1, HCDR2, and HCDR3 comprised in SEQ ID NO: 340, and a light chain variable domain (VL) comprising LCDR1, LCDR2, and LCDR3 that is identical to the amino acids of LCDR1, LCDR2, and LCDR3 comprised in SEQ ID NO: 343; or
      (j) a heavy chain variable domain (VH) comprising HCDR1, HCDR2, and HCDR3 that is identical to the amino acids of HCDR1, HCDR2, and HCDR3 comprised in SEQ ID NO: 340, and a light chain variable domain (VL) comprising LCDR1, LCDR2, and LCDR3 that is identical to the amino acids of LCDR1, LCDR2, and LCDR3 comprised in SEQ ID NO: 344; or
      (k) a heavy chain variable domain (VH) comprising HCDR1, HCDR2, and HCDR3 that is identical to the amino acids of HCDR1, HCDR2, and HCDR3 comprised in SEQ ID NO: 341, and a light chain variable domain (VL) comprising LCDR1, LCDR2, and LCDR3 that is identical to the amino acids of LCDR1, LCDR2, and LCDR3 comprised in SEQ ID NO: 344; or
      (l) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 191;
wherein Lx links VH and the CH1 domain and comprises any one of the following sequences:
   (i) an amino acid sequence of SEQ ID NO: 199; or
   (ii) an amino acid sequence of SEQ ID NO: 194,
wherein Cx comprises an amino acid sequence of SEQ ID NO: 75;
wherein Ly comprises an amino acid sequence of SEQ ID NO: 68;
wherein the ligand moiety is a protease resistant single-chain IL-12 fusion protein and comprises any one of the following sequences:
   (i) an amino acid sequence of SEQ ID NO: 317; or
   (ii) the amino acid sequence of SEQ ID NO: 317, wherein the tyrosine at position 16 of SEQ ID NO: 317 is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W); preferably proline (P); or
   (iii) an amino acid sequence of SEQ ID NO: 285; or
   (iv) the amino acid sequence of SEQ ID NO: 285, wherein the tyrosine at position 16 of SEQ ID NO: 285 is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W); preferably proline (P); or
   (v) an amino acid sequence of SEQ ID NO: 325; or
   (vi) an amino acid sequence of SEQ ID NO: 335.
      [A-2] The protein complex of [A-1], [A-1a], or [A-1b], wherein:
i) the two subunits are bound to each other via one or more disulfide bonds; and/or
ii) the protein complex is obtainable by a) culturing a mammalian host cell expressing the polypeptides, and b) retrieving the protein complex from the supernatant, preferably wherein the mammalian host cell is a Chinese Hamster Ovary (CHO) cell; and/or
iii) the protein complex is glycosylated and/or is produced recombinantly by expression in a mammalian host cell, preferably wherein the mammalian host cell is a Chinese Hamster Ovary (CHO) cell; and/or
iv) the CH1 domain of Cx and the CL of the first polypeptide are bound to each other via one or more disulfide bonds.
   [A-3] The protein complex of [A-1], [A-1a], [A-1b], or [A-2], wherein
(a) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(b) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(c) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(d) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(e) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(f) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(g) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(h) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(i) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(j) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(k) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(l) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317; and
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(m) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(n) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(o) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(p) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(q) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(r) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(s) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(t) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317; and
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(u) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(v) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(w) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317; and
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(x) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P.
      [A-3a] The protein complex of [A-1], [A-1a], [A-1b], or [A-2], wherein
(a) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(b) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(c) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(d) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(e) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(f) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(g) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(h) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(i) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(j) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(k) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(l) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317; and
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(m) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(n) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(o) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(p) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(q) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(r) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(s) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(t) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317; and
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(u) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(v) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(w) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317; and
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(x) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P.
      [A-4] The protein complex of any one of [A-1], [A-1a], [A-1b], or [A-2], wherein
(a) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and the ligand moiety is a protease resistant single-chain IL-12 fusion protein comprising the amino acid sequence of SEQ ID NO: 325, or comprising or consisting of the amino acid sequence of SEQ ID NO: 335;
   or
(b) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(c) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(d) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(e) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(f) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(g) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(h) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(i) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(j) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(k) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and the ligand moiety is a protease resistant single-chain IL-12 fusion protein comprising the amino acid sequence of SEQ ID NO: 325, or comprising or consisting of the amino acid sequence of SEQ ID NO: 335;
   or
(l) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
(m) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(n) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(o) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(p) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(q) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(r) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(s) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(t) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(u) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(v) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(w) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(x) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335.
      [A-5] A protein complex comprising or consisting of four polypeptides, wherein:
(a) the first and second polypeptide each comprise the amino acid sequence of SEQ ID NO: 204, and the third and fourth polypeptide each comprise the amino acid sequence of SEQ ID NO: 121; or
(b) the first and second polypeptide each comprise the amino acid sequence of SEQ ID NO: 204, and the third and fourth polypeptide each comprise the amino acid sequence of SEQ ID NO: 201; or
(c) the first and second polypeptide each comprise the amino acid sequence of SEQ ID NO: 205, and the third and fourth polypeptide each comprise the amino acid sequence of SEQ ID NO: 202; or
(d) the first and second polypeptide each comprise the amino acid sequence of SEQ ID NO: 264, and the third and fourth polypeptide each comprise the amino acid sequence of SEQ ID NO: 121; or
(e) the first and second polypeptide each comprise the amino acid sequence of SEQ ID NO: 264, and the third and fourth polypeptide each comprise the amino acid sequence of SEQ ID NO: 122; or
(f) the first and second polypeptide each comprise the amino acid sequence of SEQ ID NO: 207, and the third and fourth polypeptide each comprise the amino acid sequence of SEQ ID NO: 121; or
(g) the first and second polypeptide each comprise the amino acid sequence of SEQ ID NO: 207, and the third and fourth polypeptide each comprise the amino acid sequence of SEQ ID NO: 201; or
(h) the first and second polypeptide each comprise the amino acid sequence of SEQ ID NO: 208, and the third and fourth polypeptide each comprise the amino acid sequence of SEQ ID NO: 202; or
(i) the first and second polypeptide each comprise the amino acid sequence of SEQ ID NO: 208, and the third and fourth polypeptide each comprise the amino acid sequence of SEQ ID NO: 209; or
(j) the first and second polypeptide each comprise the amino acid sequence of SEQ ID NO: 210, and the third and fourth polypeptide each comprise the amino acid sequence of SEQ ID NO: 209; or
(k) the first and second polypeptide each comprise the amino acid sequence of SEQ ID NO: 244, and the third and fourth polypeptide each comprise the amino acid sequence of SEQ ID NO: 121; or
   (l) the first and second polypeptide each comprise the amino acid sequence of SEQ ID NO: 245, and the third and fourth polypeptide each comprise the amino acid sequence of SEQ ID NO: 121; or
   (m) the first and second polypeptide each comprise the amino acid sequence of SEQ ID NO: 249, and the third and fourth polypeptide each comprise the amino acid sequence of SEQ ID NO: 121; or
   (n) the first and second polypeptide each comprise the amino acid sequence of SEQ ID NO: 249, and the third and fourth polypeptide each comprise the amino acid sequence of SEQ ID NO: 122.
      [A-5a] A protein complex consisting of four polypeptides, wherein:
(a) the first and second polypeptide each consist of the amino acid sequence of SEQ ID NO: 204, and the third and fourth polypeptide each consist of the amino acid sequence of SEQ ID NO: 121; or
(b) the first and second polypeptide each consist of the amino acid sequence of SEQ ID NO: 204, and the third and fourth polypeptide each consist of the amino acid sequence of SEQ ID NO: 201; or
(c) the first and second polypeptide each consist of the amino acid sequence of SEQ ID NO: 205, and the third and fourth polypeptide each consist of the amino acid sequence of SEQ ID NO: 202; or
(d) the first and second polypeptide each consist of the amino acid sequence of SEQ ID NO: 264, and the third and fourth polypeptide each consist of the amino acid sequence of SEQ ID NO: 121; or
(e) the first and second polypeptide each consist of the amino acid sequence of SEQ ID NO: 264, and the third and fourth polypeptide each consist of the amino acid sequence of SEQ ID NO: 122; or
(f) the first and second polypeptide each consist of the amino acid sequence of SEQ ID NO: 207, and the third and fourth polypeptide each consist of the amino acid sequence of SEQ ID NO: 121; or
(g) the first and second polypeptide each consist of the amino acid sequence of SEQ ID NO: 207, and the third and fourth polypeptide each consist of the amino acid sequence of SEQ ID NO: 201; or
(h) the first and second polypeptide each consist of the amino acid sequence of SEQ ID NO: 208, and the third and fourth polypeptide each consist of the amino acid sequence of SEQ ID NO: 202; or
(i) the first and second polypeptide each consist of the amino acid sequence of SEQ ID NO: 208, and the third and fourth polypeptide each consist of the amino acid sequence of SEQ ID NO: 209; or
(j) the first and second polypeptide each consist of the amino acid sequence of SEQ ID NO: 210, and the third and fourth polypeptide each consist of the amino acid sequence of SEQ ID NO: 209; or
(k) the first and second polypeptide each consist of the amino acid sequence of SEQ ID NO: 244, and the third and fourth polypeptides, each consist of the amino acid sequence of SEQ ID NO: 121; or
(l) the first and second polypeptide each consist of the amino acid sequence of SEQ ID NO: 245, and the third and fourth polypeptide each consist of the amino acid sequence of SEQ ID NO: 121; or
(m) the first and second polypeptide each consist of the amino acid sequence of SEQ ID NO: 249, and the third and fourth polypeptide each consist of the amino acid sequence of SEQ ID NO: 121; or
(n) the first and second polypeptide each consist of the amino acid sequence of SEQ ID NO: 249, and the third and fourth polypeptide each consist of the amino acid sequence of SEQ ID NO: 122.
   [A-6] The protein complex of [A-5] or [A-5a], wherein:
i) the first and second polypeptides are each a heavy chain, and the third and fourth polypeptides are each a light chain, and wherein the heavy and light chains are bound to each other via one or more disulfide bonds; and/or
ii) the first and second polypeptides are each a heavy chain, and the third and fourth polypeptides are each a light chain, and the heavy chain comprises a heavy chain variable domain (VH), and the light chain comprises a light chain variable domain (VL), wherein the VH is associated with the VL; and/or
iii) the protein complex is obtainable by a) culturing a mammalian host cell expressing the polypeptides, and b) retrieving the protein complex from the supernatant, preferably wherein the mammalian host cell is a Chinese Hamster Ovary (CHO) cell; and/or
iv) the protein complex is glycosylated and/or is produced recombinantly by expression in a mammalian host cell, preferably wherein the mammalian host cell is a Chinese Hamster Ovary (CHO) cell; and/or
v) the first and second polypeptides are each a heavy chain, and the third and fourth polypeptides are each a light chain, wherein the heavy chain comprises a CH1 domain, and wherein the light chain comprises a CL domain, and wherein the CH1 domain is associated with the CL domain.
   [A-7] A protein complex comprising a protease resistant single-chain IL-12 fusion protein fused with an anti-IL-12 binding molecule complex, wherein the anti-IL-12 binding molecule complex comprises a heavy chain variable domain (VH) associated with a light chain variable domain (VL), a cleavable peptide linker comprising a protease cleavage site, and a constant domain, wherein said VH and VL comprises any one of the following sequences:
(a) a heavy chain variable domain (VH) comprising an amino acid sequence with at least 85%, 90%, 95%, 96%, 97%, 98%, or 99% sequence identity to the amino acid sequence of SEQ ID NO: 189, and a light chain variable domain (VL) comprising an amino acid sequence with at least 85%, 90%, 95%, 96%, 97%, 98%, or 99% sequence identity to the amino acid sequence of SEQ ID NO: 190; or
(b) a heavy chain variable domain (VH) comprising an amino acid sequence with at least 85%, 90%, 95%, 96%, 97%, 98%, or 99% sequence identity to the amino acid sequence of SEQ ID NO: 189, and a light chain variable domain (VL) comprising an amino acid sequence with at least 85%, 90%, 95%, 96%, 97%, 98%, or 99% sequence identity to the amino acid sequence of SEQ ID NO: 342; or
(c) a heavy chain variable domain (VH) comprising an amino acid sequence with at least 85%, 90%, 95%, 96%, 97%, 98%, or 99% sequence identity to the amino acid sequence of SEQ ID NO: 340, and a light chain variable domain (VL) comprising an amino acid sequence with at least 85%, 90%, 95%, 96%, 97%, 98%, or 99% sequence identity to the amino acid sequence of SEQ ID NO: 343; or
(d) a heavy chain variable domain (VH) comprising an amino acid sequence with at least 85%, 90%, 95%, 96%, 97%, 98%, or 99% sequence identity to the amino acid sequence of SEQ ID NO: 340, and a light chain variable domain (VL) comprising an amino acid sequence with at least 85%, 90%, 95%, 96%, 97%, 98%, or 99% sequence identity to the amino acid sequence of SEQ ID NO: 344; or
(e) a heavy chain variable domain (VH) comprising an amino acid sequence with at least 85%, 90%, 95%, 96%, 97%, 98%, or 99% sequence identity to the amino acid sequence of SEQ ID NO: 341, and a light chain variable domain (VL) comprising an amino acid sequence with at least 85%, 90%, 95%, 96%, 97%, 98%, or 99% sequence identity to the amino acid sequence of SEQ ID NO: 344; or
(f) a heavy chain variable domain (VH) comprising an amino acid sequence with at least 85%, 90%, 95%, 96%, 97%, 98%, or 99% sequence identity to the amino acid sequence of SEQ ID NO: 189, and a light chain variable domain (VL) comprising an amino acid sequence with at least 85%, 90%, 95%, 96%, 97%, 98%, or 99% sequence identity to the amino acid sequence of SEQ ID NO: 191; or
(g) a heavy chain variable domain (VH) comprising the amino acid sequence of SEQ ID NO: 189, and a light chain variable domain (VL) comprising the amino acid sequence of SEQ ID NO: 190; or
(h) a heavy chain variable domain (VH) comprising the amino acid sequence of SEQ ID NO: 189, and a light chain variable domain (VL) comprising the amino acid sequence of SEQ ID NO: 342; or
(i) a heavy chain variable domain (VH) comprising the amino acid sequence of SEQ ID NO: 340, and a light chain variable domain (VL) comprising the amino acid sequence of SEQ ID NO: 343; or
(j) a heavy chain variable domain (VH) comprising the amino acid sequence of SEQ ID NO: 340, and a light chain variable domain (VL) comprising the amino acid sequence of SEQ ID NO: 344; or
(k) a heavy chain variable domain (VH) comprising the amino acid sequence of SEQ ID NO: 341, and a light chain variable domain (VL) comprising the amino acid sequence of SEQ ID NO: 344; or
(l) a heavy chain variable domain (VH) comprising the amino acid sequence of SEQ ID NO: 189, and a light chain variable domain (VL) comprising the amino acid sequence of SEQ ID NO: 191;
wherein the cleavable peptide linker is located between the boundary of VH and the constant domain, and comprises any one of the following sequences:
   (i) an amino acid sequence of SEQ ID NO: 199; or
   (ii) an amino acid sequence of SEQ ID NO: 194,
wherein the constant domain comprises an amino acid sequence SEQ ID NO: 75;
wherein the protease resistant single-chain IL-12 fusion protein is fused to the C-terminus of the constant domain and comprises one of the following sequences:
   (i) an amino acid sequence of SEQ ID NO: 317; or
   (ii) the amino acid sequence of SEQ ID NO: 317, wherein the tyrosine at position 16 of SEQ ID NO: 317 is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W); preferably proline (P);
   (iii) an amino acid sequence of SEQ ID NO: 285; or
   (iv) the amino acid sequence of SEQ ID NO: 285, wherein the tyrosine at position 16 of SEQ ID NO: 285 is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W); preferably proline (P); or
   (v) an amino acid sequence of SEQ ID NO: 325; or
   (vi) an amino acid sequence of SEQ ID NO: 335.
      [A-8] A protein complex comprising or consisting of two protein complexes of [A-7].
      [A-9] The protein complex of [A-8], wherein the complex is a homodimer.
      [A-10] A protein complex comprising two polypeptides, wherein each polypeptide is a fusion protein, and each polypeptide is represented by the general formula (II), from the N- to the C-terminus:

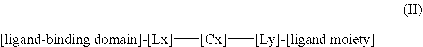

wherein:
   Lx represents a cleavable peptide linker comprising a protease cleavage site,
   Cx represents a constant domain comprising, from N- to C-terminus, a CH1 domain, a second peptide linker, a CH2 domain, and a CH3 domain, and
   Ly represents a third peptide linker,
   wherein the ligand-binding domain comprises a heavy chain variable domain (VH) associated with a light chain variable domain (VL), wherein VL is associated with a light chain constant domain (CL), wherein CL is associated with Cx, and wherein VH and VL comprise any one of the following sequences:
      (a) a heavy chain variable domain (VH) comprising an amino acid sequence with at least 85%, 90%, 95%, 96%, 97%, 98%, or 99% sequence identity to the amino acid sequence of SEQ ID NO: 189, and a light chain variable domain (VL) comprising an amino acid sequence with at least 85%, 90%, 95%, 96%, 97%, 98%, or 99% sequence identity to the amino acid sequence of SEQ ID NO: 190; or
      (b) a heavy chain variable domain (VH) comprising an amino acid sequence with at least 85%, 90%, 95%, 96%, 97%, 98%, or 99% sequence identity to the amino acid sequence of SEQ ID NO: 189, and a light chain variable domain (VL) comprising an amino acid sequence with at least 85%, 90%, 95%, 96%, 97%, 98%, or 99% sequence identity to the amino acid sequence of SEQ ID NO: 342; or
      (c) a heavy chain variable domain (VH) comprising an amino acid sequence with at least 85%, 90%, 95%, 96%, 97%, 98%, or 99% sequence identity to the amino acid sequence of SEQ ID NO: 340, and a light chain variable domain (VL) comprising an amino acid sequence with at least 85%, 90%, 95%, 96%, 97%, 98%, or 99% sequence identity to the amino acid sequence of SEQ ID NO: 343; or
      (d) a heavy chain variable domain (VH) comprising an amino acid sequence with at least 85%, 90%, 95%, 96%, 97%, 98%, or 99% sequence identity to the amino acid sequence of SEQ ID NO: 340, and a light chain variable domain (VL) comprising an amino acid sequence with at least 85%, 90%, 95%, 96%, 97%, 98%, or 99% sequence identity to the amino acid sequence of SEQ ID NO: 344; or
      (e) a heavy chain variable domain (VH) comprising an amino acid sequence with at least 85%, 90%, 95%, 96%, 97%, 98%, or 99% sequence identity to the amino acid sequence of SEQ ID NO: 341, and a light chain variable domain (VL) comprising an amino acid sequence with at least 85%, 90%, 95%, 96%, 97%, 98%, or 99% sequence identity to the amino acid sequence of SEQ ID NO: 344; or
      (f) a heavy chain variable domain (VH) comprising an amino acid sequence with at least 85%, 90%, 95%, 96%, 97%, 98%, or 99% sequence identity to the amino acid sequence of SEQ ID NO: 189, and a light chain variable domain (VL) comprising an amino acid sequence with at least 85%, 90%, 95%, 96%, 97%, 98%, or 99% sequence identity to the amino acid sequence of SEQ ID NO: 191; or
      (g) a heavy chain variable domain (VH) comprising the amino acid sequence of SEQ ID NO: 189, and a light chain variable domain (VL) comprising the amino acid sequence of SEQ ID NO: 190; or
      (h) a heavy chain variable domain (VH) comprising the amino acid sequence of SEQ ID NO: 189, and a light chain variable domain (VL) comprising the amino acid sequence of SEQ ID NO: 342; or
      (i) a heavy chain variable domain (VH) comprising the amino acid sequence of SEQ ID NO: 340, and a light chain variable domain (VL) comprising the amino acid sequence of SEQ ID NO: 343; or
      (j) a heavy chain variable domain (VH) comprising the amino acid sequence of SEQ ID NO: 340, and a light chain variable domain (VL) comprising the amino acid sequence of SEQ ID NO: 344; or
      (k) a heavy chain variable domain (VH) comprising the amino acid sequence of SEQ ID NO: 341, and a light chain variable domain (VL) comprising the amino acid sequence of SEQ ID NO: 344; or
      (l) a heavy chain variable domain (VH) comprising the amino acid sequence of SEQ ID NO: 189, and a light chain variable domain (VL) comprising the amino acid sequence of SEQ ID NO: 191;
wherein Lx links VH and the CH1 domain and comprises any one of the following sequences:
   (i) an amino acid sequence of SEQ ID NO: 199; or
   (ii) an amino acid sequence of SEQ ID NO: 194,
wherein Cx comprises an amino acid sequence of SEQ ID NO: 75;
wherein Ly comprises an amino acid sequence of SEQ ID NO: 68;
wherein the ligand moiety is a protease resistant single-chain IL-12 fusion protein and comprises any one of the following sequences:
   (i) an amino acid sequence of SEQ ID NO: 317; or
   (ii) the amino acid sequence of SEQ ID NO: 317, wherein the tyrosine at position 16 of SEQ ID NO: 317 is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W); preferably proline (P); or
   (iii) an amino acid sequence of SEQ ID NO: 285; or
   (iv) the amino acid sequence of SEQ ID NO: 285, wherein the tyrosine at position 16 of SEQ ID NO: 285 is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W); preferably proline (P); or
   (v) an amino acid sequence of SEQ ID NO: 325; or
   (vi) an amino acid sequence of SEQ ID NO: 335.
      [A-11] The protein complex of [A-10], wherein:
i) the two fusion proteins are bound to each other via one or more disulfide bonds; and/or
ii) the protein complex is obtainable by a) culturing a mammalian host cell expressing the polypeptides, and b) retrieving the protein complex from the supernatant, preferably wherein the mammalian host cell is a Chinese Hamster Ovary (CHO) cell; and/or
iii) the protein complex is glycosylated and/or is produced recombinantly by expression in a mammalian host cell, preferably wherein the mammalian host cell is a Chinese Hamster Ovary (CHO) cell; and/or
iv) each fusion protein comprises a light chain consisting of the VL and a CL light chain constant domain bound to a CH1 heavy chain constant domain via one or more disulfide bonds.
   [A-12] The protein complex of any one of [A-7] to [A-11], wherein
(a) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(b) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(c) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and the ligand moiety is a protease resistant single-chain IL-12 fusion protein comprising the amino acid sequence of SEQ ID NO: 285, or comprising or consisting of the amino acid sequence of SEQ ID NO: 317;
   or
(d) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(e) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(f) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(g) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(h) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(i) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(j) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(k) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(l) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(m) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(n) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(o) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(p) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(q) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(r) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(s) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(t) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(u) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(v) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(w) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(x) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P.
      [A-12a] The protein complex of any one of [A-7] to [A-11], wherein
(a) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(b) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(c) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(d) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(e) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
(f) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(g) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(h) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(i) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(j) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(k) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(l) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(m) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(n) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(o) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(p) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(q) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(r) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(s) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(t) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(u) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(v) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(w) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(x) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P.
      [A-13] The protein complex of any one of [A-7] to [A-11], wherein
(a) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(b) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(c) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(d) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(e) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(f) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(g) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(h) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(i) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(j) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(k) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(l) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
(m) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(n) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(o) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(p) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(q) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(r) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(s) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(t) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(u) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(v) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(w) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(x) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335.
      [A-14] A polypeptide defined by any one of the sequences selected from the group consisting of: SEQ ID NO: 277, 278, 279, 280, 281, 282, 283, 284, 285, 286, 287, 288, 289, 290, 291, 292, 326, 327, and 328, and further wherein the tyrosine at position 16 of SEQ ID NO: 277, 278, 279, 280, 281, 282, 283, 284, 285, 286, 287, 288, 289, 290, 291, 292, 326, 327, and 328, is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W), preferably proline (P).
      [A-14a] A polypeptide defined by any one of the sequences selected from the group consisting of: SEQ ID NO: 277, 278, 279, 280, 281, 282, 283, 284, 285, 286, 287, 288, 289, 290, 291, 292, 326, 327, and 328.
      [A-15] A polypeptide defined by SEQ ID NO: 285, and further wherein the tyrosine at position 16 of SEQ ID NO: 285 is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W), preferably proline (P).
      [A-15a] A polypeptide defined by any one of the sequences selected from the group consisting of SEQ ID NO: 265, 266, 267, 268, 325, 326, 327, 328, and 329.
      [A-15b] A polypeptide comprising an amino acid sequence that is at least 90%, or at least 95%, or at least 97%, or at least 98%, or at least 99% identical to SEQ ID NO: 44, wherein said polypeptide comprises an amino acid substitution at amino acid residue Y16 of SEQ ID NO: 44, wherein (a) the amino acid substitution at the position corresponding to Y16 is selected from the group consisting of phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W), preferably proline (P).
      [A-16] An IL-12 protein complex or protein comprising a heterodimer of the polypeptide of [A-14] or [A-14a] or [A-15] or [A-15a], or [A-15b], and a polypeptide of SEQ ID NO: 45 or SEQ ID NO: 345.
      [A-17] An IL-12 fusion protein comprising the polypeptide of [A-14] or [A-14a] or [A-15] or [A-15a], or [A-15b], linked to a polypeptide of SEQ ID NO: 45 or SEQ ID NO:345 via a linker.
      [A-18] The IL-12 fusion protein of [A-17], wherein the linker comprises (Gly Gly Gly Gly Ser (GGGGS (SEQ ID NO: 6))n, wherein n is 1 or larger.
      [A-19] An IL-12 fusion protein defined by any one of the sequences selected from the group consisting of: SEQ ID NO: 269, 270, 271, 272, 273, 274, 275, 276, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 330, 331, 332, 333, 334, 335, 336, 337, 338 and 339.
      [A-20] An anti-IL-12 binding molecule or anti-IL-12 binding molecule complex comprising a heavy chain variable domain (VH) and a light chain variable domain (VL), wherein said VH and VL comprises any one of the following sequences:
(a) a heavy chain variable domain (VH) comprising the amino acid sequence of SEQ ID NO: 189, and a light chain variable domain (VL) comprising the amino acid sequence of SEQ ID NO: 190; or
(b) a heavy chain variable domain (VH) comprising the amino acid sequence of SEQ ID NO: 189, and a light chain variable domain (VL) comprising the amino acid sequence of SEQ ID NO: 342; or
(c) heavy chain variable domain (VH) comprising the amino acid sequence of SEQ ID NO: 340, and a light chain variable domain (VL) comprising the amino acid sequence of SEQ ID NO: 343; or
(d) a heavy chain variable domain (VH) comprising the amino acid sequence of SEQ ID NO: 340, and a light chain variable domain (VL) comprising the amino acid sequence of SEQ ID NO: 344; or
(e) heavy chain variable domain (VH) comprising the amino acid sequence of SEQ ID NO: 341, and a light chain variable domain (VL) comprising the amino acid sequence of SEQ ID NO: 344; or
(f) a heavy chain variable domain (VH) comprising the amino acid sequence of SEQ ID NO: 189, and a light chain variable domain (VL) comprising the amino acid sequence of SEQ ID NO: 191.
   [A-21] The anti-IL-12 binding molecule complex or anti-IL-12 binding molecule of [A-20], wherein the anti-IL-12 binding molecule complex or anti-IL-12 binding molecule comprises an Fc region.
   [A-22] The anti-IL-12 binding molecule complex or anti-IL-12 binding molecule of [A-21], wherein the Fc region comprises the amino acid sequence of SEQ ID NO: 75, or SEQ ID NO: 111, or SEQ ID NO: 131, or SEQ ID NO: 192.
   [A-23] The anti-IL-12 binding molecule of [A-21] or [A-22], wherein the anti-IL-12 binding molecule is an antibody.
   [A-24] The anti-IL-12 binding molecule of [A-23], wherein the antibody is an IgG antibody.
   [A-25] An isolated polynucleotide or plurality of polynucleotides encoding the protein complex of any one of [A-1], [A-1a], [A-1b], [A-2] to [A-5], [A-5a], [A-6] to [A-13], or the polypeptide of [A-14], or [A-14a], or [A-15] or [A-15a], or [A-15b], or the protein complex or protein of [A-16], or the fusion protein of any one of [A-17] to [A-19], or the binding molecule complex or binding molecule of any one of [A-20] to [A-24].
   [A-26] A vector comprising the polynucleotide or plurality of polynucleotides of [A-25].
   [A-27] A host cell comprising the polynucleotide or plurality of polynucleotides of [A-25], or the vector of [A-26].
   [A-28] A method of producing the protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], comprising the steps of
a) culturing the host cell of [A-27] under conditions suitable for the expression of the protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], and
b) recovering the protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24].
   [A-29] A protein complex, or a polypeptide, or a protein, or a fusion protein, or a binding molecule complex or binding molecule produced by, or producible by, the method of [A-28].
   [A-30] A pharmaceutical composition comprising the protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], and a pharmaceutically acceptable carrier.
   [A-31] The protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], or the pharmaceutical composition of [A-30], for use as a medicament.
   [A-32] The protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], or the pharmaceutical composition of [A-30], for use in the treatment or prevention of a disease or disorder in an individual in need thereof.
   [A-33] The protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], or the pharmaceutical composition of [A-30], for use in the treatment or prevention of an IL-12 mediated disease or disorder.
   [A-34] The protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], or the pharmaceutical composition of [A-30], for use in the treatment or prevention of cancer.
   [A-35] The protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], or the pharmaceutical composition of [A-30], for use in the treatment or prevention of an inflammatory disease or disorder.
   [A-36] Use of the protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], or the pharmaceutical composition of [A-30], in the manufacture of a medicament for the treatment or prevention of a disease or disorder in an individual in need thereof.
   [A-37] A method of treating a disease or disorder in an individual in need thereof, comprising administering to said individual a therapeutically effective amount of a composition comprising the protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], or the pharmaceutical composition of [A-30].
   [A-38] The use of [A-36], or the method of [A-37], wherein the disease or disorder is an IL-12 mediated disease or disorder.
   [A-38a] The use of [A-36], or the method of [A-37], wherein the disease or disorder is susceptible of being improved or prevented by an increase or enhancement in IL-12 signaling.
   [A-39] The use of [A-36], or the method of [A-37], wherein the disease or disorder is cancer.
   [A-40] The use of [A-36], or the method of [A-37], wherein the disease or disorder is an inflammatory disease or disorder.
   [A-40a] A method of targeted treatment of cancer, comprising administering to an individual in need thereof the protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], or the pharmaceutical composition of [A-30].
   [A-40b] A method of localized delivery of an IL-12 protein, comprising administering to an individual in need thereof the protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], or the pharmaceutical composition of [A-30].
   [A-40c] A method of administering to the subject an IL-12 therapy with reduced systemic exposure and/or reduced systemic toxicity, comprising administering to an individual in need thereof the protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], or the pharmaceutical composition of [A-30].
   [A-40d] A method of locally inducing an immune response in a target tissue, comprising administering to an individual in need thereof the protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], or the pharmaceutical composition of [A-30].
   [A-41] A kit comprising the pharmaceutical composition of [A-30].
   [A-42] The kit of [A-41] comprising a package insert, wherein the package insert comprises instructions including a step of administrating to an individual in need thereof the pharmaceutical composition, to treat or delay the progression of an IL-12 mediated disease or disorder, or a disease or disorder susceptible of being improved or prevented by an increase or enhancement in IL-12 signaling, or a cancer, or an inflammatory disease or disorder.
   [A-43] The kit of [A-41] comprising a package insert, wherein the package insert comprises instructions including a step of administrating to an individual in need thereof the pharmaceutical composition, to prevent an IL-12 mediated disease or disorder, or a disease or disorder susceptible of being improved or prevented by an increase or enhancement in IL-12 signaling, or a cancer, or an inflammatory disease or disorder.
   [A-44] The protein complex of any one of [A-1] to [A-13], or the polypeptide of [A-14] to [A-15b], or the protein complex or protein of [A-16], or the fusion protein of any one of [A-17] to [A-19], or the binding molecule complex or binding molecule of any one of [A-20] to [A-24], comprising an Fc region with reduced effector function, wherein the effector function is selected of one or more of binding to all human Fc gamma receptors, ADCC and CDC.
   [A-45] The protein complex, or the polypeptide, or the protein complex or protein, or the fusion protein, or the binding molecule complex or binding molecule of [A-44], wherein the effector function selected of one or more of binding to all human Fc gamma receptors, ADCC and CDC is abolished and/or substantially abolished and/or wherein the effector function is reduced as compared to the corresponding native human Fc portion and/or wherein the binding to the human FcRn is retained in the Fc region.
   [B-1] A protein complex comprising or consisting of two subunits associated with each other, wherein each subunit comprises a first polypeptide and a second polypeptide, wherein the first polypeptide comprises or consists of a light chain variable domain (VL) and a light chain constant domain (CL), and the second polypeptide is a fusion protein represented by the general formula (I), from the N- to the C-terminus:

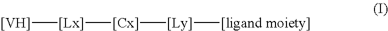

wherein:
Lx represents a peptide linker comprising a protease cleavage site,
Cx represents a constant domain associated with the CL of the first polypeptide, optionally,
wherein Cx comprises or consists of, from the N- to the C-terminus, a CH1 domain, a second peptide linker, a CH2 domain, and a CH3 domain, and
Ly represents a third peptide linker, and
wherein the VH and the VL associates with each other to form the ligand-binding domain,
wherein (a) in a first state, the ligand moiety is bound by the ligand-binding domain and the biological activity of the ligand moiety is attenuated, and in a second state, the biological activity of the ligand moiety is restored, and (b) the protein complex in the first state has a longer half-life in blood than in the second state, and (c) switching from the first state to the second state is mediated by the presence of a protease that cleaves said protease cleavage site.
   [B-2] The protein complex of [B-1], wherein the second peptide linker is positioned so that disulfide bond formation between Cys at position 220 (C220) of the second polypeptide chain (according to EU numbering) and Cys at position 214 (C214) of the first polypeptide chain (according to EU numbering) is promoted.
   [B-3] The protein complex of [B-1], wherein Cx comprises at least one amino acid modification wherein amino acid residues in the first polypeptide chain and the second polypeptide chain are modified so that no disulfide bond is formed between position 220 of the second polypeptide chain (according to EU numbering) and position 214 of the first polypeptide chain (according to EU numbering).
   [B-4] The protein complex of [B-3], wherein the second polypeptide chain comprises C220S modification (according to EU numbering), and the first polypeptide comprises C214S modification (according to EU numbering).
   [B-5] The protein complex of [B-3], wherein the second polypeptide chain is modified to allow disulfide bond formation between position 131 of the second polypeptide chain (according to EU numbering) and position 214 of the first polypeptide chain (according to EU numbering).
   [B-6] The protein complex of [B-5], wherein the second polypeptide chain comprises S131C and C220S modifications (according to EU numbering).
   [B-7] The protein complex of any of [B-1] to [B-6], wherein Cx comprises a sequence selected from the group consisting of SEQ ID NO: 31 (C1), SEQ ID NO: 34 (C2), SEQ ID NO: 73 (C3), SEQ ID NO: 75 (C4), SEQ ID NO: 41 (C5), SEQ ID NO: 111 (C6), SEQ ID NO: 131 (C7), SEQ ID NO: 192 (C8), and SEQ ID NO: 196 (C9).
   [B-8] The protein complex of [B-7], wherein Cx comprises a sequence of SEQ ID NO: 75 (C4).
   [B-9] The protein complex of any of [B-1] to [B-8], wherein Ly comprises a glycine-serine polymer.
   [B-10] The protein complex of [B-9], wherein the glycine-serine polymer is selected from the group consisting of (a) to (ee):

(a)
Ser;
(b)
Gly Ser (GS);
(c)
Ser Gly (SG);
(d)
Gly Gly Ser (GGS);
(e)
Gly Ser Gly (GSG);
(f)
Ser Gly Gly (SGG);
(g)
Gly Ser Ser (GSS);
(h)
Ser Ser Gly (SSG);
(i)
Ser Gly Ser (SGS);
(j)
Gly Gly Gly Ser (GGGS, SEQ ID NO: 1);
(k)
Gly Gly Ser Gly (GGSG, SEQ ID NO: 2);
(l)
Gly Ser Gly Gly (GSGG, SEQ ID NO: 3);
(m)
Ser Gly Gly Gly (SGGG, SEQ ID NO: 4);
(n)
Gly Ser Ser Gly (GSSG, SEQ ID NO: 5);
(o)
Gly Gly Gly Gly Ser (GGGGS, SEQ ID NO: 6);
(p)
Gly Gly Gly Ser Gly (GGGSG, SEQ ID NO: 7);
(q)
Gly Gly Ser Gly Gly (GGSGG, SEQ ID NO: 8);
(r)
Gly Ser Gly Gly Gly (GSGGG, SEQ ID NO: 9);
(s)
Gly Ser Gly Gly Ser (GSGGS, SEQ ID NO: 10);
(t)
Ser Gly Gly Gly Gly (SGGGG, SEQ ID NO: 11);
(u)
Gly Ser Ser Gly Gly (GSSGG, SEQ ID NO: 12);
(v)
Gly Ser Gly Ser Gly (GSGSG, SEQ ID NO: 13);
(w)
Ser Gly Gly Ser Gly (SGGSG, SEQ ID NO: 14);
(x)
Gly Ser Ser Ser Gly (GSSSG, SEQ ID NO: 15);
(y)
Gly Gly Gly Gly Gly Ser (GGGGGS, SEQ ID NO: 16);
(z)
Ser Gly Gly Gly Gly Gly (SGGGGG, SEQ ID NO: 17);
(aa)
Gly Gly Gly Gly Gly Gly Ser (GGGGGGS, SEQ ID NO: 18);
(bb)
Ser Gly Gly Gly Gly Gly Gly (SGGGGGG, SEQ ID NO: 19);
(cc)
(Gly Gly Gly Gly Ser (GGGGS, SEQ ID NO: 6))n;
(dd)
(Ser Gly Gly Gly Gly (SGGGG, SEQ ID NO: 11))n;
and
(ee)
(Gly Gly Ser Gly Gly (GGSGG, SEQ ID NO: 8))n;
wherein n is an integer of 1 or larger.

[B-11] The protein complex of [B-10], wherein Ly comprises a sequence of

(SEQ ID NO: 32)
GGSGGSGGSGGSGGSGGS.

[B-12] The protein complex of any of [B-1] to [B-11], wherein the ligand moiety comprises a cytokine or a chemokine.
[B-13] The protein complex of [B-12], wherein the ligand moiety is selected from the group consisting of CXCL9, CXCL10, CXCL11, IL-2, IL-7, IL-12, IL-15, IL-18, IL-21, IL-22, IFN-alpha, IFN-beta, IFN-gamma, MIG, I-TAC, RANTES, MIP-1a, MIP-1b, IL-1R1, IL-1R2, IL-1RAcP and IL-1Ra.
[B-14] The protein complex of [B-13], wherein the ligand moiety is IL-12 or IL-22.
[B-15] The protein complex of [B-14], wherein the IL-12 comprises at least one amino acid modification that prevents proteolytic degradation when exposed to protease.
[B-16] The protein complex of [B-15], wherein the IL-12 does not comprise the amino acid sequence of KSKREK (SEQ ID NO: 197), or KSKRE (SEQ ID NO: 361).
[B-17] The protein complex of [B-14] or [B-15], wherein the at least one amino acid modification is performed at the interface between IL-12 and the ligand-binding domain.
[B-18] The protein complex of [B-17], wherein after performing the at least one amino acid modification, the IL-12 comprises a modified sequence selected from the group consisting of (a) to (p):

(a)
(SEQ ID NO: 157)
KSHRE;
(b)
(SEQ ID NO: 158)
KSHHE;
(c)
(SEQ ID NO: 159)
KSHKE;
(d)
(SEQ ID NO: 160)
KSHSE;
(e)
(SEQ ID NO: 161)
KSKHRE;
(f)
(SEQ ID NO: 162)
KSKQRE;
(g)
(SEQ ID NO: 163)
KSKERE;
(h)
(SEQ ID NO: 164)
KSKPRE;
(i)
(SEQ ID NO: 165)
KHKE;
(j)
(SEQ ID NO: 166)
KHHE;
(k)
(SEQ ID NO: 167)
KHRE;
(l)
(SEQ ID NO: 168)
KKHE;
(m)
(SEQ ID NO: 169)
KRHE;
(n)
(SEQ ID NO: 170)
KRE;
(o)
(SEQ ID NO: 171)
KHE;
and
(p)
(SEQ ID NO: 172)
KKE.

[B-19] The protein complex of any of [B-15] to [B-18], wherein the IL-12 comprises the amino acid sequence, or that is at least 70%, 80%, 90%, 95%, 96%, 97%, 98%, or 99% identical to, SEQ ID NO: 173, or SEQ ID NO: 174, or SEQ ID NO: 175, or SEQ ID NO: 176, or SEQ ID NO: 177, or SEQ ID NO: 178, or SEQ ID NO: 179, or SEQ ID NO: 180, or SEQ ID NO: 181, or SEQ ID NO: 182, or SEQ ID NO: 183, or SEQ ID NO: 184, or SEQ ID NO: 185, or SEQ ID NO: 186, or SEQ ID NO: 187, or SEQ ID NO: 188, or SEQ ID NO: 293, or SEQ ID NO: 294, or SEQ ID NO: 295, or SEQ ID NO: 296, or SEQ ID NO: 297, or SEQ ID NO: 298, or SEQ ID NO: 299, or SEQ ID NO: 300, or SEQ ID NO: 301, or SEQ ID NO: 302, or SEQ ID NO: 303, or SEQ ID NO: 304, or SEQ ID NO: 305, or SEQ ID NO: 306, or SEQ ID NO: 307, or SEQ ID NO: 308, or SEQ ID NO: 309, or SEQ ID NO: 310, or SEQ ID NO: 311, or SEQ ID NO: 312, or SEQ ID NO: 313, or SEQ ID NO: 314, or SEQ ID NO: 315, or SEQ ID NO: 316, or SEQ ID NO: 317, or SEQ ID NO: 318, or SEQ ID NO: 319, or SEQ ID NO: 320, or SEQ ID NO: 321, or SEQ ID NO: 322, or SEQ ID NO: 323, or SEQ ID NO: 324.
[B-20] The protein complex of any of [B-15] to [B-19], wherein the IL-12 comprises the amino acid sequence SEQ ID NO: 277, or SEQ ID NO: 278, or SEQ ID NO: 279, or SEQ ID NO: 280, or SEQ ID NO: 281, or SEQ ID NO: 282, or SEQ ID NO: 283, or SEQ ID NO: 284, or SEQ ID NO: 285, or SEQ ID NO: 286, or SEQ ID NO: 287, or SEQ ID NO: 288, or SEQ ID NO: 289, or SEQ ID NO: 290, or SEQ ID NO: 291, or SEQ ID NO: 292, or SEQ ID NO: 325, or SEQ ID NO: 326, or SEQ ID NO: 327, or SEQ ID NO: 328, or SEQ ID NO: 329, or SEQ ID NO: 330, or SEQ ID NO: 331, or SEQ ID NO: 332, or SEQ ID NO: 333, or SEQ ID NO: 334, or SEQ ID NO: 335, or SEQ ID NO: 336, or SEQ ID NO: 337, or SEQ ID NO: 338, or SEQ ID NO: 339.
[B-21] The protein complex of [B-19] or [B-20], wherein the IL-12 comprises the amino acid sequence selected from SEQ ID NO: 173, or SEQ ID NO: 174, or SEQ ID NO: 181, or SEQ ID NO: 182, or SEQ ID NO: 183, or SEQ ID NO: 184, or SEQ ID NO: 185, or SEQ ID NO: 277, or SEQ ID NO: 278, or SEQ ID NO: 285, or SEQ ID NO: 286, or SEQ ID NO: 287, or SEQ ID NO: 288, or SEQ ID NO: 289, or SEQ ID NO: 293, or SEQ ID NO: 294, or SEQ ID NO: 301, or SEQ ID NO: 302, or SEQ ID NO: 303, or SEQ ID NO: 304, or SEQ ID NO: 305, or SEQ ID NO: 309, or SEQ ID NO: 310, or SEQ ID NO: 317, or SEQ ID NO: 318, or SEQ ID NO: 319, or SEQ ID NO: 320, or SEQ ID NO: 321.
[B-21a] The protein complex of any of [B-14], wherein the IL-12 comprises an attenuated IL-12.
[B-21b] The protein complex of any of [B-21a], wherein the IL-12 comprises a p40 subunit and a p35 subunit, wherein the p40 subunit comprises an asparagine (N), glutamic acid (E), glycine (G) or proline (P) residue at the amino acid position corresponding to residue 16 of SEQ ID NO: 44.
[B-21c] The protein complex of [B-21a] or [B-21b], wherein the IL-12 comprises the amino acid sequence SEQ ID NO: 265, or SEQ ID NO: 266, or SEQ ID NO: 267, or SEQ ID NO: 268, or SEQ ID NO: 269, or SEQ ID NO: 270, or SEQ ID NO: 271, or SEQ ID NO: 272, or SEQ ID NO: 273, or SEQ ID NO: 274, or SEQ ID NO: 275, or SEQ ID NO: 276.
[B-21d] The protein complex of any of [B21-a] to [B-21c], wherein the IL-12 further does not comprise the amino acid sequence of KSKREK (SEQ ID NO: 197), or KSKRE (SEQ ID NO: 361).
[B-21e] The protein complex of [B-21d], wherein the IL-12 comprises the amino acid sequence of SEQ ID NO: 325, or SEQ ID NO: 326, or SEQ ID NO: 327, or SEQ ID NO: 328, or SEQ ID NO: 329, or SEQ ID NO: 330, or SEQ ID NO: 331, or SEQ ID NO: 332, or SEQ ID NO: 333, or SEQ ID NO: 334, or SEQ ID NO: 335, or SEQ ID NO: 336, or SEQ ID NO: 337, or SEQ ID NO: 338, or SEQ ID NO: 339.
[B-22] The protein complex of any of [B-1] to [B-21e], wherein the protein complex comprises two protease cleavage sites, and wherein each protease cleavage site is independently cleavable by a protease specific to a target tissue.
[B-23] The protein complex of [B-22], wherein the target tissue is a cancer tissue or inflammatory tissue.
[B-24] The protein complex of any of [B-1] to [B-23], wherein each protease cleavage site is cleavable by the same protease.
[B-25] The protein complex of [B-24], wherein each protease cleavage site comprises the same protease cleavage sequence.
[B-26] The protein complex of any of [B-1] to [B-25] wherein each protease cleavage site is independently cleavable by a protease selected from the group consisting of matriptase, urokinase-type plasminogen activator (uPA) and matrix metalloprotease (MMP).
[B-27] The protein complex of any of [B-1] to [B-26], wherein the ligand-binding domain comprises at least one amino acid modification that reduces association between VH and VL in the second state than in the first state.
[B-28] The protein complex of [B-27], wherein the at least one amino acid modification is a substitution of an amino acid present at the interface between the VH and the VL, and wherein said amino acid residue for modification resides in the Framework region (FR).
[B-29] The protein complex of [B-28], wherein the at least one amino acid modification comprises any one of (i) to (iii):
(i) one or more than one substitution on the VH and no substitution on the VL;
(ii) one or more than one substitution on the VL and no substitution on the VH; and
(iii) one or more than one substitution on the VH and one or more than one substitution on the VL, optionally wherein the more than one substitution on the VH and more than one substitution on the VL are pair substitutions.
   [B-30] The protein complex of [B-29], wherein the one or more than one substitution on the VH is selected from positions 37, 45, 91 or 103 on the VH (according to Kabat numbering).
   [B-31] The protein complex of [B-29], wherein the one or more than one substitution on the VL is selected from positions 43, 46, 49 or 87 on the VL (according to Kabat numbering).
   [B-32] The protein complex of [B-30], wherein the one or more than one substitution on the VH is/are selected from positions V37, L45, H91, Y91 or W103 on the VH (according to Kabat numbering).
   [B-33] The protein complex of [B-31], wherein the one or more than one substitution on the VL is/are selected from positions A43, L46, Y49 or Y87 on the VL (according to Kabat numbering).
   [B-34] The protein complex of [B-32] or [B-33], wherein each of the positions is independently substituted to any of A, D, E, F, G, H, I, L, M, N, P, Q, R, S, T, V, W, or Y.
   [B-35] The protein complex of any of [B-28] to [B-34], wherein the substitution is selected from
(i) to (viii) below, wherein each of the positions are according to Kabat numbering:
(i) V37S on the VH;
(ii) L45Q on the VH;
(iii) Y91M, or H91A on the VH;
(iv) W103I, W103L or W103M on the VH;
(v) A43Q on the VL;
(vi) L46Q on the VL;
(vii) Y49A on the VL; and
(viii) Y87L on the VL.
   [B-36] The protein complex of any of [B-28] to [B-35], wherein the substitutions further comprise at least one modification in an amino acid present at the interface between the ligand-binding domain and the ligand moiety, wherein said amino acid residue for modification resides in the complementarity determining region (CDR).
   [B-37] The protein complex of [B-36], wherein the ligand moiety is IL-12, the further modification comprises one, or more than one, substitutions selected from (i) to (iii) below, wherein each of the positions are according to Kabat numbering:
(i) position 30 on the VH;
(ii) position 100a on the VH; and
(iii) positions 30 and 100a on the VH.
   [B-38] The protein complex of [B-37], wherein the substitution is selected from (i) to (iii) below, wherein each of the positions are according to Kabat numbering:
(i) S30V on the VH;
(ii) F100aI on the VH; and
(iii) S30V and F100aI on the VH.
   [B-39] The protein complex of any of [B-28] to [B-38], wherein the substitutions are selected from the group consisting of any one of the following combinations (a) to (z), each of the positions are according to Kabat numbering:
(a) L46Q and Y49A on the VL;
(b) H91A on the VH, and L46Q and Y49A on the VL;
(c) Y91M on the VH, and A43Q and Y49A on the VL;
(d) Y91M on the VH, and A43Q, L46Q, and Y49A on the VL;
(e) W103M on the VH, and A43Q and Y49A on the VL;
(f) W103M on the VH, and L46Q and Y49A on the VL;
(g) V37S on the VH, and A43Q and Y49A on the VL;
(h) V37S on the VH, and L46Q and Y49A on the VL;
(i) L45Q on the VH, and A43Q and Y49A on the VL;
(j) L45Q on the VH, and L46Q and Y49A on the VL;
(k) F100aI on the VH, and A43Q and Y49A on the VL;
(l) F100aI on the VH, and A43Q, L46Q, and Y49A on the VL;
(m) W103L on the VH, and S30V, L46Q, and Y49A on the VL;
(n) W103M on the VH, and S30V, L46Q, and Y49A on the VL;
(o) V37S and F100aI on the VH, and S30V, A43Q, and Y49A on the VL;
(p) V37S and F100aI on the VH, and S30V, L46Q, and Y49A on the VL;
(q) W103L on the VH, and L46Q and Y49A on the VL;
(r) W103I on the VH, and L46Q and Y49A on the VL;
(s) W103M on the VH, and Y49A and Y87L on the VL;
(t) W103L on the VH, and Y49A and Y87L on the VL;
(u) W103L on the VH, and S30V, Y49A, and Y87L on the VL;
(v) V37S and F100aI on the VH, and L46Q and Y49A on the VL;
(w) V37S and F100aI on the VH, and Y49A and Y87L on the VL;
(x) V37S and F100aI on the VH, and S30V, Y49A, and Y87L on the VL;
(y) V37S, F100aI and W103M on the VH, and L46Q and Y49A on the VL; and
(z) V37S, F100aI and W103L on the VH, and L46Q and Y49A on the VL.
   [B-40] The protein complex of [B-39], wherein the substitutions are selected from the group consisting of any one of the following combinations (a) to (g), each of the positions are according to Kabat numbering:
(a) W103M on the VH, and L46Q and Y49A on the VL;
(b) W103L on the VH, and S30V, L46Q, and Y49A on the VL;
(c) V37S and F100aI on the VH, and S30V, L46Q, and Y49A on the VL;
(d) W103L on the VH, and L46Q and Y49A on the VL;
(e) V37S and F100aI on the VH, and L46Q and Y49A on the VL;
(f) V37S, F100aI and W103M on the VH, and L46Q and Y49A on the VL; and
(g) V37S, F100aI and W103L on the VH, and L46Q and Y49A on the VL.
   [B-41] The protein complex of any of [B-1]-[B-40], wherein the molecular weight of the protein complex in the second state is smaller than the molecular weight of the protein complex in the first state.
   [B-42] The protein complex of any of [B-1]-[B-41], wherein when the cleavage site is cleaved, a portion of the ligand-binding domain is released from the protein complex.
   [B-43] The protein complex of [B-42], wherein the molecular weight of the portion of ligand-binding domain released from the protein complex is 26 kDa, or 13 kDa, or smaller.
   [B-44] The protein complex of any of [B-41] to [B-43] wherein the ratio of the molecular weight of the protein complex in the first state and the molecular weight of the protein complex in the second state is 10:9.
   [B-45] The protein complex of any of [B-41]-[B-44], wherein the molecular weight of the protein complex in the second state is 9/10 that of the molecular weight of the protein complex in the first state.
   [B-46] The protein complex of any of [B-41] to [B-44], wherein the percentage reduction in molecular weight of the protein complex in the second state compared to the protein complex in the first state is 10%.
   [B-47] The protein complex of any of [B-42]-[B-46], wherein the portion of ligand-binding domain released from the protein complex comprises VL or VH, or preferably is VH.
   [B-48] The protein complex of any of [B-27] to [B-47], wherein the reduction in association between VH and VL in the second state than in the first state can be represented by a percentage reduction in maximum response unit (RU) of less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7% or is less than or equivalent to 8%, or is less than or equivalent to 9%, or is less than or equivalent to 10%, or is less than or equivalent to 11%, or is less than or equivalent to 12%, or is less than or equivalent to 13%, or is less than or equivalent to 14%, or is less than or equivalent to 15%, or is less than or equivalent to 16%, or is less than or equivalent to 17%, or is less than or equivalent to 18%, or is less than or equivalent to 19%, or is less than or equivalent to 20%, as measured under surface plasma resonance (SPR) comparing RU of the protein complex before and after protease cleavage.
   [B-49] The protein complex of any of [B-27] to [B-48], wherein the reduction in association between VH and VL in the second state than in the first state can be represented by a percentage reduction in maximum response unit (RU) of less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, as measured under surface plasma resonance (SPR) comparing RU of the protein complex before and after protease cleavage.
   [B-50] The protein complex of any of [B-27] to [B-48], wherein the reduction in association between VH and VL in the second state than in the first state can be represented by a percentage reduction in maximum response unit (RU) of less than or equivalent to surface plasma resonance (SPR) comparing RU of the protein complex before and after protease cleavage.
   [B-51] The protein complex of any of [B-48] to [B-50], wherein SPR conditions comprises a contact duration of the protein complex in the first state with 400 nM of uPA for a duration of 30 mins.
   [B-52] The protein complex of any of [B-48] to [B-50], wherein the percentage of VH released is directly proportional with the percentage change in response unit (RU) of the protein complex as measured under SPR in the second state compared to the first state according to formula (II):

VH release %=% reduction in RU×100/D (II), wherein D corresponds to 0.01×percentage of molecular weight of VH compared to the molecular weight of the protein complex in the first state respectively.

[B-53] The protein complex of [B-52], wherein the percentage of VH released is directly proportional with the percentage change in response unit (RU) of the protein complex measured under SPR in the second state compared to the first state according to formula (II-1):

VHrelease %=% reduction in RU×100/10 (II-1).

[B-54] The protein complex of [B-53], wherein the percentage of VH released is directly proportional with the percentage change in response unit (RU) of the protein complex measured under SPR in the second state compared to the first state according to formula (II-2):

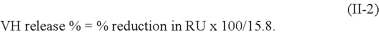

[B-55] The protein complex of any of [B-52] to [B-54], wherein the percentage of VH released is more than or equivalent to 10%, or more than or equivalent to 20%, or more than or equivalent to 30%, or more than or equivalent to 40%, or more than or equivalent to 50%, or more than or equivalent to 60%, or more than or equivalent to 70%, or more than or equivalent to 80%, or more than or equivalent to 90%, or more than or equivalent to 100%.
[B-56] The protein complex of any of [B-1] to [B-55], wherein the ligand moiety in the first and second state remains bound to the constant region via the third peptide linker.
[B-57] The protein complex of any of [B-1] to [B-55] for activating IL-12 signaling in a specific target tissue, or for increasing or enhancing IL-12 signaling in a specific target tissue.
[B-58] An isolated polynucleotide or plurality of polynucleotides encoding the protein complex of any one of [B-1] to [B-57].
[B-59] A vector comprising the polynucleotide or plurality of polynucleotides of [B-58].
[B-60] A host cell comprising the polynucleotide or plurality of polynucleotides of [B-58], or the vector of [B-59].
[B-61] A method of producing the protein complex of any one of [B-1] to [B-57], comprising the steps of
a) culturing the host cell of [B-60] under conditions suitable for the expression of the protein complex of any one of [B-1] to [B-57], and
b) recovering the protein complex of any one of [B-1] to [B-57].
   [B-62] A protein complex produced by, or producible by, the method of [B-61].
   [B-63] A pharmaceutical composition comprising the protein complex of any one of [B-1] to [B-57], and a pharmaceutically acceptable carrier.
   [B-64] The protein complex of any one of [B-1] to [B-57] or the pharmaceutical composition of [B-63], for use as a medicament.
   [B-65] The protein complex of any one of [B-1] to [B-57] or the pharmaceutical composition of [B-63], for use in the treatment or prevention of a disease or disorder in an individual in need thereof.
   [B-66] The protein complex of any one of [B-1] to [B-57] or the pharmaceutical composition of [B-63], for use in the treatment or prevention of an IL-12 mediated disease or disorder.
   [B-67] The protein complex of any one of [B-1] to [B-57] or the pharmaceutical composition of [B-63], for use in the treatment or prevention of cancer.
   [B-68] The protein complex of any one of [B-1] to [B-57] or the pharmaceutical composition of [B-63], for use in the treatment or prevention of an inflammatory disease or disorder.
   [B-69] Use of the protein complex of any one of [B-1] to [B-57] or the pharmaceutical composition of [B-63], in the manufacture of a medicament for the treatment or prevention of a disease or disorder in an individual in need thereof.
   [B-70] A method of treating a disease or disorder in an individual in need thereof, comprising administering to said individual a therapeutically effective amount of a composition comprising the protein complex of any one of [B-1] to [B-57], or the pharmaceutical composition of [B-63].
   [B-71] The use of [B-69], or the method of [B-70], wherein the disease or disorder is an IL-12 mediated disease or disorder.
   [B-72] The use of [B-69], or the method of [B-70], wherein the disease or disorder is susceptible of being improved or prevented by an increase or enhancement in IL-12 signaling.
   [B-73] The use of [B-69], or the method of [B-70], wherein the disease or disorder is cancer.
   [B-74] The use of [B-69], or the method of [B-70], wherein the disease or disorder is an inflammatory disease or disorder.
   [B-75] A method of targeted treatment of cancer, comprising administering to an individual in need thereof the protein complex of any one of [B-1] to [B-57] or the pharmaceutical composition of [B-63].
   [B-76] A method of localized delivery of an IL-12 protein, comprising administering to an individual in need thereof the protein complex of any one of [B-1] to [B-57] or the pharmaceutical composition of [B-63].
   [B-77] A method of administering to the subject an IL-12 therapy with reduced systemic exposure and/or reduced systemic toxicity, comprising administering to an individual in need thereof the protein complex of any one of [B-1] to [B-57] or the pharmaceutical composition of [B-63].
   [B-78] A method of locally inducing an immune response in a target tissue, comprising administering to an individual in need thereof the protein complex of any one of [B-1] to [B-57] or the pharmaceutical composition of [B-63].
   [C-1] An isolated protease-resistant Interleukin-12 (IL-12) protein, wherein the protein comprises at least one amino acid modification that prevents proteolytic degradation of IL-12 when exposed to protease.
   [C-2] The protease-resistant IL-12 protein according to [C-1], wherein the protein does not comprise the amino acid sequence of KSKREK (SEQ ID NO: 197), or KSKRE (SEQ ID NO: 361).
   [C-3] The protease-resistant IL-12 protein according to [C-1] or [C-2], wherein the at least one amino acid modification is performed at the interface between IL-12 protein and heparin binding site of IL-12 protein.
   [C-4] The protease-resistant IL-12 protein according to [C-3], wherein after performing the at least one amino acid modification, the IL-12 protein comprises a modified sequence selected from the group consisting of (a) to (p):

(a)
(SEQ ID NO: 157)
KSHRE;
(b)
(SEQ ID NO: 158)
KSHHE;
(c)
(SEQ ID NO: 159)
KSHKE;
(d)
(SEQ ID NO: 160)
KSHSE;
(e)
(SEQ ID NO: 161)
KSKHRE;
(f)
(SEQ ID NO: 162)
KSKQRE;
(g)
(SEQ ID NO: 163)
KSKERE;
(h)
(SEQ ID NO: 164)
KSKPRE;
(i)
(SEQ ID NO: 165)
KHKE;
(j)
(SEQ ID NO: 166)
KHHE;
(k)
(SEQ ID NO: 167)
KHRE;
(l)
(SEQ ID NO: 168)
KKHE;
(m)
(SEQ ID NO: 169)
KRHE;
(n)
(SEQ ID NO: 170)
KRE;
(o)
(SEQ ID NO: 171)
KHE;
and
(p)
(SEQ ID NO: 172)
KKE.

[C-5] The protease-resistant IL-12 protein according to any one of [C-1] to [C4], wherein the protease is selected from the group consisting of: matriptase (MT-SP1), urokinase-type plasminogen activator (uPA) and matrix metalloprotease (MMP).
[C-6] The protease-resistant IL-12 protein according to any one of [C-1] to [C5], wherein the protein comprises a heterodimer comprising the modified sequence according to [C-4] and a p35 subunit comprising an amino acid sequence of SEQ ID NO: 45, or SEQ ID NO: 345.
[C-7] The protease-resistant IL-12 protein according to any one of [C-1] to [C5], wherein the protein comprises a heterodimer comprising the modified sequence according to [C-4] and a p35 subunit comprising SEQ ID NO: 45, or SEQ ID NO: 345, wherein the modified p40 subunit and the p35 subunit is linked via a linker.
[C-8] The protease-resistant IL-12 protein according to [C-7], wherein the linker comprises a glycine-serine polymer.
[C-9] The protease-resistant IL-12 protein according to [C-8], wherein the linker the glycine-serine polymer is selected from the group consisting of (a) to (ee):

(a)
Ser;
(b)
Gly Ser (GS);
(c)
Ser Gly (SG);
(d)
Gly Gly Ser (GGS);
(e)
Gly Ser Gly (GSG);
(f)
Ser Gly Gly (SGG);
(g)
Gly Ser Ser (GSS);
(h)
Ser Ser Gly (SSG);
(i)
Ser Gly Ser (SGS);
(j)
Gly Gly Gly Ser (GGGS, SEQ ID NO: 1);
(k)
Gly Gly Ser Gly (GGSG, SEQ ID NO: 2);
(l)
Gly Ser Gly Gly (GSGG, SEQ ID NO: 3);
(m)
Ser Gly Gly Gly (SGGG, SEQ ID NO: 4);
(n)
Gly Ser Ser Gly (GSSG, SEQ ID NO: 5);
(o)
Gly Gly Gly Gly Ser (GGGGS, SEQ ID NO: 6);
(p)
Gly Gly Gly Ser Gly (GGGSG, SEQ ID NO: 7);
(q)
Gly Gly Ser Gly Gly (GGSGG, SEQ ID NO: 8);
(r)
Gly Ser Gly Gly Gly (GSGGG, SEQ ID NO: 9);
(s)
Gly Ser Gly Gly Ser (GSGGS, SEQ ID NO: 10);
(t)
Ser Gly Gly Gly Gly (SGGGG, SEQ ID NO: 11);
(u)
Gly Ser Ser Gly Gly (GSSGG, SEQ ID NO: 12);
(v)
Gly Ser Gly Ser Gly (GSGSG, SEQ ID NO: 13);
(w)
Ser Gly Gly Ser Gly (SGGSG, SEQ ID NO: 14);
(x)
Gly Ser Ser Ser Gly (GSSSG, SEQ ID NO: 15);
(y)
Gly Gly Gly Gly Gly Ser (GGGGGS, SEQ ID NO: 16);
(z)
Ser Gly Gly Gly Gly Gly (SGGGGG, SEQ ID NO: 17);
(aa)
Gly Gly Gly Gly Gly Gly Ser (GGGGGGS, SEQ ID NO: 18);
(bb)
Ser Gly Gly Gly Gly Gly Gly (SGGGGGG, SEQ ID NO: 19);
(cc)
(Gly Gly Gly Gly Ser (GGGGS, SEQ ID NO: 6))n;
(dd)
(Ser Gly Gly Gly Gly (SGGGG, SEQ ID NO: 11))n;
and
(ce)
(Gly Gly Ser Gly Gly (GGSGG, SEQ ID NO: 8))n;
wherein n is an integer of 1 or larger.

[C-10] The protease-resistant IL-12 protein according to [C-9], wherein the linker comprises (Gly Gly Gly Gly Ser (GGGGS (SEQ ID NO: 6))n, wherein n is 1 or larger.
[C-11] The protease-resistant IL-12 protein according to any one of [C-1] to [C-10], wherein the IL-12 comprises the amino acid sequence, or that is at least 70%, 80%, 90%, 95%, 96%, 97%, 98%, or 99% identical to, SEQ ID NO: 173, or SEQ ID NO: 174, or SEQ ID NO: 175, or SEQ ID NO: 176, or SEQ ID NO: 177, or SEQ ID NO: 178, or SEQ ID NO: 179, or SEQ ID NO: 180, or SEQ ID NO: 181, or SEQ ID NO: 182, or SEQ ID NO: 183, or SEQ ID NO: 184, or SEQ ID NO: 185, or SEQ ID NO: 186, or SEQ ID NO: 187, or SEQ ID NO: 188, or SEQ ID NO: 293, or SEQ ID NO: 294, or SEQ ID NO: 295, or SEQ ID NO: 296, or SEQ ID NO: 297, or SEQ ID NO: 298, or SEQ ID NO: 299, or SEQ ID NO: 300, or SEQ ID NO: 301, or SEQ ID NO: 302, or SEQ ID NO: 303, or SEQ ID NO: 304, or SEQ ID NO: 305, or SEQ ID NO: 306, or SEQ ID NO: 307, or SEQ ID NO: 308, or SEQ ID NO: 309, or SEQ ID NO: 310, or SEQ ID NO: 311, or SEQ ID NO: 312, or SEQ ID NO: 313, or SEQ ID NO: 314, or SEQ ID NO: 315, or SEQ ID NO: 316, or SEQ ID NO: 317, or SEQ ID NO: 318, or SEQ ID NO: 319, or SEQ ID NO: 320, or SEQ ID NO: 321, or SEQ ID NO: 322, or SEQ ID NO: 323, or SEQ ID NO: 324.
[C-12] The protease-resistant IL-12 protein according to any one of [C-1] to [C-11], wherein the IL-12 comprises the amino acid sequence SEQ ID NO: 277, or SEQ ID NO: 278, or SEQ ID NO: 279, or SEQ ID NO: 280, or SEQ ID NO: 281, or SEQ ID NO: 282, or SEQ ID NO: 283, or SEQ ID NO: 284, or SEQ ID NO: 285, or SEQ ID NO: 286, or SEQ ID NO: 287, or SEQ ID NO: 288, or SEQ ID NO: 289, or SEQ ID NO: 290, or SEQ ID NO: 291, or SEQ ID NO: 292.
[C-12a] The protease-resistant IL-12 protein according to [C-10] or [C-11], wherein the IL-12 comprises the amino acid sequence selected from SEQ ID NO: 173, or SEQ ID NO: 174, or SEQ ID NO: 181, or SEQ ID NO: 182, or SEQ ID NO: 183, or SEQ ID NO: 184, or SEQ ID NO: 185, or SEQ ID NO: 277, or SEQ ID NO: 278, or SEQ ID NO: 285, or SEQ ID NO: 286, or SEQ ID NO: 287, or SEQ ID NO: 288, or SEQ ID NO: 289, or SEQ ID NO: 293, or SEQ ID NO: 294, or SEQ ID NO: 301, or SEQ ID NO: 302, or SEQ ID NO: 303, or SEQ ID NO: 304, or SEQ ID NO: 305, or SEQ ID NO: 309, or SEQ ID NO: 310, or SEQ ID NO: 317, or SEQ ID NO: 318, or SEQ ID NO: 319, or SEQ ID NO: 320, or SEQ ID NO: 321.
[C-13] An isolated attenuated Interleukin-12 (IL-12) protein, wherein the protein comprises a modified p40 subunit.
[C-14] The attenuated IL-12 protein according to [C-13], wherein the binding affinity of the attenuated IL-12 protein to the human IL-12R is weaker when compared to the binding affinity of a wildtype IL-12 protein that comprises a wildtype p40 subunit comprising an amino acid sequence that is identical to SEQ ID NO: 44 to human IL-12R.
[C-15] The attenuated IL-12 protein according to [C-14], wherein the binding affinity of the modified p40 subunit to human IL-12R is weaker when compared to the binding affinity of wildtype p40 subunit comprising an amino acid sequence of SEQ ID NO: 44 to human IL-12R.
[C-16] The attenuated IL-12 protein according to [C-14] or [C-15], comprising a heterodimer of a modified p40 subunit and a p35 subunit comprising an amino acid sequence of SEQ ID NO: 45, or SEQ ID NO: 345.
[C-17] The attenuated IL-12 protein according to [C-14] or [C-15], comprising a modified p40 subunit and a p35 subunit comprising SEQ ID NO: 45, or SEQ ID NO: 345, wherein the modified p40 subunit and the p35 subunit is linked via a linker.
[C-18] The attenuated IL-12 protein according to [C-17], wherein the linker comprises a glycine-serine polymer.
[C-19] The attenuated IL-12 protein according to [C-18], wherein the linker the glycine-serine polymer is selected from the group consisting of (a) to (ee):

(a)
Ser;
(b)
Gly Ser (GS);
(c)
Ser Gly (SG);
(d)
Gly Gly Ser (GGS);
(e)
Gly Ser Gly (GSG);
(f)
Ser Gly Gly (SGG);
(g)
Gly Ser Ser (GSS);
(h)
Ser Ser Gly (SSG);
(i)
Ser Gly Ser (SGS);
(j)
Gly Gly Gly Ser (GGGS, SEQ ID NO: 1);
(k)
Gly Gly Ser Gly (GGSG, SEQ ID NO: 2);
(l)
Gly Ser Gly Gly (GSGG, SEQ ID NO: 3);
(m)
Ser Gly Gly Gly (SGGG, SEQ ID NO: 4);
(n)
Gly Ser Ser Gly (GSSG, SEQ ID NO: 5);
(o)
Gly Gly Gly Gly Ser (GGGGS, SEQ ID NO: 6);
(p)
Gly Gly Gly Ser Gly (GGGSG, SEQ ID NO: 7);
(q)
Gly Gly Ser Gly Gly (GGSGG, SEQ ID NO: 8);
(r)
Gly Ser Gly Gly Gly (GSGGG, SEQ ID NO: 9);
(s)
Gly Ser Gly Gly Ser (GSGGS, SEQ ID NO: 10);
(t)
Ser Gly Gly Gly Gly (SGGGG, SEQ ID NO: 11);
(u)
Gly Ser Ser Gly Gly (GSSGG, SEQ ID NO: 12);
(v)
Gly Ser Gly Ser Gly (GSGSG, SEQ ID NO: 13);
(w)
Ser Gly Gly Ser Gly (SGGSG, SEQ ID NO: 14);
(x)
Gly Ser Ser Ser Gly (GSSSG, SEQ ID NO: 15);
(y)
Gly Gly Gly Gly Gly Ser (GGGGGS, SEQ ID NO: 16);
(z)
Ser Gly Gly Gly Gly Gly (SGGGGG, SEQ ID NO: 17);
(aa)
Gly Gly Gly Gly Gly Gly Ser (GGGGGGS, SEQ ID NO: 18);
(bb)
Ser Gly Gly Gly Gly Gly Gly (SGGGGGG, SEQ ID NO: 19);
(cc)
(Gly Gly Gly Gly Ser (GGGGS, SEQ ID NO: 6))n;
(dd)
(Ser Gly Gly Gly Gly (SGGGG, SEQ ID NO: 11))n;
and
(ee)
(Gly Gly Ser Gly Gly (GGSGG, SEQ ID NO: 8))n;
wherein n is an integer of 1 or larger.

[C-20] The attenuated IL-12 protein according to [C-19], wherein the linker comprises (Gly Gly Gly Gly Ser (GGGGS (SEQ ID NO: 6))n, wherein n is 1 or larger.
[C-21] The attenuated IL-12 protein according to any one of [C-13] to [C-20], wherein the modified p40 subunit comprises a substitution to tyrosine at position 16 according to SEQ ID NO: 44, wherein the tyrosine is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W).
[C-22] The attenuated IL-12 protein according to [C-21], comprising a substitution to tyrosine at position 16 according to SEQ ID NO: 44, wherein the tyrosine is substituted with proline (P).
[C-23] The attenuated IL-12 protein according to any one of [C-13] to [C-22], wherein the attenuated IL-12 protein binds to the IL-12R with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of a wildtype IL-12 protein comprising a p40 subunit comprising an amino acid sequence of SEQ ID NO: 44.
[C-24] The attenuated IL-12 protein according to any one of [C-13] to [C-23], wherein the attenuated IL-12 protein comprises greater than 10% but less than 100%, greater than 20% but less than 100%, greater than 30% but less than 100%, greater than 40% but less than 100%, greater than 50% but less than 100%, greater than 60% but less than 100%, greater than 70% but less than 100%, greater than 80% but less than 100%, or greater than 90% but less than 100%, of a wildtype IL-12 protein comprising a wildtype p40 subunit comprising an amino acid sequence of SEQ ID NO: 44 when evaluated in the same condition in a given assay.
[C-25] The attenuated IL-12 protein according to any one of [C-13] to [C-24], wherein the protein comprises the amino acid sequence, or that is at least 70%, 80%, 90%, 95%, 96%, 97%, 98%, or 99% identical to, SEQ ID NO: 265, or SEQ ID NO: 266, or SEQ ID NO: 267, or SEQ ID NO: 268, or SEQ ID NO: 269, or SEQ ID NO: 270, or SEQ ID NO: 271, or SEQ ID NO: 272, or SEQ ID NO: 273, or SEQ ID NO: 274, or SEQ ID NO: 275, or SEQ ID NO: 276.
[C-26] A modified IL-12 protein wherein the IL-12 does not comprise the amino acid sequence of KSKREK (SEQ ID NO: 197) or KSKRE (SEQ ID NO: 361), and comprises the amino acid sequence, or that is at least 90%, 95%, or 100% identical to, SEQ ID NO: 293, or SEQ ID NO: 294, or SEQ ID NO: 295, or SEQ ID NO: 296, or SEQ ID NO: 297, or SEQ ID NO: 298, or SEQ ID NO: 299, or SEQ ID NO: 300, or SEQ ID NO: 301, or SEQ ID NO: 302, or SEQ ID NO: 303, or SEQ ID NO: 304, or SEQ ID NO: 305, or SEQ ID NO: 306, or SEQ ID NO: 307, or SEQ ID NO: 308, or SEQ ID NO: 309, or SEQ ID NO: 310, or SEQ ID NO: 311, or SEQ ID NO: 312, or SEQ ID NO: 313, or SEQ ID NO: 314, or SEQ ID NO: 315, or SEQ ID NO: 316, or SEQ ID NO: 317, or SEQ ID NO: 318, or SEQ ID NO: 319, or SEQ ID NO: 320, or SEQ ID NO: 321, or SEQ ID NO: 322, or SEQ ID NO: 323, or SEQ ID NO: 324.
[C-26a] An isolated attenuated IL-12 protein, wherein the IL-12 comprises the amino acid sequence SEQ ID NO: 326, or SEQ ID NO: 327, or SEQ ID NO: 328, or SEQ ID NO: 331, or SEQ ID NO: 332, or SEQ ID NO: 333, or SQ ID NO: 336, or SEQ ID NO: 337, or SEQ ID NO: 338.
[C-27] A modified IL-12 protein wherein the IL-12 does not comprise the amino acid sequence of KSKREK (SEQ ID NO: 197) or KSKRE (SEQ ID NO: 361), and comprises a substitution to tyrosine at position 16 according to SEQ ID NO: 44, wherein the tyrosine is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W).
[C-28] The modified IL-12 protein according to [C-27], wherein the protein comprises any one of the following:
(i) an amino acid sequence that is at least 90%, 95%, or 100% identical to SEQ ID NO: 325;
(ii) an amino acid sequence that is at least 90%, 95%, or 100% identical to SEQ ID NO: 329;
(iii) an amino acid sequence that is at least 90%, 95%, or 100% identical to SEQ ID NO: 330;
(vi) an amino acid sequence that is at least 90%, 95%, or 100% identical to SEQ ID NO: 334;
(v) an amino acid sequence that is at least 90%, 95%, or 100% identical to SEQ ID NO: 335; and
(vi) an amino acid sequence that is at least 90%, 95%, or 100% identical to SEQ ID NO: 339.
   [C-29] An isolated polynucleotide or plurality of polynucleotides encoding the IL-12 protein of any one of [C-1] to [C-28].
   [C-30] A vector comprising the polynucleotide or plurality of polynucleotides of [C-29].
   [C-31] A host cell comprising the polynucleotide or plurality of polynucleotides of [C-29], or the vector of [C-30].
   [C-32] A method of producing the IL-12 protein of any one of [C-1] to [C-28], comprising the steps of:
a) culturing the host cell of [C-31] under conditions suitable for the expression of the IL-12 protein, and
b) recovering the IL-12 protein of any one of [C-1] to [C-28].
   [C-33] An IL-12 protein of any one of [C-1] to [C-28] produced by, or producible by, the method of [C-32].
   [C-34] A pharmaceutical composition comprising an IL-12 protein of any one of [C-1] to [C-28], and a pharmaceutically acceptable carrier.
   [C-35] The IL-12 protein of any one of [C-1] to [C-28], or the pharmaceutical composition of [C-34] for use as a medicament.
   [C-36] The IL-12 protein of any one of [C-1] to [C-28], or the pharmaceutical composition of [C-34] for use in the treatment or prevention of a disease or disorder in an individual in need thereof.
   [C-37] The IL-12 protein of any one of [C-1] to [C-28], or the pharmaceutical composition of [C-34] for use in the treatment or prevention of an IL-12 mediated disease or disorder.
   [C-38] The IL-12 protein of any one of [C-1] to [C-28], or the pharmaceutical composition of [C-34] for use in the treatment or prevention of cancer for use in the treatment or prevention of cancer.
   [C-39] The IL-12 protein of any one of [C-1] to [C-28], or the pharmaceutical composition of [C-34] for use in the treatment or prevention of an inflammatory disease or disorder.
   [C-40] Use of the IL-12 protein of any one of [C-1] to [C-28], or the pharmaceutical composition of [C-34] in the manufacture of a medicament for the treatment or prevention of a disease or disorder in an individual in need thereof.
   [C-41] A method of treating a disease or disorder in an individual in need thereof, comprising administering to said individual a therapeutically effective amount of a composition comprising the IL-12 protein of any one of [C-1] to [C-28], or the pharmaceutical composition of [C-34].
   [C-42] The use of [C-40], or the method of [C-41], wherein the disease or disorder is an IL-12 mediated disease or disorder.
   [C-43] The use of [C-40], or the method of [C-41], wherein the disease or disorder is susceptible of being improved or prevented by an increase or enhancement in IL-12 signaling.
   [C-44] The use of [C-40], or the method of [C-41], wherein the disease or disorder is cancer.
   [C-45] The use of [C-40], or the method of [C-41], wherein the disease or disorder is an inflammatory disease or disorder.
   [C-46] A method of targeted treatment of cancer, comprising administering to an individual in need thereof the IL-12 protein of any one of [C-1] to [C-28], or the pharmaceutical composition of [C-34].
   [C-47] A method of localized delivery of an IL-12 protein, comprising administering to an individual in need thereof the IL-12 protein of any one of [C-1] to [C-28], or the pharmaceutical composition of [C-34].
   [C-48] A method of administering to the subject an IL-12 therapy with reduced systemic exposure and/or reduced systemic toxicity, comprising administering to an individual in need thereof the IL-12 protein of any one of [C-1] to [C-28], or the pharmaceutical composition of [C-34].
   [C-49] A method of locally inducing an immune response in a target tissue, comprising administering to an individual in need thereof the IL-12 protein of any one of [C-1] to [C-28], or the pharmaceutical composition of [C-34].
   [D-1] A method of producing a protein complex comprising or consisting of two subunits associated with each other, wherein each subunit comprises a first polypeptide and a second polypeptide, wherein the first polypeptide comprises or consists of a light chain variable domain (VL) and a light chain constant domain (CL), and the second polypeptide is a fusion protein represented by the general formula (I), from the N- to the C-terminus:

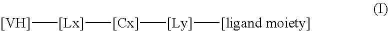

wherein:
Lx represents a peptide linker comprising a protease cleavage site,
Cx represents a constant domain associated with the CL of the first polypeptide, optionally,
wherein Cx comprises or consists of, from the N- to the C-terminus, a CH1 domain, a second peptide linker, a CH2 domain, and a CH3 domain, and
Ly represents a third peptide linker, and
wherein the VH and the VL associates with each other to form the ligand-binding domain, comprising the steps of: (a) introducing at least one amino acid modification or at least one pair of amino acid modification at the interface between VH and VL in said protein complex, and optionally introducing at least one amino acid modification at the interface between the ligand and the ligand binding domain, that promote(s) dissociation of VH from the protein complex, (b) confirming that step (a) does not disrupt binding of ligand to ligand-binding domain, (c) confirming that step (a) reduced association of VH and VL upon protease cleavage at the protease cleavage site,
(d) obtaining a polynucleotide or a plurality of polynucleotides encoding said protein of step (c),
(e) culturing a host cell comprising the polynucleotide or a plurality of polynucleotides of step (d), and
(f) producing and recovering the protein complex from the host cell in step (e).
   [D-2] The method according to [D-1], wherein the one or more modification(s) is/are substitution(s) are selected from one of the following:
(a) wherein the substitution(s) is/are selected from positions on the VH;
(b) wherein the substitution(s) is/are selected from positions on the VL; and
(c) wherein the substitutions are selected from positions on both the VH and the VL, and optionally wherein the substitutions selected from positions on both the VH and the VL are pair substitutions.
   [D-3] The method according to [D-2], wherein the one or more than one substitution on the VH is selected from positions 37, 45, 91 or 103 on the VH (according to Kabat numbering).
   [D-4] The method according to [D-2], wherein the one or more than one substitution on the VL is selected from positions 43, 46, 49 or 87 on the VL (according to Kabat numbering).
   [D-5] The method according to [D-3], wherein the one or more than one substitution on the VH is/are selected from positions V37, L45, H91, Y91 or W103 on the VH (according to Kabat numbering).
   [D-6] The method according to [D-4], wherein the one or more than one substitution on the VL is/are selected from positions A43, L46, Y49 or Y87 on the VL (according to Kabat numbering).
   [D-7] The method according to [D-5] or [D-6], wherein each of the positions is independently substituted to any of A, D, E, F, G, H, I, L, M, N, P, Q, R, S, T, V, W, or Y.
   [D-8] The method according to any of [D-3] to [D-7], wherein the substitution is selected from
(i) to (viii) below, wherein each of the positions are according to Kabat numbering:
(i) V37S on the VH;
(ii) L45Q on the VH;
(iii) Y91M, or H91A on the VH;
(iv) W103I, W103L or W103M on the VH;
(v) A43Q on the VL;
(vi) L46Q on the VL;
(vii) Y49A on the VL; and
(viii) Y87L on the VL.
   [D-9] The method according to any of [D-2] to [D-8], wherein the substitutions further comprise at least one modification in an amino acid present at the interface between the ligand-binding domain and the ligand moiety, wherein said amino acid residue for modification resides in the complementarity determining region (CDR).
   [D-10] The method according to [D-9], wherein the ligand moiety is IL-12, the further modification comprises one, or more than one, substitutions selected from (i) to (iii) below, wherein each of the positions are according to Kabat numbering:
(i) position 30 on the VH;
(ii) position 100a on the VH; and
(iii) positions 30 and 100a on the VH.
   [D-11] The method according to [D-10], wherein the substitution is selected from (i) to (iii) below, wherein each of the positions are according to Kabat numbering:
(i) S30V on the VH;
(ii) F100aI on the VH; and
(iii) S30V and F100aI on the VH.
   [D-12] The method according to any of [D-2] to [D-11], wherein the substitutions are selected from the group consisting of any one of the following combinations (a) to (z), each of the positions are according to Kabat numbering:
(a) L46Q and Y49A on the VL;
(b) H91A on the VH, and L46Q and Y49A on the VL;
(c) Y91M on the VH, and A43Q and Y49A on the VL;
(d) Y91M on the VH, and A43Q, L46Q, and Y49A on the VL;
(e) W103M on the VH, and A43Q and Y49A on the VL;
(f) W103M on the VH, and L46Q and Y49A on the VL;
(g) V37S on the VH, and A43Q and Y49A on the VL;
(h) V37S on the VH, and L46Q and Y49A on the VL;
(i) L45Q on the VH, and A43Q and Y49A on the VL;
(j) L45Q on the VH, and L46Q and Y49A on the VL;
(k) F100aI on the VH, and A43Q and Y49A on the VL;
(l) F100aI on the VH, and A43Q, L46Q, and Y49A on the VL;
(m) W103L on the VH, and S30V, L46Q, and Y49A on the VL;
(n) W103M on the VH, and S30V, L46Q, and Y49A on the VL;
(o) V37S and F100aI on the VH, and S30V, A43Q, and Y49A on the VL;
(p) V37S and F100aI on the VH, and S30V, L46Q, and Y49A on the VL;
(q) W103L on the VH, and L46Q and Y49A on the VL;
(r) W103I on the VH, and L46Q and Y49A on the VL;
(s) W103M on the VH, and Y49A and Y87L on the VL;
(t) W103L on the VH, and Y49A and Y87L on the VL;
(u) W103L on the VH, and S30V, Y49A, and Y87L on the VL;
(v) V37S and F100aI on the VH, and L46Q and Y49A on the VL;
(w) V37S and F100aI on the VH, and Y49A and Y87L on the VL;
(x) V37S and F100aI on the VH, and S30V, Y49A, and Y87L on the VL;
(y) V37S, F100aI and W103M on the VH, and L46Q and Y49A on the VL; and
(z) V37S, F100aI and W103L on the VH, and L46Q and Y49A on the VL.
   [D-13] The method according to [D-12], wherein the substitutions are selected from the group consisting of any one of the following combinations (a) to (g), each of the positions are according to Kabat numbering:
(a) W103M on the VH, and L46Q and Y49A on the VL;
(b) W103L on the VH, and S30V, L46Q, and Y49A on the VL;
(c) V37S and F100aI on the VH, and S30V, L46Q, and Y49A on the VL;
(d) W103L on the VH, and L46Q and Y49A on the VL;
(e) V37S and F100aI on the VH, and L46Q and Y49A on the VL;
(f) V37S, F100aI and W103M on the VH, and L46Q and Y49A on the VL; and
(g) V37S, F100aI and W103L on the VH, and L46Q and Y49A on the VL.
   [E-1] A method for screening for a protein complex comprising or consisting of two subunits associated with each other, wherein each subunit comprises a first polypeptide and a second polypeptide, wherein the first polypeptide comprises or consists of a light chain variable domain (VL) and a light chain constant domain (CL), and the second polypeptide is a fusion protein represented by the general formula (I), from the N- to the C-terminus:

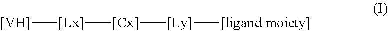

wherein:
Lx represents a peptide linker comprising a protease cleavage site,
Cx represents a constant domain associated with the CL of the first polypeptide, optionally,
wherein Cx comprises or consists of, from the N- to the C-terminus, a CH1 domain, a second peptide linker, a CH2 domain, and a CH3 domain, and
Ly represents a third peptide linker, and
wherein the VH and the VL associates with each other to form the ligand-binding domain,
wherein (a) in a first state, the ligand moiety is bound by the ligand-binding domain and the biological activity of the ligand moiety is attenuated, and in a second state, the biological activity of the ligand moiety is restored, and (b) the protein complex in the first state has a longer half-life in blood than in the second state, and (c) switching from the first state to the second state is mediated by the presence of a protease that cleaves said protease cleavage site,
wherein the protein complex comprises mutations that reduce association between VH and VL in the cleaved state or second state compared to the uncleaved state or first state, comprising comparing the maximum response unit recorded for the protein complex before and after protease cleavage under surface plasma resonance (SPR) and selecting mutations that result in a reduction in response unit of less than or equivalent to 1%, or less than or equivalent to 2%, or less than or equivalent to 3%, or less than or equivalent to 4%, or less than or equivalent to 5%, or less than or equivalent to 6%, or less than or equivalent to 7%, or less than or equivalent to 8%, or less than or equivalent to 9%, or less than or equivalent to 10%, before and after protease cleavage.
   [E-2] A method for screening for a protein complex comprising or consisting of two subunits associated with each other, wherein each subunit comprises a first polypeptide and a second polypeptide, wherein the first polypeptide comprises or consists of a light chain variable domain (VL) and a light chain constant domain (CL), and the second polypeptide is a fusion protein represented by the general formula (I), from the N- to the C-terminus:

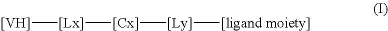

wherein:
Lx represents a peptide linker comprising a protease cleavage site,
Cx represents a constant domain associated with the CL of the first polypeptide, optionally,
wherein Cx comprises or consists of, from the N- to the C-terminus, a CH1 domain, a second peptide linker, a CH2 domain, and a CH3 domain, and
Ly represents a third peptide linker, and
wherein the VH and the VL associates with each other to form the ligand-binding domain,
wherein (a) in a first state, the ligand moiety is bound by the ligand-binding domain and the biological activity of the ligand moiety is attenuated, and in a second state, the biological activity of the ligand moiety is restored, and (b) the protein complex in the first state has a longer half-life in blood than in the second state, and (c) switching from the first state to the second state is mediated by the presence of a protease that cleaves said protease cleavage site,
wherein the protein complex comprises substitutions that reduce association between VH and VL in the cleaved state or second state compared to the uncleaved state or first state, comprising the steps:
(a) introducing at least one amino acid substitution or at least one pair of amino acid substitution at the interface between VH and VL in said protein complex and optionally introducing at least one amino acid substitution at the interface between the ligand and the ligand-binding domain, that promote dissociation of VH domain from the protein complex;
(b) determining a first response unit (RU1) of immobilised protein complex of step (a) in a BIACORE® surface plasma resonance (SPR) assay in the absence of protease;
(c) determining a second response unit (RU2) of immobilised protein complex of step (a) in the same BIACORE® surface plasma resonance (SPR) assay in the presence of protease;
(d) selecting the substitution(s) in step (a) if the percentage difference between RU1 and RU2 is less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, or is less than or equivalent to 8%, or is less than or equivalent to 9%, or is less than or equivalent to 10%, before and after protease cleavage.
   [E-3] The method according to [E-1] or [E-2], wherein the percentage reduction in response unit corresponds to the percentage reduction in molecular weight resulting from the release of VH from the protein complex.
   [E-4] The method according to [E-1] to [E-3], wherein the method further comprises the steps:
i. determining the biological activity of a protein complex according to [A-1] to [A-13], or [B-1] to [B-57] before protease cleavage;
ii. determining the biological activity of a protein complex of step (i) after protease cleavage;
iii. introducing at least one amino acid modification or at least one pair of amino acid modification at the interface between VH and VL in the protein complex in step (i), and optionally introducing at least one amino acid modification at the interface between the ligand and the ligand-binding domain, wherein the amino acid modification(s) promote dissociation of VH or VL from the protein complex upon protease cleavage in the presence of a protease;
iv. determining the biological activity of the protein complex in step (iii), before protease cleavage;
v. determining the biological activity of the protein complex in step (iii), after protease cleavage; and
vi. selecting amino acid modification(s) wherein the biological activity of the protein complex in step (v) is greater than the biological activity of the protein complex in step (iv).
   [E-5] The method of [E-4], further comprising the step of:
(a) determining the difference in biological activity of the protein complex between (i) and (ii), “V1”, and the difference in biological activity of the protein complex between (iv) and (v), “V2”; and;
(b) selecting the amino acid modification(s) wherein the value of V2 is greater than V1.
   [E-6] A library of amino acid substitutions for reducing association between VH and VL in a protein complex according to [A-1] to [A-13], or [B-1] to [B-57], comprising the substitutions selected in [E-1] to [E-3].
   [F-1] An attenuated IL-12 polypeptide comprising a p40 subunit and a p35 subunit, wherein the p40 subunit comprises an asparagine (N), glutamic acid (E), glycine (G) or proline (P) residue at the amino acid position corresponding to residue 16 of SEQ ID NO: 44.
   [F-2] The attenuated IL-12 polypeptide of [F-1] characterized by one or more of
(a) wherein the p40 subunit does not comprise the amino acid sequence of KSKREK (SEQ ID NO: 197), or KSKRE (SEQ ID NO: 361);
(b) wherein the attenuated IL-12 polypeptide is a single chain IL-12 polypeptide; and
(c) wherein the attenuated IL-12 polypeptide comprises the amino acid sequence of SEQ ID NO: 325 or 335.
   [F-3] A fusion protein or protein complex comprising the attenuated IL-12 polypeptide of [F-1] or [F-2].
   [F-4] A polynucleotide encoding the attenuated IL-12 polypeptide of [F-1] or [F-2] or a plurality of polynucleotides encoding the fusion protein or protein complex of [F-3].
   [F-5] A recombinant host cell comprising a polynucleotide or a plurality of polynucleotides encoding the attenuated IL-12 polypeptide of [F-1] or [F-2] or the fusion protein or protein complex of [F-3].
   [F-6] A method of producing the attenuated IL-12 polypeptide of [F-1] or [F-2] or the fusion protein or protein complex of [F-3] comprising (a) culturing a recombinant host cell comprising a polynucleotide or a plurality of polynucleotides encoding the attenuated IL-12 polypeptide or fusion protein or protein complex under conditions suitable for the expression of the attenuated IL-12 polypeptide or fusion protein or protein complex and (b) recovering the attenuated IL-12 polypeptide or fusion protein or protein complex.
   [F-7] A method of treating a disease or disorder in an individual in need thereof, comprising administering to said individual a therapeutically effective amount of the attenuated IL-12 polypeptide of [F-1] or [F-2] or the fusion protein or protein complex of [F-3].
   [G-1] A protein complex comprising two subunits associated with each other, wherein each subunit comprises a first polypeptide and a second polypeptide, wherein the first polypeptide comprises a light chain variable domain (VL) and a light chain constant domain (CL), and the second polypeptide is a fusion protein represented by the general formula (I), from the N- to the C-terminus:

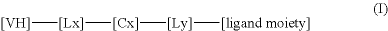

wherein:
VH represents a heavy chain variable domain (VH), wherein the VH is associated with the VL of the first polypeptide to form a ligand-binding domain that binds to the ligand moiety,
Lx represents a cleavable peptide linker comprising a protease cleavage site,
Cx represents a constant domain associated with the CL of the first polypeptide, wherein Cx comprises, from the N- to the C-terminus, a CH1 domain, a second peptide linker, a CH2 domain, and a CH3 domain,
Ly represents a third peptide linker, and
the ligand moiety is a protease resistant attenuated single-chain IL-12 fusion protein comprising a p40 subunit and a p35 subunit, wherein the p40 subunit comprises an asparagine (N), glutamic acid (E), glycine (G) or proline (P) residue at the amino acid position corresponding to residue 16 of SEQ ID NO: 44 and does not comprise the amino acid sequence of KSKREK (SEQ ID NO: 197), or KSKRE (SEQ ID NO: 361).
   [G-2] The protein complex of [G-1], wherein the protease resistant single-chain IL-12 fusion protein comprises the amino acid sequence of SEQ ID NO: 325 or 335.
   [G-3] The protein complex of [G-1] or [G-2], wherein
(a) the VH and VL comprises HCDR1; HCDR2, HCDR3, LCDR1, LCDR2, and LCDR3 comprising SEQ ID NO: 346, 348, 350, 352, 353, and 355, respectively, or SEQ ID NO: 346, 347, 349, 351, 353, and 355, respectively, or SEQ ID NO: 346, 347, 349, 351, 354, and 355, respectively, or SEQ ID NO: 346, 348, 350, 351, 353, and 355, respectively, or SEQ ID NO: 346, 348, 350, 351, 354, and 355, respectively, or SEQ ID NO: 346, 347, 349, 352, 353, and 355, respectively; or
(b) the VH and VL comprises SEQ ID NO: 189 and 190, respectively, or SEQ ID NO: 340 and 343, respectively, or SEQ ID NO: 340 and 344, respectively, or SEQ ID NO: 341 and 344, respectively, or SEQ ID NO: 189 and 342, respectively, or SEQ ID NO: 189 and 191, respectively.
   [G-4] The protein complex of any one of [G-1] to [G-3] characterized by one or more of
(a) wherein Lx comprises the amino acid sequence of SEQ ID NO: 194 or 199;
(b) wherein Cx comprises the amino acid sequence of SEQ ID NO: 75; and
(c) wherein Ly comprises the amino acid sequence of SEQ ID NO: 68.
   [G-5] The protein complex of any one of [G-1] to [G-4] comprising two first polypeptides and two second polypeptides, wherein the first and second polypeptide comprises the amino acid sequence of SEQ ID NO: 249 and 121, respectively; or SEQ ID NO: 249 and 122, respectively; or SEQ ID NO: 264 and 121, respectively; or SEQ ID NO: 264 and 122, respectively; or SEQ ID NO: 247 and 121, respectively; or SEQ ID NO: 248 and 121, respectively; or SEQ ID NO: 250 and 121, respectively.
   [G-6] A polynucleotide or a plurality of polynucleotides encoding the protein complex of any one of [G-1] to [G-5].
   [G-7] A recombinant host cell comprising a polynucleotide or a plurality of polynucleotides encoding the protein complex of any one of [G-1] to [G-5].
   [G-8] A method of producing the protein complex of any one of [G-1] to [G-5] comprising (a) culturing a recombinant host cell comprising a polynucleotide or a plurality of polynucleotides encoding the protein complex under conditions suitable for the expression of the protein complex and (b) recovering the protein complex.
   [G-9] A method of treating a disease or disorder in an individual in need thereof, comprising administering to said individual a therapeutically effective amount of the protein complex of any one of [G-1] to [G-5].
   [H-1] An antibody that binds to human IL-12 and comprises:
(a) a HCDR1 of SEQ ID NO: 346;
(b) a HCDR2 of SEQ ID NO: 347 or 348;
(c) a HCDR3 of SEQ ID NO: 349 or 350;
(d) a LCDR1 of SEQ ID NO: 351 or 352;
(e) a LCDR2 of SEQ ID NO: 353 or 354; and
(f) a LCDR3 of SEQ ID NO: 355,
wherein the HCDR1, HCDR2, HCDR3, LCDR1, LCDR2 and LCDR3 does not comprise SEQ ID NO: 346, 348, 350, 352, 353 and 355, respectively.
   [H-2] The antibody of [H-1], wherein the HCDR1, HCDR2, HCDR3, LCDR1, LCDR2 and LCDR3 comprises SEQ ID NO: 346, 347, 349, 351, 353 and 355, respectively, or SEQ ID NO: 346, 347, 349, 351, 354 and 355, respectively.
   [H-3] The antibody of [H-1] or [H-2], wherein the VH comprises SEQ ID NO: 189, 340 or 341 and the VL comprises SEQ ID NO: 190, 191, 342, 343 or 344, wherein the VH and VL does not comprise SEQ ID NO: 189 and 190, respectively.
   [H-4] The antibody of any one of [H-1] to [H-3], wherein the VH and VL comprises SEQ ID NO: 189 and 342, respectively, SEQ ID NO: 340 and 343, respectively, SEQ ID NO: 340 and 344, respectively, SEQ ID NO: 341 and 344, respectively, or SEQ ID NO: 189 and 191, respectively.
   [H-5] The antibody of any one of [H-1] to [H-3] comprising a heavy chain (HC) and a light chain (LC), wherein the HC and the LC comprises SEQ ID NO: 198 and 121, respectively, or SEQ ID NO: 198 and 201, respectively, or SEQ ID NO: 203 and 202, respectively.
   [H-6] The antibody of any one of [H-1] to [H-4] which comprises an IgG antibody, IgG1 antibody, scFv, single-chain antibody, Fv, scFv2, Fab or F(ab′)2.
   [H-7] A fusion protein or protein complex comprising the antibody of any one of [H-1] to [H-6] and an IL-12 polypeptide.
   [H-8] A polynucleotide encoding the antibody of any one of [H-1] to [H-6] or a plurality of polynucleotides encoding the fusion protein or protein complex of [H-7].
   [H-9] A method of treating an IL-12 mediated disease or disorder in an individual in need thereof, comprising administering to said individual a therapeutically effective amount of the antibody of any one of [H-1] to [H-6].
   [I-1] A protein complex comprising two subunits associated with each other, wherein each subunit comprises a first polypeptide and a second polypeptide, wherein the first polypeptide comprises a light chain variable domain (VL) and a light chain constant domain (CL), and the second polypeptide is a fusion protein represented by the general formula (I), from the N- to the C-terminus:

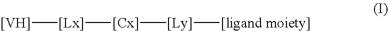

wherein:
VH represents a heavy chain variable domain (VH), wherein the VH is associated with the VL of the first polypeptide to form an affinity matured ligand-binding domain that binds to IL-12, and wherein the VH and VL comprises SEQ ID NO: 189 and 342, respectively, SEQ ID NO: 340 and 343, respectively, SEQ ID NO: 340 and 344, respectively, SEQ ID NO: 341 and 344, respectively, or SEQ ID NO: 189 and 191, respectively,
Lx represents a cleavable peptide linker comprising a protease cleavage site,
Cx represents a constant domain associated with the CL of the first polypeptide, wherein Cx comprises, from the N- to the C-terminus, a CH1 domain, a second peptide linker, a CH2 domain, and a CH3 domain,
Ly represents a third peptide linker, and
the ligand moiety is a protease resistant single-chain IL-12 fusion protein.
   [I-2] The protein complex of [I-1] characterized by one or more of
(a) wherein Lx comprises the amino acid sequence of SEQ ID NO: 194 or 199;
(b) wherein Cx comprises the amino acid sequence of SEQ ID NO: 75; and
(c) wherein Ly comprises the amino acid sequence of SEQ ID NO: 68.
   [I-3] The protein complex of [I-1] or [I-2], wherein the protease resistant single-chain IL-12 fusion protein comprises:
(i) the amino acid sequence of SEQ ID NO: 317;
(ii) an amino acid sequence of SEQ ID NO: 317, wherein the tyrosine at position 16 of SEQ ID NO: 317 is substituted with an amino acid selected from the group consisting of asparagine (N), glutamic acid (E), glycine (G) and proline (P), preferably wherein the tyrosine at position 16 of SEQ ID NO: 317 is substituted with P;
(iii) the amino acid sequence of SEQ ID NO: 285;
(iv) an amino acid sequence of SEQ ID NO: 285, wherein the tyrosine at position 16 of SEQ ID NO: 285 is substituted with an amino acid selected from the group consisting of asparagine (N), glutamic acid (E), glycine (G) and proline (P), preferably wherein the tyrosine at position 16 of SEQ ID NO: 285 is substituted with P;
(v) the amino acid sequence of SEQ ID NO: 325; or
(vi) the amino acid sequence of SEQ ID NO: 335.
   [I-4] The protein complex of any one of [I-1] to [I-3] comprising two first polypeptides and two second polypeptides, wherein the first and second polypeptide comprises the amino acid sequence of SEQ ID NO: 204 and 201, respectively; SEQ ID NO: 205 and 202, respectively; SEQ ID NO: 264 and 122, respectively; SEQ ID NO: 207 and 201, respectively; SEQ ID NO: 208 and 202, respectively; SEQ ID NO: 208 and 209, respectively; SEQ ID NO: 210 and 209, respectively; or SEQ ID NO: 249 and 122, respectively.
   [I-5] A polynucleotide or a plurality of polynucleotides encoding the protein complex of any one of [I-1] to [I-4].
   [I-6] A recombinant host cell comprising a polynucleotide or a plurality of polynucleotides encoding the protein complex of any one of [I-1] to [I-4].
   [I-7] A method of producing the protein complex of any one of [I-1] to [I-4] comprising (a) culturing a recombinant host cell comprising a polynucleotide or a plurality of polynucleotides encoding the protein complex under conditions suitable for the expression of the protein complex and (b) recovering the protein complex.
   [I-8] A method of treating a disease or disorder in an individual in need thereof, comprising administering to said individual a therapeutically effective amount of the protein complex of any one of [I-1] to [I-4].
   [J-1] A protein complex comprising two subunits associated with each other, wherein each subunit comprises a first polypeptide and a second polypeptide, wherein the first polypeptide comprises a light chain variable domain (VL) and a light chain constant domain (CL), and the second polypeptide is a fusion protein represented by the general formula (I), from the N- to the C-terminus:

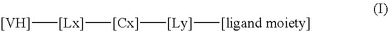

wherein:
VH represents a heavy chain variable domain (VH), wherein the VH is associated with the VL of the first polypeptide to form a ligand-binding domain that binds to the ligand moiety,
Lx represents a peptide linker comprising a protease cleavage site,
Cx represents a constant domain associated with the CL of the first polypeptide, wherein Cx comprises, from the N- to the C-terminus, a CH1 domain, a second peptide linker, a CH2 domain, and a CH3 domain,
Ly represents a third peptide linker, and
the ligand moiety comprises a cytokine or chemokine, and
wherein (a) in a first state, the ligand moiety is bound by the ligand-binding domain and the biological activity of the ligand moiety is attenuated, and in a second state, the biological activity of the ligand moiety is restored, and (b) the protein complex in the first state has a longer half-life in blood than in the second state, and (c) switching from the first state to the second state is mediated by the presence of a protease that cleaves the protease cleavage site.
   [J-2] The protein complex of [J-1], characterized by one or more of
(a) the protease cleavage site is cleavable by a protease selected from the group consisting of matriptase (MT-SP1), urokinase-type plasminogen activator (uPA) and matrix metalloprotease (MMP);
(b) Lx represents a peptide linker comprising an amino acid sequence selected from SEQ ID NO: 194, 199, and 214 to 219;
(c) the ligand moiety is selected from the group consisting of IL-12, IL-22, IL-2, IL-7, IL-15, IL-18, IL-21, CXCL9, CXCL10, CXCL11, IFN-alpha, IFN-beta, IFN-gamma, MIG, I-TAC, RANTES, MIP-1a, MIP-1b, IL-1R1, IL-1R2, IL-1RAcP and IL-1Ra; and
(d) the VH comprises a substitution at the amino acid position corresponding to a position selected from residue 37, 45, 91, 100a, or 103 of SEQ ID NO: 116 (according to Kabat numbering), and/or the VL comprises a substitution at the amino acid position corresponding to a position selected from residue 30, 43, 46, 49 or 87 of SEQ ID NO: 114 (according to Kabat numbering).
   [J-3] A polynucleotide or a plurality of polynucleotides encoding the protein complex of [J-1] or [J-2].
   [J-4] A method of treating a disease or disorder in an individual in need thereof, comprising administering to said individual a therapeutically effective amount of the protein complex of [J-1] or [J-2].
   The protein complex of [G-1], [I-1], or [J-1], characterized by a feature as defined in any of [A-1] to [A-14], and [B-1] to [B-57].
   The attenuated IL-12 polypeptide of [F-1], characterized by a feature as defined in any one of [C1-] to [C-28].

### BRIEF DESCRIPTION OF THE FIGURES

FIG. 1 Expected Profile of IL-12 fusion proteins (also referenced herein as protein complexes). As inactive molecule, biological activity of IL-12 should be inhibited and the IL-12 fusion protein should have long systemic half-life. As an activated molecule, due to cleavage by disease specific proteases, IL-12 biological activity is restored. In addition, the fusion protein should retain in high concentrations in disease tissue and should exhibit short systemic half-life.

FIG. 2 Panel (A) shows the molecular format of monovalent IL-12 fusion proteins (also referenced herein as protein complexes) used to evaluate the effect of ligand binding domain on the pharmacokinetics of the inactive IL-12 fusion proteins. Panel (B) shows the pharmacokinetics of inactive IL-12 fusion protein in non-tumor bearing mice. Upper graph shows plasma concentrations of monovalent IL-12 release FP1 (closed circle), FP2 (closed triangle), and FP3 (cross mark) after a single intravenous dose in non-tumor bearing mice (n=3). Lower table shows pharmacokinetic parameters of each fusion protein. C₀: back-extrapolated concentration immediately after intravenous injection, t_{1/2}: elimination half-life, AUC_{inf}: area under the plasma concentration-time curve from time zero extrapolated to infinity, CL: total clearance, Vₛₛ: volume of distribution at steady state.

FIG. 3 Different formats of IL-12 fusion proteins (also referenced herein as protein complexes). Panel (A) shows IL-12 release type fusion proteins, where cleavable linker was introduced into elbow hinge region between VH and CH1 regions. Single-chain IL-12 was attached to C-terminal of Fc domain via cleavable linker. Digestion of cleavable linkers results in release of active IL-12. Panel (B) shows IL-12 fusion type fusion proteins, where cleavable linker was introduced into elbow hinge region between VH and CH1 regions. The IL-12 fusion type fusion proteins and protein complexes of the invention exhibit this structure. GS linker was inserted in hinge region and single-chain IL-12 was attached to C-terminal of Fc domain via GS linker. Digestion of cleavable linkers results in release of active IL-12 fused to Fc.

FIG. 4 Bivalent IL-12 release FP4 (A), and Bivalent IL-12 fusion FP5 (B) (also referenced herein as a protein complex) were subjected to the IL-12 luciferase assay. Both variants showed lower IL-12 bioactivity than hIL-12_His tag in the absence of MT-SP1, and the IL-12 bioactivity was restored to the same level as hIL-12_His tag upon MT-SP1 treatment.

FIG. 5 Different formats of activated IL-12 fusion protein (also referenced herein as a protein complex) upon protease cleavage. (A) In release format, freely dissociated IL-12 molecule is the representative activated molecule, or surrogate activated molecule, i.e. recombinant IL-12. (B) In the fusion format, KLH Bivalent fusion FP6 is the representative activated molecule, or surrogate activated molecule. The IL-12 fusion type fusion proteins of the invention exhibit the fusion format.

FIG. 6 IL-12 concentrations in tumor lysate and tumor interstitial fluid of recombinant IL-12 or KLH-Bivalent IL12 fusion FP6 in human T cell injected LS1034 tumor bearing mice model after intra-tumor injection, repeated for a total of six times. Tumor retention levels comparing the surrogate activated forms of release and fusion format shows higher retention concentrations for the fusion format in both tumor lysate and interstitial fluid than the release format.

FIG. 7 Time course of plasma KLH-Bivalent IL-12 fusion FP6 concentration after intravenous administration in cynomolgus monkey. KLH-Bivalent IL-12 fusion FP6 was eliminated rapidly and the clearance was 1975 mL/day/kg, which is approximately 13-times faster than recombinant IL-12 clearance reported in literature which is 6.23 mL/hour/kg (150 mL/day/kg) (Pharmacology 2010; 85:319-327).

FIG. 8 Bivalent IL-12 fusion protein FP14 (also referenced herein as a protein complex) was subjected to in vitro activity assessment to check the differences in activity between non-cleaved form and active form. Differences in EC50 value of non-cleaved (inactive) form vs cleaved (active) form was found to be ˜34.7×.

FIG. 9 Bivalent IL-12 fusion proteins (also referenced herein as protein complexes) comprising IL-12 affinity enhancing mutations in the variable region, i.e. IL-12 affinity enhanced variants, demonstrated higher VH release tendency than the variant without VH release mutations.

FIG. 10A Among molecules comprising the same cleavable linker, differences in EC50 value of non-cleaved (inactive) form vs cleaved (active) form of Bivalent IL-12 fusion proteins (also referenced herein as protein complexes) comprising IL-12 affinity enhancing mutations in the variable region, i.e. IL-12 affinity enhanced variants is higher compared to those without the IL-12 affinity enhanced mutations, i.e. control. FIG. 10A shows the difference in EC50 values of non-cleaved (inactive) vs cleaved (active) forms of Bivalent IL-12 fusion protein FP15 and FP16.

FIG. 10B Among molecules comprising the same cleavable linker, differences in EC50 value of non-cleaved (inactive) form vs cleaved (active) form of Bivalent IL-12 fusion proteins (also referenced herein as protein complexes) comprising IL-12 affinity enhancing mutations in the variable region, i.e. IL-12 affinity enhanced variants is higher compared to those without the IL-12 affinity enhanced mutations, i.e. control. FIG. 10B shows the difference in EC50 values of non-cleaved (inactive) vs cleaved (active) forms of Bivalent IL-12 fusion protein FP17 and FP14.

FIG. 10C Among molecules comprising the same cleavable linker, differences in EC50 value of non-cleaved (inactive) form vs cleaved (active) form of Bivalent IL-12 fusion proteins (also referenced herein as protein complexes) comprising IL-12 affinity enhancing mutations in the variable region, i.e. IL-12 affinity enhanced variants is higher compared to those without the IL-12 affinity enhanced mutations, i.e. control. FIG. 10C shows the difference in EC50 values of non-cleaved (inactive) vs cleaved (active) forms of Bivalent IL-12 fusion protein FP19 and FP20.

FIG. 10D Among molecules comprising the same cleavable linker, differences in EC50 value of non-cleaved (inactive) form vs cleaved (active) form of Bivalent IL-12 fusion proteins (also referenced herein as protein complexes) comprising IL-12 affinity enhancing mutations in the variable region, i.e. IL-12 affinity enhanced variants is higher compared to those without the IL-12 affinity enhanced mutations, i.e. control. FIG. 10D shows the difference in EC50 values of non-cleaved (inactive) vs cleaved (active) forms of Bivalent IL-12 fusion protein FP21 and FP22.

FIG. 11A Within each PBMC donor, non-cleaved bivalent IL-12 fusion protein (also referenced herein as a protein complex) variants showed IL-12 neutralization to varying degrees. Following protease digestion, IL-12 activity of each molecule was increased between 17-160-fold relative to the respective non-cleaved form. FIG. 11A shows IL-12 activity of Bivalent IL-12 fusion protein FP14.

FIG. 11B Within each PBMC donor, non-cleaved bivalent IL-12 fusion protein (also referenced herein as a protein complex) variants showed IL-12 neutralization to varying degrees. Following protease digestion, IL-12 activity of each molecule was increased between 17-160-fold relative to the respective non-cleaved form. FIG. 11B shows IL-12 activity of Bivalent IL-12 fusion protein FP20.

FIG. 11C Within each PBMC donor, non-cleaved bivalent IL-12 fusion protein (also referenced herein as a protein complex) variants showed IL-12 neutralization to varying degrees. Following protease digestion, IL-12 activity of each molecule was increased between 17-160-fold relative to the respective non-cleaved form. FIG. 11C shows IL-12 activity of Bivalent IL-12 fusion protein FP21.

FIG. 11D Within each PBMC donor, non-cleaved bivalent IL-12 fusion protein (also referenced herein as a protein complex) variants showed IL-12 neutralization to varying degrees. Following protease digestion, IL-12 activity of each molecule was increased between 17-160-fold relative to the respective non-cleaved form. FIG. 11D shows IL-12 activity of Bivalent IL-12 fusion protein FP22.

FIG. 12A The cleavage ratio (%) of the cleavable variants by huPA, hMT-SP1 and human plasma. All cleavable variants demonstrated higher cleavage ratio (%) in huPA (FIG. 12A) and hMT-SP1 (FIG. 12B) compared to human plasma.

FIG. 12B The cleavage ratio (%) of the cleavable variants by huPA, hMT-SP1 and human plasma. All cleavable variants demonstrated higher cleavage ratio (%) in huPA (FIG. 12A) and hMT-SP1 (FIG. 12B) compared to human plasma.

FIG. 13A Accumulation of active IL12 in tumor and tumor interstitial fluid was observed for Bivalent IL-12 fusion protein FP17 and Bivalent IL-12 fusion protein FP20, which possess a cleavable linker, whereas Bivalent IL-12 fusion protein FP25 which possesses non-cleavable linker did not show detectable active IL-12 accumulation in tumor compared to Bivalent IL-12 fusion protein FP17 and Bivalent IL-12 fusion protein FP20 (also referenced herein as a protein complex). FIG. 13A shows accumulation in mice with huPA-overexpressing NCI-H446 human lung carcinoma cells.

FIG. 13B Accumulation of active IL12 in tumor and tumor interstitial fluid was observed for Bivalent IL-12 fusion protein FP17 and Bivalent IL-12 fusion protein FP20, which possess a cleavable linker, whereas Bivalent IL-12 fusion protein FP25 which possesses non-cleavable linker did not show detectable active IL-12 accumulation in tumor compared to Bivalent IL-12 fusion protein FP17 and Bivalent IL-12 fusion protein FP20 (also referenced herein as a protein complex). FIG. 13B shows accumulation in mice with Ls174T human colorectal carcinoma cells.

FIG. 14A Bivalent IL-12 fusion protein FP17 and Bivalent IL-12 fusion protein FP20 showed higher percentage of cleavage compared to Bivalent IL-12 fusion protein FP25 which possesses non-cleavable linker. Bivalent IL-12 fusion protein FP17 and Bivalent IL-12 fusion protein FP20 (also referenced herein as a protein complex) with cleavable linkers showed higher percentage of cleavage in tumor and tumor interstitial fluid compared to plasma. FIG. 14A shows cleavage in mice with huPA-overexpressing NCI-H446 human lung carcinoma cells.

FIG. 14B Bivalent IL-12 fusion protein FP17 and Bivalent IL-12 fusion protein FP20 showed higher percentage of cleavage compared to Bivalent IL-12 fusion protein FP25 which possesses non-cleavable linker. Bivalent IL-12 fusion protein FP17 and Bivalent IL-12 fusion protein FP20 (also referenced herein as a protein complex) with cleavable linkers showed higher percentage of cleavage in tumor and tumor interstitial fluid compared to plasma. FIG. 14B shows cleavage in mice with Ls174T human colorectal carcinoma cells.

FIG. 15 Based on the in vitro activity assessment of Bivalent IL12 fusion proteins (also referenced herein as protein complexes), with (“Cleaved”) and without (“Non-cleaved”) protease digestion, IL12 variants with lower activity have improved window (“fold change”).

FIG. 16 Compared to bivalent IL-12 fusion protein (also referenced herein as a protein complex) harboring wild-type IL-12 (bivalent IL-12 fusion protein FP14), all IL-12 variant fusion proteins displayed wider fold change in IL-12 activity between inactive (“non-cleaved”) and “active” (cleaved) forms. Fusion protein FP31 comprising a Y16P mutation in the IL-12 achieved more than 25-fold improvement compared to FP14.

FIG. 17 Within each PBMC donor, non-cleaved bivalent IL-12 fusion protein (also referenced herein as a protein complex) variants showed IL-12 neutralization to varying degrees. The fusion proteins comprising IL-12 variants showed significantly improved fold-change in IL-12 activity window between non-cleaved (“Non-cleaved”) and cleaved (“Active”) proteins. Fusion protein FP31 comprising a Y16P mutation in the IL-12 achieved approximately 6-fold improvement compared to FP14.

FIG. 18A Accumulation of active IL12 in tumor was observed for Bivalent IL-12 fusion protein FP20, Bivalent IL-12 fusion protein FP22 and Bivalent IL-12 fusion protein FP31 which possess a cleavable linker, whereas Bivalent IL-12 fusion protein FP25 (also referenced herein as a protein complex) which possesses a non-cleavable linker did not show significant active IL12 accumulation in tumor. FIG. 18A shows accumulation in mice with huPA-overexpressing NCI-H446 human lung carcinoma cells.

FIG. 18B Accumulation of active IL12 in tumor was observed for Bivalent IL-12 fusion protein FP20, Bivalent IL-12 fusion protein FP22 and Bivalent IL-12 fusion protein FP31 which possess a cleavable linker, whereas Bivalent IL-12 fusion protein FP25 (also referenced herein as a protein complex) which possesses a non-cleavable linker did not show significant active IL12 accumulation in tumor. FIG. 18B shows accumulation in mice with Ls174T human colorectal carcinoma cells.

FIG. 19A Bivalent IL-12 fusion protein FP20, Bivalent IL-12 fusion protein FP22 and Bivalent IL-12 fusion protein FP31 which possess a cleavable linker showed higher percentage of cleavage rate compared to Bivalent IL-12 fusion protein FP25 which possesses a non-cleavable linker. Bivalent IL-12 fusion protein FP20, Bivalent IL-12 fusion protein FP22 and Bivalent IL-12 fusion protein FP31 (also referenced herein as a protein complex) with cleavable linkers showed higher percentage of cleavage in tumor and tumor interstitial fluid compared to plasma. FIG. 19A shows cleavage in mice with huPA-overexpressing NCI-H446 human lung carcinoma cells.

FIG. 19B Bivalent IL-12 fusion protein FP20, Bivalent IL-12 fusion protein FP22 and Bivalent IL-12 fusion protein FP31 which possess a cleavable linker showed higher percentage of cleavage rate compared to Bivalent IL-12 fusion protein FP25 which possesses a non-cleavable linker. Bivalent IL-12 fusion protein FP20, Bivalent IL-12 fusion protein FP22 and Bivalent IL-12 fusion protein FP31 (also referenced herein as a protein complex) with cleavable linkers showed higher percentage of cleavage in tumor and tumor interstitial fluid compared to plasma. FIG. 19B shows cleavage in mice with Ls174T human colorectal carcinoma cells.

FIG. 20(A) Time course analysis of KLH bivalent fusion FP7 showed a clearance of 335 ml/day/kg in SCID mice. (B) Time course analysis of inactive and active form of the IL-12 fusion protein (also referenced herein as a protein complex). Clearance levels between inactive and active form of the IL-12 fusion protein was similar.

FIG. 21A Schematic representation of BIACORE® assay to evaluate percentage dissociation of VH from an antibody. Representation of assay conducted for anti-IL-12 antibodies that bind IL-12.

FIG. 21B Schematic representation of BIACORE® assay to evaluate percentage dissociation of VH from the fusion protein (also referenced herein as a protein complex). Representation of assay conducted for bivalent IL-12 fusion protein.

FIG. 22A Screening and evaluation of amino acid modifications at the VH/VL interface that promote VH dissociation from bivalent IL-12 fusion proteins (also referenced herein as protein complexes).

FIG. 22B Screening and evaluation of amino acid modifications at the VH/VL interface that promote VH dissociation from bivalent IL-12 fusion proteins (also referenced herein as protein complexes).

FIG. 23 Time course of plasma concentrations of IL-12 fusion proteins (also referenced herein as protein complexes) with VH release modifications in SCID mice. Inclusion of VH/VL interface modification(s) have led to greater dissociation of IL-12 from the digested products of IL-12 fusion proteins and led to faster clearance compared to digested product of IL-12 fusion proteins without any modification(s) in the VH/VL interface.

FIG. 24 Time course analysis of KLH bivalent fusion FP7 with and without MT-SP1 digestion. Digestion with MT-SP1 unexpectedly led to slower clearance of KLH bivalent fusion FP7. This could affect the profile of the activated IL12 molecule.

FIG. 25 SDS-PAGE analysis showing MT-SP1 mediated digestion using KLH bivalent fusion variants with protease resistant modifications in the heparin binding region of p40. Top panel shows undigested and digested samples with 1-hour incubation with MT-SP1. Bottom panel shows digested samples with 4-hour and 24-hour incubation with MT-SP1.

FIG. 26 SDS-PAGE analysis showing MT-SP1 mediated digestion using KLH bivalent fusion variants with protease resistant modifications in the heparin binding region of p40. Top panel shows undigested and digested samples with 1-hour incubation with MT-SP1. Bottom panel shows digested samples with 4-hour and 24-hour incubation with MT-SP1.

FIG. 27 IL-12 activity of protease resistant IL-12 variants was evaluated using Luciferase assay. All the protease resistant IL-12 variants indicated similar activity to hIL12_His tag regardless of protease treatment.

FIG. 28 Time course of plasma concentrations of protease resistant IL-12 variants as KLH-bivalent fusion in SCID mice. All the protease resistant variants demonstrated slower elimination than control (KLH-Bivalent IL12006v1).

FIG. 29 Bivalent IL-12 fusion proteins FP8, FP11 and FP12 (also referenced herein as protein complexes) were subjected to the IL-12 luciferase assay. All three fusion proteins showed lower IL-12 bioactivity than hIL-12-His tag in the absence of MT-SP1, and the IL-12 bioactivity was restored to the same level as hIL-12_His tag upon MT-SP1 treatment.

FIG. 30 Tumor growth inhibition by treatment with Bivalent IL-12 fusion protein FP31 (also referenced herein as a protein complex) and IL12-Fc protein FP32 were evaluated in a colon38 tumor-bearing mice. FP31 and FP32 treatment both reduced tumor volume to similar extents (A), however, while the body weights of the mice in the FP31 treatment group were substantially the same as PBS treatment (control) group, the body weights of the mice in FP32 treatment group were lower than the FP31 and PBS treatment group (B).

FIG. 31 Aspartate transaminase (AST) and alanine transaminase (ALT), markers indicative of hepatocellular damage, were assessed in the tumor-bearing mice. Spleen (Sp) weight was also assessed as another systemic toxicity indicator. ALT and AST levels and spleen (Sp) weight were significantly increased in mice of the FP32 treatment group, however, this ALT and AST elevation and increase of spleen (Sp) weight was not observed in mice of the FP31 treatment group.

FIG. 32(A) IL-12 activity of Bivalent IL-12 fusion proteins (also referenced herein as protein complexes) comprising IL-12 de-glycosylated or wild-type (non-de-glycosylated) protease resistance IL-12 were evaluated using Luciferase assay. Both bivalent IL-12 fusion proteins exhibited similar IL-12 activity regardless of the glycosylation status of the IL-12. (B) In addition, both bivalent IL-12 fusion proteins showed more than 50% VH release. IL-12 glycosylation status did not affect the release of VH.

### DETAILED DESCRIPTION OF EMBODIMENTS OF THE INVENTION

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds 1987, and periodic updates); PCR: The Polymerase Chain Reaction, (Mullis et al., ed., 1994); A Practical Guide to Molecular Cloning (Perbal Bernard V., 1988); Phage Display: A Laboratory Manual (Barbas et al., 2001).

The definitions and detailed description below are provided to facilitate understanding of the present disclosure illustrated herein. All references mentioned herein are specifically incorporated by reference.

### I. DEFINITIONS

### Protein/Polypeptide

As used herein, the term “polypeptide” refers to a molecule composed of monomers (amino acids) linearly linked by amide bonds (also known as peptide bonds). The term “polypeptide” usually refers to a peptide having a length on the order of 4 amino acids or longer, and does not refer to a specific length of the product. As used herein, the term also includes fragments of polypeptides. Thus, peptides, dipeptides, tripeptides, oligopeptides, “protein”, “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide as described herein may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations and are referred to as unfolded.

### Protein Complex

As used herein, the term “protein complex” refers to molecular construct that is formed by association of at least two proteins or polypeptides. The association of the polypeptides in the complex does not occur via peptide bonds and thus allows the separation of the polypeptides without destroying the primary structure of the involved proteins. For example, the polypeptides in the protein complex can be associated via one or more disulfide bonds or non-covalently. Exemplary embodiments of a protein complex of the present invention comprise the protein complex of [A-1], [A-1a], [A-1b], [B-1], [G-1], [I-1] or [J-1], wherein two subunits are associated with each other, the protein complex of [A-7] comprising an anti-IL-12 binding molecule complex, wherein a heavy chain variable domain is associated with a light chain variable domain, the protein complex of [A-10] comprising a ligand-binding domain, wherein the ligand-binding domain comprises a heavy chain variable domain associated with a light chain variable domain. Further examples are the IL-12 protein complex of [A-16], the IL-12 fusion protein of [A-17], and the IL-12 polypeptide of [F-1], wherein polypeptides representing the two subunits p35 and p40 are associated with each other, and the anti-IL-12 binding molecule complex of [A-20], wherein a heavy chain variable domain is associated with a light chain variable domain.

### Fusion Protein

As used herein, the term “fusion protein” refers to a single-chain protein, i.e. a protein formed by a single chain of amino acids, formed by the combination of at least two (poly)peptides. In embodiments, the combination occurs via peptide bonds. In embodiments, the combination occurs via peptide bonds provided by a peptide linker. An example for a fusion protein of the present invention is the second polypeptide of [A-1], [A-1a], [A-1b], [B-1], [G-1], [I-1] or [J-1], wherein a constant domain is linked to a ligand moiety via a peptide linker. The second polypeptide of [A-1], [A-1a], [A-1b], [B-1], [G-1], [I-1] or [J-1], comprises three further peptide linkers. The first peptide linker links the heavy chain variable domain to the heavy chain constant domain, preferably with the CH1 domain. The second peptide linker links two heavy chain constant domains with each other, preferably CH1 with CH2. The third peptide linker is present in another example of a fusion protein of the present invention, which is the ligand moiety itself, which is the single chain IL12 fusion protein of [A-1], [A-1a], [A-1b], [B-1], [G-1], [I-1] or [J-1], wherein the two polypeptides representing the two subunits p35 and p40 of IL-12 are linked via a peptide linker. The second polypeptide of [A-1], [A-1a], [A-1b], [B-1], [G-1], [I-1] or [J-1], therefore comprises four peptide linkers, and is thus the result of four fusion processes. The fusion protein or the fusion protein complex of the present invention comprises an antigen binding-domain that may bind an antigen, e.g. Interleukin-12 (IL-12).

The terms “a fusion protein that binds IL-12”, “a polypeptide that binds IL-12”, or “an IL-12 binding molecule”, “an antibody that binds to IL-12” or “anti-IL-12 antibody” refer to measurable and reproducible interactions between the protein, molecule, or antibody, with IL-12, which is determinative of the presence of its antigen, e.g. IL-12 in the presence of a heterogenous population of molecules including biological molecules. The fusion protein or antibody that binds to its antigen with greater affinity, avidity, more readily, and/or with greater duration than it binds to other antigens. In one embodiment, the extent of binding of the fusion protein or the antibody to an unrelated antigen is less than about 10% of the binding of the fusion protein or the antibody to the antigen as measured, e.g., by a radioimmunoassay (RIA). In certain embodiments, a fusion protein or an antibody that specifically binds to an antigen/target has a dissociation constant (Kd) of 1 micromolar (micro M) or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻³ M, e.g., from 10⁻⁹ M to 10⁻³ M).

### Cleavable Peptide Linkers

Herein, the terms “a peptide linker comprising/having a protease cleavage site/sequence”, “a protease cleavable linker”, “a cleavable peptide linker”, and “a cleavable linker” as used interchangeably herein, refer to a peptide linker containing a cleavage site/sequence that can be selectively cleaved by a protease.

### Amino Acids

Herein, amino acids are described by one-letter code or three-letter code, or both, as represented by, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V. For expressing an amino acid located at a particular position, an expression using a number representing the particular position in combination with the one-letter code or the three-letter code of the amino acid can be appropriately used. For example, an amino acid 37V, which is an amino acid contained in a variable region of an antibody, represents Val located at position 37 defined by the Kabat numbering.

### Amino Acid Modification

The terms “amino acid modification”, “amino acid alteration” or “amino acid mutation” as used interchangeably herein, refer to the alteration of an amino acid in the amino acid sequence of a protein or polypeptide by a method known in the art that can be appropriately adopted such as site-directed mutagenesis (Kunkel et al. (Proc. Natl. Acad. Sci. USA (1985) 82, 488-492)) or overlap extension PCR. Several methods known in the art can also be adopted as alteration methods for substituting an amino acid by an amino acid other than a natural amino acid (Annu. Rev. Biophys. Biomol. Struct. (2006) 35, 225-249; and Proc. Natl. Acad. Sci. U.S.A. (2003) 100 (11), 6353-6357). For example, a tRNA-containing cell-free translation system (Clover Direct (Protein Express)) having a non-natural amino acid bound with amber suppressor tRNA complementary to UAG codon (amber codon), which is a stop codon, is also preferably used. In the present specification, examples of an amino acid modification at a specified position include the substitution or deletion of the specified residue, or the insertion of at least one amino acid residue adjacent the specified residue or any combination of substitution, deletion and insertion thereof. Insertion “adjacent” to a specified residue means insertion within one to two residues thereof. The insertion may be N-terminal or C-terminal to the specified residue. The preferred amino acid modification herein is a substitution.

### Substitution

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). Preferably, the replacement residue is not cysteine. Substitution with one or more non-naturally occurring amino acid residues is also encompassed by the definition of an amino acid substitution herein. A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

### Insertion

An “amino acid insertion” refers to the incorporation of at least one amino acid into a predetermined amino acid sequence. While the insertion will usually consist of the insertion of one or two amino acid residues, the present application contemplates larger “peptide insertions”, e.g. insertion of about three to about five or even up to about ten amino acid residues. The inserted residue(s) may be naturally occurring or non-naturally occurring as disclosed above.

### Deletion

An “amino acid deletion” refers to the removal of at least one amino acid residue from a predetermined amino acid sequence.

The term “and/or” as used herein when referring to a site of amino acid alteration includes every combination appropriately represented by “and/or”. Specifically, for example, the phrase “amino acids at positions 37, 45, and/or 47 are substituted” includes the following variations of amino acid alteration: (a) position 37, (b) position 45, (c) position 47, (d) positions 37 and 45, (e) positions 37 and 47, (f) positions 45 and 47, and (g) positions 37, 45 and 47.

In the present specification, expression in which the one-letter codes or three-letter-codes of amino acids before and after alteration are used previous and next to a number representing a particular position can be appropriately used for representing amino acid alteration. For example, an alteration F37V or Phe37Val used for substituting an amino acid contained in an antibody variable region represents the substitution of Phe at position 37 defined by the Kabat numbering by Val. Specifically, the number represents an amino acid position defined by the Kabat numbering; the one-letter code or three-letter code of the amino acid previous to the number represents the amino acid before the substitution; and the one-letter code or three-letter code of the amino acid next to the number represents the amino acid after the substitution. Likewise, an alteration P238A or Pro238Ala used for substituting an amino acid in a Fc region contained in an antibody constant region represents the substitution of Pro at position 238 defined by the EU numbering by Ala. Specifically, the number represents an amino acid position defined by the EU numbering; the one-letter code or three-letter code of the amino acid previous to the number represents the amino acid before the substitution; and the one-letter code or three-letter code of the amino acid next to the number represents the amino acid after the substitution.

### Percent (%) Amino Acid Sequence Identity

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity.

Alignment for purposes of determining percent amino acid sequence identity can be achieved in various ways that are within the skill in the art, for instance, using publicly available computer software such as BLAST, BLAST-2, ALIGN, Megalign (DNASTAR) software, or GENETYX (registered trademark) (Genetyx Co., Ltd.). Those skilled in the art can determine appropriate parameters for aligning sequences, including any algorithms needed to achieve maximal alignment over the full length of the sequences being compared. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

$$100 times the fraction X / Y$$

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Ligand-Binding Moiety/Molecule

In some embodiments, the protein complex, polypeptide, binding molecule or binding molecule complex, or fusion protein is a polypeptide complex comprising a ligand-binding moiety or ligand-binding molecule further comprising a ligand-binding domain. The terms “ligand-binding moiety” or “ligand-binding molecule” as used herein, refer to a moiety or molecule that is capable of binding to a ligand, and particularly refers to a moiety or molecule that binds to a ligand when the moiety or molecule is in the uncleaved state. In this context, the “binding” usually refers to binding through interaction based mainly on a noncovalent bond such as electrostatic force, van der Waals' force, or a hydrogen bond. Preferred examples of the binding mode of the ligand-binding moiety or molecule include, but are not limited to, antigen-antibody reaction through which an antigen-binding domain, an antigen-binding molecule, an antibody, an antibody fragment, or the like binds to the antigen. In certain embodiments, the ligand-binding moiety or molecule includes, but is not limited to, antibody fragments, antibodies, and molecules formed from antibody fragments. In embodiments, the terms “ligand-binding moiety” comprises a ligand-binding domain, which includes antibody fragments. In embodiments, the terms “ligand-binding moiety” and “anti-IL-12 binding molecule complex” may be used interchangeably herein. In embodiments, the ligand-binding moiety comprises a light chain variable domain (VL) and a light chain constant domain (CL) in a first polypeptide, and a heavy chain variable domain (VH) in a second polypeptide, wherein the VH is associated with the VL of the first polypeptide to form a ligand-binding domain. In embodiments, the ligand-binding moiety comprises a light chain variable domain (VL) and a light chain constant domain (CL) in a first polypeptide, and a heavy chain variable domain (VH) and a heavy chain constant domain in a second polypeptide, wherein said heavy chain constant domain is associated with the CL of the first polypeptide, and wherein the VH of the second polypeptide is associated with the VL of the first polypeptide to form a ligand-binding domain. In embodiments, the heavy chain constant domain comprises or consists of, from the N- to the C-terminus, a CH1 domain, a second peptide linker, a CH2 domain and a CH3 domain. In embodiments, the CH1 domain of a heavy chain constant domain and a CL are bound to each other via one or more disulfide bonds.

### Ligand-Binding Domain

The term “ligand-binding domain” as used herein, refers to a portion of a ligand-binding moiety or molecule which binds only to a portion of a ligand (epitope) when the ligand-binding moiety/molecule binds to the ligand. Examples of the ligand-binding domain include, but are not limited to, an antigen-binding domain, an antibody heavy chain variable region (VH), an antibody light chain variable region (VL), an antibody Fv region, and a single-domain antibody (sdAb). In embodiments, the ligand-binding domain comprises a light chain variable domain (VL) and a heavy chain variable domain (VH), wherein the VH is associated with the VL.

### Antigen-Binding Domain

The term “antigen-binding domain” as used herein, refers to a region that specifically binds to or partially complements an antigen. As used herein, an antigen binding molecule comprises an antigen-binding domain. If the molecular weight of the antigen is large, the antigen-binding domain can only bind to a specific part of the antigen. The specific part is called an epitope. In one embodiment, the antigen-binding domain comprises an antibody fragment that binds to a particular antigen. The antigen-binding domain may be provided by one or more antibody variable domains. In one non-limiting embodiment, the antigen-binding domain comprises an antibody light chain variable region (VL) and an antibody heavy chain variable region (VH). Examples of such antigen-binding domains include “scFv (single-chain Fv)”, “single-chain antibody (single-chain antibody)”, “Fv”, “scFv2 (single-chain Fv 2)”, “Fab” or “F(ab′)2”, and the like. In another embodiment, the antigen-binding domain comprises a non-antibody protein or a fragment thereof that binds to a particular antigen. In some embodiments, the antigen is a ligand. As used herein, where the antigen is a ligand, the terms “antigen-binding domain” and “ligand-binding domain” may be used interchangeably to refer to the region that specifically or partially binds the ligand as the antigen. In embodiments, the antigen-binding domain comprises a light chain variable domain (VL) and a heavy chain variable domain (VH), wherein the VH is associated with a VL. In embodiments, the antigen-binding moiety/molecule comprises a light chain variable domain (VL) and a light chain constant domain (CL) in a first polypeptide, and a heavy chain variable domain (VH), wherein the VH is associated with the VL of the first polypeptide to form an antigen-binding domain. In embodiments, the antigen-binding moiety/molecule comprises a light chain variable domain (VL) and a light chain constant domain (CL) in a first polypeptide, and a heavy chain variable domain (VH) and a heavy chain constant domain in a second polypeptide, wherein said heavy chain constant domain is associated with the CL of the first polypeptide, and wherein the VH of the second polypeptide is associated with the VL of the first polypeptide to form an antigen-binding domain. In embodiments, the heavy chain constant domain comprises or consists of, from the N- to the C-terminus, a CH1 domain, a second peptide linker, a CH2 domain and a CH3 domain. In embodiments, the CH1 domain of a heavy chain constant domain and a CL are bound to each other via one or more disulfide bonds.

As used herein, the term “binding to the same epitope” means that the epitopes to which two antigen binding domains bind overlap at least partially. The degree of overlap is not limited, but is at least 10% or more, preferably 20% or more, 30% or more, 40% or more, 50% or more, 60% or more, 70% or more, 80% or more, and particularly preferably 90% or more. Most preferably 100% overlap.

### Ligand/Ligand Moiety/Antigen

As used herein, the term “ligand” (which may alternatively be called “ligand moiety”) and “antigen” may be used interchangeably. The term “ligand” is limited only by containing an epitope to which the ligand-binding domain or antigen-binding domain binds. The term “ligand” and “antigen” refer to all molecules that can be specifically bound by the ligand-binding domain or antigen binding domain. In embodiments, the term “ligand” or “ligand moiety” refers to an IL-12 polypeptide or IL-12 protein. In embodiments, the ligand moiety is a protease resistant single-chain IL-12 fusion protein.

### And/or

The term “and/or” herein is used to indicate any one of the subjects shown before and after “and/or”, or any combination thereof. For example, “A, B, and/or C” includes the individual subjects “A”, “B”, and “C”, and also combinations “A and B”, “A and C”, “B and C”, and “A and B and C”.

### Specificity

As used herein, the term “specificity” refers to a property by which one of specifically binding molecules does not substantially bind to a molecule other than its one or more binding partner molecules. This term is also used when the ligand-binding domain, or antigen-binding domain has specificity for an epitope contained in a particular ligand, or antigen. The term is also used when the ligand-binding domain, or antigen-binding domain has specificity for a particular epitope among a plurality of epitopes contained in a ligand, or an antigen. In this context, the term “not substantially bind” is determined according to the method described in the section about binding activity and means that the binding activity of a specific binding molecule for a molecule other than the binding partner(s) is 80% or less, usually 50% or less, preferably 30% or less, particularly preferably 15% or less, of its binding activity for the binding partner molecule(s).

### Affinity

The term “affinity” as used herein, refers to the strength of the sum total of noncovalent interactions between a single binding site of a molecule (e.g., a ligand-binding molecule, a ligand, an antigen-binding molecule or an antibody) and its binding partner (e.g., a ligand, a ligand receptor, or an antigen). Unless indicated otherwise, as used herein, “binding affinity” refers to intrinsic binding affinity which reflects a 1:1 interaction between members of a binding pair (e.g., a ligand-binding molecule and a ligand, a ligand and a ligand receptor, an antigen-binding molecule and an antigen or an antibody and an antigen). The affinity of a molecule X for its partner Y can generally be represented by the dissociation constant (Kd), which is the ratio of dissociation and association rate constants (Koff and Kon, respectively). Affinity can be measured by common methods known in the art, including those described herein. Specific illustrative and exemplary embodiments for measuring binding affinity are described in the following.

### Antibody, Antibody Fragments, and Antibody Constructs

In one embodiment, the ligand-binding moiety of the presently claimed fusion protein or protein complex comprises an antibody as defined herein. As used herein, the term “antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. The term an “antibody fragment” as used herein, refers to a molecule other than an intact antibody that comprises a portion of an intact antibody that binds the antigen to which the intact antibody binds. Examples of antibody fragments include but are not limited to VH, VL, Fv, Fab, Fab′, Fab′-SH, F(ab′)2; diabodies; linear antibodies; single-chain antibody molecules (e.g. scFv); and multispecific antibodies formed from antibody fragments.

### Native Antibody

The term “native antibodies” as used herein, refer to naturally occurring immunoglobulin molecules with varying structures. For example, native IgG antibodies are heterotetrameric glycoproteins of about 150,000 Daltons, composed of two identical light chains and two identical heavy chains that are disulfide bonded. From N- to C-terminus, each heavy chain has a variable region (VH), also called a variable heavy domain or a heavy chain variable domain (VH), or an antibody heavy chain variable domain (VH), or antibody heavy chain variable region (VH), followed by three constant domains (CH1, CH2, and CH3). Similarly, from N- to C-terminus, each light chain has a variable region (VL), also called a variable light domain (VL) or a light chain variable domain (VL), or an antibody light chain variable domain (VH), or antibody light chain variable region (VL), followed by a constant light (CL) domain. The light chain of an antibody may be assigned to one of two types, called kappa and lambda, based on the amino acid sequence of its constant domain. As used herein, the terms “CL”, or “CL region”, or “CL domain” are used interchangeably, and the same is applicable to the other domains, “VH”, “VL”, “CH1”, “CH2” and “CH3”, when each of these terms are paired with “region” or “domain” in reference.

### Monoclonal Antibody

The term “monoclonal antibody” as used herein, refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

### Chimeric, Humanized and Human Antibody

The term “chimeric” antibody as used herein, refers to an antibody in which a portion of the heavy and/or light chain is derived from a particular source or species, while the remainder of the heavy and/or light chain is derived from a different source or species. A “humanized” (“humanized”) antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

### Affinity Matured Antibody

The term “affinity matured” antibody as used herein, refers to an antibody with one or more alterations in one or more hypervariable regions (HVRs), compared to a parent antibody which does not possess such alterations, such alterations resulting in an improvement in the affinity of the antibody for antigen.

### Antibody Classes

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

### Substantially Similar

The term “substantially similar” or “substantially the same,” as used herein, refers to a sufficiently high degree of similarity between two numeric values (for example, one associated with a protein complex or an antibody of the invention and the other associated with a reference/comparator protein complex or antibody), such that one of skill in the art would consider the difference between the two values to be of little or no biological and/or statistical significance within the context of the biological characteristic measured by said values (e.g., Kd values).

### Substantially Reduced

The phrase “substantially reduced” or “substantially different,” as used herein, refers to a sufficiently high degree of difference between two numeric values (generally one associated with a molecule and the other associated with a reference/comparator molecule) such that one of skill in the art would consider the difference between the two values to be of statistical significance within the context of the biological characteristic measured by said values (e.g., Kd values).

### Constant and Fc Regions

The terms “constant region” or “constant domain” as used herein refer to a region or a domain other than variable regions in an antibody. For example, an IgG antibody is a heterotetrameric glycoprotein of approximately 150,000 Da constituted by two identical light chains and two identical heavy chains connected through disulphide bonds. Each heavy chain has a variable region (VH) also called variable heavy chain domain or heavy chain variable domain, followed by a heavy chain constant region (CH) containing a CH1 domain, a hinge region, a CH2 domain, and a CH3 domain, from the N terminus toward the C terminus. Likewise, each light chain has a variable region (VL) also called variable light chain domain or light chain variable domain, followed by a constant light chain (CL) domain, from the N terminus toward the C terminus. The light chains of natural antibodies may be attributed to one of two types called kappa and lambda on the basis of the amino acid sequences of their constant domains. As used herein, the terms “CH1”, “CH1 domain”, “CH1 region” are used interchangeably. As used herein, the terms “CL”, “CL domain”, “CL region” are used interchangeably. In embodiments herein, the term “constant region” or “constant domain” encompasses a constant region wherein the heavy chain constant domain comprises a peptide linker connecting one constant domain with a further constant domain. In embodiments herein, the term “constant region” or “constant domain” encompasses a constant region wherein the heavy chain constant domain comprises a non-native peptide linker connecting CH1 with CH2.

### Fc Region

The term “Fc region” herein is used to define a C-terminal region of an immunoglobulin heavy chain that contains at least a portion of the constant region. The term includes native sequence Fc regions and variant Fc regions. A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991. The Fc region may be functional or non-functional. Preferably, it is non-functional, i.e. it does not bind to any Fc gamma receptor.

### Variant Fc Region

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

### Variant Constant Region

A “variant constant region” comprises an amino acid sequence which differs from that of a native sequence constant region by virtue of at least one amino acid modification, preferably one or more amino acid substitution(s). Preferably, the variant constant region has at least one amino acid substitution compared to a native sequence constant region or to the constant region of a parent polypeptide, e.g. from about one to about ten amino acid substitutions, and preferably from about one to about five amino acid substitutions in a native sequence constant region or in the constant region of the parent polypeptide. The variant constant region herein will preferably possess at least about 80% homology with a native sequence constant region and/or with a constant region of a parent polypeptide, and most preferably at least about 90% homology therewith, more preferably at least about 95% homology therewith.

### Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.). Binding to human FcRn in vivo and plasma half-life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001). In embodiments, an FcR is an Fc gamma R. In embodiments, an FcR includes Fc gamma RI R, Fc gamma RIIA R, Fc gamma RIIB R, Fc gamma RIIIa R and Fc gamma RIIIB. In an embodiment, a reference to an FcR is a reference to Fc gamma receptors.

### Fc Region-Comprising Antibody

The term “Fc region-comprising antibody” refers to an antibody that comprises an Fc region. The term “Fc region-comprising” when used in reference to a protein or a polypeptide or a protein complex, or a fusion protein, refers to the protein complex, polypeptide, fusion protein, or protein of the present disclosure comprising an Fc region. The C-terminal lysine (residue 447 according to the EU numbering system) or C-terminal glycine-lysine (residues 446-447) of the Fc region may be removed, for example, during purification of the antibody or polypeptide or by recombinant engineering of the nucleic acid encoding the antibody or polypeptide. Accordingly, a composition comprising an antibody, or a polypeptide having an Fc region according to this invention can comprise an antibody, or polypeptide with G446-K447, with G446 and without K447, with all G446-K447 removed, or a mixture of three types of antibodies, or polypeptides described above.

### Functional Fc Region

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Exemplary “effector functions” include C1q binding; CDC; Fc gamma receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein. In embodiments, effector function is Fc gamma receptor binding, ADCC or CDC. In embodiments, effector function is binding to all human Fc gamma receptors, ADCC or CDC.

### Human Effector Cells

“Human effector cells” refer to leukocytes that express one or more FcRs and perform effector functions. In embodiments, “Human effector cells” refer to leukocytes that express one or more Fc gamma Rs and perform effector functions. In certain embodiments, the cells express at least Fc gamma RIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

### Antibody-Dependent Cell-Mediated Cytotoxicity

“Antibody-dependent cell-mediated cytotoxicity” or “ADCC” refers to a form of cytotoxicity in which secreted Ig bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express Fc gamma RIII only, whereas monocytes express Fc gamma RI, Fc gamma RII, and Fc gamma RIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998).

### Complement Dependent Cytotoxicity

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

### Variable Region

The terms “variable region” or “variable domain” as used herein, refer to the domain of an antibody heavy or light chain that is involved in binding the antibody to antigen, including wherein the antigen is a ligand. Accordingly, the terms also refer to the ligand binding domain that is involved in binding the protein complex or fusion protein to the ligand. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W. H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991). As used herein, “heavy chain variable domain (VH)” is used interchangeably with “antibody heavy chain variable domain (VH)”, or “antibody heavy chain variable region (VH)”, or “VH”, or “antibody VH” or “VH domain”, and “light chain variable domain (VL)” is used interchangeably with “antibody light chain variable domain (VH)”, or “antibody light chain variable region (VL)” or “VL”, or “antibody VL” or “VL domain”.

### HVR or CDR

The terms “hypervariable region” or “HVR” as used herein, refer to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).
Unless otherwise indicated, HVR residues and other residues in the variable domain (e.g., FR residues) are numbered herein according to Kabat et al., supra.

### Framework

The terms “Framework” or “FR” as used herein, refer to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

### Isolated Antibody/Protein

An “isolated” antibody, or protein/polypeptide is one which has been separated from a component of its natural environment. In some embodiments, an antibody, or a protein/polypeptide is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody, or protein/polypeptide purity, see, e.g., Flatman et al., J. Chromatogr. B 848:79-87 (2007).

### Oligonucleotide

“Oligonucleotide,” as used herein, refers to generally single-stranded, synthetic polynucleotides that are generally, but not necessarily, less than about 200 nucleotides in length. The terms “oligonucleotide” and “polynucleotide” are not mutually exclusive. The description above for polynucleotides is equally and fully applicable to oligonucleotides.

### Polynucleotide (Nucleic Acid)

“Polynucleotide” or “nucleic acid” as used interchangeably herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. A sequence of nucleotides may be interrupted by non-nucleotide components. A polynucleotide may comprise modification(s) made after synthesis, such as conjugation to a label. Other types of modifications include, for example, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotides(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl-, 2′-fluoro- or 2′-azido-ribose, carbocyclic sugar analogs, alpha-anomeric sugars, epimeric sugars such as arabinose, xyloses or lyxoses, pyranose sugars, furanose sugars, sedoheptuloses, acyclic analogs, and basic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO, or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

### Isolated Nucleic Acid

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location. “Isolated nucleic acid encoding an anti-IL-12 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell. “Isolated nucleic acid encoding a fusion polypeptide (also referenced herein as a protein complex) that binds IL-12” refers to one or more nucleic acid molecules encoding the polypeptide of formula I or II (or fragments thereof), including such nucleic acid molecule(s) in a single vector or separate vectors, and such nucleic acid molecule(s) present at one or more locations in a host cell.

### Vector and Host Cell

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

### Individual/Subject

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

### Pharmaceutical Formulation

The term “pharmaceutical formulation” refers to a preparation which is in such form as to permit the biological activity of an active ingredient contained therein to be effective, and which contains no additional components which are unacceptably toxic to a subject to which the formulation would be administered. The “pharmaceutical formulation” may alternatively be called “pharmaceutical composition”.

### Pharmaceutically Acceptable Carrier

A “pharmaceutically acceptable carrier” refers to an ingredient in a pharmaceutical formulation/composition, other than an active ingredient, which is nontoxic to a subject. A pharmaceutically acceptable carrier includes, but is not limited to, a buffer, excipient, stabilizer, or preservative.

### Effective Amount

An “effective amount” of an agent, e.g., a pharmaceutical formulation, refers to an amount effective, at dosages and for periods of time necessary, to achieve the desired therapeutic or prophylactic result.

### Package Insert

The term “package insert” is used to refer to instructions customarily included in commercial packages of therapeutic products, that contain information about the indications, usage, dosage, administration, combination therapy, contraindications and/or warnings concerning the use of such therapeutic products.

### Treatment

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, a protein complex, polypeptide, fusion protein, or protein of the present disclosure are used to delay development of a disease or to slow the progression of a disease.

### Cancer

The terms “cancer” and “cancerous” as used herein, refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukemia and other lymphoproliferative disorders, and various types of head and neck cancer.

### Cell Proliferative Disorder

The terms “cell proliferative disorder” and “proliferative disorder” as used herein, refer to disorders that are associated with some degree of abnormal cell proliferation. In one embodiment, the cell proliferative disorder is cancer.

### B-Cell Neoplasms/Hodgkin's Disease

“B cell neoplasms” include Hodgkin's disease including lymphocyte predominant Hodgkin's disease (LPHD); non-Hodgkin's lymphoma (NHL); follicular center cell (FCC) lymphomas; acute lymphocytic leukemia (ALL); chronic lymphocytic leukemia (CLL); and Hairy cell leukemia. The non-Hodgkins lymphoma include low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS-related lymphoma and Waldenstrom's macroglobulinemia. Treatment of relapses of these cancers are also contemplated. LPHD is a type of Hodgkin's disease that tends to relapse frequently despite radiation or chemotherapy treatment. CLL is one of four major types of leukemia. A cancer of mature B-cells called lymphocytes, CLL is manifested by progressive accumulation of cells in blood, bone marrow and lymphatic tissues. Indolent lymphoma is a slow-growing, incurable disease in which the average patient survives between six and 10 years following numerous periods of remission and relapse.

### Breast Tumor

The term “breast tumor” or “breast cancer” refers to any tumor or cancer of the breast, including, e.g., adenocarcinomas, such as invasive or in situ ductal carcinoma, invasive or in situ lobular carcinoma, medullary carcinoma, colloid carcinoma, and papillary carcinoma; and less prevalent forms, such as cystosarcoma phylloides, sarcomas, squamous cell carcinomas, and carcinosarcomas.

### Colon Tumor

The term “colon tumor” or “colon cancer” refers to any tumor or cancer of the colon (the large intestine from the cecum to the rectum).

### Colorectal Tumor

The term “colorectal tumor” or “colorectal cancer” refers to any tumor or cancer of the large bowel, which includes the colon (the large intestine from the cecum to the rectum) and the rectum, including, e.g., adenocarcinomas and less prevalent forms, such as lymphomas and squamous cell carcinomas.

### Non-Hodgkin's Lymphoma

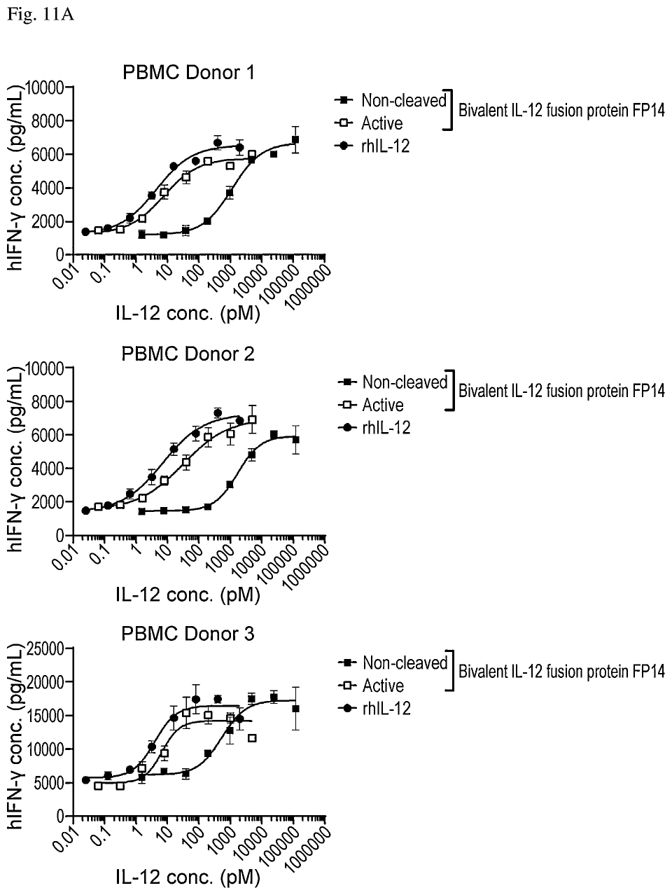

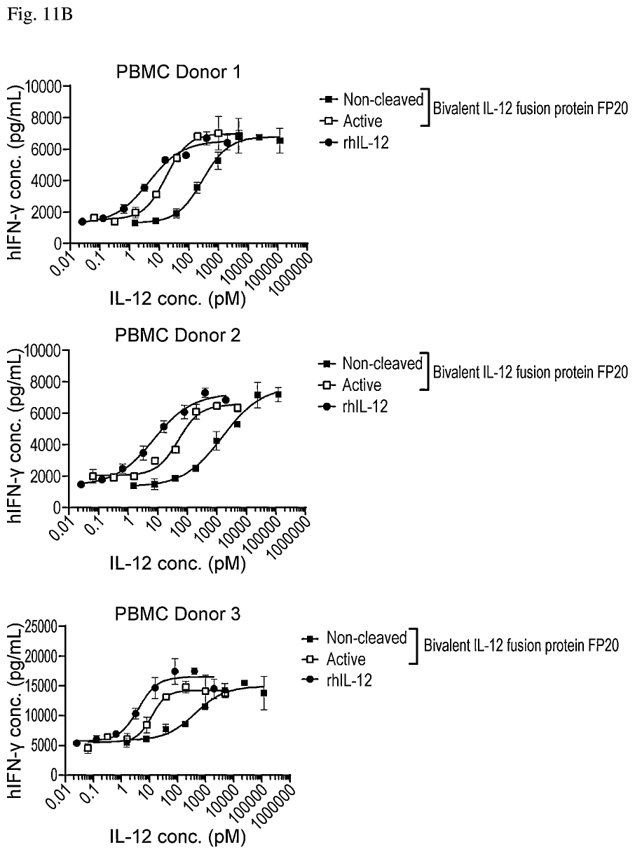

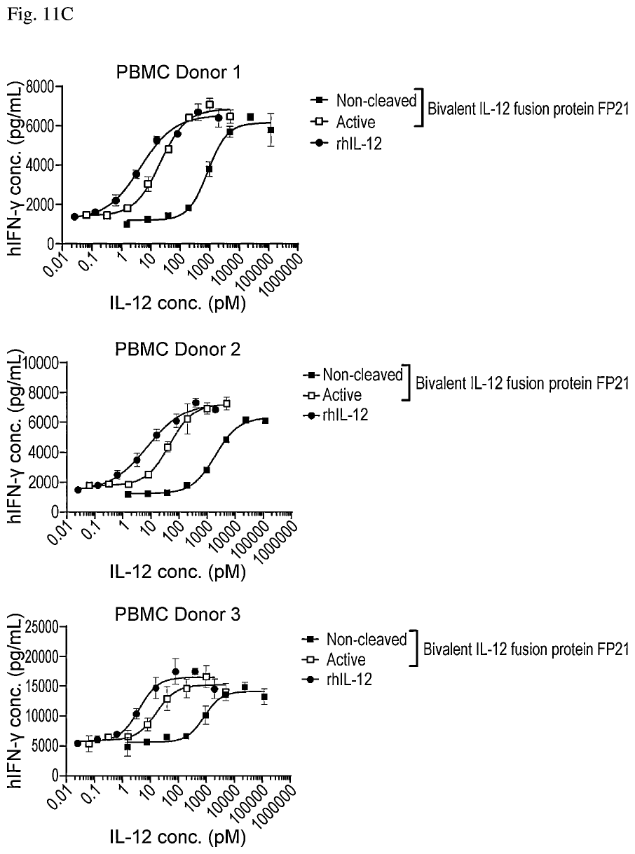

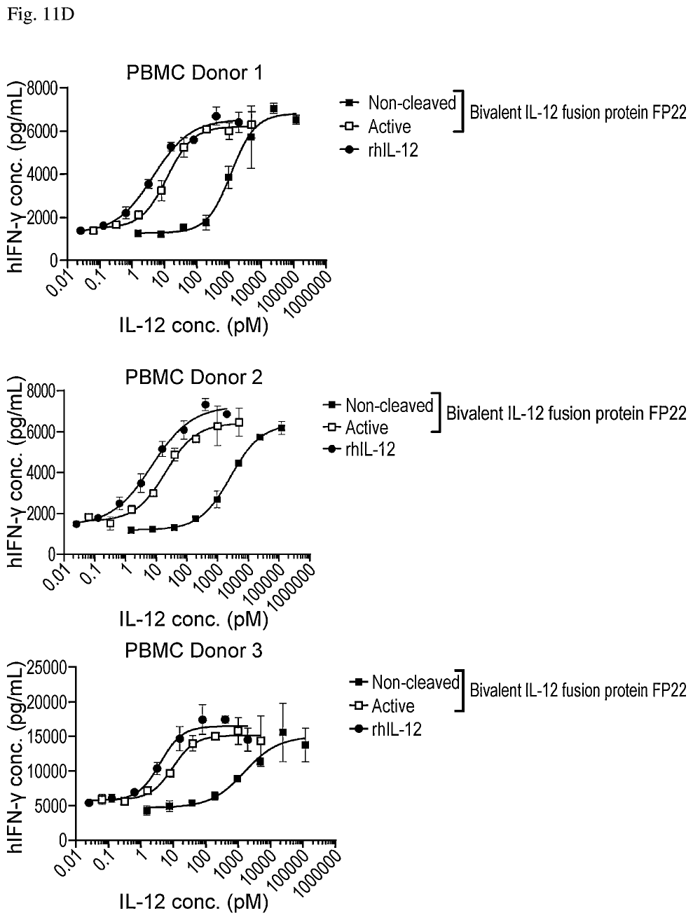

The term “non-Hodgkin's lymphoma” or “NHL”, as used herein, refers to a cancer of the lymphatic system other than Hodgkin's lymphomas. Hodgkin's lymphomas can generally be distinguished from non-Hodgkin's lymphomas by the presence of Reed-Sternberg cells in Hodgkin's lymphomas and the absence of said cells in non-Hodgkin's lymphomas. Examples of non-Hodgkin's lymphomas encompassed by the term as used herein include any that would be identified as such by one skilled in the art (e.g., an oncologist or pathologist) in accordance with classification schemes known in the art, such as the Revised European-American Lymphoma (REAL) scheme as described in Colour Atlas of Clinical Haematology, Third Edition; A. Victor Hoffbrand and John E. Pettit (eds.) (Harcourt Publishers Limited 2000) (see, in particular FIG. 11.57, 11.58 and/or 11.59). More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B cell chronic lymphocytic leukemia and/or prolymphocytic leukemia and/or small lymphocytic lymphoma, B-cell prolymphocytic lymphoma, immunocytoma and/or lymphoplasmacytic lymphoma, marginal zone B cell lymphoma, splenic marginal zone lymphoma, extranodal marginal zone—MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) T-cell lymphoblastic leukemia and/or lymphoma, adult T-cell lymphoma and/or leukemia, T cell chronic lymphocytic leukemia and/or prolymphacytic leukemia, large granular lymphocytic leukemia, mycosis fungoides and/or Sezary syndrome, extranodal natural killer/T-cell (nasal type) lymphoma, enteropathy type T-cell lymphoma, hepatosplenic T-cell lymphoma, subcutaneous panniculitis like T-cell lymphoma, skin (cutaneous) lymphomas, anaplastic large cell lymphoma, angiocentric lymphoma, intestinal T cell lymphoma, peripheral T-cell (not otherwise specified) lymphoma and angioimmunoblastic T-cell lymphoma.

### Ovarian Cancer

“Ovarian cancer” refers to a heterogeneous group of malignant tumors derived from the ovary. Approximately 90% of malignant ovarian tumors are epithelial in origin; the remainder are germ cell and stromal tumors. Epithelial ovarian tumors are classified into the following histological subtypes: serous adenocarcinomas (constituting about 50% of epithelial ovarian tumors); endometrioid adenocarcinomas (about 20%); mucinous adenocarcinomas (about 10%); clear cell carcinomas (about 5-10%); Brenner (transitional cell) tumors (relatively uncommon). The prognosis for ovarian cancer, which is the sixth most common cancer in women, is usually poor, with five-year survival rates ranging from 5-30%. For reviews of ovarian cancer, see Fox et al. (2002) “Pathology of epithelial ovarian cancer,” in Ovarian Cancer ch. 9 (Jacobs et al., eds., Oxford University Press, New York); Morin et al. (2001) “Ovarian Cancer,” in Encyclopaedic Reference of Cancer, pp. 654-656 (Schwab, ed., Springer-Verlag, New York). The present invention contemplates methods of diagnosing or treating any of the epithelial ovarian tumor subtypes described above, and in particular, the serous adenocarcinoma subtype.

### Relapsed

“Relapsed” refers to the regression of the patient's illness back to its former diseased state, especially the return of symptoms following an apparent recovery or partial recovery. Unless otherwise indicated, relapsed state refers to the process of returning to or the return to illness before the previous treatment including, but not limited to, chemotherapies and stem cell transplantation treatments.

### Refractory

“Refractory” refers to the resistance or non-responsiveness of a disease or condition to a treatment (e.g., the number of neoplastic plasma cells increases even though treatment is given). Unless otherwise indicated, the term “refractory” refers to a resistance or non-responsiveness to any previous treatment including, but not limited to, chemotherapies and stem cell transplantation treatments.

### Stomach Tumor

The term “stomach tumor” or “stomach cancer” as used herein, refers to any tumor or cancer of the stomach, including, e.g., adenocarcinomas (such as diffuse type and intestinal type), and less prevalent forms such as lymphomas, leiomyosarcomas, and squamous cell carcinomas.

### Tumor

The term “tumor” (or “tumor”) as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

### Inhibiting Cell Growth or Proliferation/Suppressing Cell Growth

“Inhibiting cell growth or proliferation” or “suppressing cell growth” means decreasing a cell's growth or proliferation by at least 10%, 20%, 30%, 40%, 50%, 60%, 70%, 80%, 90%, 95%, or 100%, and includes inducing cell death.

### Chemotherapeutic Agent

A “chemotherapeutic agent” refers to a chemical compound useful in the treatment of cancer. Examples of chemotherapeutic agents include alkylating agents such as thiotepa and cyclophosphamide (CYTOXAN (registered trademark)); alkyl sulfonates such as busulfan, improsulfan and piposulfan; aziridines such as benzodopa, carboquone, meturedopa, and uredopa; ethylenimines and methylol melamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL (registered trademark)); beta-lapachone; lapachol; colchicines; betulinic acid; a camptothecin (including the synthetic analogue topotecan (HYCAMTIN (registered trademark)), CPT-11 (irinotecan, CAMPTOSAR (registered trademark)), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, anthramycin, azaserine, bleomycins, cactinomycin, carubicin, carminomycin, carzinophilin, chromomycins, dactinomycin, daunorubicin, detorubicin, 6-diazo-5-oxo-L-norleucine, doxorubicin (including ADRIAMYCIN (registered trademark), morpholino-doxorubicin, cyanomorpholino-doxorubicin, 2-pyrrolino-doxorubicin, doxorubicin HCl liposome injection (DOXIL (registered trademark)), liposomal doxorubicin TLC D-99 (MYOCET (registered trademark)), peglylated liposomal doxorubicin (CAELYX (registered trademark)), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR (registered trademark)), tegafur (UFTORAL (registered trademark)), capecitabine (XELODA (registered trademark)), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatrexate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitraerine; pentostatin; phenamet; pirarubicin; losoxantrone; 2-ethylhydrazide; procarbazine; PSK (registered trademark) polysaccharide complex (JHS Natural Products, Eugene, OR); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE (registered trademark), FILDESIN (registered trademark)); dacarbazine; mannomustine; mitobronitol; mitolactol; pipobroman; gacytosine; arabinoside (“Ara-C”); thiotepa; taxoid, e.g., paclitaxel (TAXOL (registered trademark)), albumin-engineered nanoparticle formulation of paclitaxel (ABRAXANE™), and docetaxel (TAXOTERE (registered trademark)); chlorambucil; 6-thioguanine; mercaptopurine; methotrexate; platinum agents such as cisplatin, oxaliplatin (e.g., ELOXATIN (registered trademark)), and carboplatin; vincas, which prevent tubulin polymerization from forming microtubules, including vinblastine (VELBAN (registered trademark)), vincristine (ONCOVIN (registered trademark)), vindesine (ELDISINE (registered trademark), FILDESIN (registered trademark)), and vinorelbine (NAVELBINE (registered trademark)); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN (registered trademark)); bisphosphonates such as clodronate (for example, BONEFOS (registered trademark) or OSTAC (registered trademark)), etidronate (DIDROCAL (registered trademark)), NE-58095, zoledronic acid/zoledronate (ZOMETA (registered trademark)), alendronate (FOSAMAX (registered trademark)), pamidronate (AREDIA (registered trademark)), tiludronate (SKELID (registered trademark)), or risedronate (ACTONEL (registered trademark)); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE (registered trademark) vaccine and gene therapy vaccines, for example, ALLOVECTIN (registered trademark) vaccine, LEUVECTIN (registered trademark) vaccine, and VAXID (registered trademark) vaccine; topoisomerase 1 inhibitor (e.g., LURTOTECAN (registered trademark)); rmRH (e.g., ABARELIX (registered trademark)); BAY439006 (sorafenib; Bayer); SU-11248 (sunitinib, SUTENT (registered trademark), Pfizer); perifosine, COX-2 inhibitor (e.g. celecoxib or etoricoxib), proteosome inhibitor (e.g. PS341); bortezomib (VELCADE (registered trademark)); CCI-779; tipifarnib (R11577); sorafenib, ABT510; Bcl-2 inhibitor such as oblimersen sodium (GENASENSE (registered trademark)); pixantrone; EGFR inhibitors (see definition below); tyrosine kinase inhibitors (see definition below); serine-threonine kinase inhibitors such as rapamycin (sirolimus, RAPAMUNE (registered trademark)); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX (registered trademark)), 4-hydroxytamoxifen, toremifene (FARESTON (registered trademark)), idoxifene, droloxifene, raloxifene (EVISTA (registered trademark)), trioxifene, keoxifene, and selective estrogen receptor modulators (SERMs) such as SERM3; pure anti-estrogens without agonist properties, such as fulvestrant (FASLODEX (registered trademark)), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN (registered trademark)), and nonsteroidal aromatase inhibitors such as anastrazole (ARIMIDEX (registered trademark)), letrozole (FEMARA (registered trademark)) and aminoglutethimide, and other aromatase inhibitors include vorozole (RIVISOR (registered trademark)), megestrol acetate (MEGASE (registered trademark)), fadrozole, and 4(5)-imidazoles; lutenizing hormone-releasing hormone agonists, including leuprolide (LUPRON (registered trademark) and ELIGARD (registered trademark)), goserelin, buserelin, and triptorelin; sex steroids, including progestins such as megestrol acetate and medroxyprogesterone acetate, estrogens such as diethylstilbestrol and premarin, and androgens/retinoids such as fluoxymesterone, all transretinoic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

### Cytotoxic Agent

The term “cytotoxic agent” as used herein refers to a substance that inhibits or prevents a cellular function and/or causes cell death or destruction. Cytotoxic agents include, but are not limited to, radioactive isotopes (e.g., ²¹¹At, ¹³¹I, ¹²⁵I, ⁹⁰Y, ¹⁸⁶Re, ¹⁸⁸Re, ¹⁵³Sm, ²¹²Bi, ³²P, ²¹²Pb and radioactive isotopes of Lu); chemotherapeutic agents or drugs (e.g., methotrexate, adriamycin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

### Cytostatic Agent/Cell-Growth Suppressing Agent

The term “cytostatic agent” or “cell-growth suppressing agent” as used herein, interchangeably, refers to a compound or composition which arrests growth of a cell either in vitro or in vivo. Thus, a cytostatic agent may be one which significantly reduces the percentage of cells in S phase. Further examples of cytostatic agents include agents that block cell cycle progression by inducing G0/G1 arrest or M-phase arrest. The humanized anti-Her2 antibody trastuzumab (HERCEPTIN (registered trademark)) is an example of a cytostatic agent that induces G0/G1 arrest. Classical M-phase blockers include the vincas (vincristine and vinblastine), taxanes, and topoisomerase II inhibitors such as doxorubicin, epirubicin, daunorubicin, etoposide, and bleomycin. Certain agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W. B. Saunders, Philadelphia, 1995), e.g., p. 13. The taxanes (paclitaxel and docetaxel) are anticancer drugs both derived from the yew tree. Docetaxel (TAXOTERE (registered trademark), Rhone-Poulenc Rorer), derived from the European yew, is a semisynthetic analogue of paclitaxel (TAXOL (registered trademark), Bristol-Myers Squibb). Paclitaxel and docetaxel promote the assembly of microtubules from tubulin dimers and stabilize microtubules by preventing depolymerization, which results in the inhibition of mitosis in cells.

### Angiogenic Disorder

An “angiogenic disorder” refers to any dysregulation of angiogenesis, including both non-neoplastic and neoplastic conditions. Neoplastic conditions include but are not limited those described above [[see “Cancer” ]]. Non-neoplastic disorders include but are not limited to undesired or aberrant hypertrophy, arthritis, rheumatoid arthritis (RA), psoriasis, psoriatic plaques, sarcoidosis, atherosclerosis, atherosclerotic plaques, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertrophic bone formation, osteoarthritis (OA), refractory ascites, polycystic ovarian disease, endometriosis, 3rd spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), hemophilic joints, hypertrophic scars, inhibition of hair growth, Osler-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

### Anti-Angiogenic Agent

An “anti-angiogenic agent” refers to a compound which blocks, or interferes with to some degree, the development of blood vessels. An anti-angiogenic agent may, for instance, be a small molecule or antibody that binds to a growth factor or growth factor receptor involved in promoting angiogenesis. In one embodiment, an anti-angiogenic agent is an antibody that binds to vascular endothelial growth factor (VEGF), such as bevacizumab (AVASTIN (registered trademark)).

### Autoimmune Disease

“Autoimmune disease” refers to a non-malignant disease or disorder arising from and directed against an individual's own tissues. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobulinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

### Immunosuppressive Agents/Anti-Inflammatories

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL (registered trademark) methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine or cytokine receptor antibodies including anti-interferon-alpha, -beta, or -gamma antibodies, anti-tumor necrosis factor (TNF)-alpha antibodies (infliximab (REMICADE (registered trademark)) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists (such as ACTEMRA™ (tocilizumab)); anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; transforming growth factor-beta (TGF-beta); streptodornase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as BAFF antibodies and BR3 antibodies and zTNF4 antagonists (for review, see Mackay and Mackay, Trends Immunol., 23:113-5 (2002) and see also definition below); biologic agents that interfere with T cell helper signals, such as anti-CD40 receptor or anti-CD40 ligand (CD154), including blocking antibodies to CD40-CD40 ligand (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9. Some preferred immunosuppressive agents herein include cyclophosphamide, chlorambucil, azathioprine, leflunomide, MMF, or methotrexate.

### II. Exemplary Embodiments of the Present Invention

In one embodiment, the present invention relates to a protein complex comprising or consisting of two subunits associated with each other, wherein each subunit comprises a first polypeptide and a second polypeptide, wherein the first polypeptide comprises or consists of a light chain variable domain (VL) and a light chain constant domain (CL), and the second polypeptide is a fusion protein represented by the general formula (I), from the N- to the C-terminus:

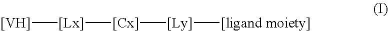

wherein:
Lx represents a cleavable peptide linker comprising a protease cleavage site as defined herein,
Cx represents a constant domain as defined herein associated with the CL of the first polypeptide, optionally, wherein Cx comprises or consists of, from the N- to the C-terminus, a CH1 domain, a second peptide linker, a CH2 domain, and a CH3 domain, and
optionally wherein one or more amino acid residues are modified from or to cysteine;
Ly represents a third peptide linker as defined herein,
VH represents a heavy chain variable domain (VH), wherein the VH is associated with the VL of the first polypeptide to form a ligand-binding domain, wherein the VH and the VL are characterized by their amino acid sequences as defined in this application, and
wherein the ligand moiety is a protease resistant single-chain IL-12 fusion protein as defined herein. In one embodiment, the protein complex is a bivalent homodimer fusion protein complex.

In one embodiment, the present invention relates to a protein complex comprising two polypeptides, wherein each polypeptide is a fusion protein, and each polypeptide is represented by the general formula (II), from the N- to the C-terminus:

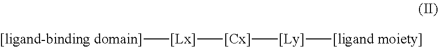

wherein:
Lx represents a cleavable peptide linker comprising a protease cleavage site as defined herein,
Cx represents a constant domain as defined herein comprising, from the N- to the C-terminus, a CH1 domain, a second peptide linker, a CH2 domain, and a CH3 domain, and
optionally wherein one or more amino acid residues are modified from or to cysteine;
Ly represents a third peptide linker as defined herein,
wherein the ligand-binding domain comprises a heavy chain variable domain (VH) associated with a light chain variable domain (VL), wherein VL is associated with a light chain constant domain (CL), wherein CL is associated with Cx, and wherein VH and VL are characterized by their amino acid sequences as defined in this application; and
wherein the ligand moiety is a protease resistant single-chain IL-12 fusion protein as defined herein. In one embodiment, the protein complex is a bivalent homodimer fusion protein complex.

In a first state, the ligand moiety of the present invention is bound by the ligand binding domain and the biological activity of the ligand is attenuated, and in a second state, the biological activity of the ligand is restored, and the protein complex in the first state has a longer half-life in blood than in the second state, and switching from the first state to the second state is mediated by the presence of a protease.

The difference between the “first state” and “second state” in the present invention is the absence/presence of protease cleavage. The phrase “in a first state” may be rephrased as “before the protease cleavage site is cleaved by the protease” or “when the protease cleavage site is uncleaved by the protease”, or “uncleaved state”, or “non-cleaved state”, or “inactive state”. The phrase “in a second state” may be rephrased as “after the protease cleavage site is cleaved by the protease” or “when the protease cleavage site is cleaved by the protease”, or “cleaved state”, or “active state”. The same applies to other embodiments described herein.

In aspects of the present invention, upon cleavage of Lx, the heavy chain variable domain (VH) dissociates from the light chain variable domain (VL). The dissociation is promoted by at least one amino acid modification performed at the interface between said portion or domain and a corresponding interacting portion or domain. For example, where the domain is VH, as in the aspects herein, a corresponding interacting domain thereof is VL, and where the domain is VL, a corresponding interacting domain thereof is VH. The process of dissociation upon cleavage is also called VH release, as VH will be removed from the protein complex upon cleavage. Upon VH release the interaction between the ligand binding domain and the ligand moiety becomes weaker or is even abolished, whereupon the ligand moiety is released from the ligand binding domain.

Additional protein complexes are disclosed in International Publication No. WO2023/002952, which is incorporated herein by reference in its entirety for all purposes.

### Protease Cleavage Site

In the present invention, the protein complex comprises at least one protease cleavage site. The protease cleavage site of the present invention is comprised in the cleavable peptide linker Lx as defined herein. Upon protease cleavage, the ligand becomes released or unbound from the ligand-binding domain and the biological activity of the ligand to bind its binding partner is restored. As used herein, the phrase “release/releasing the ligand moiety/molecule” or “the ligand moiety/molecule is released” means that the ligand moiety/molecule becomes able to exert and/or increase its biological activity through interacting with a binding partner thereof compared with the biological activity of the ligand moiety/molecule bound with the uncleaved ligand-binding domain/moiety/molecule, but does not refer to or pose limitations on any particular level of release or any particular mode of action by which the ligand moiety/molecule is released. In the present invention, the released ligand still remains linked to the rest of the protein complex, as it is part of a single-chain polypeptide.

Protease cleavage at the protease cleavage site can affect, e.g., restore, the biological activity of a ligand which can be bound by the ligand-binding domain. As used herein, for example, the phrase “biological activity is restored” refers to the state when the ligand transits from a (first) state when it is bound to the ligand-binding domain in the uncleaved state and unable to interact with a binding partner (i.e., the biological activity is attenuated due to the absence of the interaction) to a (second) state when it is not bound to the ligand-binding domain in the cleaved state and able to interact with a binding partner and exert its biological activity thereof. The physiological activity of the ligand to bind its binding partner is attenuated in the first state when it is bound by the ligand-binding domain and is restored in the second state when it is unbound from the ligand-binding domain in the presence of protease.

In one embodiment, the ligand-binding domain binds to the ligand moiety/molecule more weakly (i.e. ligand binding is attenuated) in a cleaved state compared with an uncleaved state. In a preferred embodiment, the ligand-binding moiety/molecule does not bind to the ligand or ligand moiety (i.e., ligand binding is abolished) in a cleaved state compared with an uncleaved state. In an embodiment in which the ligand-binding moiety/molecule binds to the ligand moiety/molecule by antigen-antibody reaction, the attenuation of the ligand binding or lack thereof, can be evaluated on the basis of the biological activity of the ligand-binding moiety/molecule. In a preferred embodiment of the present invention, the ligand binding in a cleaved state is abolished. This is due to the structure of the interface between the VH domain and the VL domain, which has been optimized by mutations of the amino acid sequence for efficient promotion of dissociation of VH from VL upon protease cleavage.

The phrase “ligand binding is attenuated” means that the amount of a test ligand binding molecule bound with the ligand is, for example, 90% or less, 80% or less, 70% or less, 60% or less, 50% or less, preferably 45% or less, 40% or less, 35% or less, 30% or less, 20% or less, or 15% or less, particularly preferably 10% or less, 9% or less, 8% or less, 7% or less, 6% or less, 5% or less, 4% or less, 3% or less, 2% or less, or 1% or less, of the amount of a control ligand binding molecule bound with the ligand on the basis of the measurement method described herein. The desired index may be appropriately used as an index for binding activity. For example, a dissociation constant (KD) may be used. In the case of using a dissociation constant (KD) as an index for evaluating binding activity, a larger dissociation constant (KD) of the test ligand binding molecule for the ligand than that of a control ligand binding molecule for the ligand means that the test ligand binding molecule has weaker binding activity against the ligand than that of the control ligand binding molecule. The phrase “ligand binding ability is attenuated” means that the dissociation constant (KD) of the test ligand binding molecule for the ligand is, for example, at least 2 times, preferably at least 5 times or at least 10 times, particularly preferably at least 100 times the dissociation constant (KD) of the control ligand binding molecule for the ligand.

Examples of the control ligand binding molecule include an uncleaved form of the ligand-binding moiety/molecule.

In the present invention, the biological activity of the ligand moiety/molecule is attenuated by binding to the ligand-binding domain of the uncleaved ligand-binding moiety/molecule. Examples of the embodiments in which the biological activity of the ligand is attenuated include, but are not limited to, embodiments in which the binding of the ligand moiety/molecule to the ligand-binding domain of the uncleaved ligand binding moiety/molecule substantially or significantly interferes or competes with the binding of the ligand to its binding partner. In the case of using one or more antibody fragment(s) having ligand neutralizing activity as the ligand-binding moiety/molecule, the ligand-binding moiety/molecule bound with the ligand is capable of attenuating, and to the larger extent of attenuating, i.e. inhibiting the biological activity of the ligand by exerting its neutralizing activity.

In one embodiment of the present invention, preferably, the uncleaved ligand-binding moiety/molecule can sufficiently neutralize the biological activity of the ligand moiety by binding to the ligand moiety. Specifically, the biological activity of the ligand moiety/molecule when bound with the uncleaved ligand-binding moiety/molecule is preferably lower than that of the ligand moiety/molecule when unbound from the cleaved ligand-binding moiety/molecule. The biological activity of the ligand when bound with the uncleaved ligand binding molecule can be, for example, 90% or less, preferably 80% or less, 70% or less, 60% or less, 50% or less, 40% or less, or 30% or less, particularly preferably 20% or less, 10% or less, 9% or less, 8% or less, 7% or less, 6% or less, 5% or less, 4% or less, 3% or less, 2% or less, or 1% or less, of the biological activity of the ligand when unbound from the cleaved ligand binding molecule, though not limited thereto. The administration of the ligand-binding moiety/molecule, which sufficiently neutralizes the biological activity of the ligand, can be expected to prevent the ligand from exerting its biological activity before arriving at a target tissue.

In one embodiment of the present invention, the binding activity of the cleaved ligand binding moiety or molecule against the ligand moiety or molecule is preferably lower than that of an in vivo natural ligand binding partner (e.g., natural receptor for the ligand) against the ligand. The binding activity of the cleaved ligand binding moiety/molecule against the ligand moiety/molecule exhibits, for example, 90% or less, preferably 80% or less, 70% or less, 60% or less, 50% or less, 40% or less, or 30% or less, particularly preferably 20% or less, 10% or less, 9% or less, 8% or less, 7% or less, 6% or less, 5% or less, 4% or less, 3% or less, 2% or less, or 1% or less, of the amount of the ligand bound with the in vivo natural binding partner (per unit binding partner), though not limited thereto. The desired index may be appropriately used as an index for binding activity. For example, a dissociation constant (KD) may be used. In the case of using a dissociation constant (KD) as an index for evaluating binding activity, a larger dissociation constant (KD) of the cleaved ligand binding moiety/molecule for the ligand than that of the in vivo natural binding partner for the ligand means that the cleaved ligand binding molecule has weaker binding activity against the ligand than that of the in vivo natural binding partner. The dissociation constant (KD) of the cleaved ligand binding molecule for the ligand is, for example, at least 1.1 times, preferably at least 1.5 times, at least 2 times, at least 5 times, or at least 10 times, particularly preferably at least 100 times the dissociation constant (KD) of the in vivo natural binding partner for the ligand. The ligand binding molecule having only low binding activity against the ligand or hardly having binding activity against the ligand after cleavage guarantees that the ligand is released by the cleavage of the ligand binding molecule and can be expected to be prevented from binding to another ligand molecule again.

The ligand desirably restores the suppressed biological activity after cleavage of the ligand binding molecule. Desirably, the ligand binding of the cleaved ligand binding molecule is attenuated so that the ligand biological activity-inhibiting function of the ligand binding molecule is also attenuated. Those skilled in the art can confirm the biological activity of the ligand by a known method, for example, a method of detecting the binding of the ligand to its binding partner as disclosed herein.

In the present specification, the phrase “attenuated binding activity” as used herein, when referring to the binding activity of the ligand for its binding partner, refers to reduced or decreased binding activity when compared with the binding activity of the ligand in the cleaved state of the fusion polypeptide (also referenced herein as a protein complex), and the degree of reduction or decrease is not limited, and includes complete abolishment of activity. Similarly, the phrase “suppressed biological activity” and “neutralizing the biological activity of the ligand” may be used herein interchangeably to demonstrate a reduction, including and not limited to complete elimination, of the binding activity of the ligand for its binding partner when the ligand is bound to the ligand-binding domain of the fusion polypeptide before protease cleavage.

In the present specification, the phrase “biological activity is restored” refers to the state when the ligand transits from a (first) state when it is bound to the ligand-binding moiety in the uncleaved state and unable to interact with a binding partner to a (second) state when it is not bound to the ligand binding moiety in the cleaved state and able to interact with a binding partner and exert its biological activity thereof. The term “restored” refers to the return of the ability of the ligand to interact with a binding partner and exert its biological activity thereof, where this ability had been inhibited when the ligand was bound to the ligand binding domain in the uncleaved state. It includes any degree of interaction or increased interaction with a binding partner sufficient to exert its biological activity thereof upon binding. In the present invention, the ligand-binding moiety/molecule comprises a protease cleavage site placed within or near the ligand-binding domain in the ligand-binding moiety. In the presence of protease, the ligand becomes unbound to the ligand-binding moiety and free to interact with a binding partner and exert its biological activity. In the present invention, the interaction of ligand to a binding partner to exert its biological activity occurs while the ligand remains bound by a non-cleavable peptide linker to the C-terminal end of the Fc region of the ligand-binding moiety. The term “biological activity” as used herein, includes but is not limited to, the physiological activity of the ligand (e.g. ligand interaction with its natural binding partner such as a ligand receptor).

The biological activity of the ligand to bind its ligand binding partner can be confirmed by well-known methods such as FACS, ELISA, BIACORE® using ALPHA (ALPHA (amplified luminescent proximity homogeneous assay) screening or surface plasmon resonance (SPR) phenomena, or BLI (bio-layer interferometry) (Octet) (Proc. Natl. Acad. Sci. USA (2006) 103 (11), 4005-4010). The ALPHA screening is carried out on the basis of the following principle according to ALPHA technology using two beads, a donor and an acceptor. Luminescence signals are detected only when these two beads are located in proximity through the interaction between a molecule bound with the donor bead and a molecule bound with the acceptor bead. A laser-excited photosensitizer in the donor bead converts ambient oxygen to singlet oxygen in an excited state. The singlet oxygen diffuses around the donor bead and reaches the acceptor bead located in proximity thereto to thereby cause chemiluminescent reaction in the bead, which finally emits light. In the absence of the interaction between the molecule bound with the donor bead and the molecule bound with the acceptor bead, no chemiluminescent reaction occurs because singlet oxygen produced by the donor bead does not reach the acceptor bead.

For example, a biotin-labeled ligand binding partner is bound to the donor bead, while a glutathione S transferase (GST)-tagged ligand is bound to the acceptor bead. In the absence of an untagged competitor ligand binding partner, the ligand binding partner interacts with the ligand to generate signals of 520 to 620 nm. The untagged ligand binding partner competes with the tagged ligand binding partner for the interaction with the ligand. Decrease in fluorescence resulting from the competition can be quantified to determine relative binding affinity. The biotinylation of the ligand binding partner such as an antibody using sulfo-NHS-biotin or the like is known in the art. A method which involves, for example: fusing a polynucleotide encoding the ligand in flame with a polynucleotide encoding GST; expressing a GST-fused ligand from cells or the like carrying a vector that permits expression of the resulting fusion gene; and purifying the GST-fused ligand using a glutathione column can be appropriately adopted as a method for tagging the ligand with GST. The obtained signals are preferably analyzed using, for example, software GRAPHPAD PRISM (GraphPad Software, Inc., San Diego) adapted to a one-site competition model based on nonlinear regression analysis.

One (ligand) of the substances between which the interaction is to be observed is immobilized onto a thin gold film of a sensor chip. The sensor chip is irradiated with light from the back such that total reflection occurs at the interface between the thin gold film and glass. As a result, a site having a drop in reflection intensity (SPR signal) is formed in a portion of reflected light. The other (analyte) of the substances between which the interaction is to be observed is flowed on the surface of the sensor chip and bound to the ligand so that the mass of the immobilized ligand molecule is increased to change the refractive index of the solvent on the sensor chip surface. This change in the refractive index shifts the position of the SPR signal (on the contrary, the dissociation of the bound molecules gets the signal back to the original position). The BIACORE® system plots on the ordinate the amount of the shift, i.e., change in mass on the sensor chip surface, and displays time-dependent change in mass as assay data (sensorgram). Kinetics: an association rate constant (ka) and a dissociation rate constant (kd) are determined from the curve of the sensorgram, and a dissociation constant (KD) is determined from the ratio between these constants. Inhibition assay or equilibrium analysis is also preferably used in the BIACORE® method. Examples of the inhibition assay are described in Proc. Natl. Acad. Sci. USA (2006) 103 (11), 4005-4010, and examples of the equilibrium analysis are described in Method Enzymol. 2000; 323: 325-40.

The phrase “biological activity is restored” as used herein, does not place a limitation on the degree of binding of the ligand to its binding partner as long as biological activity resulting from the binding is observed in any measurement methods, such as those described herein. It can include 1% or more, 2% or more, 3% or more, 4% or more, 5% or more, 6% or more, 7%, or more 8% or more, 9% or more, 10% or more, 20% or more, 30% or more, 40% or more, 50% or more, 60% or more, 70% or more, 80% or more, or 90% or more increase in interaction between the ligand and its binding partner when comparing the ligand in a cleaved state and uncleaved state. The desired index may be appropriately used as an index for binding activity. For example, an association rate constant (Kon) may be used. In the case of using an association constant (Kon), a larger association constant of the ligand binding partner for a test ligand (i.e. restored ligand in a cleaved state) than that of a control ligand (i.e. ligand in an uncleaved state) means stronger ligand binding activity of the test ligand for the ligand binding partner than that of the control ligand. In some embodiments, the association constant is at least 2 times, preferably at least 5 times, or at least 10 times, particularly preferably at least 100 times that of the control ligand for the ligand binding partner.

In the case of detecting the biological activity of the ligand using, for example, Octet, the antibody for ligand detection that recognizes the ligand is biotinylated and contacted with a biosensor. Then, binding to the ligand in a sample can be measured to detect the restoration of the ligand binding activity. Specifically, the amount of the ligand is measured in a sample containing the ligand binding molecule before protease treatment or after protease treatment and the ligand, using the antibody for ligand detection. The amount of the ligand detected in the sample can be compared between before and after protease treatment to detect the release of the ligand. Alternatively, the amount of the ligand is measured in a sample containing protease, the ligand binding molecule, and the ligand and a sample containing the ligand binding molecule and the ligand without containing protease, using the antibody for ligand detection. The amount of the ligand detected in the sample can be compared between the presence and absence of protease to determine the restoration of ligand binding ability of the ligand moiety. As the ligand binding molecule is fused with the ligand to form a fusion protein (also referenced herein as a protein complex), the amount of the ligand is measured in a sample containing the fusion protein before protease treatment or after protease treatment, using the antibody for ligand detection. The amount of the ligand detected in the sample can be compared between before and after protease treatment to determine the restoration of ligand binding ability of the ligand. Alternatively, the amount of the ligand is measured in a sample containing protease and the fusion protein and a sample containing the fusion protein without containing protease, using the antibody for ligand detection. The amount of the ligand detected in the sample can be compared between the presence and absence of protease to determine restoration of the ligand binding ability of the ligand. More specifically, the restoration of ligand binding activity of the ligand can be detected by a method described in Examples of the present application.

In some embodiments, the physiological activity of the ligand (i.e. ligand interaction with its natural binding partner such as the ligand receptor) is attenuated upon binding to the ligand-binding domain, the restoration of this physiological activity of the ligand can be detected by a method of measuring the physiological activity of the ligand in a sample. Specifically, the physiological activity of the ligand can be measured in a sample containing the ligand binding molecule before protease treatment or after protease treatment and compared between before and after protease treatment to detect the restoration of its binding ability. Alternatively, the physiological activity of the ligand can be measured in a sample containing protease, the ligand binding molecule, and a sample containing the ligand binding molecule without containing protease and compared between these samples to detect the restoration of the binding ability of the ligand. When the ligand binding molecule is bound to the ligand to form a protein complex, the physiological activity of the ligand can be measured in a sample containing the protein complex before protease treatment or after protease treatment and compared between before and after protease treatment to detect the restoration of its binding ability. Alternatively, the physiological activity of the ligand can be measured in a sample containing protease and the protein complex and a sample containing the protein complex without containing protease and compared between these samples to detect the restoration of its binding ability.

In some embodiments of the present invention, the uncleaved ligand binding molecule forms a complex with the ligand through antigen-antibody binding. In a more specific embodiment, the complex of the ligand binding molecule and the ligand is formed through a noncovalent bond, for example, antigen-antibody binding, between the ligand binding molecule and the ligand.

In the present invention, an uncleaved ligand binding molecule is bound to a ligand molecule to form a protein complex. The ligand binding domain of the ligand binding moiety and the ligand moiety in the protein complex further interact with each other through antigen-antibody binding. The antigen-antibody binding is attenuated by the cleavage of the ligand binding moiety/molecule. In short, the ligand binding of the ligand binding moiety/molecule is attenuated.

In the present invention, the ligand moiety or molecule is connected to a C-terminal region of the ligand-binding moiety or molecule via a peptide linker. As used herein, the term “C-terminal region” refers to a region of a polypeptide that extends from an internal amino acid residue in the polypeptide to the C-terminal amino acid residue of the polypeptide. In certain embodiments where the ligand-binding moiety/molecule is, for example, in the form of an antibody or in the form of an antibody fragment that contains an Fc region, the C-terminal region of the ligand-binding moiety/molecule typically refers to a region of the 1st to 250th amino acid residues from the C-terminus of the ligand-binding moiety/molecule. In a preferred embodiment, the ligand moiety/molecule is connected to the C-terminal amino acid residue of the ligand-binding moiety/molecule via a peptide linker. The peptide linker may be attached to the ligand moiety/molecule and to the C-terminal region of the ligand-binding moiety/molecule by any covalent bonds such as peptide bonds. The length of the peptide linker is not particularly limited as long as it allows the ligand moiety/molecule to bind to the ligand-binding domain in the ligand-binding moiety/molecule. The above-mentioned peptide linker may or may not contain a protease cleavage site. In a preferred embodiment, the above-mentioned peptide linker does not contain a protease cleavage site.

In the present invention, the ligand moiety/molecule of the present invention is IL-12. In one embodiment, the ligand moiety/molecule of the present invention is IL-12 and the IL-12 is connected with C-terminal amino acid residue of the ligand-binding moiety/molecule via a peptide linker attached to p35 subunit of IL-12 or p40 subunit of IL-12. In a preferred embodiment, the IL-12 is connected with C-terminal amino acid residue of the ligand-binding moiety/molecule via a peptide linker attached to p40 subunit of IL-12.

In one embodiment, the ligand moiety/molecule of the present invention is IL-12 and the IL-12 is connected with C-terminal amino acid residue of the ligand-binding moiety/molecule via a peptide linker attached to the N-terminus of the p35 subunit of IL-12 or the p40 subunit of IL-12. In one embodiment, the ligand moiety/molecule of the present invention is IL-12 and the IL-12 is connected with C-terminal amino acid residue of the ligand-binding moiety/molecule via a peptide linker attached to the N-terminus of the p40 subunit of IL-12. In one embodiment, the IL-12 is a single chain IL12 wherein the p35 subunit of IL-12 is linked to the p40 subunit via a peptide linker. In one embodiment, the ligand-binding domain of the present invention is connected to a hinge region comprised in the ligand-binding moiety via a peptide linker. In a preferred embodiment, the ligand-binding moiety of the present invention may further comprise a CH1 region which is connected to a hinge region via a peptide linker. The peptide linker can be inserted between CH1 and hinge on either side of the linker. In embodiments, the fusion protein (or ligand-binding moiety) of the invention comprises a constant region comprising a peptide linker. In some embodiments, the constant region comprises a hinge region comprising a peptide linker. The peptide linker may be comprised at any position before/within the hinge region. The peptide linker may be comprised between CH1 and the hinge region, i.e., before the amino acid sequence EPKSC (SEQ ID NO: 101 or 360) in the hinge region (note: the initial residue (E) is at position 216 (EU numbering)). The peptide linker may be comprised after the amino acid sequence EPKSC (SEQ ID NO: 101 or 360) in the hinge region. Examples of the position of the peptide linker include, but are not limited to, the following:

[Peptide linker]EPKSCDKTHTCPPCP
(see SEQ ID NO: 31; examples include ″C1″ type);
EPKSC[Peptide linker]DKTHTCPPCP
(see SEQ ID NO: 34; examples include ″C2″ type);
and
[Peptide linker]EPKSSDKTHTCPPCP
(see SEQ ID NOs: 37 and 39; examples include ″C3″
and ″C4″ types);
and
EPKSCDKTHT[Peptide linker]CPPCP
(see SEQ ID NO: 41; examples include ″C5″ type).

In some embodiments, the peptide linker ([Peptide linker] indicated above) is a GS linker mentioned herein, such as (GS)₂, (GGGGS: SEQ ID NO: 6)₂.
The suitable peptide linker above may be readily selected and can be preferably selected from among different lengths such as 1 amino acid (Gly, etc.) to 300 amino acids, 2 amino acids to 200 amino acids, or 3 amino acids to 100 amino acids including 4 amino acids to 100 amino acids, 5 amino acids to 100 amino acids, 5 amino acids to 50 amino acids, 5 amino acids to 30 amino acids, 5 amino acids to 25 amino acids, or 5 amino acids to 20 amino acids.
Examples of the peptide linker include, but are not limited to, glycine polymers (G)n, glycine-serine polymers (including e.g., (GS)n, (GGGGS: SEQ ID NO: 6)n and (GGGS: SEQ ID NO: 1)n, wherein n is an integer of at least 1), glycine-alanine polymers, alanine-serine polymers, and other flexible linkers well known in conventional techniques.
Examples of the peptide linker can include, but are not limited to,

(GGGGS, SEQ ID NO: 6)
Gly Gly Gly Gly Ser
(Gly Gly Gly Gly Ser (GGGGS, SEQ ID NO: 6))n
(GGGGA, SEQ ID NO: 23)
Gly Gly Gly Gly Ala
(GGGGE, SEQ ID NO: 24)
Gly Gly Gly Gly Glu
(GGGS, SEQ ID NO: 1)
Gly Gly Gly Ser
(Gly Gly Gly Ser (GGGS, SEQ ID NO: 1))n
(GGGA, SEQ ID NO: 25)
Gly Gly Gly Ala
(GGGE, SEQ ID NO: 26)
Gly Gly Gly Glu
(QQQG, SEQ ID NO: 27)
Gln Gln Gln Gly
(QQQQG, SEQ ID NO: 28)
Gln Gln Gln Gln Gly
(SSSG, SEQ ID NO: 29)
Ser Ser Ser Gly
(SSSSG, SEQ ID NO: 30)
Ser Ser Ser Ser Gly

wherein n is an integer of 1 or larger.
In embodiments herein, n may be 1, 2, 3, 4, 5, 6, 7, 8, 9, 10, or more.

However, the length and sequence of the peptide linker can be appropriately selected by those skilled in the art according to the purpose. The presence of a linker (such as the GS linker) in the hinge region between the Fab and Fc may result in heterogeneity in the disulfide bond formation between HC (heavy-chain constant region) and LC (light-chain constant region). In some embodiments, a fusion protein of the invention is a homodimer of a light chain and heavy chain.

In the present invention, the heavy chain comprises a cleavable peptide linker (“Lx”, e.g., SEQ ID NO: 199 or SEQ ID NO:194) introduced between the boundary of heavy-chain variable region and Constant Region 1 (“C1”, e.g., SEQ ID NO: 31, or SEQ ID NO: 66), or Constant Region 2 (“C2”, e.g. SEQ ID NO: 34, or SEQ ID NO: 70), or Constant Region 3 (“C3”, e.g. SEQ ID NO: 37, or SEQ ID NO: 73), or Constant Region 4 (“C4”, e.g. SEQ ID NO: 39/or SEQ ID NO: 75), or Constant Region 5 (“C5”, e.g. SEQ ID NO: 41, or SEQ ID NO: 97), or Constant Region 6 (“C6”, e.g. SEQ ID NO: 111), or Constant Region 7 (“C7”, e.g. SEQ ID NO: 131), or Constant Region 8 (“C8”, e.g. SEQ ID NO: 192), or Constant Region 9 (“C9”, e.g. SEQ ID NO: 196). A single-chain IL-12 may be attached to the C-terminus of Fc domain via a linker such as the GS linker (“L4”, e.g., SEQ ID NO: 32 or SEQ ID NO: 68; or “L5”, e.g. SEQ ID NO: 40, or SEQ ID NO: 92).

In some embodiments, a “C1” variant is used. In some embodiments, the variant is Ab1-L1-C1-L4-IL12 (Bivalent IL-12 fusion Ab1) which is a homodimer comprising a light chain of SEQ ID NO: 20 and a heavy chain of SEQ ID NO: 22. To promote homogeneity, improved forms (further variants) may be generated as follows.

In some embodiments, a “C2” variant is used. The heavy chain of this variant may comprise a linker such as a cleavable peptide linker (“Lx”, e.g., SEQ ID NO: 199 or SEQ ID NO:194) introduced between the boundary of heavy-chain variable region and Constant Region 2 (“C2”, e.g., SEQ ID NO: 34). In the Constant Region 2, a non-limiting example of the positional shift of a linker (e.g., the GS linker (GGGGSGGGGS (SEQ ID NO: 6)₂) present in the hinge region) is shown below:

from [GGGGSGGGGSEPKSCDKTHTCPPCP] (SEQ ID NO: 42)
to [EPKSCGGGGSGGGGSDKTHTCPPCP] (SEQ ID NO: 100 or 359) (the initial residue (E) is at position 216 (EU numbering)). The shifted position of the linker can be appropriately selected or designed by those skilled in the art according to the purpose, i.e., to promote homogeneity. The positional shift of the linker can promote or facilitate disulfide (cysteine-cysteine (Cys-Cys)) bond formation between Cys at position 220 (C220) (EU numbering) of the heavy chain and Cys at position 214 (C214) (EU numbering) of the light chain. A single-chain ligand (such as IL-12) may be attached to the C-terminus of Fc domain via a linker such as the GS linker (“L4”, e.g., SEQ ID NO: 32); alternatively, the linker may be a cleavable linker (“L3”, e.g., SEQ ID NO: 21). In some embodiments, the variant is Ab1-L1-C2-L4-IL12 which is a homodimer comprising a light chain of SEQ ID NO: 20 and a heavy chain of SEQ ID NO: 33.

In some embodiments, a “C3” variant is used. In this variant, the light chain may comprise C214S (EU numbering) modification, and the heavy chain may comprise C220S (EU numbering) modification which result in no disulfide bond formation between the heavy chain and light chain, i.e., between position 220 (EU numbering) of the heavy chain and position 214 (EU numbering) of the light chain. The heavy chain of this variant may comprise a linker such as a cleavable peptide linker (“Lx”, e.g., SEQ ID NO: 199 or SEQ ID NO:194) introduced into between the boundary of heavy-chain variable region and Constant Region 3 (“C3”, e.g., SEQ ID NO: 37). A single-chain IL-12 may be attached to the C-terminus of Fc domain via a linker such as the GS linker (“L4”, e.g., SEQ ID NO: 32). In some embodiments, the variant is Ab1-L1-C3-L4-IL12 which is a homodimer comprising a light chain of SEQ ID NO: 35 and heavy chain of SEQ ID NO: 36.

In some embodiments, a C4 variant is used. The use of a “C4” variant is preferred herein. In this variant, the light chain may not comprise the above-mentioned modification(s), while the heavy chain may comprise S131C (EU numbering) and C220S (EU numbering) modifications which result in disulfide bond formation between the heavy chain and light chain, i.e., between Cys at position 131 (C131) (EU numbering) of the heavy chain and Cys at position 214 (C214) (EU numbering) of the light chain. The heavy chain of this variant may comprise a linker such as a cleavable peptide linker (“Lx”, e.g., SEQ ID NO: 199 or SEQ ID NO:194) introduced between the boundary of heavy-chain variable region and Constant Region 4 (“C4”, e.g., SEQ ID NO: 39 or 75). A single-chain IL-12 may be attached to the C-terminus of Fc domain via a linker such as the GS linker (L4, e.g., SEQ ID NO: 32). In some embodiments, the variant is Ab1-L1-C4-L4-IL12 which is a homodimer comprising a light chain of SEQ ID NO: 20 and a heavy chain of SEQ ID NO: 38.

In some embodiments, a “C5” variant is used. The heavy chain of this variant may comprise a linker such as a cleavable peptide linker (“Lx”, e.g., SEQ ID NO: 199 or SEQ ID NO:194) introduced into between the boundary of heavy-chain variable region and Constant Region 5 (“C5”, e.g., SEQ ID NO: 41). In the Constant Region 5, a non-limiting example of the positional shift of a linker (e.g., the GS linker (GGGGSGGGGS (SEQ ID NO: 6)₂) present in the hinge region) is shown below:

from [GGGGSGGGGSEPKSCDKTHTCPPCP] (SEQ ID NO: 42)
to [EPKSCDKTHTGGGGSGGGGSCPPCP] (SEQ ID NO: 43) (the initial residue (E) is at position 216 (EU numbering)). The shifted position of the linker can be appropriately selected or designed by those skilled in the art according to the purpose, i.e., to promote homogeneity. The positional shift of the linker can promote or facilitate disulfide (cysteine-cysteine (Cys-Cys)) bond formation between Cys at position 220 (C220) (EU numbering) of the heavy chain and Cys at position 214 (C214) (EU numbering) of the light chain. A single-chain ligand IL-12 may be attached to the C-terminus of Fc domain via a linker such as the GS linker (“L5”, e.g., SEQ ID NO: 40).

In embodiments, a constant region Cx as specified in aspects and embodiments herein, comprises, or consists of, an amino acid sequence of SEQ ID NO: 75 (“C4”). In one embodiment, the fusion protein or protein complex is a bivalent ligand-binding protein complex which comprises two sets (e.g., two identical sets) of the ligand-binding domain, the ligand moiety, the cleavable peptide linker, the constant (or Fc) region, and the non-cleavable peptide linker. When the protein complex comprises two Fc region sequences, the Fc region sequences dimerize with each other to form a ligand-binding moiety, or a binding molecule complex. In embodiments, the Fc region sequences dimerize with each other in the same manner as the dimerization of two Fc region sequences of antibody heavy chain sequences to form the Fc region of an antibody.

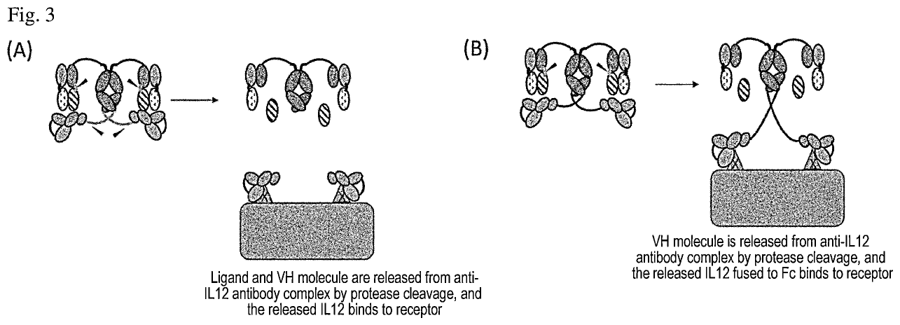

In one embodiment of the present invention, the ligand moiety is released from the ligand-binding domain of the ligand-binding moiety by protease cleavage of the fusion protein. In this context for reference, when the ligand moiety is connected with the C-terminal region of the ligand binding moiety via a peptide linker having a protease cleavage site, the ligand moiety may be completely released from the fusion protein. Herein, this type of fusion protein is referred to as “release type” (see e.g., FIG. 3A). On the other hand, when the ligand moiety is connected with the C-terminal region of the ligand binding moiety via a peptide linker having no protease cleavage site according to the present invention, the ligand moiety may be released from the ligand-binding domain while remaining fused to the C-terminal region of the ligand binding moiety via the peptide linker. Herein, this type of fusion protein of aspects of the invention is referred to as “fusion type” (see e.g., FIG. 3B).

A method for detecting release of the ligand moiety or molecule from the ligand-binding domain by cleavage of the protease cleavage site(s) includes a method of detecting the ligand using, for example, an antibody for ligand detection that recognizes the ligand. When the ligand binding moiety/molecule is an antibody fragment, the antibody for ligand detection preferably binds to the same epitope as that for the ligand binding domain. The ligand detected using the antibody for ligand detection can be confirmed by a well-known method such as FACS, an ELISA format, a BIACORE® method using ALPHA (amplified luminescent proximity homogeneous assay) screening or surface plasmon resonance (SPR) phenomena, or BLI (bio-layer interferometry) (Octet) (Proc. Natl. Acad. Sci. USA (2006) 103 (11), 4005-4010).

In the case of detecting the release of the ligand IL-12 using, for example, Octet, the antibody for ligand detection that recognizes the ligand is biotinylated and contacted with a biosensor. The antibody for ligand detection in this case can be a conventional type anti-IL-12 antibody, i.e. anti-IL-12 binding molecule or anti-IL-12 antibody. Then, binding to the ligand in a sample can be measured to detect the release of the ligand. Specifically, the amount of the ligand is measured in a sample containing the ligand binding molecule before protease treatment or after protease treatment and the ligand, using the antibody for ligand detection. The amount of the ligand detected in the sample can be compared between before and after protease treatment to detect the release of the ligand. When the ligand binding molecule is bound to the ligand to form a protein complex, the amount of the ligand is measured in a sample containing the protein complex before protease treatment or after protease treatment, using the antibody for ligand detection. The amount of the ligand detected in the sample can be compared between before and after protease treatment to detect the release of the ligand. More specifically, the release of the ligand can be detected by a method described in Examples of the present application.

In an embodiment in which the physiological activity of the ligand is attenuated upon binding to the ligand-binding domain, the release from the ligand-binding molecule can be detected by a method of measuring the physiological activity of the ligand in a sample. Specifically, the physiological activity of the ligand can be measured in a sample containing the ligand-binding molecule before protease treatment or after protease treatment and compared between before and after protease treatment to detect the release of the ligand. As the ligand-binding molecule is bound to the ligand to form a protein complex, the physiological activity of the ligand can be measured in a sample containing the protein complex before protease treatment or after protease treatment and compared between before and after protease treatment to detect the release of the ligand. Alternatively, the physiological activity of the ligand can be measured in a sample containing protease and the protein complex and a sample containing the protein complex without containing protease and compared between these samples to detect the release of the ligand.

In the present invention, the fusion protein comprising the ligand binding domain comprises a protease cleavage site comprising a protease cleavage sequence as defined in SEQ ID NO: 199 or SEQ ID NO: 194, and is cleavable by a protease. In some embodiments of the present invention, the protease cleavage sites may also comprise one or more amino acid residues at one or both ends of the protease cleavage sequence as long as those residues do not inhibit recognition and cleavage of the protease cleavage sequence by the protease.

Additional protease cleavage sites are disclosed in International Publication No. WO2023/002952, which is incorporated herein by reference in its entirety for all purposes.

### Protease

In the present specification, the term “protease” refers to an enzyme such as endopeptidase or exopeptidase which hydrolyses a peptide bond, and typically refers to endopeptidase. The protease used in the present invention is limited only by its capability of cleaving a protease cleavage sequence and is not limited to any particular type of protease. In some embodiments, target tissue specific protease is used. The target tissue specific protease can refer to, for example, any of
(1) protease that is expressed at a higher level in the target tissue than in normal tissues,
(2) protease that has higher activity in the target tissue than in normal tissues,
(3) protease that is expressed at a higher level in the target cells than in normal cells, and
(4) protease that has higher activity in the target cells than in normal cells.
   In a more specific embodiment, a cancer tissue specific protease or an inflammatory tissue specific protease is used. In a more specific embodiment, a cancer tissue specific protease is used. In embodiments, the cancer tissue specific protease includes a Matriptase (MT-SP1) and human urokinase-type Plasminogen Activator (huPA).

In the present specification, the term “target tissue” means a tissue containing at least one target cell. In some embodiments of the present invention, the target tissue is a cancer tissue. In some embodiments of the present invention, the target tissue is an inflammatory tissue.

The term “cancer tissue” means a tissue containing at least one cancer cell. Thus, considering that, for example, the cancer tissue contains cancer cells and vascular vessels, every cell type that contributes to the formation of tumor mass containing cancer cells and endothelial cells is included in the scope of the present invention. In the present specification, the tumor mass refers to a foci of tumor tissue. The term “tumor” is generally used to mean benign neoplasm or malignant neoplasm.

In the present specification, examples of the “inflammatory tissue” include the following:
a joint tissue in rheumatoid arthritis or osteoarthritis,
a lung (alveolus) tissue in bronchial asthma or Chronic obstructive pulmonary disease (COPD),
a digestive organ tissue in inflammatory bowel disease, Crohn's disease, or ulcerative colitis,
a fibrotic tissue in fibrosis in the liver, the kidney, or the lung,
a tissue under rejection of organ transplantation,
a vascular vessel or heart (cardiac muscle) tissue in arteriosclerosis or heart failure,
a visceral fat tissue in metabolic syndrome,
a skin tissue in atopic dermatitis and other dermatitis,
a spinal nerve tissue in disk herniation or chronic lumbago, and
any tissue that is infiltrated with immune cells.

Specifically expressed or specifically activated protease, or protease considered to be related to the disease condition of a target tissue (target tissue specific protease) is known for some types of target tissues. In embodiments herein, the target tissue specific protease refer to cancer tissue specific protease. Examples of the cancer tissue specific protease include specifically expressed in a cancer tissue disclosed in for example, International Publication Nos. WO2013/128194, WO2010/081173, and WO2009/025846 disclose protease specifically expressed in a cancer tissue. Also, J Inflamm (Lond). 2010; 7: 45, Nat Rev Immunol. 2006 July; 6 (7): 541-50, Nat Rev Drug Discov. 2014 December; 13 (12): 904-27, Respir Res. 2016 Mar. 4; 17: 23, Dis Model Mech. 2014 February; 7 (2): 193-203, and Biochim Biophys Acta. 2012 January; 1824 (1): 133-45 disclose protease considered to be related to inflammation.

In addition to the protease specifically expressed in a target tissue, there also exists protease specifically activated in a target tissue. For example, protease may be expressed in an inactive form and then converted to an active form. Many tissues contain a substance inhibiting active protease and control the activity by the process of activation and the presence of the inhibitor (Nat Rev Cancer. 2003 July; 3 (7): 489-501). In a target tissue, the active protease may be specifically activated by escaping inhibition. The active protease can be measured by use of a method using an antibody recognizing the active protease (PNAS 2013 January 2; 110 (1): 93-98) or a method of fluorescently labelling a peptide recognizable by protease so that the fluorescence is quenched before cleavage, but emitted after cleavage (Nat Rev Drug Discov. 2010 September; 9 (9): 690-701. doi: 10.1038/nrd3053).

From one viewpoint, the term “target tissue specific protease” can refer to any of
(i) protease that is expressed at a higher level in the target tissue than in normal tissues,
(ii) protease that has higher activity in the target tissue than in normal tissues,
(iii) protease that is expressed at a higher level in the target cells than in normal cells, and
(iv) protease that has higher activity in the target cells than in normal cells.

Specific examples of the protease include, but are not limited to, cysteine protease (including cathepsin families B, L, S, etc.), aspartyl protease (cathepsins D, E, K, O, etc.), serine protease (including matriptase (including MT-SP1), cathepsins A and G, thrombin, plasmin, urokinase-type plasminogen activator (uPA), tissue plasminogen activator (tPA), elastase, proteinase 3, thrombin, kallikrein, tryptase, and chymase), metalloproteinase (metalloproteinase (MMP1-28) including both membrane-bound forms (MMP14-17 and MMP24-25) and secreted forms (MMP1-13, MMP18-23 and MMP26-28), A disintegrin and metalloproteinase (ADAM), A disintegrin and metallo-proteinase with thrombospondin motifs (ADAMTS), meprin (meprin alpha and meprin beta), CD10 (CALLA), prostate-specific antigen (PSA), legumain, TMPRSS3, TMPRSS4, human neutrophil elastase (HNE), beta secretase (BACE), fibroblast activation protein alpha (FAP), granzyme B, guanidinobenzoatase (GB), hepsin, neprilysin, NS3/4A, HCV-NS3/4, calpain, ADAMDEC1, renin, cathepsin B, cathepsin C, cathepsin D, cathepsin E, cathepsin G, cathepsin K, cathepsin L, cathepsin V/L2, cathepsin X/Z/P, cruzipain, otubain 2, kallikrein-related peptidases (KLKs (KLK3, KLK4, KLK5, KLK6, KLK7, KLK8, KLK10, KLK11, KLK13, and KLK14)), bone morphogenetic protein 1 (BMP-1), activated protein C, blood coagulation-related protease (Factor VIIa, Factor IXa, Factor Xa, Factor XIa, and Factor XIIa), HtrA1, lactoferrin, marapsin, PACE4, DESC1, dipeptidyl peptidase 4 (DPP-4), TMPRSS2, cathepsin F, cathepsin H, cathepsin L2, cathepsin 0, cathepsin S, granzyme A, Gepsin calpain 2, glutamate carboxypeptidase 2, AMSH-like proteases, AMSH, gamma secretase, antiplasmin cleaving enzyme (APCE), decysin 1, N-acetylated alpha-linked acidic dipeptidase-like 1 (NAALADL1), and furin.

From another viewpoint, in embodiments herein, the target tissue specific protease can refer to cancer tissue specific protease or inflammatory tissue specific protease.

Examples of the cancer tissue specific protease include protease specifically expressed in a cancer tissue disclosed in International Publication Nos. WO2013/128194, WO2010/081173, and WO2009/025846.

As for the type of the cancer tissue specific protease, the protease having higher expression specificity in the cancer tissue to be treated is more effective for reducing adverse reactions. Preferable cancer tissue specific protease has a concentration in the cancer tissue at least 5 times, more preferably at least 10 times, further preferably at least 100 times, particularly preferably at least 500 times, most preferably at least 1000 times higher than its concentration in normal tissues. Also, preferable cancer tissue specific protease has activity in the cancer tissue at least 2 times, more preferably at least 3 times, at least 4 times, at least 5 times, or at least 10 times, further preferably at least 100 times, particularly preferably at least 500 times, most preferably at least 1000 times higher than its activity in normal tissues.

The cancer tissue specific protease may be in a form bound with a cancer cell membrane or may be in a form secreted extracellularly without being bound with a cell membrane. When the cancer tissue specific protease is not bound with a cancer cell membrane, it is preferred that the cancer tissue specific protease should exist within or in the vicinity of the cancer tissue. In the present specification, the “vicinity of the cancer tissue” means to fall within the scope of location where the protease cleavage sequence specific for the cancer tissue is cleaved so that the effect of reducing the ligand-binding activity is exerted.

From an alternative viewpoint, in embodiments herein, cancer tissue specific protease is any of
(i) protease that is expressed at a higher level in the cancer tissue than in normal tissues,
(ii) protease that has higher activity in the cancer tissue than in normal tissues,
(iii) protease that is expressed at a higher level in the cancer cells than in normal cells, and
(iv) protease that has higher activity in the cancer cells than in normal cells.
   One type of cancer tissue specific protease may be used alone, or two or more types of cancer tissue specific proteases may be combined. The number of types of the cancer tissue specific protease can be appropriately set by those skilled in the art in consideration of the cancer type to be treated.

From these viewpoints, in embodiments herein, the cancer tissue specific protease is preferably serine protease or metalloproteinase, more preferably matriptase (including MT-SP1), urokinase-type plasminogen activator (uPA), or metalloproteinase, further preferably MT-SP1, uPA, MMP-2, or MMP-9, among the proteases listed above, particular preferably MMP-2, or MMP-9, among the proteases listed above.

As for the type of inflammatory tissue specific protease, the protease having higher expression specificity in the inflammatory tissue to be treated is more effective for reducing adverse reactions. Preferable inflammatory tissue specific protease has a concentration in the inflammatory tissue at least 5 times, more preferably at least 10 times, further preferably at least 100 times, particularly preferably at least 500 times, most preferably at least 1000 times higher than its concentration in normal tissues. Also, preferable inflammatory tissue specific protease has activity in the inflammatory tissues at least 2 times, more preferably at least 3 times, at least 4 times, at least 5 times, or at least 10 times, further preferably at least 100 times, particularly preferably at least 500 times, most preferably at least 1000 times higher than its activity in normal tissues.

The inflammatory tissue specific protease may be in a form bound with an inflammatory cell membrane or may be in a form secreted extracellularly without being bound with a cell membrane. When the inflammatory tissue specific protease is not bound with an inflammatory cell membrane, it is preferred that the inflammatory tissue specific protease should exist within or in the vicinity of the inflammatory tissue. In the present specification, the “vicinity of the inflammatory tissue” means to fall within the scope of location where the protease cleavage sequence specific for the inflammatory tissue is cleaved so that the effect of reducing the ligand binding activity is exerted.

From an alternative viewpoint, inflammatory tissue specific protease is any of
(i) protease that is expressed at a higher level in the inflammatory tissue than in normal tissues,
(ii) protease that has higher activity in the inflammatory tissue than in normal tissues,
(iii) protease that is expressed at a higher level in the inflammatory cells than in normal cells, and
(iv) protease that has higher activity in the inflammatory cells than in normal cells.
   One type of inflammatory tissue specific protease may be used alone, or two or more types of inflammatory tissue specific proteases may be combined. The number of types of the inflammatory tissue specific protease can be appropriately set by those skilled in the art in consideration of the pathological condition to be treated.

From these viewpoints, the inflammatory tissue specific protease is preferably metalloproteinase among the proteases listed above. The metalloproteinase is more preferably ADAMTS5, MMP-1, MMP-2, MMP-3, MMP-7, MMP-9, MMP11, or MMP-13.

### Protease Cleavage Sequence

The protease cleavage sequence is a particular amino acid sequence that is specifically recognized by target tissue specific protease when the polypeptide is hydrolyzed by the target tissue specific protease in an aqueous solution. The protease cleavage sequence is preferably an amino acid sequence that is hydrolyzed with high specificity by target tissue specific protease more specifically expressed in the target tissue or cells to be treated or more specifically activated in the target tissue/cells to be treated, from the viewpoint of reduction in adverse reactions.

The protease cleavage sequence is more preferably an amino acid sequence that is specifically hydrolyzed by suitable target tissue specific protease as mentioned above. The amino acid sequence that is specifically hydrolyzed by target tissue specific protease is or comprises preferably any of the following amino acid sequences:

(SEQ ID NO: 369, cleavable by MT-SP1 or uPA),
LSGRSDNH
(SEQ ID NO: 370, cleavable by MMP-2 or MMP-9), and
PLGLAG
(SEQ ID NO: 371, cleavable by MMP-7).
VPLSLTMG
(SEQ ID NO: 372, cleavable by MT-SP1 or uPA),
ISSGLLSGRSDNH
(SEQ ID NO: 373, cleavable by MT-SP1 or uPA),
AVGLLAPPGGLSGRSDNH
(SEQ ID NO: 374, cleavable by MMP-1),
GAGVPMSMRGGAG
(SEQ ID NO: 375, cleavable by MMP-2),
GAGIPVSLRSGAG
(SEQ ID NO: 376, cleavable by MMP-2),
GPLGIAGQ
(SEQ ID NO: 377, cleavable by MMP-2),
GGPLGMLSQS
(SEQ ID NO: 378, cleavable by MMP-2),
PLGLWA
(SEQ ID NO: 379, cleavable by MMP-3),
GAGRPFSMIMGAG
(SEQ ID NO: 380, cleavable by MMP-7),
GAGVPLSLTMGAG
(SEQ ID NO: 381, cleavable by MMP-9),
GAGVPLSLYSGAG
(SEQ ID NO: 382, cleavable by MMP-11),
AANLRN
(SEQ ID NO: 383, cleavable by MMP-11),
AQAYVK
(SEQ ID NO: 384, cleavable by MMP-11),
AANYMR
(SEQ ID NO: 385, cleavable by MMP-11),
AAALTR
(SEQ ID NO: 386, cleavable by MMP-11),
AQNLMR
(SEQ ID NO: 387, cleavable by MMP-11),
AANYTK
(SEQ ID NO: 388, cleavable by MMP-13),
GAGPQGLAGQRGIVAG
(SEQ ID NO: 389, cleavable by pro-urokinase),
PRFKIIGG
(SEQ ID NO: 390, cleavable by pro-urokinase),
PRFRIIGG
(SEQ ID NO: 391, cleavable by uPA),
GAGSGRSAG
(SEQ ID NO: 392, cleavable by uPA),
SGRSA
(SEQ ID NO: 393, cleavable by uPA),
GSGRSA
(SEQ ID NO: 394, cleavable by uPA),
SGKSA
(SEQ ID NO: 395, cleavable by uPA),
SGRSS
(SEQ ID NO: 396, cleavable by uPA),
SGRRA
(SEQ ID NO: 397, cleavable by uPA),
SGRNA
(SEQ ID NO: 398, cleavable by uPA),
SGRKA
(SEQ ID NO: 399, cleavable by tPA),
QRGRSA
(SEQ ID NO: 400, cleavable by cathepsin B)
GAGSLLKSRMVPNFNAG
(SEQ ID NO: 401, cleavable by cathepsin B),
TQGAAA
(SEQ ID NO: 402, cleavable by cathepsin B),
GAAAAA
(SEQ ID NO: 403, cleavable by cathepsin B),
GAGAAG
(SEQ ID NO: 404, cleavable by cathepsin B),
AAAAAG
(SEQ ID NO: 405, cleavable by cathepsin B),
LCGAAI
(SEQ ID NO: 406, cleavable by cathepsin B),
FAQALG
(SEQ ID NO: 407, cleavable by cathepsin B),
LLQANP
(SEQ ID NO: 408, cleavable by cathepsin B),
LAAANP
(SEQ ID NO: 409, cleavable by cathepsin B),
LYGAQF
(SEQ ID NO: 410, cleavable by cathepsin B),
LSQAQG
(SEQ ID NO: 411, cleavable by cathepsin B),
ASAASG
(SEQ ID NO: 412, cleavable by cathepsin B),
FLGASL
(SEQ ID NO: 413, cleavable by cathepsin B),
AYGATG
(SEQ ID NO: 414, cleavable by cathepsin B),
LAQATG
(SEQ ID NO: 415, cleavable by cathepsin L),
GAGSGVVIATVIVITAG
(SEQ ID NO: 416, cleavable by meprin alpha or meprin beta),
APMAEGGG
(SEQ ID NO: 417, cleavable by meprin alpha or meprin beta),
EAQGDKII
(SEQ ID NO: 418, cleavable by meprin alpha or meprin beta),
LAFSDAGP
(SEQ ID NO: 419, cleavable by meprin alpha or meprin beta),
YVADAPK
(SEQ ID NO: 420, cleavable by furin),
RRRRR
(SEQ ID NO: 421, cleavable by furin),
RRRRRR
(SEQ ID NO: 422, cleavable by furin),
GQSSRHRRAL
(SEQ ID NO: 423),
SSRHRRALD
(SEQ ID NO: 424, cleavable by plasminogen),
RKSSIIIRMRDVVL
(SEQ ID NO: 425, cleavable by staphylokinase),
SSSFDKGKYKKGDDA
(SEQ ID NO: 426, cleavable by staphylokinase),
SSSFDKGKYKRGDDA
(SEQ ID NO: 427, cleavable by Factor IXa),
IEGR
(SEQ ID NO: 428, cleavable by Factor IXa),
IDGR
(SEQ ID NO: 429, cleavable by Factor IXa),
GGSIDGR
(SEQ ID NO: 430, cleavable by collagenase),
GPQGIAGQ
(SEQ ID NO: 431, cleavable by collagenase),
GPQGLLGA
(SEQ ID NO: 432, cleavable by collagenase),
GIAGQ
(SEQ ID NO: 433, cleavable by collagenase),
GPLGIAG
(SEQ ID NO: 434, cleavable by collagenase),
GPEGLRVG
(SEQ ID NO: 435, cleavable by collagenase),
YGAGLGVV
(SEQ ID NO: 436, cleavable by collagenase),
AGLGVVER
(SEQ ID NO: 437, cleavable by collagenase),
AGLGISST
(SEQ ID NO: 438, cleavable by collagenase),
EPQALAMS
(SEQ ID NO: 439, cleavable by collagenase),
QALAMSAI
(SEQ ID NO: 440, cleavable by collagenase),
AAYHLVSQ
(SEQ ID NO: 441, cleavable by collagenase),
MDAFLESS
(SEQ ID NO: 442, cleavable by collagenase),
ESLPVVAV
(SEQ ID NO: 443, cleavable by collagenase),
SAPAVESE
(SEQ ID NO: 444, cleavable by collagenase),
DVAQFVLT
(SEQ ID NO: 445, cleavable by collagenase),
VAQFVLTE
(SEQ ID NO: 446, cleavable by collagenase),
AQFVLTEG
(SEQ ID NO: 447, cleavable by collagenase),
PVQPIGPQ
(SEQ ID NO: 448, cleavable by thrombin).
LVPRGS

The sequences shown in Table 37 may also be used as protease cleavage sequences.

TABLE 37
Protease cleavage sequences (cleavable by uPA
and MT-SP1)

| SEQ | | SEQ | |
|---|---|---|---|
| ID | Cleavage | ID | Cleavage |
| NO | Sequence | NO | Sequence |
| 449 | TSGSGRSANARG | 808 | TSQSGRSENPRG |
| 450 | TSQSGRSANQRG | 809 | TSPSGRSENPRG |
| 451 | TSPSGRSAYPRG | 810 | TSASGRSENPRG |
| 199 | TSGSGRSATPRG | 811 | TSHSGRSENPRG |
| 452 | TSQSGRSATPRG | 812 | TSTSGRSENQRG |
| 453 | TSASGRSATPRG | 813 | TSTSGRSENARG |
| 454 | TSYSGRSAVPRG | 814 | TSTSGRSENIRG |
| 455 | TSYSGRSANFRG | 815 | TSTSGRSENFRG |
| 456 | TSSSGRSATPRG | 816 | TSTSGRSENSRG |
| 457 | TSTTGRSASPRG | 817 | TSYSGRSAEPRG |
| 458 | TSASGRSANPRG | 818 | TSGSGRSAEPRG |
| 459 | TSESGRSANPRG | 819 | TSQSGRSAEPRG |
| 460 | TSFSGRSANPRG | 820 | TSPSGRSAEPRG |
| 461 | TSGSGRSANPRG | 821 | TSASGRSAEPRG |
| 462 | TSHSGRSANPRG | 822 | TSHSGRSAEPRG |
| 463 | TSKSGRSANPRG | 823 | TSTSGRSAEQRG |
| 464 | TSMSGRSANPRG | 824 | TSTSGRSAEARG |
| 465 | TSNSGRSANPRG | 825 | TSTSGRSAEIRG |
| 466 | TSPSGRSANPRG | 215 | TSTSGRSAEFRG |
| 467 | TSQSGRSANPRG | 826 | TSTSGRSAESRG |
| 468 | TSWSGRSANPRG | 827 | TSGTGRSANPRG |
| 469 | TSYSGRSANPRG | 828 | TSGKGRSANPRG |
| 470 | TSTAGRSANPRG | 829 | TSGSGRSAIPRG |
| 471 | TSTDGRSANPRG | 830 | TSGSGRSASPRG |
| 472 | TSTEGRSANPRG | 831 | TSGSGRSAHPRG |
| 473 | TSTFGRSANPRG | 832 | TSGSGRSANYRG |
| 214 | TSTLGRSANPRG | 833 | TSGSGRSANVRG |
| 474 | TSTMGRSANPRG | 834 | TSGSGRSANHRG |
| 475 | TSTPGRSANPRG | 835 | TSQTGRSANPRG |
| 476 | TSTQGRSANPRG | 836 | TSQKGRSANPRG |
| 477 | TSTVGRSANPRG | 837 | TSQSGRSAIPRG |
| 478 | TSTWGRSANPRG | 838 | TSQSGRSASPRG |
| 479 | TSTSARSANPRG | 839 | TSQSGRSAHPRG |
| 480 | TSTSERSANPRG | 840 | TSQSGRSANYRG |
| 481 | TSTSFRSANPRG | 841 | TSQSGRSANVRG |
| 482 | TSTSHRSANPRG | 842 | TSQSGRSANHRG |
| 483 | TSTSIRSANPRG | 217 | TSPTGRSANPRG |
| 484 | TSTSKRSANPRG | 843 | TSPKGRSANPRG |
| 485 | TSTSLRSANPRG | 844 | TSPSGRSAIPRG |
| 486 | TSTSMRSANPRG | 845 | TSPSGRSATPRG |
| 487 | TSTSNRSANPRG | 846 | TSPSGRSASPRG |
| 488 | TSTSPRSANPRG | 847 | TSPSGRSAHPRG |
| 489 | TSTSQRSANPRG | 848 | TSPSGRSANYRG |
| 490 | TSTSRRSANPRG | 849 | TSPSGRSANVRG |
| 491 | TSTSTRSANPRG | 850 | TSPSGRSANHRG |
| 492 | TSTSVRSANPRG | 851 | TSATGRSANPRG |
| 493 | TSTSWRSANPRG | 852 | TSAKGRSANPRG |
| 494 | TSTSYRSANPRG | 853 | TSASGRSAIPRG |
| 495 | TSTSGRAANPRG | 854 | TSASGRSASPRG |
| 496 | TSTSGRDANPRG | 855 | TSASGRSAHPRG |
| 497 | TSTSGREANPRG | 856 | TSASGRSANYRG |
| 498 | TSTSGRGANPRG | 857 | TSASGRSANVRG |
| 499 | TSTSGRHANPRG | 858 | TSASGRSANHRG |
| 500 | TSTSGRIANPRG | 859 | TSYTGRSANPRG |
| 501 | TSTSGRKANPRG | 860 | TSYKGRSANPRG |
| 502 | TSTSGRLANPRG | 861 | TSYSGRSAIPRG |
| 503 | TSTSGRMANPRG | 862 | TSYSGRSATPRG |
| 504 | TSTSGRNANPRG | 863 | TSYSGRSASPRG |
| 505 | TSTSGRPANPRG | 864 | TSYSGRSAHPRG |
| 506 | TSTSGRQANPRG | 865 | TSYSGRSANARG |
| 507 | TSTSGRRANPRG | 866 | TSYSGRSANYRG |
| 508 | TSTSGRTANPRG | 867 | TSYSGRSANVRG |
| 509 | TSTSGRVANPRG | 868 | TSYSGRSANHRG |
| 510 | TSTSGRWANPRG | 869 | TSSTGRSANPRG |
| 511 | TSTSGRYANPRG | 870 | TSSKGRSANPRG |
| 512 | TSTSGRSENPRG | 871 | TSSSGRSAVPRG |
| 513 | TSTSGRSFNPRG | 872 | TSSSGRSAIPRG |
| 514 | TSTSGRSKNPRG | 873 | TSSSGRSASPRG |
| 515 | TSTSGRSMNPRG | 874 | TSSSGRSAHPRG |
| 516 | TSTSGRSNNPRG | 875 | TSSSGRSANARG |
| 517 | TSTSGRSPNPRG | 876 | TSSSGRSANFRG |
| 518 | TSTSGRSQNPRG | 877 | TSSSGRSANYRG |
| 519 | TSTSGRSRNPRG | 878 | TSSSGRSANVRG |
| 520 | TSTSGRSSNPRG | 879 | TSSSGRSANHRG |
| 521 | TSTSGRSWNPRG | 880 | TSITGRSANPRG |
| 522 | TSTSGRSYNPRG | 881 | TSIKGRSANPRG |
| 523 | TSTSGRSAAPRG | 882 | TSISGRSAVPRG |
| 524 | TSTSGRSADPRG | 883 | TSISGRSAIPRG |
| 525 | TSTSGRSAEPRG | 884 | TSISGRSATPRG |
| 526 | TSTSGRSAFPRG | 885 | TSISGRSASPRG |
| 527 | TSTSGRSAGPRG | 886 | TSISGRSAHPRG |
| 528 | TSTSGRSAKPRG | 887 | TSISGRSANARG |
| 529 | TSTSGRSALPRG | 888 | TSISGRSANFRG |
| 530 | TSTSGRSAMPRG | 889 | TSISGRSANYRG |
| 531 | TSTSGRSAPPRG | 890 | TSISGRSANVRG |
| 532 | TSTSGRSAQPRG | 891 | TSISGRSANHRG |
| 533 | TSTSGRSAVPRG | 892 | TSTTGRSAVPRG |
| 534 | TSTSGRSAWPRG | 893 | TSTTGRSAIPRG |
| 535 | TSTSGRSAYPRG | 894 | TSTTGRSATPRG |
| 536 | TSTSGRSANARG | 895 | TSTTGRSAHPRG |
| 537 | TSTSGRSANDRG | 896 | TSTTGRSANARG |
| 538 | TSTSGRSANERG | 897 | TSTTGRSANFRG |
| 539 | TSTSGRSANFRG | 898 | TSTTGRSANYRG |
| 540 | TSTSGRSANGRG | 899 | TSTTGRSANVRG |
| 541 | TSTSGRSANIRG | 900 | TSTTGRSANHRG |
| 542 | TSTSGRSANKRG | 901 | TSTKGRSAVPRG |
| 543 | TSTSGRSANNRG | 902 | TSTKGRSAIPRG |
| 544 | TSTSGRSANQRG | 903 | TSTKGRSATPRG |
| 545 | TSTSGRSANSRG | 904 | TSTKGRSASPRG |
| 546 | TSTSGRSANTRG | 905 | TSTKGRSAHPRG |
| 547 | TSTSGRSANWRG | 906 | TSTKGRSANARG |
| 548 | TSDSGRSANPRG | 907 | TSTKGRSANFRG |
| 549 | TSISGRSANPRG | 908 | TSTKGRSANYRG |
| 550 | TSSSGRSANPRG | 909 | TSTKGRSANVRG |
| 551 | TSTHGRSANPRG | 910 | TSTKGRSANHRG |
| 552 | TSTKGRSANPRG | 911 | TSTSGRSAVYRG |
| 553 | TSTTGRSANPRG | 912 | TSTSGRSAVVRG |
| 554 | TSTYGRSANPRG | 913 | TSTSGRSAVHRG |
| 555 | TSTSDRSANPRG | 914 | TSTSGRSAIYRG |
| 556 | TSTSSRSANPRG | 915 | TSTSGRSAIVRG |
| 557 | TSTSGRFANPRG | 916 | TSTSGRSAIHRG |
| 558 | TSTSGRSDNPRG | 917 | TSTSGRSASYRG |
| 559 | TSTSGRSHNPRG | 918 | TSTSGRSASVRG |
| 560 | TSTSGRSINPRG | 919 | TSTSGRSASHRG |
| 561 | TSTSGRSLNPRG | 920 | TSTSGRSAHYRG |
| 562 | TSTSGRSTNPRG | 921 | TSTSGRSAHVRG |
| 563 | TSTSGRSVNPRG | 922 | TSTSGRSAHHRG |
| 564 | TSTSGRSAHPRG | 923 | TSPSGRSEVPRG |
| 565 | TSTSGRSAIPRG | 924 | TSPSGRSAEPRG |
| 566 | TSTSGRSARPRG | 925 | TSPSGRSAGPRG |
| 567 | TSTSGRSASPRG | 926 | TSASGRSENARG |
| 568 | TSTSGRSATPRG | 927 | TSASGRSAEARG |
| 569 | TSTSGRSANHRG | 928 | TSASGRSAGARG |
| 570 | TSTSGRSANLRG | 929 | TSGTGRSATPRG |
| 571 | TSTSGRSANMRG | 930 | TSGSGRSATYRG |
| 572 | TSTSGRSANRRG | 931 | TSGSGRSATVRG |
| 573 | TSTSGRSANVRG | 932 | TSGSGRSATHRG |
| 574 | TSTSGRSANYRG | 933 | TSGTGRSATYRG |
| 575 | TSGSGRSAVPRG | 934 | TSGTGRSATVRG |
| 576 | TSGSGRSAYPRG | 935 | TSGTGRSATHRG |
| 577 | TSGSGRSANQRG | 936 | TSGSGRSETPRG |
| 578 | TSGSGRSANIRG | 937 | TSGTGRSETPRG |
| 579 | TSGSGRSANFRG | 938 | TSGSGRSETYRG |
| 580 | TSGSGRSANSRG | 939 | TSGSGRSETVRG |
| 581 | TSQSGRSAVPRG | 940 | TSGSGRSETHRG |
| 582 | TSQSGRSAYPRG | 941 | TSYTGRSAVPRG |
| 583 | TSQSGRSANARG | 942 | TSYSGRSAVYRG |
| 584 | TSQSGRSANIRG | 943 | TSYSGRSAVVRG |
| 585 | TSQSGRSANFRG | 944 | TSYSGRSAVHRG |
| 586 | TSQSGRSANSRG | 218 | TSYTGRSAVYRG |
| 587 | TSPSGRSAVPRG | 219 | TSYTGRSAVVRG |
| 588 | TSPSGRSANQRG | 945 | TSYTGRSAVHRG |
| 589 | TSPSGRSANARG | 946 | TSYSGRSEVPRG |
| 590 | TSPSGRSANIRG | 947 | TSYTGRSEVPRG |
| 591 | TSPSGRSANFRG | 948 | TSYSGRSEVYRG |
| 592 | TSPSGRSANSRG | 949 | TSYSGRSEVVRG |
| 593 | TSASGRSAVPRG | 950 | TSYSGRSEVHRG |
| 594 | TSASGRSAYPRG | 951 | TSYTGRSAVPGG |
| 595 | TSASGRSANQRG | 952 | TSYSGRSAVYGG |
| 596 | TSASGRSANARG | 953 | TSYSGRSAVVGG |
| 597 | TSASGRSANIRG | 954 | TSYSGRSAVHGG |
| 598 | TSASGRSANFRG | 955 | TSYTGRSAVYGG |
| 599 | TSASGRSANSRG | 956 | TSYTGRSAVVGG |
| 600 | TSYSGRSENPRG | 957 | TSYTGRSAVHGG |
| 601 | TSGSGRSENPRG | 958 | TSGRSENI |
| 602 | ASGRSANP | 959 | TSGRSENF |
| 603 | ESGRSANP | 960 | TSGRSENS |
| 604 | FSGRSANP | 961 | YSGRSAEP |
| 605 | GSGRSANP | 962 | GSGRSAEP |
| 606 | HSGRSANP | 963 | QSGRSAEP |
| 607 | KSGRSANP | 964 | PSGRSAEP |
| 608 | MSGRSANP | 965 | ASGRSAEP |
| 609 | NSGRSANP | 966 | HSGRSAEP |
| 610 | PSGRSANP | 967 | TSGRSAEQ |
| 611 | QSGRSANP | 968 | TSGRSAEA |
| 612 | WSGRSANP | 969 | TSGRSAEI |
| 613 | YSGRSANP | 970 | TSGRSAEF |
| 614 | TAGRSANP | 971 | TSGRSAES |
| 615 | TDGRSANP | 972 | GTGRSANP |
| 616 | TEGRSANP | 973 | GKGRSANP |
| 617 | TFGRSANP | 974 | GSGRSAIP |
| 618 | TLGRSANP | 975 | GSGRSATP |
| 619 | TMGRSANP | 976 | GSGRSASP |
| 620 | TPGRSANP | 977 | GSGRSAHP |
| 621 | TQGRSANP | 978 | GSGRSANY |
| 622 | TVGRSANP | 979 | GSGRSANV |
| 623 | TWGRSANP | 980 | GSGRSANH |
| 624 | TSARSANP | 981 | QTGRSANP |
| 625 | TSERSANP | 982 | QKGRSANP |
| 626 | TSFRSANP | 983 | QSGRSAIP |
| 627 | TSHRSANP | 984 | QSGRSATP |
| 628 | TSIRSANP | 985 | QSGRSASP |
| 629 | TSKRSANP | 986 | QSGRSAHP |
| 630 | TSLRSANP | 987 | QSGRSANY |
| 631 | TSMRSANP | 988 | QSGRSANV |
| 632 | TSNRSANP | 989 | QSGRSANH |
| 633 | TSPRSANP | 990 | PTGRSANP |
| 634 | TSQRSANP | 991 | PKGRSANP |
| 635 | TSRRSANP | 992 | PSGRSAIP |
| 636 | TSTRSANP | 993 | PSGRSATP |
| 637 | TSVRSANP | 994 | PSGRSASP |
| 638 | TSWRSANP | 995 | PSGRSAHP |
| 639 | TSYRSANP | 996 | PSGRSANY |
| 640 | TSGRAANP | 997 | PSGRSANV |
| 641 | TSGRDANP | 998 | PSGRSANH |
| 642 | TSGREANP | 999 | ATGRSANP |
| 643 | TSGRGANP | 1000 | AKGRSANP |
| 644 | TSGRHANP | 1001 | ASGRSAIP |
| 645 | TSGRIANP | 1002 | ASGRSATP |
| 646 | TSGRKANP | 1003 | ASGRSASP |
| 647 | TSGRLANP | 1004 | ASGRSAHP |
| 648 | TSGRMANP | 1005 | ASGRSANY |
| 649 | TSGRNANP | 1006 | ASGRSANV |
| 650 | TSGRPANP | 1007 | ASGRSANH |
| 651 | TSGRQANP | 1008 | YTGRSANP |
| 652 | TSGRRANP | 1009 | YKGRSANP |
| 653 | TSGRTANP | 1010 | YSGRSAVP |
| 654 | TSGRVANP | 1011 | YSGRSAIP |
| 655 | TSGRWANP | 1012 | YSGRSATP |
| 656 | TSGRYANP | 1013 | YSGRSASP |
| 65' | TSGRSENP | 1014 | YSGRSAHP |
| 658 | TSGRSFNP | 1015 | YSGRSANA |
| 659 | TSGRSKNP | 1016 | YSGRSANF |
| 660 | TSGRSMNP | 1017 | YSGRSANY |
| 661 | TSGRSNNP | 1018 | YSGRSANV |
| 662 | TSGRSPNP | 1019 | YSGRSANH |
| 663 | TSGRSQNP | 1020 | STGRSANP |
| 664 | TSGRSRNP | 1021 | SKGRSANP |
| 665 | TSGRSSNP | 1022 | SSGRSAVP |
| 666 | TSGRSWNP | 1023 | SSGRSAIP |
| 667 | TSGRSYNP | 1024 | SSGRSATP |
| 668 | TSGRSAAP | 1025 | SSGRSASP |
| 669 | TSGRSADP | 1026 | SSGRSAHP |
| 670 | TSGRSAEP | 1027 | SSGRSANA |
| 671 | TSGRSAFP | 1028 | SSGRSANF |
| 672 | TSGRSAGP | 1029 | SSGRSANY |
| 673 | TSGRSAKP | 1030 | SSGRSANV |
| 674 | TSGRSALP | 1031 | SSGRSANH |
| 675 | TSGRSAMP | 1032 | ITGRSANP |
| 676 | TSGRSAPP | 1033 | IKGRSANP |
| 677 | TSGRSAQP | 1034 | ISGRSAVP |
| 678 | TSGRSAVP | 1035 | ISGRSAIP |
| 679 | TSGRSAWP | 1036 | ISGRSATP |
| 680 | TSGRSAYP | 1037 | ISGRSASP |
| 681 | TSGRSANA | 1038 | ISGRSAHP |
| 682 | TSGRSAND | 1039 | ISGRSANA |
| 683 | TSGRSANE | 1040 | ISGRSANF |
| 684 | TSGRSANF | 1041 | ISGRSANY |
| 685 | TSGRSANG | 1042 | ISGRSANV |
| 686 | TSGRSANI | 1043 | ISGRSANH |
| 687 | TSGRSANK | 1044 | TTGRSAVP |
| 688 | TSGRSANN | 1045 | TTGRSAIP |
| 689 | TSGRSANQ | 1046 | TTGRSATP |
| 690 | TSGRSANS | 1047 | TTGRSASP |
| 691 | TSGRSANT | 1048 | TTGRSAHP |
| 692 | TSGRSANW | 1049 | TTGRSANA |
| 693 | DSGRSANP | 1050 | TTGRSANF |
| 694 | ISGRSANP | 1051 | TTGRSANY |
| 695 | SSGRSANP | 1052 | TTGRSANV |
| 696 | THGRSANP | 1053 | TTGRSANH |
| 697 | TKGRSANP | 1054 | TKGRSAVP |
| 698 | TTGRSANP | 1055 | TKGRSAIP |
| 699 | TYGRSANP | 1056 | TKGRSATP |
| 700 | TSDRSANP | 1057 | TKGRSASP |
| 701 | TSSRSANP | 1058 | TKGRSAHP |
| 702 | TSGRFANP | 1059 | TKGRSANA |
| 703 | TSGRSDNP | 1060 | TKGRSANF |
| 704 | TSGRSHNP | 1061 | TKGRSANY |
| 705 | TSGRSINP | 1062 | TKGRSANV |
| 706 | TSGRSLNP | 1063 | TKGRSANH |
| 707 | TSGRSTNP | 1064 | TSGRSAVY |
| 708 | TSGRSVNP | 1065 | TSGRSAVV |
| 709 | TSGRSAHP | 1066 | TSGRSAVH |
| 710 | TSGRSAIP | 1067 | TSGRSAIY |
| 711 | TSGRSARP | 1068 | TSGRSAIV |
| 712 | TSGRSASP | 1069 | TSGRSAIH |
| 713 | TSGRSATP | 1070 | TSGRSASY |
| 714 | TSGRSANH | 1071 | TSGRSASV |
| 715 | TSGRSANL | 1072 | TSGRSASH |
| 716 | TSGRSANM | 1073 | TSGRSAHY |
| 717 | TSGRSANR | 1074 | TSGRSAHV |
| 718 | TSGRSANV | 1075 | TSGRSAHH |
| 719 | TSGRSANY | 1076 | PSGRSEVP |
| 720 | GSGRSAVP | 1077 | PSGRSAEP |
| 721 | GSGRSAYP | 1078 | PSGRSAGP |
| 722 | GSGRSANQ | 1079 | ASGRSENA |
| 723 | GSGRSANA | 1080 | ASGRSAEA |
| 724 | GSGRSANI | 1081 | ASGRSAGA |
| 725 | GSGRSANF | 1082 | GTGRSATP |
| 726 | GSGRSANS | 1083 | GSGRSATY |
| 727 | QSGRSAVP | 1084 | GSGRSATV |
| 728 | QSGRSAYP | 1085 | GSGRSATH |
| 729 | QSGRSANQ | 1086 | GTGRSATY |
| 730 | QSGRSANA | 1087 | GTGRSATV |
| 731 | QSGRSANI | 1088 | GTGRSATH |
| 732 | QSGRSANF | 1089 | GSGRSETP |
| 733 | QSGRSANS | 1090 | GTGRSETP |
| 734 | PSGRSAVP | 1091 | GSGRSETY |
| 735 | PSGRSAYP | 1092 | GSGRSETV |
| 736 | PSGRSANQ | 1093 | GSGRSETH |
| 737 | PSGRSANA | 1094 | YTGRSAVP |
| 738 | PSGRSANI | 1095 | YSGRSAVY |
| 739 | PSGRSANF | 1096 | YSGRSAVV |
| 740 | PSGRSANS | 1097 | YSGRSAVH |
| 741 | ASGRSAVP | 1098 | YTGRSAVY |
| 742 | ASGRSAYP | 1099 | YTGRSAVV |
| 743 | ASGRSANQ | 1100 | YTGRSAVH |
| 744 | ASGRSANA | 1101 | YSGRSEVP |
| 745 | ASGRSANI | 1102 | YTGRSEVP |
| 746 | ASGRSANF | 1103 | YSGRSEVY |
| 747 | ASGRSANS | 1104 | YSGRSEVV |
| 748 | YSGRSENP | 1105 | YSGRSEVH |
| 749 | GSGRSENP | 1106 | YTGRSAVP |
| 750 | QSGRSENP | 1107 | YSGRSAVY |
| 751 | PSGRSENP | 1108 | YSGRSAVV |
| 752 | ASGRSENP | 1109 | YSGRSAVH |
| 753 | HSGRSENP | 1110 | YTGRSAVY |
| 754 | TSGRSENQ | 1111 | YTGRSAVV |
| 755 | TSGRSENA | 1112 | YTGRSAVH |
| 46 | TSTSGRSANPRG | 1113 | TSYSGRSAIPLG |
| 756 | TSTSGRSANPAG | 1114 | TSISGRSANYLG |
| 757 | TSTSGRSANPHG | 1115 | TSPSGRSAGPLG |
| 758 | TSTSGRSANPIG | 1116 | TSYTGRSAVPLG |
| 759 | TSTSGRSANPLG | 1117 | TSYTGRSAVYLG |
| 760 | TSTSGRSANPSG | 1118 | TSYTGRSAVVLG |
| 761 | ISTSGRSANPIG | 1119 | TSYTGRSAVHLG |
| 762 | YSTSGRSANPIG | 1120 | TSYSGRSAVPSG |
| 763 | TSYSGRSAVPAG | 1121 | TSPSGRSANISG |
| 764 | TSPSGRSANIAG | 1122 | TSPSGRSANFSG |
| 765 | TSPSGRSANFAG | 1123 | TSPTGRSANPSG |
| 766 | TSPTGRSANPAG | 1124 | TSPSGRSAIPSG |
| 767 | TSPSGRSAIPAG | 1125 | TSYTGRSANPSG |
| 768 | TSYTGRSANPAG | 1126 | TSYSGRSAIPSG |
| 769 | TSYSGRSAIPAG | 1127 | TSISGRSANYSG |
| 770 | TSISGRSANYAG | 1128 | TSPSGRSAGPSG |
| 771 | TSPSGRSAGPAG | 1129 | TSYTGRSAVPSG |
| 772 | TSYTGRSAVPAG | 1130 | TSYTGRSAVYSG |
| 773 | TSYTGRSAVYAG | 1131 | TSYTGRSAVVSG |
| 774 | TSYTGRSAVVAG | 1132 | TSYTGRSAVHSG |
| 775 | TSYTGRSAVHAG | 1133 | ISYSGRSAVPIG |
| 776 | TSYSGRSAVPHG | 1134 | ISPSGRSANIIG |
| 777 | TSPSGRSANIHG | 1135 | ISPSGRSANFIG |
| 778 | TSPSGRSANFHG | 1136 | ISPTGRSANPIG |
| 779 | TSPTGRSANPHG | 1137 | ISPSGRSAIPIG |
| 780 | TSPSGRSAIPHG | 1138 | ISYTGRSANPIG |
| 781 | TSYTGRSANPHG | 1139 | ISYSGRSAIPIG |
| 782 | TSYSGRSAIPHG | 1140 | ISISGRSANYIG |
| 783 | TSISGRSANYHG | 1141 | ISPSGRSAGPIG |
| 784 | TSPSGRSAGPHG | 1142 | ISYTGRSAVPIG |
| 785 | TSYTGRSAVPHG | 1143 | ISYTGRSAVYIG |
| 786 | TSYTGRSAVYHG | 1144 | ISYTGRSAVVIG |
| 787 | TSYTGRSAVVHG | 1145 | ISYTGRSAVHIG |
| 788 | TSYTGRSAVHHG | 1146 | YSYSGRSAVPIG |
| 789 | TSYSGRSAVPIG | 1147 | YSPSGRSANIIG |
| 790 | TSPSGRSANIIG | 1148 | YSPSGRSANFIG |
| 791 | TSPSGRSANFIG | 1149 | YSPTGRSANPIG |
| 792 | TSPTGRSANPIG | 1150 | YSPSGRSAIPIG |
| 793 | TSPSGRSAIPIG | 1151 | YSYTGRSANPIG |
| 794 | TSYTGRSANPIG | 1152 | YSYSGRSAIPIG |
| 795 | TSYSGRSAIPIG | 1153 | YSISGRSANYIG |
| 796 | TSISGRSANYIG | 1154 | YSPSGRSAGPIG |
| 797 | TSPSGRSAGPIG | 1155 | YSYTGRSAVPIG |
| 798 | TSYTGRSAVPIG | 1156 | YSYTGRSAVYIG |
| 799 | TSYTGRSAVYIG | 1157 | YSYTGRSAVVIG |
| 800 | TSYTGRSAVVIG | 216 | YSYTGRSAVHIG |
| 801 | TSYTGRSAVHIG | 194 | YAYTGRSANPRG |
| 802 | TSYSGRSAVPLG | 1158 | YSATGRSANPRG |
| 803 | TSPSGRSANILG | 1159 | TSYTGRSAVPRG |
| 804 | TSPSGRSANFLG | 1160 | TSYSGRSAVVRG |
| 805 | TSPTGRSANPLG | 1161 | TSYTGRSAVYRG |
| 806 | TSPSGRSAIPLG | 1162 | TSYTGRSAVHRG |
| 807 | TSYTGRSANPLG | | |

The following sequence may also be used as a protease cleavage sequence:

(SEQ ID NO: 1163)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; and X8 is an amino acid selected from A, D, E, F, G, H, I, K, L, M, N, P, Q, R, S, T, V, W and Y.

The following sequence may also be used as a protease cleavage sequence:

(SEQ ID NO: 1164)
X1-X2-X3-X4-X5-X6-X7-X8

wherein, X1 to X8 each represent a single amino acid, X1 is an amino acid selected from A, E, F, G, H, K, M, N, P, Q, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 1165)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, F, L, M, P, Q, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 1166)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, E, F, H, I, K, L, M, N, P, Q, R, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 1167)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, G, H, I, K, L, M, N, Q, R, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 1168)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from E, F, K, M, N, P, Q, R, S and W; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 1169)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, F, G, L, M, P, Q, V and W; X8 is an amino acid selected from A, D, E, F, G, H, I, K, L, M, N, P, Q, R, S, T, V, W and Y.

The following sequence may also be used as a protease cleavage sequence:

(SEQ ID NO: 1170)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, I, K, N, T and W

The following sequence may also be used as a protease cleavage sequence:

(SEQ ID NO: 1171)
X1-X2-X3-X4-X5-X6-X7-X8

wherein, X1 to X8 each represent a single amino acid, X1 is an amino acid selected from A, G, I, P, Q, S and Y; X2 is an amino acid selected from K or T; X3 is G; X4 is R; X5 is S; X6 is A; X7 is an amino acid selected from H, I and V; X8 is an amino acid selected from H, V and Y.

The following sequence may also be used as a protease cleavage sequence:

(SEQ ID NO: 1172)
X1-X2-X3-X4-X5-X6-X7-X8

wherein, X1 to X8 each represent a single amino acid, X1 is Y; X2 is an amino acid selected from S and T; X3 is G; X4 is R; X5 is S; X6 is an amino acid selected from A and E; X7 is an amino acid selected from N and V; X8 is an amino acid selected from H, P, V and Y.

The following sequence may also be used as a protease cleavage sequence:

(SEQ ID NO: 1173)
X1-X2-X3-X4-X5-X6-X7-X8-X9

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1174)
X1-X2-X3-X4-X5-X6-X7-X8-X9

wherein, X1 to X9 each represent a single amino acid, X1 is an amino acid selected from A, E, F, G, H, K, M, N, P, Q, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1175)
X1-X2-X3-X4-X5-X6-X7-X8-X9

wherein, X1 to X9 each represent a single amino acid, X1 is an amino acid selected from A, D, E, F, G, H, I, K, M, N, P, Q, S, T, W and Y; X2 is an amino acid selected from A, D, F, L, M, P, Q, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1176)
X1-X2-X3-X4-X5-X6-X7-X8-X9

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, E, F, H, I, K, L, M, N, P, Q, R, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1177)
X1-X2-X3-X4-X5-X6-X7-X8-X9

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, G, H, I, K, L, M, N, Q, R, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1178)
X1-X2-X3-X4-X5-X6-X7-X8-X9

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from E, F, K, M, N, P, Q, R, S and W; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1179)
X1-X2-X3-X4-X5-X6-X7-X8-X9

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, F, G, L, M, P, Q, V and W; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1180)
X1-X2-X3-X4-X5-X6-X7-X8-X9

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, I, K, N, T and W; X9 is an amino acid selected from A, G, H, I, L and R.

The following sequence may also be used as a protease cleavage sequence:

(SEQ ID NO: 1181)
X1-X2-X3-X4-X5-X6-X7-X8-X9

wherein, X1 to X9 each represent a single amino acid, X1 is an amino acid selected from A, G, I, P, Q, S and Y; X2 is an amino acid selected from K or T; X3 is G; X4 is R; X5 is S; X6 is A; X7 is an amino acid selected from H, I and V; X8 is an amino acid selected from H, V and Y; X9 is an amino acid selected from A, G, H, I, L and R.

The following sequence may also be used as a protease cleavage sequence:

(SEQ ID NO: 1182)
X1-X2-X3-X4-X5-X6-X7-X8-X9

wherein, X1 to X9 each represent a single amino acid, X1 is Y; X2 is an amino acid selected from S and T; X3 is G; X4 is R; X5 is S; X6 is an amino acid selected from A and E; X7 is an amino acid selected from N and V; X8 is an amino acid selected from H, P, V and Y; X9 is an amino acid selected from A, G, H, I, L and R.

The following sequence may also be used as a protease cleavage sequence:

(SEQ ID NO: 1183)
X10-X11-X1-X2-X3-X4-X5-X6-X7-X8

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 1184)
X10-X11-X1-X2-X3-X4-X5-X6-X7-X8

wherein, X1 to X11 each represent a single amino acid, X10 is an amino acid selected from I, T and Y; X11 is S; X1 is an amino acid selected from A, E, F, G, H, K, M, N, P, Q, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 1185)
X10-X11-X1-X2-X3-X4-X5-X6-X7-X8

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, F, L, M, P, Q, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 1186)
X10-X11-X1-X2-X3-X4-X5-X6-X7-X8

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, E, F, H, I, K, L, M, N, P, Q, R, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 1187)
X10-X11-X1-X2-X3-X4-X5-X6-X7-X8

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, G, H, I, K, L, M, N, Q, R, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 1188)
X10-X11-X1-X2-X3-X4-X5-X6-X7-X8

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from E, F, K, M, N, P, Q, R, S and W; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 1189)
X10-X11-X1-X2-X3-X4-X5-X6-X7-X8

wherein, X1 to X1I each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, F, G, L, M, P, Q, V and W; X8 is an amino acid selected from A, D, E, F, G, H, I, K, L, M, N, P, Q, R, S, T, V, W and Y.

The following sequence may also be used as a protease cleavage sequence:

(SEQ ID NO: 1190)
X10-X11-X1-X2-X3-X4-X5-X6-X7-X8

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, I, K, N, T and W.

The following sequence may also be used as a protease cleavage sequence:

(SEQ ID NO: 1191)
X10-X11-X1-X2-X3-X4-X5-X6-X7-X8

wherein, X1 to X11 each represent a single amino acid, X10 is an amino acid selected from I, T and Y; X11 is S; X1 is an amino acid selected from A, G, I, P, Q, S and Y; X2 is an amino acid selected from K or T; X3 is G; X4 is R; X5 is S; X6 is A; X7 is an amino acid selected from H, I and V; X8 is an amino acid selected from H, V and Y.

The following sequence may also be used as a protease cleavage sequence:

(SEQ ID NO: 1192)
X10-X11-X1-X2-X3-X4-X5-X6-X7-X8

wherein, X1 to X11 each represent a single amino acid, X10 is an amino acid selected from I, T and Y; X11 is S; X1 is Y; X2 is an amino acid selected from S and T; X3 is G; X4 is R; X5 is S; X6 is an amino acid selected from A and E; X7 is an amino acid selected from N and V; X8 is an amino acid selected from H, P, V and Y.

The following sequence may also be used as a protease cleavage sequence:

(SEQ ID NO: 1193)
X10-X11-X1-X2-X3-X4-X5-X6-X7-X8-X9

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1194)
X10-X11-X1-X2-X3-X4-X5-X6-X7-X8-X9

wherein, X1 to X11 each represent a single amino acid, X10 is an amino acid selected from I, T and Y; X11 is S; X1 is an amino acid selected from A, E, F, G, H, K, M, N, P, Q, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1195)
X10-X11-X1-X2-X3-X4-X5-X6-X7-X8-X9

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, F, L, M, P, Q, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1196)
X10-X11-X1-X2-X3-X4-X5-X6-X7-X8-X9

wherein, X1 to X1I each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, E, F, H, I, K, L, M, N, P, Q, R, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1197)
X10-X11-X1-X2-X3-X4-X5-X6-X7-X8-X9

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, G, H, I, K, L, M, N, Q, R, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1198)
X10-X11-X1-X2-X3-X4-X5-X6-X7-X8-X9

wherein, X1 to X1I each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from E, F, K, M, N, P, Q, R, S and W; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1199)
X10-X11-X1-X2-X3-X4-X5-X6-X7-X8-X9

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, F, G, L, M, P, Q, V and W; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 1200)
X10-X11-X1-X2-X3-X4-X5-X6-X7-X8-X9

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, I, K, N, T and W; X9 is an amino acid selected from A, G, H, I, L and R.

The following sequence may also be used as a protease cleavage sequence:

(SEQ ID NO: 1201)
X10-X11-X1-X2-X3-X4-X5-X6-X7-X8-X9

wherein, X1 to X1I each represent a single amino acid, X10 is an amino acid selected from I, T and Y; X1I is S; X1 is an amino acid selected from A, G, I, P, Q, S and Y; X2 is an amino acid selected from K or T; X3 is G; X4 is R; X5 is S; X6 is A; X7 is an amino acid selected from H, I and V; X8 is an amino acid selected from H, V and Y; X9 is an amino acid selected from A, G, H, I, L and R.

The following sequence may also be used as a protease cleavage sequence:

(SEQ ID NO: 1202)
X10-X11-X1-X2-X3-X4-X5-X6-X7-X8-X9

wherein, X1 to X11 each represent a single amino acid, X10 is an amino acid selected from I, T and Y; X1I is S; X1 is Y; X2 is an amino acid selected from S and T; X3 is G; X4 is R; X5 is S; X6 is an amino acid selected from A and E; X7 is an amino acid selected from N and V; X8 is an amino acid selected from H, P, V and Y; X9 is an amino acid selected from A, G, H, I, L and R.

Examples of the protease cleavage sequence that can be used include, but are not limited to, sequences disclosed in WO2019/107380, WO2019/107384, WO2020/246567, WO2015/116933, WO2015/048329, WO2016/118629, WO2016/179257, WO2016/179285, WO2016/179335, WO2016/179003, WO2016/046778, WO2016/014974, U.S. Patent Publication No. US2016/0289324, U.S. Patent Publication No. US2016/0311903, PNAS (2000) 97: 7754-7759, Biochemical Journal (2010) 426: 219-228, and Beilstein J Nanotechnol. (2016) 7: 364-373.

In addition to using the above-mentioned protease cleavage sequences, novel protease cleavage sequences may also be obtained by screening. For example, based on the result of crystal structure analysis of a known protease cleavage sequence, novel protease cleavage sequences can be explored by changing the interaction of active residues/recognition residues of the cleavage sequence and the enzyme. Novel protease cleavage sequences can also be explored by altering amino acids in a known protease cleavage sequence and examining interaction between the altered sequence and the protease. As another example, protease cleavage sequences can be explored by examining interaction of the protease with a library of peptides displayed using an in vitro display method such as phage display and ribosome display, or with an array of peptides immobilized on a chip or beads. Interaction between a protease cleavage sequence and a protease can be examined by testing cleavage of the sequence by the protease in vitro or in vivo.

For example, the protease cleavage sequences shown in Table 37 have all been disclosed in WO2019/107384. Polypeptides containing these protease cleavage sequences are all useful as protease substrates which are hydrolyzed by the action of proteases. Thus, the present invention provides protease substrates comprising a sequence selected from those described herein such as SEQ ID NOs: 369-448, and 1163-1202 above, and the sequences listed in Table 37.

The protease substrates of the present invention can be utilized as, for example, a library from which one with properties that suit the purpose can be selected to incorporate into a ligand-binding moiety or molecule. Specifically, in order to cleave the ligand-binding moiety/molecule selectively by a protease localized in the lesion, the substrates can be evaluated for sensitivity to that protease. When a ligand-binding moiety/molecule connected with a ligand moiety/molecule is administered in vivo, the molecule may come in contact with various proteases before reaching the lesion. Therefore, the molecule should preferably have sensitivity to the protease localized to the lesion and also as high resistance as possible to the other proteases. In order to select a desired protease cleavage sequence depending on the purpose, each protease substrate can be analyzed in advance for sensitivity to various proteases exhaustively to find its protease resistance. Based on the obtained protease resistance spectra, it is possible to find a protease cleavage sequence with necessary sensitivity and resistance. Alternatively, a ligand-binding molecule into which a protease cleavage sequence has been incorporated undergoes not only enzymatic actions by proteases but also various environmental stresses such as pH changes, temperature, and oxidative/reductive stress, before reaching the lesion. Resistance to these external factors can also be compared among the protease substrates, and this comparative information can be used to select a protease cleavage sequence with desired properties depending on the purpose.

Examples of the protease cleavage sequence include target sequences comprising an amino acid sequence as defined by SEQ ID NO: 194, 199, 215, 216, 217, 218, and 219. Specific examples of the protease cleavage sequence include target sequences comprising an amino acid sequence as defined by SEQ ID NO:199 or SEQ ID NO:194.

These specific examples of the protease cleavage sequence have been shown by the present inventors to be specifically hydrolyzed by the above-listed protease specifically expressed in a cancer tissue disclosed in International Publication Nos. WO2013/128194, WO2010/081173, and WO2009/025846, the inflammatory tissue specific protease, and the like. The target sequences comprising an amino acid sequence as defined by SEQ ID NO:199 or SEQ ID NO:194 were obtained by appropriately introducing an amino acid mutation to a target sequence that is specifically hydrolyzed by known protease.

Additional protease cleavage sequences are disclosed in International Publication No. WO2023/002952, which is incorporated herein by reference in its entirety for all purposes.

### Peptide Linker(s)

In one embodiment of the present invention, a flexible linker is further attached to one end or both ends of each protease cleavage site. The flexible linker attached to one end of the first protease cleavage site is referred to as “first flexible linker”, and the flexible linker attached to the other end as “second flexible linker”. In one embodiment of the present invention, a flexible linker is further attached to the C-terminal of the constant region, or C-terminal of Cx.

In a particular embodiment, the protease cleavage site and the flexible linker have any of the following formulas:
(protease cleavage site),
(first flexible linker)-(protease cleavage site),
(protease cleavage site)-(second flexible linker), and
(first flexible linker)-(protease cleavage site)-(second flexible linker).

The flexible linker according to the present embodiment is preferably a peptide linker. The first flexible linker and the second flexible linker each independently and arbitrarily exist and are identical or different flexible linkers each containing at least one flexible amino acid (Gly, etc.). The flexible linker contains, for example, a sufficient number of residues (amino acids arbitrarily selected from Arg, Ile, Gln, Glu, Cys, Tyr, Trp, Thr, Val, His, Phe, Pro, Met, Lys, Gly, Ser, Asp, Asn, Ala, etc., particularly Gly, Ser, Asp, Asn, and Ala, in particular, Gly and Ser, especially Gly, etc.) for the protease cleavage sequence to obtain the desired protease accessibility.

The flexible linker suitable for use at both ends of the protease cleavage sequence is usually a flexible linker that improves the access of protease to the protease cleavage sequence and elevates the cleavage efficiency of the protease. A suitable flexible linker may be readily selected and can be preferably selected from among different lengths such as 1 amino acid (Gly, etc.) to 20 amino acids, 2 amino acids to 15 amino acids, or 3 amino acids to 12 amino acids including 4 amino acids to 10 amino acids, 5 amino acids to 9 amino acids, 6 amino acids to 8 amino acids, or 7 amino acids to 8 amino acids. In some embodiments of the present invention, the flexible linker is a peptide linker of 1 to 7 amino acids.

Examples of the flexible linker include, but are not limited to, glycine polymers (G)n, glycine-serine polymers (including e.g., (GS)n, (GSGGS: SEQ ID NO: 10)n and (GGGS: SEQ ID NO: 1)n, wherein n is an integer of at least 1), glycine-alanine polymers, alanine-serine polymers, and other flexible linkers well known in conventional techniques.

Among them, glycine and glycine-serine polymers are receiving attention because these amino acids are relatively unstructured and easily function as neutral tethers between components.

Examples of the flexible linker consisting of the glycine-serine polymer can include, but are not limited to,

Ser
Gly Ser (GS)
Ser Gly (SG)
Gly Ser (GGS)
Gly Ser Gly (GSG)
Ser Gly Gly (SGG)
Gly Ser Ser (GSS)
Ser Ser Gly (SSG)
Ser Gly Ser (SGS)
Gly Gly Gly Ser (GGGS, SEQ ID NO: 1)
Gly Gly Ser Gly (GGSG, SEQ ID NO: 2)
Gly Ser Gly Gly (GSGG, SEQ ID NO: 3)
Ser Gly Gly Gly (SGGG, SEQ ID NO: 4)
Gly Ser Ser Gly (GSSG, SEQ ID NO: 5)
Gly Gly Gly Gly Ser (GGGGS, SEQ ID NO: 6)
Gly Gly Gly Ser Gly (GGGSG, SEQ ID NO: 7)
Gly Gly Ser Gly Gly (GGSGG, SEQ ID NO: 8)
Gly Ser Gly Gly Gly (GSGGG, SEQ ID NO: 9)
Gly Ser Gly Gly Ser (GSGGS, SEQ ID NO: 10)
Ser Gly Gly Gly Gly (SGGGG, SEQ ID NO: 11)
Gly Ser Ser Gly Gly (GSSGG, SEQ ID NO: 12)
Gly Ser Gly Ser Gly (GSGSG, SEQ ID NO: 13)
Ser Gly Gly Ser Gly (SGGSG, SEQ ID NO: 14)
Gly Ser Ser Ser Gly (GSSSG, SEQ ID NO: 15)
Gly Gly Gly Gly Gly Ser (GGGGGS, SEQ ID NO: 16)
Ser Gly Gly Gly Gly Gly (SGGGGG, SEQ ID NO: 17)
Gly Gly Gly Gly Gly Gly Ser (GGGGGGS, SEQ ID NO: 18)
Ser Gly Gly Gly Gly Gly Gly (SGGGGGG, SEQ ID NO: 19)
(Gly Gly Gly Gly Ser (GGGGS, SEQ ID NO: 6))n
(Ser Gly Gly Gly Gly (SGGGG, SEQ ID NO: 11))n
(Gly Gly Ser Gly Gly (GGSGG, SEQ ID NO: 8))n

wherein n is an integer of 1 or larger.

However, the length and sequence of the peptide linker can be appropriately selected by those skilled in the art according to the purpose.

Additional peptide linkers are disclosed in International Publication No. WO2023/002952, which is incorporated herein by reference in its entirety for all purposes.

In some embodiments of the present invention, the ligand-binding moiety or molecule or protein complex comprises a ligand-binding domain comprising a heavy chain variable domain (VH) and a light chain variable domain (VL). In embodiments, the ligand-binding domain comprises a light chain variable domain (VL) and a heavy chain variable domain (VH), wherein the VH is associated with the VL. In embodiments, the ligand-binding moiety/molecule comprises a light chain variable domain (VL) and a light chain constant domain (CL) in a first polypeptide, and a heavy chain variable domain (VH), wherein the VH is associated with the VL of the first polypeptide to form a ligand-binding domain. In embodiments, the ligand-binding moiety/molecule comprises a light chain variable domain (VL) and a light chain constant domain (CL) in a first polypeptide, and a heavy chain variable domain (VH) and a heavy chain constant domain in a second polypeptide is associated with the CL of the first polypeptide, and wherein the VH of the second polypeptide is associated with the VL of the first polypeptide to form a ligand-binding domain. In embodiments, the heavy chain constant domain comprises or consists of, from the N- to the C-terminus, a CH1 domain, a second peptide linker, a CH2 domain and a CH3 domain. In embodiments, the CH1 domain of a heavy chain constant domain and a CL are bound to each other via one or more disulfide bonds.

In some embodiments of the present invention, the ligand-binding moiety or molecule or protein complex contains a Fc region. In embodiments herein, the Fc region is a human IgG antibody Fc region. its type is not limited, and, for example, human IgG1, IgG2, IgG3, or IgG4 Fc region may be used. For example, a Fc region containing one sequence selected from the amino acid sequences represented by SEQ ID NOs: 362, 363, 364, and 365, or a Fc region mutant prepared by adding an alteration to the Fc region may be used. In some embodiments of the present invention, the ligand binding moiety/molecule comprises an antibody constant region. For instance, the heavy chain constant region of human IgG1, human IgG2, human IgG3, and human IgG4 are shown in SEQ ID NOs: 362 to 365, respectively. For instance, the Fc region of human IgG1, human IgG2, human IgG3, and human IgG4 are shown as a partial sequence of SEQ ID NOs: 362 to 365. In embodiments herein, the Cx region comprises or consists of the amino acid sequence of SEQ ID NO: 75.

In the above-mentioned embodiments, the protein complex of the present invention preferably comprises two ligand moieties (bivalent) which are connected with the C-terminal region of the antibody moiety via one or two peptide linkers.

In some embodiments of the present invention, a domain having ligand binding activity is separated from the ligand-binding moiety/molecule by the cleavage of the protease cleavage site or the protease cleavage sequence in the ligand-binding moiety/molecule so that the binding to the ligand is attenuated or abolished.

In some embodiments of the present invention, the protein complex is designed such that the protease cleavage site or protease cleavage sequence is provided in the ligand-binding moiety or molecule comprising a ligand-binding domain comprising a VH and a VL, and whereas the two peptides in the Fab structure have entire heavy chain-light chain interaction with each other before cleavage, the cleavage of the protease cleavage site or protease cleavage sequence results in attenuation or abolishment of the interaction between the peptide containing the VH (or a portion of the VH) and the peptide containing the VL (or a portion of the VL), reducing or abolishing the association between the VH and the VL.

### VH and VL Domains

In the present specification, the term “association” or “interaction” as used herein can refer to, for example, a state where two or more polypeptide regions associate or interact with each other. In general, a hydrophobic bond, a hydrogen bond, an ionic bond, or the like is formed between the intended polypeptide regions to form an associate. As one example of common association, an antibody typified by a natural antibody is known to retain a paired structure of a heavy chain variable region (VH) and a light chain variable region (VL) through a noncovalent bond or the like therebetween. In embodiments, association is retained by one or more disulfide bonds between a CL and a CH1 region.

In some embodiments of the present invention, VH and VL contained in the ligand-binding domain associate with each other. The association between the VH and the VL may be abolished, for example, by the cleavage at the cleavage site or the protease cleavage sequence. The abolishment of the association can be used interchangeably with, for example, the whole or partial abolishment of the state where two or more polypeptide regions interact with each other. For the abolishment of the association between the VH and the VL, the interaction between the VH and the VL may be wholly abolished, or the interaction between the VH and the VL may be partially abolished. In some embodiments, the ligand-binding moiety or molecule encompasses a ligand-binding domain in which the association between VL and VH is abolished by the cleavage at the protease cleavage site or protease cleavage sequence.

In some embodiments, the molecular weight of the protein complex after protease cleavage at the protease cleavage site (“in the second state”) is smaller than the molecular weight of the protein complex before protease cleavage at the protease cleavage site (“in the first state”). In some embodiments, the molecular weight of the VH, VL, or a portion of ligand-binding domain released upon cleavage at the protease cleavage sequence is approximately 26 kDa, or 13 kDa, or smaller. In some embodiments, the VH, or VL is released upon cleavage at the protease cleavage site, and the molecular weight of the released VH, or VL, is approximately 26 kDa, i.e. full-length VH or VL. In some embodiments, the ratio of the molecular weight of the protein complex in the first state and the molecular weight of the protein complex in the second state is 10:9, or the molecular weight of the protein complex in the second state is 9/10 that of the molecular weight of the protein complex in the first state, or the percentage reduction in molecular weight of the protein complex in the second state compared to the protein complex in the first state is 10%.

The term “dissociation” as used herein can refer to, the whole or partial abolishment of the abovementioned interactions. In some embodiments, the term “reduced association between VH and VL”, or “reduced interaction between VH and VL” may also be used interchangeably to refer to the whole or partial abolishment or attenuation of the association between the peptide containing the VH (or a portion of the VH) and the peptide containing the VL (or a portion of the VL). In further embodiments, reducing association or interaction is complete, leading to an abolishment of any association or interaction between VH and VL. In such cases, VH or VL completely dissociates from each other, also referred herein as “VH release”, or “VL release”. As used herein, “VH release” and “VL release” refers to the release of antibody VH, or fragment thereof, or fragment of the cleaved protein comprising VH, or fragment thereof, and the release of antibody VL, or fragment thereof, or fragment of the cleaved protein comprising VL, or fragment thereof, respectively. In a preferred embodiment, abolishment of the association between VH and VL results in VH completely dissociated from VL, i.e. VH is released.

In some embodiments of the present invention, the ligand-binding moiety/molecule comprises a ligand-binding domain comprising VH and VL, and the VH and the VL are associated with each other in a state where the protease cleavage site or the protease cleavage sequence of the ligand-binding moiety/molecule is uncleaved, whereas the association between the VH and the VL is abolished by the cleavage at the cleavage site or the protease cleavage sequence. The cleavage site or the protease cleavage sequence may be placed at any position in the ligand-binding moiety/molecule as long as the ligand binding ability of the ligand-binding moiety/molecule can be attenuated or abolished by the cleavage of the cleavage site or the protease cleavage sequence.

In one embodiment of the present invention, the protease cleavage site or the protease cleavage sequence is located near the boundary between the antibody VH and the antibody constant region. The phrase “near the boundary between the antibody VH and the antibody heavy chain constant region” can refer to between amino acid position 101 (Kabat numbering) of the antibody VH and amino acid position 140 (EU numbering) of the antibody heavy chain constant region and can preferably refer to between amino acid position 109 (Kabat numbering) of the antibody VH and amino acid position 122 (EU numbering) of the antibody heavy chain constant region, or between amino acid position 111 (Kabat numbering) of the antibody VH and amino acid position 122 (EU numbering) of the antibody heavy chain constant region. In embodiments herein, the protein or polypeptide complexes herein comprise a polypeptide having the structure represented by the partial formula taken from formula (I) or (II) above [VH]-[Lx]-[Cx] wherein each of VH, Lx and Cx are characterized to comprise or consist of specific sequences.

In the present invention, the ligand-binding moiety/molecule of the invention such as a fusion protein or protein complex comprises a ligand-binding domain further comprising at least one amino acid modification that reduces association between VH and VL in the cleaved state (“second state”) compared to the uncleaved state (“first state”), particularly the modification of an amino acid present at the interface between the VH and the VL

The dissociation of VH and VL from the fusion protein or protein complex or the reduction in association between VH and VL comprised within said fusion protein or protein complex is promoted by at least one amino acid modification performed at the interface between VH and VL that reduces association between VH and VL or reduces the interaction of VH with VL in the cleaved state (“second state”) compared to the uncleaved state “(first state”).

The term “interface” as used herein, refers to a face at which two regions (of amino acid residues) associate or interact with each other. Amino acid residues forming the interface are usually one or a plurality of amino acid residues contained in each polypeptide region subjected to association and dissociation and more preferably refer to amino acid residues that approach each other upon association or distance from each other during dissociation and participate in interaction. Specifically, the interaction includes noncovalent bonds such as a hydrogen bond, electrostatic interaction, or salt bridge formation between the amino acid residues.

The phrase “amino acid residues forming the interface” as used herein, specifically refers to amino acid residues contained in polypeptide regions constituting the interface. As one example, the polypeptide regions constituting the interface refer to polypeptide regions responsible for intramolecular or intermolecular selective binding in antibodies, ligands, receptors, substrates, etc. In some embodiments of the present invention, examples of such polypeptide regions include the VH and VL regions, particularly, the framework regions (FR) within the VH and VL regions. The amino acid residues forming the interface can be identified, for example, by analyzing the conformations of polypeptides and examining the amino acid sequences of polypeptide regions forming the interface upon association or dissociation of the polypeptides.

In some embodiments, an amino acid residue(s) forming the interface can be altered in order to promote the dissociation of, or reduce the interaction or association between, the heavy chain variable region (VH) and the light chain variable region (VL). In a preferred embodiment, the amino acid residue(s) forming the interface can be altered by a method of introducing a mutation(s) to the interface amino acid residue(s) such that two or more amino acid residues forming the interface have the same charge(s). The alteration of the amino acid residue(s) to result in the same charge(s) includes the alteration of a positively charged amino acid residue(s) to a negatively charged amino acid residue(s) or an uncharged amino acid residue, the alteration of a negatively charged amino acid residue to a positively charged amino acid residue(s) or an uncharged amino acid residue(s), and the alteration of an uncharged amino acid residue(s) to a positively or negatively charged amino acid residue(s). Such an amino acid alteration is performed for the purpose of promoting the dissociation or reducing the interaction or association and is not limited by the position of the amino acid alteration or the type of the amino acid as long as the purpose of promoting the dissociation or reducing interaction can be achieved. Examples of the alteration include, but are not limited to, substitution.

In the present specification, the amino acid residues forming the interface between the VH and the VL include, but are not limited to, amino acid residues at positions 36, 37, 38, 44, 45, 46, 47, 49, 87, 91, 98, and 103 (J. Mol. Biol. (2005) 350, 112-125, Sci Reports. (2017) 7, 12276). Altering the amino acid residue(s) forming the interface between the VH and the VL, particularly, substitution of amino acid residue(s) can promote the dissociation of or reduce the interaction or association between the VH and VL. Examples of modifiable amino acid position(s) for substitution(s) include, but are not limited to 37, 38, 39, 44, 45, 46, 47, 91, and 103 on the VH, and, 36, 37, 38, 43, 44, 46, 49, 87 and 98 on the VL (according to Kabat numbering). Examples of such amino acid substitution(s) include, but are not limited to V37, R38, Q39, G44, L45, E46, W47, H91, Y91, and W103 on the VH, and, Y36, Q37, R38, A43, P44, L46, Y49, Y87 and F98 on the VL (according to Kabat numbering). Examples of such amino acid substitution(s) include, but are not limited to Q39D, W47A, W47L, W47M, Y91A, Y91L, Y91M, H91A, W103A, W103I, W103L, W103M, V37S, V37Q, G44Q, L45A, and L45Q on the VH, or R38E, Y49A, Y87A, Y87L, Y87M, F98A, F98L, F98M, A43Q, P44A, P44S, P44Q, L46E, and L46Q on the VL (according to Kabat numbering). Alteration of amino acid residue(s) is not limited to VH or VL alone but also includes alteration on both VH and VL, as long as the purpose of promoting dissociation of VH or VL from the other can be achieved. Alteration of amino acid residue(s) can occur at any position(s) forming the interface between the VH and VL as long as it does not disrupt binding of the ligand to the ligand-binding domain of the protein complex. Further, alteration of amino acid residue(s) does not have to occur in pairs as long as the alteration reduces association between the VH and VL without disrupting the binding activity of the ligand to the protein complex in its uncleaved state. That is to say, alteration of amino acid residue(s) does not necessarily restrict the modifications in pairs, and includes any combination of amino acid modifications, for example, only one amino acid modification on the VH combined with two amino acid modifications on the VL or two amino acid modifications on the VH combined with three amino acid modifications on the VL. Alteration of amino acid residue can also be a single amino acid modification on the VH or the VL as long as the modification alone is capable of reducing the association between the VH and the VL but does not disrupt binding activity of the ligand or antigen to the protein complex. For the avoidance of doubt, where amino acid modifications are performed on both the VH and the VL in at least a pair, the amino acid modification(s) performed on the VH and the amino acid modification(s) performed on the VL are not necessarily identical, and not necessarily different so long as the modifications are capable of reducing the association between the VH and the VL but does not disrupt binding activity of the ligand to the protein complex in the uncleaved state. Confirmation that the amino acid modification(s) introduced at selected position(s) within the interface between VH and VL does not disrupt binding of the ligand to the ligand-binding domain of the protein complex may be conducted by any of the common methods described herein or as known to the skilled person.

In one embodiment, the amino acid modification are combinations of substitution(s) of (an) amino acid(s) present at the interface between the VH and the VL within the FR region and optionally within the CDR region. In a preferable embodiment, the combination substitutions are selected from the following groups (a) to (cc):
(a) L46Q and Y49A on the VL;
(b) Y91A on the VH, and L46Q and Y49A on the VL;
(c) Y91M on the VH, and A43Q and Y49A on the VL;
(d) Y91M on the VH, and A43Q, L46Q, and Y49A on the VL;
(e) H91A on the VH, and L46Q and Y49A on the VL;
(f) W103L on the VH, and L46Q and Y49A on the VL;
(g) W103I on the VH, and L46Q and Y49A on the VL;
(h) W103M on the VH, and A43Q and Y49A on the VL;
(i) W103M on the VH, and L46Q and Y49A on the VL;
(j) V37S on the VH, and A43Q and Y49A on the VL;
(k) V37S on the VH, and L46Q and Y49A on the VL;
(l) L45Q on the VH, and A43Q and Y49A on the VL;
(m) L45Q on the VH, and L46Q and Y49A on the VL;
(n) F100aI on the VH, and A43Q and Y49A on the VL;
(o) F100aI on the VH, and A43Q, L46Q, and Y49A on the VL;
(p) W103L on the VH, and S30V, L46Q, and Y49A on the VL;
(q) W103M on the VH, and S30V, L46Q, and Y49A on the VL;
(r) V37S and F100aI on the VH, and S30V, A43Q, and Y49A on the VL;
(s) V37S and F100aI on the VH, and S30V, L46Q, and Y49A on the VL;
(t) W103L on the VH, and L46Q and Y49A on the VL;
(u) W103I on the VH, and L46Q and Y49A on the VL;
(v) W103M on the VH, and Y49A and Y87L on the VL;
(w) W103L on the VH, and Y49A and Y87L on the VL;
(x) W103L on the VH, and S30V, Y49A, and Y87L on the VL;
(y) V37S and F100aI on the VH, and L46Q and Y49A on the VL;
(z) V37S and F100aI on the VH, and Y49A and Y87L on the VL;
(aa) V37S and F100aI on the VH, and S30V, Y49A, and Y87L on the VL;
(bb) V37S, F100aI and W103M on the VH, and L46Q and Y49A on the VL; and
(cc) V37S, F100aI and W103L on the VH, and L46Q and Y49A on the VL.

In the present invention, it has been established that certain amino acid residues forming the interface between the VH and the VL are crucial, and that their alteration leads to an improved VH release. In embodiments, the present disclosure includes combinations of these amino acid residues forming the interface between the VH and the VL, and optionally amino acid residues residing in the CDR. Examples of modifiable amino acid position(s) for substitution(s) include, but are not limited to 37 and 100aI on the VH, and, 30, 46, and 49, on the VL (according to Kabat numbering). Examples of such amino acid substitution(s) include, but are not limited to V37, and F100a on the VH, and, S30, L46, and Y49 on the VL (according to Kabat numbering). Examples of such amino acid substitution(s) include, but are not limited to V37S and F100aI on the VH, or S30V, L46Q, or Y49A on the VL (according to Kabat numbering). It has been confirmed that the amino acid modification(s) introduced at the selected position(s) within the interface between VH and VL, and optionally including an amino acid residing in the CDR, do not disrupt binding of the ligand to the ligand-binding domain or the binding of the ligand to an antigen-binding domain, and that the VH release property was enhanced.

In one embodiment, the amino acid modification are combinations of substitution of an amino acid present at the interface between the VH and the VL within the FR region, and optionally an amino acid residing in the CDR. In a preferable embodiment, the combination substitutions are selected from the following groups:
(a) L46Q and Y49A on VL;
(b) V37S and F100aI on VH;
(c) S30V, L46Q, and Y49A on VL (according to Kabat numbering).

In the present invention, the VH and VL can comprise further amino acid alterations in addition to the alterations mentioned above. The alterations mentioned above enhanced the VH release property. The further alterations lead to an affinity matured antibody or antibody construct and comprise at least one of the following substitutions selected from the following groups, or combinations thereof:
(a) G55Q and N95T on VH;
(b) P52aA, G55Q, N95T and V102Q on VH;
(c) N92Y on VL;
(d) A25S and Q27D on VL;
(e) A25S, Q27D and S56E on VL (according to Kabat numbering).

In the present invention, the VL can therefore comprise amino acid alterations selected from the following groups:
(a) L46Q and Y49A on VL; (such as in SEQ ID NO: 190)
(b) L46Q, Y49A, and N92Y on VL; (such as in SEQ ID NO: 342)
(c) A25S, Q27D, L46Q, and Y49A on VL; (such as in SEQ ID NO: 343)
(d) A25S, Q27D, L46Q, Y49A, and S56E on VL; (such as in SEQ ID NO: 344)
(e) S30V, L46Q, and Y49A on VL; (such as in SEQ ID NO: 191) (according to Kabat numbering).

In the present invention, the VH can therefore comprise amino acid alterations selected from the following groups:
(a) V37S and F100aI on VH; (such as in SEQ ID NO: 189)
(b) V37S, G55Q, N95T, and F100aI on VH; (such as in SEQ ID NO: 340)
(c) V37S, P52aA, G55Q, N95T, F100aI, and V102Q on VH; (such as in SEQ ID NO: 341) (according to Kabat numbering).

It has been verified that the amino acid modification(s) introduced at selected position(s) within the interface between VH and VL as defined above reduce association between VH and VL to a level sufficient to promote dissociation from each other. A method of detecting the release of VH from the fusion protein or protein complex includes a method of detecting VH(s) release by comparing the molecular weight of the fusion protein or protein complex before and after protease cleavage using well-known methods such as SDS-PAGE, Size Exclusion Chromatography (SEC) or BIACORE® using SPR. In an assay using BIACORE®, the fusion protein or protein complex is immobilized on R-Protein A coupled-carboxymethylated dextran biosensor chips (CM4-ProA/G, BIACORE®, Inc.) biosensor chips that are activated with N-ethyl-N′-(3-dimethylaminopropyl)-carbodiimide hydrochloride (EDC) and N-hydroxysuccinimide (NHS) according to the supplier's instructions. Protease, such as urokinase-type plasminogen activator (uPA), at a concentration of 400 nM is injected in assay buffer (HBS-EP+, Cytiva) at a flow rate of 2 microliter/min at 37 degrees C. for an association time of 1800 secs and dissociation time of 10 secs. The response unit (RU) captured before and after protease injection is compared. A percentage reduction in response unit of less than or equivalent to 1%, or less than or equivalent to 2%, or less than or equivalent to 3%, or less than or equivalent to 4%, or less than or equivalent to 5%, or less than or equivalent to 6%, or less than or equivalent to 7% or less than or equivalent to 8%, or less than or equivalent to 9%, or less than or equivalent to 10%, before and after protease cleavage indicates dissociation of a fragment from the fusion protein, or protein complex. In a preferred embodiment, a dissociation of less than or equivalent to 10% indicates complete dissociation of VH(s) or VL(s) from a bivalent fusion polypeptide (also referenced herein as a protein complex) of the present invention.

Additional VH and VL domains are disclosed in International Publication No. WO2023/002952, which is incorporated herein by reference in its entirety for all purposes.

### Ligand

In the present specification, the term “ligand moiety” or “ligand molecule” refers to a moiety or molecule having biological activity. Herein, the “ligand moiety” and “ligand molecule” may be simply referred to as “ligand”. The molecule having biological activity usually functions by interacting with a receptor on cell surface and thereby performing biological stimulation, inhibition, or modulation in other modes. These functions are usually thought to participate in the intracellular signaling pathways of cells carrying the receptor.

In the present specification, the ligand encompasses the desired molecule that exerts biological activity through interaction with a biomolecule, also referred herein as “binding partner”. For example, the ligand not only means a molecule that interacts with a receptor but also includes a molecule that exerts biological activity through interaction with the molecule, for example, a receptor that interacts with the molecule, or a binding fragment thereof. For example, a ligand binding site of a protein known as a receptor, and a protein containing an interaction site of the receptor with another molecule are included in the ligand according to the present invention. Specifically, for example, a soluble receptor, a soluble fragment of a receptor, an extracellular domain of a transmembrane receptor, and polypeptides containing them are included in the ligand according to the present invention.

The ligand of the present invention can usually exert desirable biological activity by binding to one or more binding partners. The binding partner of the ligand can be an extracellular, intracellular, or transmembrane protein. In one embodiment, the binding partner of the ligand is an extracellular protein, for example, a soluble receptor. In another embodiment, the binding partner of the ligand is a membrane-bound receptor. The ligand of the present invention can specifically bind to the binding partner with a dissociation constant (KD) of 10 micromolar (micro M), 1 micromolar, 100 nM, 50 nM, 10 nM, 5 nM, 1 nM, 500 pM, 400 pM, 350 pM, 300 pM, 250 pM, 200 pM, 150 pM, 100 pM, 50 pM, 25 pM, 10 pM, 5 pM, 1 pM, 0.5 pM, or 0.1 pM or less.

Examples of the molecule having biological activity include, but are not limited to, cytokines, chemokines, polypeptide hormones, growth factors, apoptosis inducing factors, PAMPs, DAMPs, nucleic acids, and fragments thereof. In a specific embodiment, an interleukin, an interferon, a hematopoietic factor, a member of the TNF superfamily, a chemokine, a cell growth factor, a member of the TGF-beta family, a myokine, an adipokine, or a neurotrophic factor can be used as the ligand. In a more specific embodiment, CXCL9, CXCL10, CXCL11, IL-2, IL-7, IL-12, IL-15, IL-18, IL-21, IL-22, IFN-alpha, IFN-beta, IFN-g, MIG, I-TAC, RANTES, MIP-1a, MIP-1b, IL-1R1 (Interleukin-1 receptor, type I), IL-1R2 (Interleukin-1 receptor, type II), IL-1RAcP (Interleukin-1 receptor accessory protein), or IL-1Ra (Protein Accession No. NP_776214, mRNA Accession No. NM_173842.2) can be used as the ligand. There is no limitation on the ligand used in the present disclosure. In some embodiments, the ligand may be a wild-type (or naturally occurring) ligand or a mutant ligand with any mutation(s). In the case of IL-12 or IL-22, which is a heterodimeric cytokine, in some embodiments, the ligand, IL-12, may be wild-type (or naturally-occurring) IL-12 or IL-22, or a mutant IL-12 or IL-22 with any mutation(s). In some embodiments, IL-12 may be a single-chain IL-12 in which p35 and p40 are linked to be contained in a single chain.

In some embodiments of the present invention, the ligand is a cytokine.

Cytokines are a secreted cell signaling protein family involved in immunomodulatory and inflammatory processes. These cytokines are secreted by glial cells of the nervous system and by many cells of the immune system. The cytokines can be classified into proteins, peptides and glycoproteins and encompass large diverse regulator families.

The cytokines can induce intracellular signal transduction through binding to their cell surface receptors, thereby causing the regulation of enzyme activity, upregulation or downregulation of some genes and transcriptional factors thereof, or feedback inhibition, etc.

In some embodiments, the cytokine of the present invention includes immunomodulatory factors such as interleukins (IL) and interferons (IFN). A suitable cytokine can contain a protein derived from one or more of the following types: four alpha-helix bundle families (which include the IL-2 subfamily, the IFN subfamily and IL-10 subfamily); the IL-1 family (which includes IL-1 and IL-8); and the IL-17 family. The cytokine can also include those classified into type 1 cytokines (e.g., IFNgamma and TGF-beta) which enhance cellular immune response, or type 2 cytokines (e.g., IL-4, IL-10, and IL-13) which work advantageously for antibody reaction.

In the present specification, the ligand is derived from, or is interleukin 12 (IL-12). Interleukin 12 (IL-12) is a heterodimeric cytokine consisting of disulfide-linked glycosylated polypeptide chains of 30 and 40 kD (Accession No. NP_000873.2, P29459 and Accession No. NP_002178.2, P29460).

Interleukin 12 can bind to an IL-12 receptor expressed on the cytoplasmic membranes of cells (e.g., T cells and NK cells) and thereby change (e.g., start or block) a biological process. For example, the binding of IL-12 to an IL-12 receptor stimulates the growth of preactivated T cells and NK cells, promotes the cytolytic activity of cytotoxic T cells (CTL), NK cells and LAK (lymphokine-activated killer) cells, induces the production of gamma interferon (IFNgamma) by T cells and NK cells, and induces the differentiation of naive Th0 cells into Th1 cells producing IFNgamma and IL-2. In particular, IL-12 is absolutely necessary for setting the production and cellular immune response (e.g., Th1 cell-mediated immune response) of cytolytic cells (e.g., NK and CTL). Thus, IL-12 is absolutely necessary for generating and regulating both protective immunity (e.g., eradication of infectious disease) and pathological immune response (e.g., autoimmunity).

Examples of the method for measuring the physiological activity of IL-12 include a method of measuring the cell growth activity of IL-12, STAT4 reporter assay, a method of measuring cell activation (cell surface marker expression, cytokine production, etc.) by IL-12, and a method of measuring the promotion of cell differentiation by IL-12.

Interleukin 22 (IL-22) (Accession No. NP_065386.1, Q9GZX6) is a member of the IL-10 family of cytokines. It is secreted by immune cells such as T-cells, NKT-cells, type 3 innate lymphoid cells (ILC3), and to a lesser extent by neutrophils and macrophages. IL-22 binds to its receptor IL-22R, which is a heterodimer composed of IL-22R1 and IL-10R2. IL22R is mainly expressed on non-hematopoietic cells such as epithelial cells and stromal cells. IL-22 activity is regulated by IL-22 binding protein (IL-22BP, also known as IL22RA2), which is a secreted protein with high structural homology to IL-22R1. IL-22BP binds to IL-22 with high affinity, blocking it from interacting with IL-22R1.

Binding of IL-22 to IL-22 receptor leads to activation of JAKI and TYK2 kinases, which in turn leads to activation of STAT3 signaling. IL-22 plays an important role in epithelial cell function. For example, in the gut, IL-22 promotes the integrity of the intestinal barrier by stimulating proliferation of gut epithelial cells, mucus secretion and anti-microbial peptide secretion. In the liver, IL-22 acts as a survival factor for hepatocytes during liver injury, and also stimulates hepatocytes to proliferate for liver regeneration.

Examples of the method for measuring the physiological activity of IL-22 include a method of measuring the cell growth activity of IL-22, STAT3 reporter assay, and a method of measuring cell activation (cell surface marker expression, cytokine production, etc.) by IL-22.

Interleukin 2 (IL-2) is monomeric cytokine and mainly secreted by activated CD4 T and CD8 T cells. IL-2 binds to its receptor (IL-2R), which consists of 3 subunits, alpha, beta, and gamma. IL-2R beta and gamma are involved in signal transduction and IL-2R alpha and beta are involved in binding. All three subunits are important for high affinity cytokine-receptor complex. IL-2 is essential for both promoting and regulating immune responses since it binds and activates both effector T cells and regulatory T cells.

Examples of the method for measuring the physiological activity of IL-2 include a method of measuring the cell growth activity of IL-2, a method of measuring cell activation (cell surface marker expression, cytokine production, etc.) by IL-2, and a method of measuring the promotion of cell differentiation by IL-2.

In some embodiments of the present invention, the ligand is a chemokine. Chemokines generally act as chemoattractant that mobilize immune effector cells to chemokine expression sites. This is considered beneficial for expressing a particular chemokine gene, for example, together with a cytokine gene, for the purpose of mobilizing other immune system components to a treatment site. Such chemokines include CXCL10, RANTES, MCAF, MIP1-alpha, and MIP1-beta. Those skilled in the art should know that certain cytokines also have a chemo attractive effect and acknowledge that such cytokines can be classified by the term “chemokine”.

Chemokines are a homogeneous serum protein family of 7 to 16 kDa originally characterized by their ability to induce leukocyte migration. Most of chemokines have four characteristic cysteines (Cys) and are classified into CXC or alpha, CC or beta, C or gamma and CX3C or delta chemokine classes according to motifs formed by the first two cysteines. Two disulphide bonds are formed between the first and third cysteines and between the second and fourth cysteines. In general, the disulphide bridges are considered necessary. Clark-Lewis and collaborators have reported that the disulphide bonds are crucial for the chemokine activity of at least CXCL10 (Clark-Lewis et al., J. Biol. Chem. 269: 16075-16081, 1994). The only one exception to having four cysteines is lymphotactin, which has only two cysteine residues. Thus, lymphotactin narrowly maintains its functional structure by only one disulphide bond. Subfamilies of CXC or alpha are further classified, according to the presence of an ELR motif (Glu-Leu-Arg) preceding the first cysteine, into two groups: ELR-CXC chemokines and non-ELR-CXC chemokines (see e.g., Clark-Lewis, supra; and Belperio et al., “CXC Chemokines in Angiogenesis”, J. Leukoc. Biol. 68: 1-8, 2000).

Interferon-inducible protein-10 (IP-10 or CXCL10) (Accession No. NP_001556.2, P02778) is induced by interferon-gamma and TNF-alpha and produced by keratinocytes, endothelial cells, fibroblasts and monocytes. IP-10 is considered to play a role in mobilizing activated T cells to an inflammatory site of a tissue (Dufour, et al., “IFN-gamma-inducible protein 10 (IP-10; CXCLT0)-deficient mice reveal a role for IP-10 in effector T cell generation and trafficking”, J Immunol., 168: 3195-204, 2002). Furthermore, there is a possibility that IP-10 plays a role in hypersensitive reaction. There is a possibility that IP-10 also plays a role in the occurrence of inflammatory demyelinating neuropathies (Kieseier, et al., “Chemokines and chemokine receptors in inflammatory demyelinating neuropathies: a central role for IP-10”, Brain 125: 823-34, 2002).

Research indicates the possibility that IP-10 is useful in the engraftment of stem cells following transplantation (Nagasawa, T., Int. J. Hematol. 72: 408-11, 2000), the mobilization of stem cells (Gazitt, Y., J. Hematother Stem Cell Res 10: 229-36, 2001; and Hattori et al., Blood 97: 3354-59, 2001) and antitumor hyperimmunity (Nomura et al., Int. J. Cancer 91: 597-606, 2001; and Mach and Dranoff, Curr. Opin. Immunol. 12: 571-75, 2000). For example, previous reports known to those skilled in the art discuss the biological activity of chemokine (Bruce, L. et al., “Radiolabeled Chemokine binding assays”, Methods in Molecular Biology (2000) vol. 138, pp. 129-134; Raphaele, B. et al., “Calcium Mobilization”, Methods in Molecular Biology (2000) vol. 138, pp. 143-148; and Paul D. Ponath et al., “Transwell Chemotaxis”, Methods in Molecular Biology (2000) vol. 138, pp. 113-120 Humana Press. Totowa, New Jersey).

Examples of the biological activity of CXCL10 include binding to a CXCL10 receptor (CXCR3), CXCL10-induced calcium flux, CXCL10-induced cell chemotaxis, binding of CXCL10 to glycosaminoglycan and CXCL10 oligomerization. Examples of the method for measuring the physiological activity of CXCL10 include a method of measuring the cell chemotactic activity of CXCL10, reporter assay using a cell line stably expressing CXCR3 (see PLoS One. 2010 Sep. 13; 5 (9): e12700), and PathHunter™ beta-Arrestin recruitment assay using B-arrestin recruitment induced at the early stage of GPCR signal transduction.

Programmed death 1 (PD-1) protein is an inhibitory member of the CD28 family of receptors. The CD28 family also includes CD28, CTLA-4, ICOS and BTLA. PD-1 is expressed on activated B cells, T cells and bone marrow cells (Okazaki et al., (2002) Curr. Opin. Immunol. 14: 391779-82; and Bennett et al., (2003) J Immunol 170: 711-8). CD28 and ICOS, the initial members of the family, were discovered on the basis of functional influence on the elevation of T cell growth after monoclonal antibody addition (Hutloff et al., (1999) Nature 397: 263-266; and Hansen et al., (1980) Immunogenics 10: 247-260). PD-1 was discovered by screening for differential expression in apoptotic cells (Ishida et al., (1992) EMBO J 11: 3887-95). CTLA-4 and BTLA, the other members of the family, were discovered by screening for differential expression in cytotoxic T lymphocytes and TH1 cells, respectively. CD28, ICOS and CTLA-4 all have an unpaired cysteine residue which permits homodimerization. In contrast, PD-1 is considered to exist as a monomer and lacks the unpaired cysteine residue characteristic of other members of the CD28 family.

The PD-1 gene encodes a 55 kDa type I transmembrane protein which is part of the Ig superfamily. PD-1 contains a membrane-proximal immunoreceptor tyrosine inhibitory motif (ITIM) and a membrane-distal tyrosine-based switch motif (ITSM). PD-1 is structurally similar to CTLA-4 but lacks a MYPPPY motif (SEQ ID NO: 159) important for B7-1 and B7-2 binding. Two ligands, PD-L1 and PD-L2, for PD-1 have been identified and have been shown to negatively regulate T-cell activation upon binding to PD-1 (Freeman et al., (2000) J Exp Med 192: 1027-34; Latchman et al., (2001) Nat Immunol 2: 261-8; and Carter et al., (2002) Eur J Immunol 32: 634-43). Both PD-L1 and PD-L2 are B7 homologs that bind to PD-1, but do not bind to the other members of the CD28 family. PD-L1, one of the PD-1 ligands, is abundant in various human cancers (Dong et al., (2002) Nat. Med. 8: 787-9). The interaction between PD-1 and PD-L1 results in decrease in tumor-infiltrating lymphocytes, reduction in T cell receptor-mediated growth, and immune evasion by the cancerous cells (Dong et al., (2003) J. Mol. Med. 81: 281-7; Blank et al., (2005) Cancer Immunol. Immunother. 54: 307-314; and Konishi et al., (2004) Clin. Cancer Res. 10: 5094-100). Immunosuppression can be reversed by inhibiting the local interaction of PD-1 with PD-L1, and this effect is additive when the interaction of PD-2 with PD-L2 is also inhibited (Iwai et al., (2002) Proc. Natl. Acad. Sci. USA 99: 12293-7; and Brown et al., (2003) J. Immunol. 170: 1257-66).

PD-1 is an inhibitory member of the CD28 family expressed on activated B cells, T cells, and bone marrow cells. Animals deficient in PD-1 develop various autoimmune phenotypes, including autoimmune cardiomyopathy and lupus-like syndrome with arthritis and nephritis (Nishimura et al., (1999) Immunity 11: 141-51; and Nishimura et al., (2001) Science 291: 319-22). PD-1 has been further found to play an important role in autoimmune encephalomyelitis, systemic lupus erythematosus, graft-versus-host disease (GVHD), type I diabetes mellitus, and rheumatoid arthritis (Salama et al., (2003) J Exp Med 198: 71-78; Prokunia and Alarcon-Riquelme (2004) Hum Mol Genet 13: R143; and Nielsen et al., (2004) Lupus 13: 510). In a mouse B cell tumor line, the ITSM of PD-1 has been shown to be essential for inhibiting BCR-mediated Ca2+ flux and tyrosine phosphorylation of downstream effector molecules (Okazaki et al., (2001) PNAS 98: 13866-71).

In some embodiments, the ligand may be a wild-type (or naturally occurring) ligand or a mutant ligand with any mutation(s). In the case of IL-12, which is a heterodimeric cytokine, in some embodiments, the ligand, IL-12, may be wild-type (or naturally occurring) IL-12 or a mutant IL-12 with any mutation(s). In some embodiments, IL-12 may be a single-chain IL-12 in which p35 and p40 are linked to be contained in a single chain. In a particular embodiment, the ligand moiety/molecule of the present invention is IL-12 and the IL-12 is connected with C-terminal amino acid residue of the ligand-binding moiety/molecule via a peptide linker attached to p35 subunit of IL-12 or p40 subunit of IL-12. In one embodiment, the ligand moiety/molecule of the present invention is IL-12 and the IL-12 is connected with C-terminal amino acid residue of the ligand-binding moiety/molecule via a peptide linker attached to the N-terminus of p35 subunit of IL-12 or p40 subunit of IL-12. The same applies to any ligand in general or as described herein, including IL-22, etc.

In some embodiments of the present invention, a cytokine variant, a chemokine variant, or the like (e.g., Annu Rev Immunol. 2015; 33: 139-67) or a fusion protein containing the variants (e.g., Stem Cells Transl Med. 2015 January; 4 (1): 66-73) can be used as the ligand.

In some embodiments of the present invention, the ligand is selected from CXCL9, CXCL10, CXCL11, PD-1, IL-2, IL-12, IL-22, IL-6R, IL-1R1, IL-1R2, IL-1RAcP, and IL-1Ra. The CXCL10, PD-1, IL-2, IL-12, IL-22, IL-6R, IL-1R1, IL-1R2, IL-1RAcP, and IL-1Ra may have the same sequences as those of naturally occurring CXCL10, PD-1, IL-2, IL-12, IL-22, IL-6R, IL-1R1, IL-1R2, IL-1RAcP, and IL-1Ra, respectively, or may be a ligand variant that differs in sequence from naturally occurring CXCL9, CXCL10, CXCL11, PD-1, IL-2, IL-12, IL-22, IL-6R, IL-1R1, IL-1R2, IL-1RAcP, and IL-1Ra, but retains the physiological activity of the corresponding natural ligand. In order to obtain the ligand variant, an alteration may be artificially added to the ligand sequence for various purposes. Preferably, an alteration to resist protease cleavage (protease resistance alteration) is added thereto to obtain a ligand variant.

In some embodiments, the ligand binding moiety or molecule and the ligand moiety or molecule are fused via a peptide linker. For example, an arbitrary peptide linker that can be introduced by genetic engineering, or a linker disclosed as a synthetic compound linker (see e.g., Protein Engineering, 9 (3), 299-305, 1996) can be used as the linker in the fusion of the ligand binding molecule with the ligand. The length of the peptide linker is not particularly limited and may be appropriately selected by those skilled in the art according to the purpose. Examples of the peptide linker can include, but are not limited to:

Ser
Gly Ser (GS)
Ser Gly (SG)
Gly Gly Ser (GGS)
Gly Ser Gly (GSG)
Ser Gly Gly (SGG)
Gly Ser Ser (GSS)
Ser Ser Gly (SSG)
Ser Gly Ser (SGS)
Gly Gly Gly Ser (GGGS, SEQ ID NO: 1)
Gly Gly Ser Gly (GGSG, SEQ ID NO: 2)
Gly Ser Gly Gly (GSGG, SEQ ID NO: 3)
Ser Gly Gly Gly (SGGG, SEQ ID NO: 4)
Gly Ser Ser Gly (GSSG, SEQ ID NO: 5)
Gly Gly Gly Gly Ser (GGGGS, SEQ ID NO: 6)
Gly Gly Gly Ser Gly (GGGSG, SEQ ID NO: 7)
Gly Gly Ser Gly Gly (GGSGG, SEQ ID NO: 8)
Gly Ser Gly Gly Gly (GSGGG, SEQ ID NO: 9)
Gly Ser Gly Gly Ser (GSGGS, SEQ ID NO: 10)
Ser Gly Gly Gly Gly (SGGGG, SEQ ID NO: 11)
Gly Ser Ser Gly Gly (GSSGG, SEQ ID NO: 12)
Gly Ser Gly Ser Gly (GSGSG, SEQ ID NO: 13)
Ser Gly Gly Ser Gly (SGGSG, SEQ ID NO: 14)
Gly Ser Ser Ser Gly (GSSSG, SEQ ID NO: 15)
Gly Gly Gly Gly Gly Ser (GGGGGS, SEQ ID NO: 16)
Ser Gly Gly Gly Gly Gly (SGGGGG, SEQ ID NO: 17)
Gly Gly Gly Gly Gly Gly Ser (GGGGGGS, SEQ ID NO: 18)
Ser Gly Gly Gly Gly Gly Gly (SGGGGGG, SEQ ID NO: 19)
(Gly Gly Gly Gly Ser (GGGGS, SEQ ID NO: 6))n
(Ser Gly Gly Gly Gly (SGGGG, SEQ ID NO: 11))n
(Gly Gly Ser Gly Gly (GGSGG, SEQ ID NO: 8))n

wherein n is an integer of 1 or larger.

However, the length and sequence of the peptide linker can be appropriately selected by those skilled in the art according to the purpose.

The synthetic compound linker (chemical cross-linking agent) is a cross-linking agent usually used in peptide cross-linking, for example, N-hydroxysuccinimide (NHS), disuccinimidyl suberate (DSS), bis(sulfosuccinimidyl) suberate (BS3), dithiobis(succinimidyl propionate) (DSP), dithiobis(sulfosuccinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl]sulfone (BSOCOES), or bis[2-(sulfosuccinimidoxycarbonyloxy)ethyl]sulfone (sulfo-BSOCOES). These cross-linking agents are commercially available.

In some embodiments, the IL-12 of the present invention is a mutant IL-12 with a mutation that provides protease resistance. In some embodiments, the IL-12 of the present invention is an attenuated IL-12 or low potent IL-12 with a mutation that provides weaker affinity to the IL-12R. In some embodiments, IL-12 is a single-chain IL-12 in which p35 and p40 are linked to be contained in a single chain. In a particular embodiment, the ligand moiety/molecule of the present invention is IL-12 and the IL-12 is connected with C-terminal amino acid residue of the ligand-binding moiety/molecule via a peptide linker attached to p35 subunit of IL-12 or p40 subunit of IL-12. In a particular embodiment, the ligand moiety/molecule of the present invention is IL-12 and the IL-12 is connected with C-terminal amino acid residue of the ligand-binding moiety/molecule via a peptide linker attached to p40 subunit of IL-12. In some embodiments, the ligand binding moiety or molecule and the ligand moiety or molecule are fused via the peptide linker Ly comprising an amino acid sequence of SEQ ID NO: 68. In some embodiments, the constant domain of the heavy chain and the ligand are fused via the peptide linker Ly comprising an amino acid sequence of SEQ ID NO: 68.

### Protease-Resistant IL-12

In a particular embodiment, the invention provides a protein complex or fusion protein comprising a ligand that is interleukin-12 (IL-12), wherein the IL-12 has been modified to prevent its proteolytic degradation when exposed to a protease, i.e. protease-resistant IL-12. Modifications include amino acid modifications introduced to the IL-12 that prevent proteolytic degradation in the presence of protease, particularly wherein the modification is performed at the heparin binding site, which is in close proximity to the epitope of the variable region of the IL-12 binding domain of the protein complex or IL-12 fusion protein.

Native IL-12 comprises a heparin binding site which may be cleaved by a protease such as Human Matriptase/ST14 Catalytic Domain (MT-SP1). In a particular case of an IL-12 fusion protein, the heparin binding site may be in close proximity to the epitope of the variable region of the IL-12 binding domain of the protein complex or IL-12 fusion protein. Protease cleavage at the heparin binding site may affect the clearance of activated protein complex or IL-12 fusion protein. Thus, in some embodiments, to prevent unintentional cleavage of IL-12 at the heparin binding site, at least one amino acid modifications may be introduced into the heparin binding site of IL-12.

The term “protease resistant” or “protease resistance” as used herein, refers to the ability of a molecule comprising peptide bonds, such as a peptide, polypeptide or protein, to prevent hydrolytic cleavage of one or more of its peptide bonds in the presence of a protease. The degree of protease resistance may be measured by comparison with another molecule of the same identity that is less capable of withstanding hydrolytic cleavage when subjected in the presence of the same amount of protease under the same conditions at which the hydrolytic cleavage is evaluated. Protease cleavage can be confirmed when cleaved fragments of lower molecular weight than the original uncleaved parent molecule is obtained. Any methods known to the skilled person that are capable of detection of low molecular weight fragments derived from protease cleavage of a parent molecule may be used to evaluate protease resistance. Examples include subjecting protease resistant test variants and less protease resistant control to the same concentration of protease under the same conditions, including pH, temperature and duration. Subsequently, presence or absence of fragments of lower molecular weight derived from proteolytic cleavage may be observed by subjecting the treated test and control samples to reducing SDS-PAGE.

In a preferred embodiment, recombinant human matriptase/ST14 catalytic domain (MT-SP1) (R&D Systems, Inc., 3926-SE-010) was used as the protease. 75 nM protease and 750 nM of a fusion protein comprising protease resistance IL-12 were incubated in PBS under a condition of 37 degrees Celsius (degrees C.) for 1, 4 and 24 hours. Subsequently, presence of fragments of lower molecular weight than the parent after protease incubation, i.e. protease cleavage, was evaluated by reducing SDS-PAGE. A protease resistance variant is selected when the digested molecule remains more than 50%, 55%, 60%, 65%, 70%, 75%, 80%, 85%, 90%, 95%, 96%, 97%, 98%, 99% or 100% intact after subjected to proteolytic digestion of at least 4 hours, up to 24 hours under conditions stated above. Evaluation of percentage of a molecule remaining intact may be assessed in methods by various molecular biology techniques, including for example, SDS-PAGE densitometry of the lower molecular weight fragments (or none) derived from the parent molecule after digestion compared to the original parent molecule.

In the present invention, the amino acid modification(s) are introduced to the heparin binding region of IL-12, which is prone to cleavage by proteases, including MT-SP1. Cleavage can occur in the heparin binding region between K260 and R261 of the p40 subunit of IL-12.

In some embodiments of the present application, the protease resistant IL-12 is de-glycosylated or aglycosylated. In some embodiments of the present application, the protease resistant IL-12 comprises an amino acid sequence selected from the group consisting of SEQ ID NO: 157, 158, 159, 160, 161, 162, 163, 164, 165, 166, 167, 168, 169, 173, 174, 175, 176, 177, 178, 179, 180, 181, 182, 183, 184, 185, 186, 187, 188, KRE, KHE, and KKE. In some embodiments, the protease resistant IL-12 does not comprise the amino acid sequence of KSKREK (SEQ ID NO: 197), or KSKRE (SEQ ID NO: 361).

In other embodiments of the present application, the protease resistant IL-12 is glycosylated. In some embodiments of the present application, the protease resistant IL-12 comprises an amino acid sequence selected from the group consisting of SEQ ID NO: 157, 158, 159, 160, 161, 162, 163, 164, 165, 166, 167, 168, 169, KRE, KHE, and KKE. In some embodiments of the present application, the protease resistant IL-12 comprises an amino acid sequence selected from the group consisting of SEQ ID NO: 277, 278, 279, 280, 281, 282, 283, 284, 285, 286, 287, 288, 289, 290, 291, 292, 325, 326, 327, 328, and 329. These sequences represent p40 subunits of IL-12 comprising the protease-resistant mutations. In some embodiments, the protease resistant IL-12 does not comprise the amino acid sequence of KSKREK (SEQ ID NO: 197), or KSKRE (SEQ ID NO: 361).

In some embodiments of the present application, the protease resistant IL-12 comprises an amino acid sequence selected from the group consisting of SEQ ID NO: 293, 294, 295, 296, 297, 298, 299, 300, 301, 302, 303, 304, 305, 306, 307, 308, 330, 331, 332, 333, and 334. These sequences represent single chain IL-12 protein sequences comprising the protease-resistant mutations. In some embodiments, the protease resistant IL-12 does not comprise the amino acid sequence of KSKREK (SEQ ID NO: 197), or KSKRE (SEQ ID NO: 361).

In some embodiments of the present application, the protease resistant IL-12 comprises an amino acid sequence selected from the group consisting of SEQ ID NO: 309, 310, 311, 312, 313, 314, 315, 316, 317, 318, 319, 320, 321, 322, 323, 324, 335, 336, 337, 338, and 339. These sequences represent single chain IL-12 protein sequences comprising the protease-resistant mutations, and wherein the R in the native p35 subunit of IL-12 is deleted. In some embodiments, the protease resistant IL-12 does not comprise the amino acid sequence of KSKREK (SEQ ID NO: 197), or KSKRE (SEQ ID NO: 361).

It has been established by the present inventors that a modified sequence KHKE (SEQ ID NO: 165) provides a protease-resistant IL-12, which does not undergo proteolytic degradation in the presence of protease and remains sufficiently neutralized when bound to the ligand-binding domain of the presently described protein complex, or fusion protein, in the uncleaved state. Therefore, in a preferred embodiment, the protease resistant IL-12 comprises SEQ ID NO: 317. Therefore, in a preferred embodiment, the protease resistant IL-12 comprises SEQ ID NO: 317, wherein, optionally, one or more further mutations are introduced.

Additional protease resistant IL-12 molecules are disclosed in International Publication No. WO2023/002952, which is incorporated herein by reference in its entirety for all purposes.

### Attenuated IL-12

The present invention includes an IL-12 protein complex, or fusion protein, or protein comprising a heterodimer of a polypeptide of p40 subunit of IL-12 protein and a p35 subunit of IL-12 protein, wherein the p40 subunit of IL-12 protein is a modified p40 subunit. The IL-12 protein complex, or fusion protein, or protein comprising a heterodimer of a modified p40 subunit has weaker binding affinity to the human IL-12 receptor (hIL-12R), or the human IL-12Rbeta1 subunit, and thus has a lower potency or activity than a non-modified IL-12 protein complex, or fusion protein, or protein. The IL-12 protein complex, or fusion protein, or protein with weaker binding affinity to the human IL-12R is also referred herein as an attenuated IL-12 or low potent IL-12. The attenuated IL-12 or low potent IL-12 is useful for modulating signal transduction mediated by human IL-12, including but not limited to, inducing the desired IL-12 signaling activity while minimizing undesired activity and/or intracellular signaling in other undesired cellular or tissue subtypes. In embodiments, the low potent/attenuated IL-12 of the present invention has a weaker affinity to IL-12R compared to the IL-12 not comprising the potency lowering mutation. The potency lowering mutation concerns a substitution of the tyrosine at position 16 of the mature form of the human Interleukin-12 p40 subunit (SEQ ID NO: 44). The tyrosine is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W). Preferably, the tyrosine is substituted with proline (P).

In embodiments, the invention covers an IL-12 protein complex, or fusion protein, or protein comprising a heterodimer of a polypeptide of the modified p40 subunit of the present invention and a polypeptide comprising SEQ ID NO: 45, or SEQ ID NO: 345, wherein the polypeptide of the modified p40 subunit of the present invention comprises an amino acid sequence that is at least 90%, or at least 95%, or at least 97%, or at least 98%, or at least 99% identical to SEQ ID NO: 44, wherein said polypeptide comprises one or more amino acid substitutions at positions corresponding to amino acid residue Y16 of SEQ ID NO: 44, wherein (a) the amino acid substitution at the position corresponding to Y16 is selected from the group consisting of phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W), preferably proline (P). The modified p40 subunit of the present invention comprises SEQ ID NO: 265. In an embodiment, the invention covers an IL-12 protein complex or protein comprising a heterodimer of a polypeptide comprising SEQ ID NO: 265, and a polypeptide comprising SEQ ID NO: 45, or SEQ ID NO: 345.

In a further aspect, the present invention includes an IL-12 fusion protein comprising a polypeptide of the modified p40 subunit of the present invention, and a polypeptide of SEQ ID NO: 45 or SEQ ID NO: 345. The invention also concerns an IL-12 fusion protein comprising the polypeptide of the modified p40 subunit of the present invention linked to a polypeptide of SEQ ID NO: 45 or SEQ ID NO: 345 via a linker. The linker can comprise (Gly Gly Gly Gly Ser (GGGGS (SEQ ID NO: 6))n, wherein n is 1 or larger. For example, n may be 1, 2, 3, 4, 5, 6, 7, 8, 9, or 10 or more.

In embodiments, the IL-12 of the present invention and IL-12 ligand moiety in protein complexes, proteins and polypeptides of the present invention is not only characterized by its protease resistance, but also by mutations, which lower the potency/activity of the resultant IL-12, also referred to as an attenuated IL-12 or low potent IL-12. The low potent/attenuated IL-12 of the present invention shows lower toxicity compared to an IL-12 not comprising the potency lowering mutation, while not compromising efficacy. This is due to the potency lowering mutation introduced into the amino acid sequence of the p40 subunit of IL-12. The low potent/attenuated IL-12 of the present invention has a weaker affinity to IL-12R compared to the IL-12 not comprising the potency lowering mutation. The mutation concerns a substitution of the tyrosine at position 16 of SEQ ID NO: 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 331, 332, 333, 336, 337, and 338. Position 16 in these sequences corresponds to position 16 of the mature form of the human Interleukin-12 p40 subunit. The tyrosine is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W). Preferably, the tyrosine is substituted with proline (P). In an embodiment, the tyrosine at position 16 of SEQ ID NO: 301, or 317 is substituted as defined above. In a preferred embodiment, the tyrosine at position 16 of SEQ ID NO: 317 is substituted as defined above. In an embodiment, the IL-12 is a polypeptide defined by SEQ ID NO: 330 or 335. In a preferred embodiment, the IL-12 is a polypeptide defined by SEQ ID NO: 335.

The mutations providing the protease resistance and the decreased potency/activity are within the sequence of the p40 subunit of IL-12. Therefore, in one aspect of the invention, an embodiment concerns a polypeptide comprising the p40 subunit, which is characterized by protease resistance and lowered potency/activity, also referred to as the modified p40 subunit. The polypeptide of the present invention in this aspect is thus a polypeptide defined by any one of the sequences selected from the group consisting of: SEQ ID NO: 277, 278, 279, 280, 281, 282, 283, 284, 285, 286, 287, 288, 289, 290, 291, 292, 326, 327, and 328, wherein the tyrosine at position 16 is substituted with an amino acid selected from the group consisting of: phenylalanine (F), asparagine (N), glutamic acid (E), proline (P), glycine (G), lysine (K), alanine (A), aspartic acid (D), histidine (H), isoleucine (I), leucine (L), methionine (M), glutamine (Q), arginine (R), serine (S), threonine (T), valine (V), and tryptophan (W), preferably proline (P). In a preferred embodiment, the polypeptide is a polypeptide defined by SEQ ID NO: 285, wherein the tyrosine at position 16 is substituted with an amino acid selected from the group consisting of: phenylalanine (F), asparagine (N), glutamic acid (E), proline (P), glutamic acid (G), lysine (K), alanine (A), aspartic acid (D), histidine (H), isoleucine (I), leucine (L), methionine (M), glutamine (Q), arginine (R), serine (S), threonine (T), valine (V), and tryptophan (W), preferably proline (P). In a preferred embodiment, the IL-12 is a polypeptide defined by SEQ ID NO: 325. In a preferred embodiment, the IL-12 of the present invention comprises a polypeptide defined by SEQ ID NO: 325.

The position in the p40 subunit herein is determined from the amino acid sequence of the mature form of Interleukin-12 subunit beta without the signal peptide. The sequence of the precursor of Interleukin-12 subunit beta including the signal peptide of amino acids 1-22 is disclosed with NCBI Reference Sequence Accession number NP_002178.2 (version of Dec. 14, 2022). The mature form of human p40 or human Interleukin-12 subunit beta corresponds to the peptide with amino acids 23 to 328. The mature form of human p40 or human Interleukin-12 subunit beta corresponds to SEQ ID NO: 44.

In a further aspect, the invention concerns an IL-12 protein complex or protein comprising a heterodimer of the polypeptide of the modified p40 subunit of the present invention, and a polypeptide of SEQ ID NO: 45 or SEQ ID NO: 345. The invention also concerns an IL-12 fusion protein comprising the polypeptide of the modified p40 subunit of the present invention linked to a polypeptide of SEQ ID NO: 45 or SEQ ID NO: 345 via a linker. The linker can comprise (Gly Gly Gly Gly Ser (GGGGS (SEQ ID NO: 6))n, wherein n is 1 or larger. For example, n may be 1, 2, 3, 4, 5, 6, 7, 8, 9, or 10 or more.

In one aspect, the invention concerns an IL-12 fusion protein comprising any one of the sequences selected from the group consisting of: SEQ ID NO: 265, 266, 267, and 268.

In a further aspect, the invention concerns an IL-12 fusion protein comprising any one of the sequences selected from the group consisting of: SEQ ID NO: 325, 326, 327, 328, and 329.

In one aspect, the invention concerns an IL-12 fusion protein defined by any one of the sequences selected from the group consisting of: SEQ ID NO: 269, 270, 271, 272, 273, 274, 275, and 276.

In a further aspect, the invention concerns an IL-12 fusion protein defined by any one of the sequences selected from the group consisting of: SEQ ID NO: 330, 331, 332, 333, 334, 335, 336, 337, 338 and 339.

In a further aspect, the invention concerns an IL-12 fusion protein defined by any one of the sequences selected from the group consisting of: SEQ ID NO: 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, and 324.

In a further aspect, the invention concerns an attenuated IL-12 polypeptide comprising a p40 subunit and a p35 subunit, wherein the p40 subunit comprises an asparagine (N), glutamic acid (E), glycine (G) or proline (P) residue at the amino acid position corresponding to residue 16 of SEQ ID NO: 44. In embodiments, the attenuated IL-12 polypeptide is characterized by one or more of: (a) wherein the p40 subunit does not comprise the amino acid sequence of KSKREK (SEQ ID NO: 197), or KSKRE (SEQ ID NO: 361); (b) wherein the attenuated IL-12 polypeptide is a single chain IL-12 polypeptide; and (c) wherein the attenuated IL-12 polypeptide comprises the amino acid sequence of SEQ ID NO: 325 or 335. In a further aspect, the invention concerns a fusion protein or protein complex comprising the attenuated IL-12 polypeptide. In a further aspect, the invention concerns a polynucleotide encoding the attenuated IL-12 polypeptide, or a plurality of polynucleotides encoding the fusion protein or protein comprising the attenuated IL-12 polypeptide. In a further aspect, the invention concerns a recombinant host cell comprising a polynucleotide encoding the attenuated IL-12 polypeptide, or a plurality of polynucleotides encoding the fusion protein or protein complex comprising the attenuated IL-12 polypeptide. In a further aspect, the invention concerns a method of producing the attenuated IL-12 polypeptide, or the fusion protein or protein complex comprising the attenuated IL-12 polypeptide, comprising (a) culturing a recombinant host cell comprising a polynucleotide encoding the attenuated IL-12 polypeptide, or a plurality of polynucleotides encoding the fusion protein or protein complex comprising the attenuated IL-12 polypeptide, under conditions suitable for the expression of the attenuated IL-12 polypeptide or fusion protein or protein complex and (b) recovering the attenuated IL-12 polypeptide or fusion protein or protein complex. In a further aspect, the invention concerns a method of treating a disease or disorder in an individual in need thereof, comprising administering to said individual a therapeutically effective amount of the attenuated IL-12 polypeptide or the fusion protein or protein complex.

The term “attenuated” or “low potent” as used herein, refers to a molecule or a ligand (e.g. attenuated IL-12, or low potent IL-12) comprising potency lowering mutation, that specifically binds to and activates its receptor but activates the receptor to an extent less than a molecule or ligand that does not comprises said mutation (e.g. reference IL-12). Receptor activation can be confirmed by activity assays well known in the art, also described herein. An attenuated IL-12 or low potent IL-12 may be selected when the difference in EC50 value (“fold change”) of a non-cleaved (inactive) and a cleaved (active) form of the protein complex or fusion protein or protein comprising said attenuated IL-12 or low potent IL-12 is more than a reference protein complex or fusion protein or protein comprising a reference IL-12, for example, when the fold change is at least or more than 1.5-fold, 2.0-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, 12.0-fold, 12.5-fold, 13.0-fold, 13.5-fold, 14.0-fold, 14.5-fold, 15.0-fold, 15.5-fold, 16.0-fold, 16.5-fold, 17.0-fold, 17.5-fold, 18.0-fold, 18.5-fold, 19.0-fold, 19.5-fold, 20.0-fold, 20.5-fold, 21.0-fold, 21.5-fold, 22.0-fold, 22.5-fold, 23.0-fold, 23.5-fold, 24.0-fold, 24.5-fold, 25.0-fold, or more than 25.0-fold that of a reference protein complex or fusion protein or protein comprising a reference IL-12. In an embodiment, an attenuated IL-12 or low potent IL-12 may be selected when the maximum response (Emax) produced by an attenuated or low potent IL-12 variant is less than that of a reference IL-12, for example, when the attenuated or low potent IL-12 may have greater than 10% but less than 100%, alternatively greater than 20% but less than 100%, alternatively greater than 30% but less than 100%, alternatively greater than 40% but less than 100%, alternatively greater than 50% but less than 100%, alternatively greater than 60% but less than 100%, alternatively greater than 70% but less than 100%, alternatively greater than 80% but less than 100%, or alternatively greater than 90% but less than 100%, of the reference IL-12 when evaluated at similar concentrations in a given assay. In an embodiment, an attenuated IL-12 or low potent IL-12 variant may be selected when it exhibits reduced binding to its receptor. As used herein, the term “reduced binding” when used in respect of an attenuated or low potent IL-12 variant relative to a reference IL-12 refers to one that binds to the receptor with an affinity of less than 20%, alternatively less than about 10%, alternatively less than about 8%, alternatively less than about 6%, alternatively less than about 4%, alternatively less than about 2%, alternatively less than about 1%, or alternatively less than about 0.5% of the reference IL-12 from which the attenuated IL-12 or low potent IL-12 variant was derived. Binding affinity can be confirmed by binding assays well known in the art, also described herein.

### Ligand-Binding Moiety/Molecule

In a further aspect of the invention, the fusion protein or protein complex comprises the ligand-binding moiety/molecule having ligand neutralizing activity that is capable of inhibiting the biological activity of the ligand moiety/molecule by exerting its neutralizing activity. In some embodiments, the fusion polypeptide or protein complex comprises an anti-IL-12 binding molecule complex, capable of binding to a ligand moiety/molecule, i.e. IL-12, and has IL-12 neutralizing activity that is capable of inhibiting the IL-12 activity of the IL-12 by exerting its binding activity. In one embodiment, the anti-IL-12 binding molecule complex, or ligand binding moiety/molecule comprises antibody fragments, wherein said molecule complex has moieties substantially similar in structure to constant domains or constant regions as in an IgG antibody, and moieties substantially similar in structure to variable domains or variable regions as in the IgG antibody, and having conformation substantially similar to that of the IgG antibody. In an aspect of the invention, an anti-IL-12 antibody that binds human IL-12 is included.

In a further aspect, the ligand-binding moiety/molecule such as the fusion polypeptide, or protein complex, or antibody, or binding molecule complex, or binding molecule, according to any of the above embodiments may incorporate any of the features, singly or in combination, as described in the sections A to G below, while exemplary fusion polypeptide, or protein complex, or antibody, or binding molecule complex, or binding molecule are further described in section H below.

Additional ligand-binding moieties/molecules are disclosed in International Publication No. WO2023/002952, which is incorporated herein by reference in its entirety for all purposes.

### A) Binding Affinity

In certain embodiments, a ligand binding domain or an antigen binding domain provided herein has a dissociation constant (Kd) of 1 micro M or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³ M, e.g., from 10⁻⁹ M to 10⁻¹³ M).

In one embodiment, Kd is measured by a radiolabeled ligand or antigen binding assay (RIA). In one embodiment, an RIA is performed with the Fab version of an antibody for ligand detection and its ligand. For example, solution binding affinity of Fabs for ligand is measured by equilibrating Fab with a minimal concentration of (125I)-labelled ligand in the presence of a titration series of unlabeled ligand, then capturing bound ligand with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro 1/well of scintillant (MICROSCINT-20 TM; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance (SPR) assay. For example, an assay using a BIACORE (registered trademark)-2000 or a BIACORE (registered trademark)-3000 (BIAcore, Inc., Piscataway, NJ) is performed at 25 degrees C. with immobilized antigen CM5 chips at about 10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Ligand is diluted with 10 mM sodium acetate, pH 4.8, to 5 micro g/ml (about 0.2 micro M) before injection at a flow rate of 5 micro 1/minute to achieve approximately 10 response units (RU) of coupled protein. Following the injection of ligand, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min. Association rates (kon) and dissociation rates (koff) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-ligand antibody (Fab form) in PBS, pH 7.2, in the presence of increasing concentrations of ligand as measured in a spectrometer, such as a stop-flow equipped spectrophotometer (Aviv Instruments) or a 8000-series SLM-AMINCO™ spectrophotometer (ThermoSpectronic) with a stirred cuvette. Accordingly, persons skilled in the art can carry out affinity measurements using other common methods of measuring affinity for other ligand-binding molecules, antigen-binding molecules or antibodies, towards various kind of ligands, ligand receptors and antigens.

### A.1) Substitution, Insertion, and Deletion Variants

In certain embodiments, fusion protein variants, or protein complex variants, having one or more amino acid substitutions are provided. Sites of interest for substitutional mutagenesis include the HVRs and FRs. Conservative substitutions are shown in Table 1 under the heading of “preferred substitutions”. More substantial changes are provided in Table 1 under the heading of “exemplary substitutions,” and as further described below in reference to amino acid side chain classes. Amino acid substitutions may be introduced into a fusion protein, or protein complex of the present specification and the products screened for a desired activity, e.g., retained/improved ligand or antigen binding, decreased immunogenicity, or improved ADCC or CDC, or abolished ADCC or CDC. In embodiments, amino acid substitutions are introduced to the fusion protein or protein complex of the present application, and the products screened for improved ligand binding of the ligand-binding domain in the uncleaved state, and reduced ligand binding of the ligand-binding domain in the cleaved state.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.
   Non-conservative substitutions will entail exchanging a member of one of these classes for another class.

One type of substitutional variant involves substituting one or more hypervariable region residues of a parent antigen-binding domain or ligand binding domain (e.g. a humanized or human antigen-binding domain or ligand binding domain). Generally, the resulting variant(s) selected for further study will have modifications (e.g., improvements) in certain biological properties (e.g., increased affinity, reduced immunogenicity) relative to the parent antigen-binding domain or ligand binding domain and/or will have substantially retained certain biological properties of the parent antigen-binding domain or ligand binding domain. An exemplary substitutional variant is an affinity matured antigen-binding domain or ligand binding domain, which may be conveniently generated, e.g., using phage display-based affinity maturation techniques such as those described herein. Briefly, one or more HVR residues are mutated and the variant antigen-binding domain or ligand binding domain displayed on phage and screened for a particular biological activity (e.g. binding affinity).

Alterations (e.g., substitutions) may be made in HVRs, e.g., to improve antigen-binding domain or ligand binding domain affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact ligand or antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antigen-binding domain or ligand binding domain variants with the desired affinity. Another method to introduce diversity involves HVR-directed approaches, in which several HVR residues (e.g., 4-6 residues at a time) are randomized. HVR residues involved in antigen binding or ligand binding may be specifically identified, e.g., using alanine scanning mutagenesis or modelling. CDR-H3 and CDR-L3 in particular are often targeted.

In certain embodiments, substitutions, insertions, or deletions may occur within one or more HVRs so long as such alterations do not substantially reduce the ability of the antigen-binding domain or ligand binding domain to bind antigen or ligand. For example, conservative alterations (e.g., conservative substitutions as provided herein) that do not substantially reduce binding affinity may be made in HVRs. Such alterations may, for example, be outside of antigen or ligand contacting residues in the HVRs. In certain embodiments of the variant VH and VL sequences provided above, each HVR either is unaltered, or contains no more than one, two or three amino acid substitutions.

A useful method for identification of residues or regions of an antigen-binding domain or ligand binding domain that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antigen-binding domain or ligand binding domain with antigen or ligand is affected. Further substitutions may be introduced at the amino acid locations demonstrating functional sensitivity to the initial substitutions. Alternatively, or additionally, a crystal structure of an antigen-antigen-binding domain complex may be analyzed to identify contact points between the antigen-binding domain and antigen. Also included in the present specification, a crystal structure of a ligand-ligand-binding domain complex may be analyzed to identify contact points between the ligand-binding domain and the ligand. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antigen-binding molecule or ligand binding molecule with an N-terminal methionyl residue. Other insertional variants of the antigen-binding or ligand binding molecule include the fusion of an enzyme (e.g. for ADEPT) or a polypeptide which increases the plasma half-life of the antigen-binding or ligand binding molecule to the N- or C-terminus of the molecule.

### A.2) Glycosylation Variants

In certain embodiments, an antigen-binding molecule or ligand binding molecule provided herein is altered to increase or decrease the extent to which the molecule is glycosylated. Addition or deletion of glycosylation sites to an antigen-binding molecule or ligand binding molecule may be conveniently accomplished by altering the amino acid sequence such that one or more glycosylation sites is created or removed.

Where the antigen-binding molecule or ligand binding molecule comprises an Fc region, the carbohydrate attached thereto may be altered. Native antigen-binding molecules produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antigen-binding molecule or ligand binding molecule of the present invention may be made in order to create antigen-binding molecule or ligand binding molecule variants with certain improved properties. In embodiments, the antigen-binding molecule or ligand binding molecule is glycosylated and/or has Asn297 in the Fc region.

In one embodiment, antigen-binding molecule or ligand binding molecule variants are provided having a carbohydrate structure that lacks fucose attached (directly or indirectly) to an Fc region. For example, the amount of fucose in such antigen-binding molecule or ligand binding molecule may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example, Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107). In one embodiment, antigen-binding molecule or ligand binding molecule of the present invention incorporates an aforementioned fucosylation variant.

In one embodiment, antigen-binding molecule or ligand binding molecule of the present invention incorporates Fc variants provided with bisected oligosaccharides, e.g., in which a biantennary oligosaccharide attached to the Fc region of the antigen-binding molecule or ligand binding molecule is bisected by GlcNAc. Such antigen-binding molecule or ligand binding molecule variants may have reduced fucosylation and/or improved ADCC function. Examples of such variants are described, e.g., in WO 2003/011878 (Jean-Mairet et al.); U.S. Pat. No. 6,602,684 (Umana et al.); and US 2005/0123546 (Umana et al.). Antigen-binding molecule variants with at least one galactose residue in the oligosaccharide attached to the Fc region are also provided. Such antigen-binding molecule variants may have improved CDC function. Such variants are described, e.g., in WO 1997/30087 (Patel et al.); WO 1998/58964 (Raju, S.); and WO 1999/22764 (Raju, S.). In embodiments, the protein complex, the polypeptide, the protein, the fusion protein, the binding molecule complex or the binding molecule provided herein comprises an Fc region with reduced effector function selected of one or more of binding to all human Fc gamma receptors, ADCC and CDC. In embodiments, the protein complex, the polypeptide, the protein complex, the fusion protein, the binding molecule complex or the binding molecule provided herein comprises an Fc region with reduced effector function, wherein at least one effector function selected of one or more of binding to all human Fc gamma receptors, ADCC and CDC is abolished and/or substantially abolished and/or wherein at least one effector function is reduced as compared to the corresponding native human Fc portion and/or wherein the binding to the human FcRn is retained in the Fc region.

### A.3) Fc Region Variants

In certain embodiments, one or more amino acid modifications may be introduced into the Fc region of an antigen-binding molecule or ligand binding molecule provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions. In embodiments, the Fc region variant comprises a human Fc region sequence having 1 to 10, such as 1, 2, 3, 4, 5, 6, 8, 9 or 10, amino acid modifications, in particular substitutions, in the Fc region.

In certain embodiments, the invention contemplates an antigen-binding molecule or ligand binding molecule variant that possesses some but not all effector functions, which make it a desirable candidate for applications in which the half-life of the antibody or molecule in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antigen-binding molecule or ligand binding molecule lacks Fc gamma R binding (hence likely lacking ADCC activity) but retains FcRn binding ability. The primary cells for mediating ADCC, NK cells, express Fc gamma RIII only, whereas monocytes express Fc gamma RI, Fc gamma RII and Fc gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACT1TM non-radioactive cytotoxicity assay for flow cytometry (CellTechnology, Inc. Mountain View, CA; and CytoTox 96 (registered trademark) non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antigen-binding molecule or ligand binding molecule is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations can also be performed using methods known in the art (see, e.g., Petkova, S. B. et al., Int'l. Immunol. 18(12):1759-1769 (2006)). In embodiments, the protein complex, the polypeptide, the protein, the fusion protein, the binding molecule complex or the binding molecule provided herein comprises an Fc region with reduced effector function selected of one or more of binding to all human Fc gamma receptors, ADCC and CDC. In embodiments, the protein complex, the polypeptide, the protein complex, the fusion protein, the binding molecule complex or the binding molecule provided herein comprises an Fc region with reduced effector function, wherein at least one effector function selected of one or more of binding to all human Fc gamma receptors, ADCC and CDC is abolished and/or substantially abolished and/or wherein at least one effector function is reduced as compared to the corresponding native human Fc portion and/or wherein the binding to the human FcRn is retained in the Fc region.

Antigen binding molecules with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Such substitutions result in reduced effector function of binding to all human Fc gamma receptors, ADCC and/or CDC. In one embodiment, antigen-binding molecule or ligand binding molecule of the present invention incorporates an aforementioned reduced effector function variant.

Certain antigen binding molecule variants with increased or decreased binding to FcRs are described. (See, e.g., U.S. Pat. No. 6,737,056; WO 2004/056312, and Shields et al., J. Biol. Chem. 9(2): 6591-6604 (2001).)

In certain disclosure, an antigen binding molecule variant, or ligand binding molecule comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either increased or decreased) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antigen binding molecules with increased half-lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those molecules comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). In one embodiment, antigen-binding molecule or ligand binding molecule of the present invention incorporates an aforementioned Fc region variant.

See also Duncan & Winter, Nature 322:738-40 (1988); U.S. Pat. Nos. 5,648,260; 5,624,821; and WO 94/29351 concerning other examples of Fc region variants.

### B) Recombinant Methods and Compositions

The fusion protein, protein complex, polypeptide, binding molecule complex, or binding molecule as described herein comprises an antigen/ligand binding molecule encompassing one or more antibody fragments may be produced using recombinant methods and compositions, e.g., as described in U.S. Pat. No. 4,816,567.

In one aspect, an isolated polynucleotide or plurality of polynucleotides encoding the protein complex, or the polypeptide, or the fusion protein, or the binding molecule complex or binding molecule provided herein is provided. For example, the plurality of polynucleotides may be 2, 3, 4, 5, 6, 7, 8, 9, 10, or more polynucleotides. For example, in the case of the protein complex, or the polypeptide, or the fusion protein, or the binding molecule complex or binding molecule provided herein comprises two different types of polypeptides, such as e.g. a “first polypeptide” and a “second polypeptide” in aspects herein, the plurality of polynucleotides encoding the protein complex may be 2 polynucleotides, wherein the first polynucleotide encodes the first type of polypeptide (such as e.g. a “first polypeptide” in aspects herein) and the second polynucleotide encodes the second type of polypeptide (such as e.g. a “second polypeptide” in aspects herein). In a further embodiment, one or more vectors (e.g., expression vectors) comprising the polynucleotide or plurality of polynucleotides are provided. In a further embodiment, a vector (e.g., an expression vector) comprising the polynucleotide or plurality of polynucleotides are provided. In alternative embodiments, a plurality of vectors is provided comprising the plurality of polynucleotides. For example, each polynucleotide of the plurality of polynucleotides may be comprised in a different vector (e.g., an expression vector). In a further embodiment, one or more vectors (e.g., expression vectors) comprising such nucleic acid are provided. In a further embodiment, a host cell comprising such nucleic acid is provided. In a further embodiment, a host cell comprising the polynucleotide or plurality of polynucleotides provided herein is provided. In one such embodiment, a host cell comprises (e.g., has been transformed with): (l) a vector comprising a polynucleotide that encodes an amino acid sequence of a polypeptide comprising the VL of the polypeptide, protein, fusion protein, binding molecule complex, or binding molecule, such as a light chain, and a polynucleotide that encodes an amino acid sequence comprising of a polypeptide comprising the VH of the polypeptide, protein, fusion protein, binding molecule complex, or binding molecule, such as a polypeptide corresponding to the heavy chain of a polypeptide, or a fusion protein representing a “second polypeptide” in aspects herein, or (2) a first vector comprising a polynucleotide that encodes an amino acid sequence of a polypeptide comprising the VL of the polypeptide, protein, fusion protein, binding molecule complex, or binding molecule, such as a light chain, and a second vector comprising a polynucleotide that encodes an amino acid sequence comprising of a polypeptide comprising the VH of the polypeptide, protein, fusion protein, binding molecule complex, or binding molecule, such as a polypeptide corresponding to the heavy chain of a polypeptide, or a fusion protein representing a “second polypeptide” in aspects herein. In one embodiment of any of the host cells provided herein, the host cell is eukaryotic, e.g. a Chinese Hamster Ovary (CHO) cell or lymphoid cell (e.g., Y0, NS0, Sp2/0 cell). In one embodiment of any of the host cells provided herein, the host cell is a Chinese Hamster Ovary (CHO) cell.

In an aspect, a method of producing the protein complex, the polypeptide, the protein, the fusion protein, the binding molecule complex or binding molecule of the invention herein is provided, comprising the steps of:
a) culturing the host cell of the invention under conditions suitable for the expression of the protein complex, the polypeptide, the protein, the fusion protein, the binding molecule complex, or binding molecule of the invention herein, and
b) recovering the protein complex, the polypeptide, the protein, the fusion protein, the binding molecule complex, or binding molecule of the invention herein.

In a further aspect, a protein complex, or a polypeptide, or a protein, or a fusion protein, or a binding molecule complex, or a binding molecule produced by, or producible by, the method of the aspects and embodiments herein is provided.

For recombinant production of a protein complex, the polypeptide, the protein, the fusion protein, the binding molecule complex, or binding molecule, a nucleic acid, polynucleotide or plurality of polynucleotides, encoding the protein complex, the polypeptide, the protein, the fusion protein, the binding molecule complex, or binding molecule, e.g., as described above, is/are isolated and inserted into one or more vectors for further cloning and/or expression in a host cell. Such nucleic acid, polynucleotide or plurality of polynucleotides, may be readily isolated and sequenced using conventional procedures (e.g., by using oligonucleotide probes that are capable of binding specifically to genes encoding the polypeptides, the proteins, the fusion proteins, or the protein complexes).

Suitable host cells for cloning or expression of aforementioned vectors include prokaryotic or eukaryotic cells described herein. For example, they may be produced in bacteria, in particular when glycosylation and Fc effector function are not needed. For expression in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the protein complex, the polypeptide, the protein, the fusion protein, the binding molecule complex, or the binding molecule, may be isolated from the bacterial cell paste in a soluble fraction and can be further purified.

In addition to prokaryotes, eukaryotic microbes such as filamentous fungi or yeast are suitable cloning or expression hosts for aforementioned vectors, including fungi and yeast strains whose glycosylation pathways have been “humanized,” resulting in the production of a protein complex, a polypeptide, a protein, a fusion protein, a binding molecule complex, or a binding molecule, with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated protein complex, polypeptide, protein, fusion protein, binding molecule complex, or binding molecule, are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse Sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N. Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for production, see, e.g., Yazaki and Wu, Methods in Molecular Biology, Vol. 248 (B. K. C. Lo, ed., Humana Press, Totowa, NJ), pp. 255-268 (2003). In one embodiment of any of the host cells provided herein, the host cell is a Chinese Hamster Ovary (CHO) cell.

In certain embodiments, the invention provides a protein complex, polypeptide, protein, fusion protein, binding molecule complex or binding molecule as described herein obtainable by a method set out above.

An appropriate secretory signal can be incorporated into the protein complex, polypeptide, protein, fusion protein, binding molecule complex or binding molecule of interest in order to secrete the protein complex, polypeptide, protein, fusion protein, binding molecule complex or binding molecule expressed in the host cells to the lumen of the endoplasmic reticulum, periplasmic space, or an extracellular environment. The signal may be endogenous to the fusion protein or protein complex of interest or may be a foreign signal.

When the protein complex, polypeptide, protein, fusion protein, binding molecule complex or binding molecule of the present invention is secreted into a medium, the recovery of the protein complex, polypeptide, protein, fusion protein, binding molecule complex or binding molecule in the production method is performed by the recovery of the medium. When the protein complex, polypeptide, protein, fusion protein, binding molecule complex or binding molecule of the present invention is produced into cells, the cells are first lysed, followed by the recovery of the protein complex, polypeptide, protein, fusion protein, binding molecule complex or binding molecule.

A method known in the art including ammonium sulphate or ethanol precipitation, acid extraction, anion- or cation-exchange chromatography, phosphocellulose chromatography, hydrophobic interaction chromatography, affinity chromatography, hydroxyapatite chromatography, and lectin chromatography can be used for recovering and purifying the protein complex, polypeptide, protein, fusion protein, binding molecule complex or binding molecule of the present invention from the recombinant cell cultures.

### C) Assays

In some embodiments, the fusion polypeptide or the protein complex comprises a ligand-binding moiety/molecule, or antigen-binding molecule that comprises antibody fragments capable of binding to a ligand moiety/molecule (also referred to as anti-IL-12 binding molecule complex). Such anti-IL-12 binding molecule complex provided herein may be identified, screened for, or characterized for their physical/chemical properties and/or biological activities by various assays known in the art for the screening and characterization of antibody fragments.

### C.1) Binding Assays and Other Assays

As defined in this specification, the fusion protein, protein complex, polypeptide, protein, binding molecule complex, or binding molecule provided herein encompasses antibody fragments, including an antigen/ligand-binding domain, e.g. comprising an IL-12 binding domain.

In one aspect, a protein complex, polypeptide, protein, fusion protein, binding molecule complex or binding molecule, of the invention is tested for its antigen/ligand binding activity, e.g., by known methods such as ELISA, Western blot, etc. In another aspect, competition assays may be used to identify a protein complex, polypeptide, protein, fusion protein, binding molecule complex or binding molecule that competes with other ligand-binding molecules for binding to the ligand—IL-12. In certain embodiments, such a competing protein complex, polypeptide, protein, fusion protein, binding molecule complex or binding molecule binds to the same epitope (e.g., a linear or a conformational epitope) that is bound by the ligand-binding molecules. Detailed exemplary methods for mapping an epitope to which an antigen-binding molecule, such as an antibody binds are provided in Morris (1996) “Epitope Mapping Protocols,” in Methods in Molecular Biology vol. 66 (Humana Press, Totowa, NJ). Such methods for mapping are readily adaptable to identify the epitope to which the ligand-binding molecules of the present specification binds by the person skilled in the art.

In an exemplary competition assay, immobilized ligand is incubated in a solution comprising a first labelled protein complex, polypeptide, protein, fusion protein, binding molecule complex or binding molecule that binds to ligand IL-12 (and a second unlabeled protein complex, polypeptide, protein, fusion protein, binding molecule complex or binding molecule that is being tested for its ability to compete with the first protein complex, polypeptide, protein, fusion protein, binding molecule complex or binding molecule for binding to ligand. The second protein complex, polypeptide, protein, fusion protein, binding molecule complex or binding molecule may be present in a hybridoma supernatant. As a control, immobilized ligand is incubated in a solution comprising the first labelled protein complex, polypeptide, protein, fusion protein, binding molecule complex or binding molecule but not the second unlabeled protein complex, polypeptide, protein, fusion protein, binding molecule complex or binding molecule. After incubation under conditions permissive for binding of the first protein complex, polypeptide, protein, fusion protein, binding molecule complex or binding molecule to ligand, excess unbound protein complex, polypeptide, protein, fusion protein, binding molecule complex or binding molecule is removed, and the amount of label associated with immobilized ligand is measured. If the amount of label associated with immobilized ligand is substantially reduced in the test sample relative to the control sample, then that indicates that the second protein complex, polypeptide, protein, fusion protein, binding molecule complex or binding molecule is competing with the first protein complex, polypeptide, protein, fusion protein, binding molecule complex or binding molecule for binding to ligand. See Harlow and Lane (1988) Antibodies: A Laboratory Manual ch. 14 (Cold Spring Harbour Laboratory, Cold Spring Harbour, NY).

### C.2) Activity Assays

In one aspect, assays are provided for identifying the protein complex, polypeptide, protein, fusion protein, binding molecule complex or binding molecule having biological activity when cleaved at the protease cleavage site. Biological activity may include, e.g., binding to ligand receptor and activating ligand receptor signaling. Protein complex, polypeptide, protein, fusion protein, binding molecule complex or binding molecule or having such biological activity in vivo and/or in vitro are also provided.

In certain embodiments, a protein complex, polypeptide, protein, fusion protein, binding molecule complex or binding molecule of the invention is tested for such biological activity. In vitro ligand receptor activation can be confirmed by conducting luciferase assay(s). Briefly, cells expressing ligand receptors were cultured. The protein complexes, polypeptides, proteins, fusion proteins, binding molecule complexes or binding molecules were then incubated under conditions permissive for binding of the protein complex, polypeptide, protein, fusion protein, binding molecule complex or binding molecule to the ligand receptors expressed on the cell surface. As a control, ligand is incubated under the same conditions as the test protein complexes, polypeptides, proteins, fusion proteins, binding molecule complexes or binding molecules for binding to ligand receptors expressed on the cell surface. Luciferase activity is then detected using an appropriate assay system such as the Bio-Glo luciferase assay system (Promega, G7940) according to manufacturer's instructions. Luminescence can be detected using GloMax (registered trademark) Explorer System (Promega #GM3500) according to manufacturer's instructions. If comparable levels of activity for the test protein complexes, polypeptides, proteins, fusion proteins, binding molecule complexes or binding molecules and control ligands are detected, it is demonstrated that the protein complexes, polypeptides, proteins, fusion proteins, binding molecule complexes or binding molecules are capable of binding to ligand receptors and activating ligand receptor signaling thereof.

### C.3) Assay for Protease Cleavage

In one aspect, assays are provided for identifying protease cleavage of a molecule harboring a protease cleavage sequence, e.g. the ligand binding moiety/molecule comprising a protease cleavage sequence in the present invention. Whether the ligand binding moiety/molecule is cleaved by treatment with protease appropriate for the protease cleavage sequence can be optionally confirmed. The presence or absence of the cleavage of the protease cleavage sequence can be confirmed, for example, by contacting the protease with the molecule harboring the protease cleavage sequence, and confirming the molecular weight of the protease treatment product by an electrophoresis method such as SDS-PAGE.

Furthermore, cleavage fragments after protease treatment can be separated by electrophoresis such as SDS-PAGE and quantified to evaluate the activity of the protease and the cleavage ratio of a molecule into which the protease cleavage sequence has been introduced. A non-limiting embodiment of the method of evaluating the cleavage ratio of a molecule into which a protease cleavage sequence has been introduced includes the following method: For example, when the cleavage ratio of a protein complex, polypeptide, protein, fusion protein, binding molecule complex or binding molecule into which a protease cleavage sequence has been introduced is evaluated using recombinant human u-Plasminogen Activator/Urokinase (human uPA, huPA) (R&D Systems; 1310-SE-010) or recombinant human Matriptase/ST14 Catalytic Domain (human MT-SP1, hMT-SP1) (R&D Systems; 3946-SE-010), 100 microgram/mL of the protein complex, polypeptide, protein, fusion protein, binding molecule complex or binding molecule is reacted with 40 nM huPA or 3 nM hMT-SP1 in PBS at 37 degrees C. for one hour, and then subjected to capillary electrophoresis immunoassay. Capillary electrophoresis immunoassay can be performed using Wes (Protein Simple), but the present method is not limited thereto. As an alternative to capillary electrophoresis immunoassay, SDS-PAGE and such may be performed for separation, followed by detection with Western blotting. The present method is not limited to these methods. Before and after cleavage, the heavy chain can be detected using anti-human heavy chain HRP-labelled antibody, but any antibody that can detect cleavage fragments may be used. The area of each peak obtained after protease treatment is output using software for Wes (Compass for SW; Protein Simple), and the cleavage ratio (%) of the protein complex, polypeptide, protein, fusion protein, binding molecule complex or binding molecule can be determined with the following formula:

(Peak area of cleaved heavy chain)×100/(Peak area of cleaved heavy chain+Peak area of uncleaved heavy chain)

Cleavage ratios can be determined if protein fragments can be detected before and after protease treatment. Thus, cleavage ratios can be determined not only for protein complex, polypeptide, protein, fusion protein, binding molecule complex or binding molecule herein but also for various protein molecules into which a protease cleavage sequence has been introduced.

The in vivo cleavage ratio of a molecule into which a protease cleavage sequence has been introduced can be determined by administering the molecule into animals and detecting the administered molecule in blood samples. For example, a molecule variant, such as an antigen binding molecule, into which a protease cleavage sequence has been introduced is administered to mice, and plasma is collected from their blood samples. The molecule is purified from the plasma by a method known to those skilled in the art using Dynabeads Protein A (Thermo; 10001D), and then subjected to capillary electrophoresis immunoassay to evaluate the protease cleavage ratio of the molecule variant. Capillary electrophoresis immunoassay can be performed using Wes (Protein Simple), but the present method is not limited thereto. As an alternative to capillary electrophoresis immunoassay, SDS-PAGE and such may be performed for separation, followed by detection with Western blotting. The present method is not limited to these methods. The heavy chain of the molecule variant collected from mice can be detected using anti-human heavy chain HRP-labelled antibody but any antibody that can detect cleavage fragments may be used. Once the area of each peak obtained by capillary electrophoresis immunoassay is output using software for Wes (Compass for SW; Protein Simple), the ratio of the remaining heavy chain can be calculated as [Peak area of heavy chain]/[Peak area of light chain] to determine the ratio of the full-length heavy chain that remain uncleaved in the mouse body. In vivo cleavage efficiencies can be determined if protein fragments collected from a living organism are detectable. Thus, cleavage ratios can be determined not only for protein complex, polypeptide, protein, fusion protein, binding molecule complex or binding molecule but also for various protein molecules into which a protease cleavage sequence has been introduced. Calculation of cleavage ratios by the above-mentioned methods enables, for example, comparison of the in vivo cleavage ratios of protein complex, polypeptide, protein, fusion protein, binding molecule complex or binding molecule into which different cleavage sequences have been introduced, and comparison of the cleavage ratio of a single protein complex, polypeptide, protein, fusion protein, binding molecule complex or binding molecule between different animal models such as a normal mouse model and a tumor-grafted mouse model.

### C.4) Methods of Screening

The present invention also includes a method of screening to identify amino acid substitutions in the above-described protein complex, comprising the steps:
(a) introducing at least one amino acid substitution or at least one pair of amino acid substitution at the interface between VH and VL in said protein complex, and optionally introducing at least one amino acid substitution at the interface between the ligand and the ligand-binding domain, that promote dissociation of VH domain from the protein complex;
(b) determining a first response unit (RU1) of immobilised protein complex of step (a) in a BIACORE® surface plasma resonance (SPR) assay in the absence of protease;
(c) determining a second response unit (RU2) of immobilised protein complex of step (a) in the same BIACORE® surface plasma resonance (SPR) assay in the presence of protease;
(d) selecting the substitution(s) in step (a) if the percentage difference between RU1 and RU2 is less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, or is less than or equivalent to 8%, or is less than or equivalent to 9%, or is less than or equivalent to 10%, or is less than or equivalent to 11%, or is less than or equivalent to 12%, or is less than or equivalent to 13%, or is less than or equivalent to 14%, or is less than or equivalent to 15%, or is less than or equivalent to 16%, or is less than or equivalent to 17%, or is less than or equivalent to 18%, or is less than or equivalent to 19%, or is less than or equivalent to 20%, before and after protease cleavage; wherein the percentage reduction in response unit (RU) corresponds to the percentage reduction in molecular weight resulting from the release of VH from the protein complex.

The present invention also include a method for screening for a protein complex as described in any of the above embodiments, having substitutions that reduce association between VH and VL, comprising comparing the maximum response unit recorded for any of the above protein complex before and after protease cleavage under surface plasma resonance (SPR) and selecting substitutions that result in a reduction in response unit of less than or equivalent to 1%, or less than or equivalent to 2%, or less than or equivalent to 3%, or less than or equivalent to 4%, or less than or equivalent to 5%, or less than or equivalent to 6%, or less than or equivalent to 7%, or less than or equivalent to 8%, or less than or equivalent to 9%, or less than or equivalent to 10%, or less than or equivalent to 11%, or less than or equivalent to 12%, or less than or equivalent to 13%, or less than or equivalent to 14%, or less than or equivalent to 15%, or less than or equivalent to 16%, or less than or equivalent to 17%, or less than or equivalent to 18%, or less than or equivalent to 19%, or less than or equivalent to 20%, before and after protease cleavage.

In some embodiments, the percentage reduction in response unit corresponding to less than or equivalent to 1%, or less than or equivalent to 2%, or less than or equivalent to 3%, or less than or equivalent to 4%, or less than or equivalent to 5%, or less than or equivalent to 6%, or less than or equivalent to 7%, or less than or equivalent to 8%, or less than or equivalent to 9%, or less than or equivalent to 10% corresponds to dissociation of VH from the protein complex as described in any of the above embodiments. In a preferred embodiment, a dissociation of less than or equivalent to 10% indicates complete dissociation of VH(s) from a protein complex of the present invention.

In an assay using BIACORE®, the protein complex, fusion protein, antibody, or antibody fragment is immobilised on R-Protein A coupled—carboxymethylated dextran biosensor chips (CM4-ProA/G, BIACORE, Inc.) that are activated with N-ethyl-N′-(3-dimethylaminopropyl)-carbodiimide hydrochloride (EDC) and N-hydroxysuccinimide (NHS) according to the supplier's instructions. Protease, such as urokinase-type plasminogen activator (uPA), at a concentration of 400 nM is injected in assay buffer (HBS-EP+, Cytiva) at a flow rate of 2 microliter/min at 37 degrees C. for an association time of 1800 secs and dissociation time of 10 secs. The response unit (RU) captured before and after protease injection is compared.

The methods of screening described herein further includes a step of verification of the biological activity of the protein complex of the present specification in the first state and in the second state, i.e. before and after protease cleavage. The step of verification of biological activity includes biological assays described herein, or as detailed in the Examples, or suitably employed by the skilled person, to evaluate the biological activity of the ligand before and after protease cleavage.

The methods of screening described herein further comprising the following step(s): wherein for a given concentration of ligand and a given concentration of protein complex of the present specification corresponding to said concentration of ligand;
I. determining the biological activity of a protein complex before protease cleavage;
II. determining the biological activity of the protein complex in step I after protease cleavage;
III. introducing at least one amino acid modification or at least one pair of amino acid modification at the interface between VH and VL in the protein complex in step I, and optionally introducing at least one amino acid modification at the interface between the ligand and the ligand-binding domain, wherein the amino acid modification(s) promote dissociation of VH domain or VL domain from the protein complex upon protease cleavage in the presence of a protease;
IV. determining the biological activity of the protein complex in step III, before protease cleavage;
V. determining the biological activity of the protein complex in step III, after protease cleavage;
VIa. selecting amino acid modification(s) wherein the biological activity of the protein complex of step (IV) is less than the biological activity of the protein complex of step (V) or
VIb. selecting amino acid modification(s) wherein the biological activity of the protein complex in step (V) is greater than the biological activity of the protein complex in step (IV); and/or
VIII. determining the difference in biological activity of the biological activity of the protein complex between (I) and (II), “V1”, and the difference in biological activity of the biological activity of the protein complex between (IV) and (V), “V2”; and
IX. selecting the amino acid modification(s) wherein the value of V2 is greater than V1.

For the avoidance of doubt, the amino acid modification(s) capable of being introduced in step III includes any of the modifications that reduce association between VH and VL in a protein complex as described in any of the above embodiments, or derived from following the steps (a), and/or (b), and/or (c), and/or (d), and/or (e) set out above. Further, determination of whether a given concentration of ligand and a given concentration of protein complex of the present specification corresponds to the same concentration as that of its corresponding ligand can be readily determined by performing suitable biological assays that assess the biological activity of the protein complex, as detailed in the Examples or as described in any of the above embodiments.

The present invention further includes a library of amino acid modifications, including substitutions that reduce association between VH and VL in a protein complex as described in any of the above embodiments comprising the amino acid modifications at the interface between VH and VL. The library includes any of amino acid modifications, including substitutions selected from positions Q39D, W47A, W47L, W47M, Y91A, Y91L, Y91M, H91A, W103A, W103L, W103M, V37S, V37Q, G44Q, L45A, and L45Q on the VH, or R38E, Y49A, Y87A, Y87L, Y87M, F98A, F98L, F98M, A43Q, P44A, P44S, P44Q, L46E, and L46Q on the VL (according to Kabat numbering). The library can include selection of at least one, two, three, four, five etc. amino acid substitutions selected from positions Q39D, W47A, W47L, W47M, Y91A, Y91L, Y91M, H91A, W103A, W103L, W103M, V37S, V37Q, G44Q, L45A, and L45Q on the VH and/or correspondingly at least one, two, three, four, five etc. amino acid substitutions selected from positions R38E, Y49A, Y87A, Y87L, Y87M, F98A, F98L, F98M, A43Q, P44A, P44S, P44Q, L46E, and L46Q on the VL (according to Kabat numbering). In a further aspect of the invention, the library includes at least one amino acid substitution selected from at positions 30 and 100a (according to Kabat numbering). In a further aspect, the substitution is selected from S30V and F100aI. It should be noted that the library is not limited to aforementioned substitutions but include without limitation any substitutions that may be derived from following the steps set out in the methods of screening described herein, e.g., in this “Methods of screening” section herein.

### D) Methods and Compositions for Diagnostics and Detection

In some embodiments, the protein complex, polypeptide, protein, fusion protein, binding molecule complex or binding molecule comprises a ligand-binding moiety/molecule/molecule complex that comprises a ligand-binding domain or antigen-binding domain, wherein the binding domains encompasses antibody fragments, e.g. VH, VL, CH1, CL. As provided herein, the protein complex, polypeptide, protein, fusion protein, binding molecule complex or binding molecule is useful for detecting the presence of IL-12 ligand binding partner, such as IL-12 ligand receptor in a biological sample. The term “detecting” as used herein encompasses quantitative or qualitative detection. In certain embodiments, a biological sample comprises a cell or tissue, such as cancer cell or tissue expressing ligand receptor, or inflammatory cell or tissue expressing ligand receptor, or any ligand receptor-expressing cell or tissue.

In one embodiment, the protein complex, polypeptide, protein, fusion protein, binding molecule complex or binding molecule for use in a method of diagnosis or detection is provided. In a further aspect, a method of detecting the presence of ligand binding partner, such as ligand receptor in a biological sample is provided. In certain embodiments, the method comprises contacting the biological sample with the protein complex, polypeptide, protein, fusion protein, binding molecule complex or binding molecule as described herein under conditions permissive for binding of the protein complex, polypeptide, protein, fusion protein, binding molecule complex or binding molecule to ligand receptor, and detecting whether a complex is formed between the protein complex, polypeptide, protein, fusion protein, binding molecule complex or binding molecule and the ligand IL-12 receptor. Such method may be an in vitro or in vivo method. In one embodiment, the protein complex, polypeptide, protein, fusion protein, binding molecule complex or binding molecule is used to select subjects eligible for therapy with a protein complex, polypeptide, protein, fusion protein, binding molecule complex or binding molecule, e.g. where the ligand IL-12 receptor is a biomarker for selection of patients.

In certain embodiments, labelled protein complex, polypeptide, protein, fusion protein, binding molecule complex or binding molecule are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 125I, 3H, and 131I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, those coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### E) Pharmaceutical Compositions/Formulations

Pharmaceutical compositions/formulations of the present invention as described herein are prepared by mixing such protein complex, polypeptide, protein, fusion protein, binding molecule complex or binding molecule having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

The composition/formulation herein may also contain more than one active ingredient as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the protein complex, polypeptide, protein, fusion protein, binding molecule complex or binding molecule, which matrices are in the form of shaped articles, e.g. films, or microcapsules.

The compositions/formulations to be used for in vivo administration are generally sterile. Sterility may be readily accomplished, e.g., by filtration through sterile filtration membranes.

### F) Therapeutic Methods and Compositions

The present invention also relates to a pharmaceutical composition comprising the protein complex, polypeptide, protein, fusion protein, antibody, attenuated IL-12, binding molecule complex or binding molecule of the present invention and a pharmaceutically acceptable carrier. In certain embodiments, the pharmaceutical composition of the disclosure is a cell growth-suppressing agent. In certain embodiments, the pharmaceutical composition of the disclosure is a pharmaceutical composition used for treatment and/or prevention of cancers or malignancies.

In certain embodiments, the pharmaceutical composition of the disclosure is a pharmaceutical composition used for treatment and/or prevention of inflammatory diseases. In certain embodiments, the pharmaceutical composition of the disclosure is a pharmaceutical composition used for treatment and/or prevention of gut or liver inflammatory diseases. In certain embodiments, the pharmaceutical composition of the disclosure is a pharmaceutical composition used for treatment and/or prevention of inflammatory bowel disease, alcoholic fatty liver disease, or non-alcoholic fatty liver disease. In certain embodiments, the pharmaceutical composition of the disclosure is a pharmaceutical composition used for treatment and/or prevention of Ulcerative Colitis or Crohn's Disease.

In certain embodiments, the pharmaceutical composition of the disclosure is a pharmaceutical composition used for treatment and/or prevention of autoimmune diseases. In certain embodiments, the pharmaceutical composition of the disclosure is a pharmaceutical composition used for treatment and/or prevention of rheumatoid arthritis, type 1 diabetes, and SLE.

The “treatment” (and its grammatically derived words, for example, “treat” and “treating”) used in the present specification means clinical intervention that intends to alter the natural course of an individual to be treated and can be carried out both for prevention and during the course of a clinical pathological condition. The desirable effect of the treatment includes, but is not limited to, the prevention of the development or recurrence of a disease, the alleviation of symptoms, the attenuation of any direct or indirect pathological influence of the disease, the prevention of metastasis, reduction in the rate of progression of the disease, recovery from or alleviation of a disease condition, and ameliorated or improved prognosis. In some embodiments, the ligand binding molecule of the present invention can control the biological activity of the ligand and is used for delaying the onset of a disease(s) or delaying the progression of the disease(s).

In the present invention, the pharmaceutical composition usually refers to a drug for the treatment or prevention of a disease or for examination or diagnosis.

In the present invention, the term “pharmaceutical composition comprising the protein complex, polypeptide, protein, fusion protein, antibody, attenuated IL-12, binding molecule complex or binding molecule” may be used interchangeably with a “method for treating a disease, comprising administering the protein complex, polypeptide, protein, fusion protein, antibody, attenuated IL-12, binding molecule complex or binding molecule to a subject to be treated” and may be used interchangeably with “use of the protein complex, polypeptide, protein, fusion protein, antibody, attenuated IL-12, binding molecule complex or binding molecule for the production of a drug for the treatment of a disease”. Also, the term “pharmaceutical composition comprising the protein complex, polypeptide, protein, fusion protein, antibody, attenuated IL-12, binding molecule complex or binding molecule” may be used interchangeably with “use of the protein complex, polypeptide, protein, fusion protein, antibody, attenuated IL-12, binding molecule complex or binding molecule for treating a disease”.

In the present invention, the term “pharmaceutical composition comprising the protein complex/fusion protein/polypeptide/protein/antibody/attenuated IL-12/binding molecule complex/binding molecule” may be used interchangeably with a “method for treating a disease, comprising administering the protein complex/fusion protein/polypeptide/protein/antibody/attenuated IL-12/binding molecule complex/binding molecule to a subject to be treated” and may be used interchangeably with “use of the protein complex/fusion protein/polypeptide/protein/antibody/attenuated IL-12/binding molecule complex/binding molecule for the production of a drug for the treatment of a disease”. Also, the term “pharmaceutical composition comprising the protein complex/fusion protein/polypeptide/protein/antibody/attenuated IL-12/binding molecule complex/binding molecule” may be used interchangeably with “use of the protein complex/fusion protein/polypeptide/protein/antibody/attenuated IL-12/binding molecule complex/binding molecule for treating a disease”.

In some embodiments of the present invention, the protein complex, polypeptide, protein, fusion protein, antibody, attenuated IL-12, binding molecule complex or binding molecule of the present invention can be administered to an individual. The noncovalent bond still exists between the constant region of the ligand-binding moiety/molecule and the ligand moiety.

In the case of administering the protein complex, polypeptide, protein, fusion protein, antibody, attenuated IL-12, binding molecule complex or binding molecule of the present invention to an individual, the protein complex, polypeptide, protein, fusion protein, antibody, attenuated IL-12, binding molecule complex or binding molecule is transported in vivo. In the embodiments, the ligand-binding moiety suppresses the biological activity of the ligand moiety when the ligand binding domain is bound with the ligand, and the ligand-binding moiety is cleaved specifically in a target tissue, the ligand in the protein complex, polypeptide, protein, fusion protein, binding molecule complex or binding molecule does not exert biological activity during transport and exerts biological activity only when the protein complex, polypeptide, protein, fusion protein, binding molecule complex or binding molecule is cleaved in the target tissue. As a result, the disease can be treated with less systemic adverse reactions.

In the present invention, the ligand moiety is IL-12 and the protein complex, polypeptide, protein, fusion protein, binding molecule complex or binding molecule binds IL-12R. In this aspect, an IL-12 protein complex, polypeptide, protein, fusion protein, antibody, attenuated IL-12, binding molecule complex or binding molecule of aspects and embodiments herein for use as a medicament is provided. In further aspects, a protein complex, polypeptide, protein, fusion protein, antibody, attenuated IL-12, binding molecule complex or binding molecule provided herein for use in treating an IL-12 mediated disease is provided. In certain embodiments, an IL-12 protein complex, polypeptide, protein, fusion protein, antibody, attenuated IL-12, binding molecule complex or binding molecule for use in a method of treatment is provided. In certain embodiments, the invention provides an IL-12 protein complex, polypeptide, protein, fusion protein, antibody, attenuated IL-12, binding molecule complex or binding molecule for use in a method of treating an individual having an IL-12 mediated disease, or cancer, or malignancies, or inflammatory diseases, or autoimmune diseases, comprising administering to the individual an effective amount of the IL-12 protein complex, polypeptide, protein, fusion protein, antibody, attenuated IL-12, binding molecule complex or binding molecule. In one such embodiment, the method further comprises administering to the individual an effective amount of at least one additional therapeutic agent, e.g., as described below. In further embodiments, the invention provides an IL-12 protein complex, polypeptide, protein, fusion protein, antibody, attenuated IL-12, binding molecule complex or binding molecule provided herein for use in the modulation of IL-12 signaling in an individual. In certain embodiments, the invention provides an IL-12 protein complex, polypeptide, protein, fusion protein, antibody, attenuated IL-12, binding molecule complex or binding molecule provided herein for use in a method of modulation of IL-12 signaling in an individual comprising administering to the individual an effective amount of the IL-12 protein complex, polypeptide, protein, fusion protein, antibody, attenuated IL-12, binding molecule complex or binding molecule provided herein to treat an IL-12 mediated disease, or cancer, or malignancies, or inflammatory diseases, or autoimmune diseases. An “individual” according to any of the above embodiments is preferably a human.

In a further aspect, the invention provides for the use of an IL-12 protein complex, polypeptide, protein, fusion protein, antibody, attenuated IL-12, binding molecule complex or binding molecule provided herein in the manufacture or preparation of a medicament. In one embodiment, the medicament is for treatment of an IL-12 mediated disease, or cancer, or malignancies, or inflammatory diseases, or autoimmune diseases. In a further embodiment, the medicament is for use in a method of treating an IL-12 mediated disease comprising administering to an individual having an IL-12 mediated disease, or cancer, or malignancies, or inflammatory diseases, or autoimmune diseases an effective amount of the medicament. In one such embodiment, the method further comprises administering to the individual an effective amount of at least one additional therapeutic agent. In a further embodiment, the medicament is for the modulation of IL-12 signaling in an individual. In a further embodiment, the medicament is for use in a method of the modulation of IL-12 signaling in an individual comprising administering to the individual an amount effective of the medicament to treat an IL-12 mediated disease, or cancer, or malignancies, or inflammatory diseases, or autoimmune diseases. An “individual” according to any of the above embodiments may be a human.

In a further aspect, the invention provides a method for treating an IL-12 mediated disease, or cancer, or malignancies, or inflammatory diseases, or autoimmune diseases. In one embodiment, the method comprises administering to an individual having such IL-12 mediated disease, or cancer, or malignancies, or inflammatory diseases, or autoimmune diseases an effective amount of an IL-12 protein complex, polypeptide, protein, fusion protein, antibody, attenuated IL-12, binding molecule complex or binding molecule provided herein. In one such embodiment, the method further comprises administering to the individual an effective amount of at least one additional therapeutic agent. An “individual” according to any of the above embodiments may be a human.

In a further aspect, the invention provides a method for modulation of IL-12 signaling in an individual. In one embodiment, the method comprises administering to the individual an effective amount of an IL-12 protein complex, polypeptide, protein, fusion protein, antibody, attenuated IL-12, binding molecule complex or binding molecule provided herein to modulate IL-12 signaling. In one embodiment, an “individual” is a human.

As aforementioned, treatment of an IL-12 mediated disease refers to the treatment of any disease, disorder, or condition susceptible of being improved or prevented by an increase or enhancement in IL-12 signaling. The present invention includes use of the IL-12 protein complex, polypeptide, protein, fusion protein, antibody, attenuated IL-12, binding molecule complex or binding molecule described herein for use in the treatment of any disease, disorder, or condition susceptible of being improved or prevented by an increase or enhancement in IL-12 signaling, or wherein IL-12 is helpful. In a further aspect, the invention includes use of the IL-12 protein complex, polypeptide, protein, fusion protein, antibody, attenuated IL-12, binding molecule complex or binding molecule provided herein described herein in the manufacture of a medicament for the treatment of any disease, disorder, or condition susceptible of being improved or prevented by an increase or enhancement in IL-12 signaling, or wherein IL-12 is helpful. In a further aspect, the invention includes the IL-12 protein complex, polypeptide, protein, fusion protein, antibody, attenuated IL-12, binding molecule complex or binding molecule provided herein described herein in a method of treating any disease, disorder, or condition susceptible of being improved or prevented by an increase or enhancement in IL-12 signaling, or wherein IL-12 is helpful. In a further aspect, the modulation of IL-12 signaling is an increase or enhancement of IL-12 signaling that improves or prevents a disease, disorder or condition susceptible of being improved or prevented by said increase or enhancement in IL-12 signaling, or wherein IL-12 is helpful. In a further aspect, the invention includes a method of target treatment of cancer, or a method of administering to the subject an IL-12 therapy with reduced systemic exposure and/or reduced systemic toxicity, or method of locally inducing an immune response in a target tissue, comprising administering to an individual in need thereof the IL-12 protein complex, polypeptide, protein, fusion protein, antibody, attenuated IL-12, binding molecule complex or binding molecule provided herein.

In one embodiment, the preferred cell types are IL-12 ligand receptor-expressing cells within the tumor microenvironment. In a preferred embodiment, the invention provides a method for treating a cancer or malignancy comprising administering to an individual having cancer, including but not limited to, for example, gastric cancer, head and neck cancer (H&N), esophageal cancer, lung cancer, liver cancer, ovary cancer, breast cancer, colon cancer, colorectal cancer, skin cancer, muscle tumor, pancreas cancer, prostate cancer, testis cancer, uterine cancer, cholangiocarcinoma, Merkel cell carcinoma, bladder cancer, thyroid cancer, schwannoma, adrenal cancer (adrenal gland), anus cancer, central nervous system tumor, neuroendocrine tissue tumor, penis cancer, pleura tumor, salivary gland tumor, vulva cancer, thymoma, and childhood cancer (Wilms tumor, neuroblastoma, sarcoma, hepatoblastoma, and germ cell tumor). Still more preferred cancer types include, but are not limited to, gastric cancer, head and neck cancer (H&N), esophageal cancer, lung cancer, liver cancer, ovary cancer, breast cancer, colon cancer, kidney cancer, skin cancer, muscle tumor, pancreas cancer, prostate cancer, testis cancer, and uterine cancer (Tumori. (2012) 98, 478-484; Tumor Biol. (2015) 36, 4671-4679; Am J Clin Pathol (2008) 130, 224-230; Adv Anat Pathol (2014) 21, 450-460; Med Oncol (2012) 29, 663-669; Clinical Cancer Research (2004) 10, 6612-6621; Appl Immunohistochem Mol Morphol (2009) 17, 40-46; Eur J Pediatr Surg (2015) 25, 138-144; J Clin Pathol (2011) 64, 587-591; Am J Surg Pathol (2006) 30, 1570-1575; Oncology (2007) 73, 389-394; Diagnostic Pathology (2010) 64, 1-6; Diagnostic Pathology (2015) 34, 1-6; Am J Clin Pathol (2008) 129, 899-906; Virchows Arch (2015) 466, 67-76).

In several embodiments, the individuals are patients who cannot receive standard therapy or for whom standard therapy is ineffective. In several embodiments, the cancer in which a patient has is early-stage or end-stage.

As used herein, “cancer” refers not only to epithelial malignancy such as ovary cancer or gastric cancer but also to non-epithelial malignancy including hematopoietic tumors such as chronic lymphocytic leukemia or Hodgkin's lymphoma. Herein, the terms “cancer”, “carcinoma”, “tumor”, “neoplasm” and such are not differentiated from each other and are mutually interchangeable.

In a further aspect, the invention provides pharmaceutical compositions/formulations comprising any of the protein complex, polypeptide, protein, fusion protein, antibody, attenuated IL-12, binding molecule complex or binding molecule provided herein, e.g., for use in any of the above therapeutic methods. In one embodiment, a pharmaceutical composition/formulation comprises any of the protein complex, polypeptide, protein, fusion protein, antibody, attenuated IL-12, binding molecule complex or binding molecule provided herein and a pharmaceutically acceptable carrier. In another embodiment, a pharmaceutical composition/formulation comprises any of the protein complex, polypeptide, protein, fusion protein, antibody, attenuated IL-12, binding molecule complex or binding molecule provided herein and at least one additional therapeutic agent.

Fusion proteins or protein complexes of the present invention can be used either alone or in combination with other agents in a therapy. For instance, the fusion protein or protein complex of the present invention may be co-administered with at least one additional therapeutic agent. In certain embodiments, an additional therapeutic agent may be a cytostatic agent, a chemotherapeutic agent, or immunosuppressive agent. In one embodiment, an additional therapeutic agent is an immune checkpoint inhibitor.

Such combination therapies noted above encompass combined administration (where two or more therapeutic agents are included in the same or separate formulations), and separate administration, in which case, administration of the protein complex of the invention can occur prior to, simultaneously, and/or following, administration of the additional therapeutic agent or agents. In one embodiment, administration of the fusion protein or protein complex and administration of an additional therapeutic agent occur within about one month, or within about one, two or three weeks, or within about one, two, three, four, five, or six days, of each other. Fusion protein or protein complex of the invention can also be used in combination with radiation therapy.

In a non-limiting embodiment of the present invention, pharmaceutical compositions (combination therapy) of the present invention can be used to treat patients who have cancer which is refractory to treatment with an immune checkpoint inhibitor. For example, patients with ligand-related cancer, in whom administration of an immune checkpoint inhibitor has failed to achieve a desired drug efficacy, can be treated with the pharmaceutical composition (combination therapy) of the present invention. In other words, ligand-related cancer that has been already treated with therapy using an immune checkpoint inhibitor can be treated with the pharmaceutical composition (combination therapy) of the present invention. Preferred examples of an additional therapeutic agent comprised in the pharmaceutical composition include immune checkpoint inhibitors but are not limited thereto.

In a non-limiting embodiment of the present invention, pharmaceutical compositions (combination therapy) of the present invention can be used to treat patients who have cancer which is refractory to treatment with the fusion protein or protein complex of the present invention. For example, patients with ligand-related cancer, whose cancer has become resistant to the fusion protein or protein complex of the present invention after administration of said proteins or protein complexes or in whom administration of the proteins of the present invention has failed to achieve a desired drug efficacy, can be treated with the pharmaceutical composition (combination therapy) of the present invention. In other words, ligand-related cancer that has been already treated with therapy using the fusion protein or protein complex of the present invention can be treated with the pharmaceutical composition (combination therapy) of the present invention. Preferred examples of an additional therapeutic agent comprised in the pharmaceutical composition include immune checkpoint inhibitors but are not limited thereto.

A protein complex, polypeptide, protein, fusion protein, antibody, attenuated IL-12, binding molecule complex or binding molecule of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

A protein complex, polypeptide, protein, fusion protein, antibody, attenuated IL-12, binding molecule complex or binding molecule of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The protein complex, polypeptide, protein, fusion protein, antibody, attenuated IL-12, binding molecule complex or binding molecule need not be, but is optionally formulated with one or more agents currently used to prevent or treat the disorder in question. The effective amount of such other agents depends on the amount of protein complex, polypeptide, protein, fusion protein, antibody, attenuated IL-12, binding molecule complex or binding molecule present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the protein complex, polypeptide, protein, fusion protein, antibody, attenuated IL-12, binding molecule complex or binding molecule of the invention will depend on the type of disease to be treated, the severity and course of the disease, whether the protein complex, polypeptide, protein, fusion protein, antibody, attenuated IL-12, binding molecule complex or binding molecule is administered for preventive or therapeutic purposes, previous therapy, the patient's clinical history and response to the protein complex, polypeptide, protein, fusion protein, binding molecule complex or binding molecule, and the discretion of the attending physician. The protein complex, polypeptide, protein, fusion protein, antibody, attenuated IL-12, binding molecule complex or binding molecule is suitably administered to the patient at one time or over a series of treatments. Depending on the type and severity of the disease, about 1 micro g/kg to 15 mg/kg (e.g. 0.1 mg/kg-10 mg/kg) of protein complex, polypeptide, protein, fusion protein, antibody, attenuated IL-12, binding molecule complex or binding molecule can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the protein complex, polypeptide, protein, fusion protein, antibody, attenuated IL-12, binding molecule complex or binding molecule would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the protein complex, polypeptide, protein, fusion protein, antibody, attenuated IL-12, binding molecule complex or binding molecule). An initial higher loading dose, followed by one or more lower doses may be administered. The progress of this therapy is easily monitored by conventional techniques and assays.

### G) Articles of Manufacture

In another aspect of the invention, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is the protein complex, polypeptide, protein, fusion protein, antibody, attenuated IL-12, binding molecule complex or binding molecule of the invention. The label or package insert indicates that the composition is used for treating the condition of choice. Moreover, the article of manufacture may comprise (a) a first container with a composition contained therein, wherein the composition comprises a protein complex, polypeptide, protein, fusion protein, antibody, attenuated IL-12, binding molecule complex or binding molecule of the invention; and (b) a second container with a composition contained therein, wherein the composition comprises an additional therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### H) Exemplary IL-12 Fusion Polypeptides or Protein Complexes or Polypeptides, or Proteins or Antibodies, or Binding Molecule Complexes or Binding Molecules of the Present Invention

In one aspect, the invention provides a protein complex comprising at least one, two, three, four, five, or six CDRs selected from (a) CDR-H1 comprising the amino acid sequence of SEQ ID NO: 346; (b) CDR-H2 comprising the amino acid sequence of SEQ ID NO: 347 or 348; (c) CDR-H3 comprising the amino acid sequence of SEQ ID NO: 349 or 350; (d) CDR-L1 comprising the amino acid sequence of SEQ ID NO: 351 or 352; (e) CDR-L2 comprising the amino acid sequence of SEQ ID NO: 353 or 354; and (f) CDR-L3 comprising the amino acid sequence of SEQ ID NO: 355.

In one aspect, the invention provides a protein complex comprising at least one, two, or all three, VH CDRs selected from (a) CDR-H1 comprising the amino acid sequence of SEQ ID NO: 346; (b) CDR-H2 comprising the amino acid sequence of SEQ ID NO: 347 or 348; and (c) CDR-H3 comprising the amino acid sequence of SEQ ID NO: 349 or 350. In one embodiment, the protein complex comprises CDR-H3 comprising the amino acid sequence of SEQ ID NO: 349 or 350. In another embodiment, the protein complex comprises CDR-H3 comprising the amino acid sequence of SEQ ID NO: 349 or 350, CDR-L3 comprising the amino acid sequence of SEQ ID NO: 355. In a further embodiment, the protein complex comprises CDR-H3 comprising the amino acid sequence of SEQ ID NO: 349 or 350, CDR-L3 comprising the amino acid sequence of SEQ ID NO: 355, and CDR-H2 comprising the amino acid sequence of SEQ ID NO: 347 or 348. In a further embodiment, the protein complex comprises (a) CDR-H1 comprising the amino acid sequence of SEQ ID NO: 346; (b) CDR-H2 comprising the amino acid sequence of SEQ ID NO: 347 or 348; and (c) CDR-H3 comprising the amino acid sequence of SEQ ID NO: 349 or 350.

In another aspect, the invention provides a protein complex comprising at least one, two, or all three, VL CDRs selected from (a) CDR-L1 comprising the amino acid sequence of SEQ ID NO: 351 or 352; (b) CDR-L2 comprising the amino acid sequence of SEQ ID NO: 353 or 354; and (c) CDR-L3 comprising the amino acid sequence of SEQ ID NO: 355. In one embodiment, the protein complex comprises (a) CDR-L1 comprising the amino acid sequence of SEQ ID NO: 351 or 352; (b) CDR-L2 comprising the amino acid sequence of SEQ ID NO: 353 or 354; and (c) CDR-L3 comprising the amino acid sequence of SEQ ID NO: 355.

In another aspect, a protein complex of the invention comprises (a) a VH domain comprising at least one, at least two, or all three VH CDR sequences selected from (i) CDR-H1 comprising the amino acid sequence of SEQ ID NO: 346; ii) CDR-H2 comprising the amino acid sequence of SEQ ID NO: 347 or 348; and iii) CDR-H3 comprising the amino acid sequence of SEQ ID NO: 349 or 350, and (b) a VL domain comprising at least one, at least two, or all three VL CDR sequences selected from (i) CDR-L1 comprising the amino acid sequence of SEQ ID NO: 351 or 352; (ii) CDR-L2 comprising the amino acid sequence of SEQ ID NO: 353 or 354; and (iii) CDR-L3 comprising the amino acid sequence of SEQ ID NO: 355.

In another aspect, the invention provides a protein complex comprising (a) CDR-H1 comprising the amino acid sequence of SEQ ID NO: 346; (b) CDR-H2 comprising the amino acid sequence of SEQ ID NO: 347 or 348; (c) CDR-H3 comprising the amino acid sequence of SEQ ID NO: 349 or 350; (d) CDR-L1 comprising the amino acid sequence of SEQ ID NO: 351 or 352; (e) CDR-L2 comprising the amino acid sequence of SEQ ID NO: 353 or 354; and (f) CDR-L3 comprising the amino acid sequence of SEQ ID NO: 355.

In embodiments of the present application, the ligand moiety comprises IL-12, and the protein complex comprises or consist of two subunits associated with each other, wherein each subunit comprises a first polypeptide and a second polypeptide, wherein the first polypeptide comprises or consists of a light chain variable domain (VL) and a light chain constant domain (CL), and the second polypeptide is a fusion protein represented by the general formula (I), from the N- to the C-terminus:

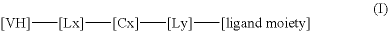

wherein:
Lx represents a cleavable peptide linker comprising a protease cleavage site,
Cx represents a constant domain associated with the CL of the first polypeptide, optionally, wherein Cx comprises or consists of, from the N- to the C-terminus, a CH1 domain, a second peptide linker, a CH2 domain, and a CH3 domain, and
Ly represents a third peptide linker,
VH represents a heavy chain variable domain (VH), wherein the VH is associated with the VL of the first polypeptide to form a ligand-binding domain, wherein the VH and the VL comprise any one of the following sequences:
(a) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
(b) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 354, and (iii) LCDR3 comprising at least 85%, 90%, 95%, 96%, 97%, 98%, or 99% sequence identity to the amino acid sequence of SEQ ID NO: 355; or
(c) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 352, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
(d) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
(e) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 354, and (iii) LCDR3 comprising at least 85%, 90%, 95%, 96%, 97%, 98%, or 99% sequence identity to the amino acid sequence of SEQ ID NO: 355; or
(f) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 352, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
(g) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 351, (ii) LCDR2 comprising the amino acid sequence of SEQ ID NO: 353, and (iii) LCDR3 comprising the amino acid sequence of SEQ ID NO: 355; or
(h) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 351, (ii) LCDR2 comprising the amino acid sequence of SEQ ID NO: 354, and (iii) LCDR3 comprising the amino acid sequence of SEQ ID NO: 355; or
(i) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 352, (ii) LCDR2 comprising the amino acid sequence of SEQ ID NO: 353, and (iii) LCDR3 comprising the amino acid sequence of SEQ ID NO: 355; or
(j) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 351, (ii) LCDR2 comprising the amino acid sequence of SEQ ID NO: 353, and (iii) LCDR3 comprising the amino acid sequence of SEQ ID NO: 355; or
(k) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 351, (ii) LCDR2 comprising the amino acid sequence of SEQ ID NO: 354, and (iii) LCDR3 comprising the amino acid sequence of SEQ ID NO: 355; or
(l) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 352, (ii) LCDR2 comprising the amino acid sequence of SEQ ID NO: 353, and (iii) LCDR3 comprising the amino acid sequence of SEQ ID NO: 355; or
(m) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 190; or
(n) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 342; or
(o) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 340, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 343; or
(p) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 340, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 344; or
(q) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 341, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 344; or
(r) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 191; or
(s) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 190; or
(t) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 342; or
(u) a heavy chain variable domain (VH) comprising HCDR1, HCDR2, and HCDR3 that is identical to the amino acids of HCDR1, HCDR2, and HCDR3 comprised in SEQ ID NO: 340, and a light chain variable domain (VL) comprising LCDR1, LCDR2, and LCDR3 that is identical to the amino acids of LCDR1, LCDR2, and LCDR3 comprised in SEQ ID NO: 343; or
(v) a heavy chain variable domain (VH) comprising HCDR1, HCDR2, and HCDR3 that is identical to the amino acids of HCDR1, HCDR2, and HCDR3 comprised in SEQ ID NO: 340, and a light chain variable domain (VL) comprising LCDR1, LCDR2, and LCDR3 that is identical to the amino acids of LCDR1, LCDR2, and LCDR3 comprised in SEQ ID NO: 344; or
(w) a heavy chain variable domain (VH) comprising HCDR1, HCDR2, and HCDR3 that is identical to the amino acids of HCDR1, HCDR2, and HCDR3 comprised in SEQ ID NO: 341, and a light chain variable domain (VL) comprising LCDR1, LCDR2, and LCDR3 that is identical to the amino acids of LCDR1, LCDR2, and LCDR3 comprised in SEQ ID NO: 344; or
(x) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 191; or
(y) a heavy chain variable domain (VH) comprising at least 85%, 90%, 95%, 96%, 97%, 98%, or 99% sequence identity to the amino acid sequence of SEQ ID NO: 189, and a light chain variable domain (VL) comprising at least 85%, 90%, 95%, 96%, 97%, 98%, or 99% sequence identity to the amino acid sequence of SEQ ID NO: 190; or
(z) a heavy chain variable domain (VH) comprising at least 85%, 90%, 95%, 96%, 97%, 98%, or 99% sequence identity to the amino acid sequence of SEQ ID NO: 189, and a light chain variable domain (VL) comprising at least 85%, 90%, 95%, 96%, 97%, 98%, or 99% sequence identity to the amino acid sequence of SEQ ID NO: 342; or
(aa) a heavy chain variable domain (VH) comprising at least 85%, 90%, 95%, 96%, 97%, 98%, or 99% sequence identity to the amino acid sequence of SEQ ID NO: 340, and a light chain variable domain (VL) comprising at least 85%, 90%, 95%, 96%, 97%, 98%, or 99% sequence identity to the amino acid sequence of SEQ ID NO: 343; or
(bb) a heavy chain variable domain (VH) comprising at least 85%, 90%, 95%, 96%, 97%, 98%, or 99% sequence identity to the amino acid sequence of SEQ ID NO: 340, and a light chain variable domain (VL) comprising at least 85%, 90%, 95%, 96%, 97%, 98%, or 99% sequence identity to the amino acid sequence of SEQ ID NO: 344; or
(cc) a heavy chain variable domain (VH) comprising at least 85%, 90%, 95%, 96%, 97%, 98%, or 99% sequence identity to the amino acid sequence of SEQ ID NO: 341, and a light chain variable domain (VL) comprising at least 85%, 90%, 95%, 96%, 97%, 98%, or 99% sequence identity to the amino acid sequence of SEQ ID NO: 344; or
(dd) a heavy chain variable domain (VH) comprising at least 85%, 90%, 95%, 96%, 97%, 98%, or 99% sequence identity to the amino acid sequence of SEQ ID NO: 189, and a light chain variable domain (VL) comprising at least 85%, 90%, 95%, 96%, 97%, 98%, or 99% sequence identity to the amino acid sequence of SEQ ID NO: 191; or (ee) a heavy chain variable domain (VH) comprising the amino acid sequence of SEQ ID NO: 189, and a light chain variable domain (VL) comprising the amino acid sequence of SEQ ID NO: 190; or
(ff) a heavy chain variable domain (VH) comprising the amino acid sequence of SEQ ID NO: 189, and a light chain variable domain (VL) comprising the amino acid sequence of SEQ ID NO: 342; or
(gg) a heavy chain variable domain (VH) comprising the amino acid sequence of SEQ ID NO: 340, and a light chain variable domain (VL) comprising the amino acid sequence of SEQ ID NO: 343; or
(hh) a heavy chain variable domain (VH) comprising the amino acid sequence of SEQ ID NO: 340, and a light chain variable domain (VL) comprising the amino acid sequence of SEQ ID NO: 344; or
(ii) a heavy chain variable domain (VH) comprising the amino acid sequence of SEQ ID NO: 341, and a light chain variable domain (VL) comprising the amino acid sequence of SEQ ID NO: 344; or
(jj) a heavy chain variable domain (VH) comprising the amino acid sequence of SEQ ID NO: 189, and a light chain variable domain (VL) comprising the amino acid sequence of SEQ ID NO: 191;
wherein Lx links VH and the CH1 domain and comprises any one of the following sequences:
(i) an amino acid sequence of SEQ ID NO: 199; or
(ii) an amino acid sequence of SEQ ID NO: 194,
wherein Cx comprises an amino acid sequence of SEQ ID NO: 75;
wherein Ly comprises an amino acid sequence of SEQ ID NO: 68;
wherein the ligand moiety is a protease resistant single-chain IL-12 fusion protein and comprises any one of the following sequences:
(i) an amino acid sequence of SEQ ID NO: 317; or
(ii) the amino acid sequence of SEQ ID NO: 317, wherein the tyrosine at position 16 of SEQ ID NO: 317 is substituted with an amino acid selected from the group consisting of: F, N, E, P, G, K, A, D, H, I, L, M, Q, R, S, T, V, and W; preferably P; or
(iii) an amino acid sequence of SEQ ID NO: 285; or
(iv) the amino acid sequence of SEQ ID NO: 285, wherein the tyrosine at position 16 of SEQ ID NO: 285 is substituted with an amino acid selected from the group consisting of: F, N, E, P, G, K, A, D, H, I, L, M, Q, R, S, T, V, and W; preferably P; or
(v) an amino acid sequence of SEQ ID NO: 325; or
(vi) the amino acid sequence of SEQ ID NO: 335.

In some embodiments of the present application, the two subunits are bound to each other via one or more disulfide bonds; and/or the CH1 domain of Cx and the CL of the first polypeptide are bound to each other via one or more disulfide bonds.

In some embodiments of the present application, the ligand moiety comprises IL-12, and the protein complex comprises two polypeptides, wherein each polypeptide is a fusion protein, and each polypeptide is represented by the general formula (II), from the N- to the C-terminus:

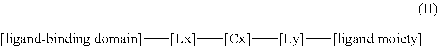

wherein:
Lx represents a cleavable peptide linker comprising a protease cleavage site,
Cx represents a constant domain comprising, from N- to C-terminus, a CH1 domain, a second peptide linker, a CH2 domain, and a CH3 domain, and
Ly represents a third peptide linker,
wherein the ligand-binding domain comprises a heavy chain variable domain (VH) associated with a light chain variable domain (VL), wherein VL is associated with a light chain constant domain (CL), wherein CL is associated with Cx, and wherein VH and VL comprise any one of the following sequences:
(a) a heavy chain variable domain (VH) comprising at least 85%, 90%, 95%, 96%, 97%, 98%, or 99% sequence identical to the amino acid sequence of SEQ ID NO: 189, and a light chain variable domain (VL) comprising at least 85%, 90%, 95%, 96%, 97%, 98%, or 99% sequence identical to the amino acid sequence of SEQ ID NO: 190; or
(b) a heavy chain variable domain (VH) comprising at least 85%, 90%, 95%, 96%, 97%, 98%, or 99% sequence identical to the amino acid sequence of SEQ ID NO: 189, and a light chain variable domain (VL) comprising at least 85%, 90%, 95%, 96%, 97%, 98%, or 99% sequence identical to the amino acid sequence of SEQ ID NO: 342; or
(c) a heavy chain variable domain (VH) comprising at least 85%, 90%, 95%, 96%, 97%, 98%, or 99% sequence identical to the amino acid sequence of SEQ ID NO: 340, and a light chain variable domain (VL) comprising at least 85%, 90%, 95%, 96%, 97%, 98%, or 99% sequence identical to the amino acid sequence of SEQ ID NO: 343; or
(d) a heavy chain variable domain (VH) comprising at least 85%, 90%, 95%, 96%, 97%, 98%, or 99% sequence identical to the amino acid sequence of SEQ ID NO: 340, and a light chain variable domain (VL) comprising at least 85%, 90%, 95%, 96%, 97%, 98%, or 99% sequence identical to the amino acid sequence of SEQ ID NO: 344; or
(e) a heavy chain variable domain (VH) comprising at least 85%, 90%, 95%, 96%, 97%, 98%, or 99% sequence identical to the amino acid sequence of SEQ ID NO: 341, and a light chain variable domain (VL) comprising at least 85%, 90%, 95%, 96%, 97%, 98%, or 99% sequence identical to the amino acid sequence of SEQ ID NO: 344; or
(f) a heavy chain variable domain (VH) comprising at least 85%, 90%, 95%, 96%, 97%, 98%, or 99% sequence identical to the amino acid sequence of SEQ ID NO: 189, and a light chain variable domain (VL) comprising at least 85%, 90%, 95%, 96%, 97%, 98%, or 99% sequence identical to the amino acid sequence of SEQ ID NO: 191; or
(g) a heavy chain variable domain (VH) comprising the amino acid sequence of SEQ ID NO: 189, and a light chain variable domain (VL) comprising the amino acid sequence of SEQ ID NO: 190; or
(h) a heavy chain variable domain (VH) comprising the amino acid sequence of SEQ ID NO: 189, and a light chain variable domain (VL) comprising the amino acid sequence of SEQ ID NO: 342; or
(i) a heavy chain variable domain (VH) comprising the amino acid sequence of SEQ ID NO: 340, and a light chain variable domain (VL) comprising the amino acid sequence of SEQ ID NO: 343; or
(j) a heavy chain variable domain (VH) comprising the amino acid sequence of SEQ ID NO: 340, and a light chain variable domain (VL) comprising the amino acid sequence of SEQ ID NO: 344; or
(k) a heavy chain variable domain (VH) comprising the amino acid sequence of SEQ ID NO: 341, and a light chain variable domain (VL) comprising the amino acid sequence of SEQ ID NO: 344; or
(l) a heavy chain variable domain (VH) comprising the amino acid sequence of SEQ ID NO: 189, and a light chain variable domain (VL) comprising the amino acid sequence of SEQ ID NO: 191;
wherein Lx links VH and the CH1 domain and comprises any one of the following sequences:
(i) an amino acid sequence of SEQ ID NO: 199; or
(ii) an amino acid sequence of SEQ ID NO: 194,
wherein Cx comprises an amino acid sequence of SEQ ID NO: 75;
wherein Ly comprises an amino acid sequence of SEQ ID NO: 68;
wherein the ligand moiety is a protease resistant single-chain IL-12 fusion protein and comprises any one of the following sequences:
(i) an amino acid sequence of SEQ ID NO: 317; or
(ii) the amino acid sequence of SEQ ID NO: 317, wherein the tyrosine at position 16 of SEQ ID NO: 317 is substituted with an amino acid selected from the group consisting of: F, N, E, P, G, K, A, D, H, I, L, M, Q, R, S, T, V, and W; preferably P; or
(iii) an amino acid sequence of SEQ ID NO: 285; or
(iv) the amino acid sequence of SEQ ID NO: 285, wherein the tyrosine at position 16 of SEQ ID NO: 285 is substituted with an amino acid selected from the group consisting of: F, N, E, P, G, K, A, D, H, I, L, M, Q, R, S, T, V, and W; preferably P; or
(v) an amino acid sequence of SEQ ID NO: 325; or
(vi) the amino acid sequence of SEQ ID NO: 335.

In some embodiments of the present application, the two fusion proteins are bound to each other via one or more disulfide bonds; and/or each fusion protein comprises a light chain consisting of the VL and a CL light chain constant domain to a CH1 heavy chain constant domain via one or more disulfide bonds.

In some embodiments of the present application, the protein complex comprises the following:
(a) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(b) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(c) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(d) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(e) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(f) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(g) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(h) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(i) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(j) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(k) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(l) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317; and
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(m) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(n) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(o) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(p) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(q) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
   or
(r) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or comprising or consisting of the amino acid sequence of SEQ ID NO: 317;
(s) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(t) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317; and
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(u) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(v) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(w) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317; and
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(x) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) comprising or consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P.

In some embodiments of the present application, the protein complex comprises the following:
(a) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(b) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(c) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(d) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(e) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(f) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 285, or consisting of the amino acid sequence of SEQ ID NO: 317;
   or
(g) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(h) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(i) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO:343;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(j) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(k) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(l) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(m) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(n) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(o) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(p) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(q) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(r) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 285, or consisting of the amino acid sequence of SEQ ID NO: 317;
   or
(s) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(t) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(u) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(v) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(w) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P;
   or
(x) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein
   (i) comprising the amino acid sequence of SEQ ID NO: 285, or (ii) consisting of the amino acid sequence of SEQ ID NO: 317;
   wherein the tyrosine at position 16 of SEQ ID NO: 285 or SEQ ID NO: 317 is substituted with an amino acid selected from the group consisting of: F, N, E, P, G, K, A, D, H, I, L, M, Q, R, S, T, V, and W; preferably P.

In some embodiments of the present application, the protein complex comprises the following:
(a) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(b) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(c) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(d) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(e) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(f) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(g) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(h) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(i) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(j) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(k) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
   or
(l) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or comprising or consisting of the amino acid sequence of SEQ ID NO: 335;
(m) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(n) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(o) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(p) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(q) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(r) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 199; and
   the ligand moiety is a protease resistant single-chain IL-12 fusion protein comprising the amino acid sequence of SEQ ID NO: 325, or consisting of the amino acid sequence of SEQ ID NO: 335;
   or
(s) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 190;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(t) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 342;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(u) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 343;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(v) said VH and VL comprise the following sequences:
   a heavy chain variable domain (VH) comprising or consisting of the amino acid sequence of SEQ ID NO: 340, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(w) said VH and VL comprise the following sequences:
   a heavy chain variable domain (VH) comprising or consisting of the amino acid sequence of SEQ ID NO: 341, and a light chain variable domain (VL) comprising or consisting of the amino acid sequence of SEQ ID NO: 344;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335;
   or
(x) said VH and VL comprise the following sequences:
   a heavy chain variable domain (VH) comprising or consisting of the amino acid sequence of SEQ ID NO: 189, and a light chain variable domain (VL) comprising or consisting of the amino acid sequence of SEQ ID NO: 191;
   the cleavable peptide linker comprises or consists of an amino acid sequence of SEQ ID NO: 194; and
   the ligand moiety is a protease resistant single-chain IL-12 fusion protein comprising the amino acid sequence of SEQ ID NO: 325, or consisting of the amino acid sequence of SEQ ID NO: 335.

In some embodiments of the present application, the ligand moiety comprises IL-12, and the protein complex comprises a protease resistant single-chain IL-12 fusion protein fused with an anti-IL-12 binding molecule complex, wherein the anti-IL-12 binding molecule complex comprises a heavy chain variable domain (VH) associated with a light chain variable domain (VL), a cleavable peptide linker comprising a protease cleavage site, and a constant domain,
wherein said VH and VL comprises any one of the following sequences:
(A) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
(B) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 354, and (iii) LCDR3 comprising at least 85%, 90%, 95%, 96%, 97%, 98%, or 99% sequence identity to the amino acid sequence of SEQ ID NO: 355; or
(C) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 352, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
(D) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
(E) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 354, and (iii) LCDR3 comprising at least 85%, 90%, 95%, 96%, 97%, 98%, or 99% sequence identity to the amino acid sequence of SEQ ID NO: 355; or
(F) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 352, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355; or
(G) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 351, (ii) LCDR2 comprising the amino acid sequence of SEQ ID NO: 353, and (iii) LCDR3 comprising the amino acid sequence of SEQ ID NO: 355; or
(H) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 351, (ii) LCDR2 comprising the amino acid sequence of SEQ ID NO: 354, and (iii) LCDR3 comprising the amino acid sequence of SEQ ID NO: 355; or
(I) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 352, (ii) LCDR2 comprising the amino acid sequence of SEQ ID NO: 353, and (iii) LCDR3 comprising the amino acid sequence of SEQ ID NO: 355; or
(J) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 351, (ii) LCDR2 comprising the amino acid sequence of SEQ ID NO: 353, and (iii) LCDR3 comprising the amino acid sequence of SEQ ID NO: 355; or
(K) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 351, (ii) LCDR2 comprising the amino acid sequence of SEQ ID NO: 354, and (iii) LCDR3 comprising the amino acid sequence of SEQ ID NO: 355; or
(L) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 352, (ii) LCDR2 comprising the amino acid sequence of SEQ ID NO: 353, and (iii) LCDR3 comprising the amino acid sequence of SEQ ID NO: 355; or
(M) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 190; or
(N) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 342; or
(O) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 340, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 343; or
(P) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 340, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 344; or
(Q) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 341, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 344; or
(R) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 191; or
(S) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 190; or
(T) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 342; or
(U) a heavy chain variable domain (VH) comprising HCDR1, HCDR2, and HCDR3 that is identical to the amino acids of HCDR1, HCDR2, and HCDR3 comprised in SEQ ID NO: 340, and a light chain variable domain (VL) comprising LCDR1, LCDR2, and LCDR3 that is identical to the amino acids of LCDR1, LCDR2, and LCDR3 comprised in SEQ ID NO: 343; or
(V) a heavy chain variable domain (VH) comprising HCDR1, HCDR2, and HCDR3 that is identical to the amino acids of HCDR1, HCDR2, and HCDR3 comprised in SEQ ID NO: 340, and a light chain variable domain (VL) comprising LCDR1, LCDR2, and LCDR3 that is identical to the amino acids of LCDR1, LCDR2, and LCDR3 comprised in SEQ ID NO: 344; or
(W) a heavy chain variable domain (VH) comprising HCDR1, HCDR2, and HCDR3 that is identical to the amino acids of HCDR1, HCDR2, and HCDR3 comprised in SEQ ID NO: 341, and a light chain variable domain (VL) comprising LCDR1, LCDR2, and LCDR3 that is identical to the amino acids of LCDR1, LCDR2, and LCDR3 comprised in SEQ ID NO: 344; or
(X) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 191; or
(a) a heavy chain variable domain (VH) comprising at least 85%, 90%, 95%, 96%, 97%, 98%, or 99% sequence identical to the amino acid sequence of SEQ ID NO: 189, and a light chain variable domain (VL) comprising at least 85%, 90%, 95%, 96%, 97%, 98%, or 99% sequence identical to the amino acid sequence of SEQ ID NO: 190; or
(b) a heavy chain variable domain (VH) comprising at least 85%, 90%, 95%, 96%, 97%, 98%, or 99% sequence identical to the amino acid sequence of SEQ ID NO: 189, and a light chain variable domain (VL) comprising at least 85%, 90%, 95%, 96%, 97%, 98%, or 99% sequence identical to the amino acid sequence of SEQ ID NO: 342; or
(c) a heavy chain variable domain (VH) comprising at least 85%, 90%, 95%, 96%, 97%, 98%, or 99% sequence identical to the amino acid sequence of SEQ ID NO: 340, and a light chain variable domain (VL) comprising at least 85%, 90%, 95%, 96%, 97%, 98%, or 99% sequence identical to the amino acid sequence of SEQ ID NO: 343; or
(d) a heavy chain variable domain (VH) comprising at least 85%, 90%, 95%, 96%, 97%, 98%, or 99% sequence identical to the amino acid sequence of SEQ ID NO: 340, and a light chain variable domain (VL) comprising at least 85%, 90%, 95%, 96%, 97%, 98%, or 99% sequence identical to the amino acid sequence of SEQ ID NO: 344; or
(e) a heavy chain variable domain (VH) comprising at least 85%, 90%, 95%, 96%, 97%, 98%, or 99% sequence identical to the amino acid sequence of SEQ ID NO: 341, and a light chain variable domain (VL) comprising at least 85%, 90%, 95%, 96%, 97%, 98%, or 99% sequence identical to the amino acid sequence of SEQ ID NO: 344; or
(f) a heavy chain variable domain (VH) comprising at least 85%, 90%, 95%, 96%, 97%, 98%, or 99% sequence identical to the amino acid sequence of SEQ ID NO: 189, and a light chain variable domain (VL) comprising at least 85%, 90%, 95%, 96%, 97%, 98%, or 99% sequence identical to the amino acid sequence of SEQ ID NO: 191; or
(g) a heavy chain variable domain (VH) comprising the amino acid sequence of SEQ ID NO: 189, and a light chain variable domain (VL) comprising the amino acid sequence of SEQ ID NO: 190; or
(h) a heavy chain variable domain (VH) comprising the amino acid sequence of SEQ ID NO: 189, and a light chain variable domain (VL) comprising the amino acid sequence of SEQ ID NO: 342; or
(i) a heavy chain variable domain (VH) comprising the amino acid sequence of SEQ ID NO: 340, and a light chain variable domain (VL) comprising the amino acid sequence of SEQ ID NO: 343; or
(j) a heavy chain variable domain (VH) comprising the amino acid sequence of SEQ ID NO: 340, and a light chain variable domain (VL) comprising the amino acid sequence of SEQ ID NO: 344; or
(k) a heavy chain variable domain (VH) comprising the amino acid sequence of SEQ ID NO: 341, and a light chain variable domain (VL) comprising the amino acid sequence of SEQ ID NO: 344; or
(l) a heavy chain variable domain (VH) comprising the amino acid sequence of SEQ ID NO: 189, and a light chain variable domain (VL) comprising the amino acid sequence of SEQ ID NO: 191;
wherein the cleavable peptide linker is located between the boundary of VH and the constant domain, and comprises any one of the following sequences:
(i) an amino acid sequence of SEQ ID NO: 199; or
(ii) an amino acid sequence of SEQ ID NO: 194,
wherein the constant domain comprises an amino acid sequence SEQ ID NO: 75;
wherein the protease resistant single-chain IL-12 fusion protein is fused to the C-terminus of the constant domain and comprises one of the following sequences:
(i) an amino acid sequence of SEQ ID NO: 317; or
(ii) the amino acid sequence of SEQ ID NO: 317, wherein the tyrosine at position 16 of SEQ ID NO: 317 is substituted with an amino acid selected from the group consisting of: F, N, E, P, G, K, A, D, H, I, L, M, Q, R, S, T, V, and W; preferably P;
(iii) an amino acid sequence of SEQ ID NO: 285; or
(iv) the amino acid sequence of SEQ ID NO: 285, wherein the tyrosine at position 16 of SEQ ID NO: 285 is substituted with an amino acid selected from the group consisting of: F, N, E, P, G, K, A, D, H, I, L, M, Q, R, S, T, V, and W; preferably P; or
(v) an amino acid sequence of SEQ ID NO: 325; or
(vi) the amino acid sequence of SEQ ID NO: 335.

In some embodiments of the present application, the protein complex comprises or consists of two protein complexes as described in [0302]; and/or the complex comprising or consisting of two protein complexes as described in [0302] is a homodimer.

In some embodiments of the present application, the ligand moiety comprises IL-12, and the fusion protein or protein complex comprises any combinations of heavy and light chains selected from the group consisting of (i) to (viii):
(i) a light chain comprising the amino acid sequence that is at least 85%, 90%, 91%, 92%, 93%, 94%, 95%, 96%, 97%, 98%, 99% or 100% identical to SEQ ID NO: 190;
(ii) a light chain comprising the amino acid sequence that is at least 85%, 90%, 91%, 92%, 93%, 94%, 95%, 96%, 97%, 98%, 99% or 100% identical to SEQ ID NO: 191;
(iii) a light chain comprising the amino acid sequence that is at least 85%, 90%, 91%, 92%, 93%, 94%, 95%, 96%, 97%, 98%, 99% or 100% identical to SEQ ID NO: 342;
(iv) a light chain comprising the amino acid sequence that is at least 85%, 90%, 91%, 92%, 93%, 94%, 95%, 96%, 97%, 98%, 99% or 100% identical to SEQ ID NO: 343;
(v) a light chain comprising the amino acid sequence that is at least 85%, 90%, 91%, 92%, 93%, 94%, 95%, 96%, 97%, 98%, 99% or 100% identical to SEQ ID NO: 344;
(vi) a heavy chain comprising the amino acid sequence that is at least 85%, 90%, 91%, 92%, 93%, 94%, 95%, 96%, 97%, 98%, 99% or 100% identical to SEQ ID NO: 189;
(vii) a heavy chain comprising the amino acid sequence that is at least 85%, 90%, 91%, 92%, 93%, 94%, 95%, 96%, 97%, 98%, 99% or 100% identical to SEQ ID NO: 340; and
(viii) a heavy chain comprising the amino acid sequence that is at least 85%, 90%, 91%, 92%, 93%, 94%, 95%, 96%, 97%, 98%, 99% or 100% identical to SEQ ID NO: 341.

In some embodiments of the present application, the ligand-binding domain or the anti-IL-12 binding molecule or molecule complex or the anti-IL-12 antibody comprises any combinations of heavy chain variable domain (VH) and light chain variable domain (VL) selected from the group consisting of (a) to (e), and (i) to (viii):
(a) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349;
(b) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350;
(c) a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355;
(d) a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 354, and (iii) LCDR3 comprising at least 85%, 90%, 95%, 96%, 97%, 98%, or 99% sequence identity to the amino acid sequence of SEQ ID NO: 355;
(e) a light chain variable domain (VL) comprising (i) LCDR1 comprising at least 85%, 90%, 95%, 96%, 97%, 98%, or 99% sequence identity to the amino acid sequence of SEQ ID NO: 352, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355;
(i) a light chain variable domain (VL) comprising an amino acid sequence with at least 85%, 90%, 95%, 96%, 97%, 98%, 99%, or 100% sequence identity to the amino acid sequence of SEQ ID NO: 190;
(ii) a light chain variable domain (VL) comprising an amino acid sequence with at least 85%, 90%, 95%, 96%, 97%, 98%, 99%, or 100% sequence identity to the amino acid sequence of SEQ ID NO: 191;
(iii) a light chain variable domain (VL) comprising an amino acid sequence with at least 85%, 90%, 95%, 96%, 97%, 98%, 99%, or 100% sequence identity to the amino acid sequence of SEQ ID NO: 342;
(iv) a light chain variable domain (VL) comprising an amino acid sequence with at least 85%, 90%, 95%, 96%, 97%, 98%, 99%, or 100% sequence identity to the amino acid sequence of SEQ ID NO: 343;
(v) a light chain variable domain (VL) comprising an amino acid sequence with at least 85%, 90%, 95%, 96%, 97%, 98%, 99%, or 100% sequence identity to the amino acid sequence of SEQ ID NO: 344;
(vi) a heavy chain variable domain (VH) comprising an amino acid sequence with at least 85%, 90%, 95%, 96%, 97%, 98%, 99%, or 100% sequence identity to the amino acid sequence of SEQ ID NO: 189;
(vii) a heavy chain variable domain (VH) comprising an amino acid sequence with at least 85%, 90%, 95%, 96%, 97%, 98%, 99%, or 100% sequence identity to the amino acid sequence of SEQ ID NO: 340; and
(viii) a heavy chain variable domain (VH) comprising an amino acid sequence with at least 85%, 90%, 95%, 96%, 97%, 98%, 99%, or 100% sequence identity to the amino acid sequence of SEQ ID NO: 341.

In some embodiments of the present application, the fusion protein or protein complex or the anti-IL-12 binding molecule or molecule complex or the anti-IL-12 antibody comprises any combinations of heavy chain variable domain (VH) and light chain variable domain (VL) selected from the group consisting of (a) to (x), and (i) to (xii):
(a) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355;
(b) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 354, and (iii) LCDR3 comprising at least 85%, 90%, 95%, 96%, 97%, 98%, or 99% sequence identity to the amino acid sequence of SEQ ID NO: 355;
(c) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 347, and (iii) HCDR3 comprising at least 85%, 90%, 95%, 96%, 97%, 98%, or 99% sequence identity to the amino acid sequence of SEQ ID NO: 349, and a light chain variable domain (VL) comprising (i) LCDR1 comprising at least 85%, 90%, 95%, 96%, 97%, 98%, or 99% sequence identity to the amino acid sequence of SEQ ID NO: 352, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355;
(d) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355;
(e) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 351, (ii) LCDR2 comprising at least 85%, 90%, 95%, 96%, 97%, 98%, or 99% sequence identity to the amino acid sequence of SEQ ID NO: 354, and (iii) LCDR3 comprising at least 85%, 90%, 95%, 96%, 97%, 98%, or 99% sequence identity to the amino acid sequence of SEQ ID NO: 355;
(f) a heavy chain variable domain (VH) comprising (i) HCDR1 comprising at least 85%, 90%, 95%, 96%, 97%, 98%, or 99% sequence identity to the amino acid sequence of SEQ ID NO: 346, (ii) HCDR2 comprising at least 85%, 90%, 95%, 96%, 97%, 98%, or 99% sequence identity to the amino acid sequence of SEQ ID NO: 348, and (iii) HCDR3 comprising at least 85%, 90%, 95%, 96%, 97%, 98%, or 99% sequence identity to the amino acid sequence of SEQ ID NO: 350, and a light chain variable domain (VL) comprising (i) LCDR1 comprising at least 85%, 90%, 95%, 96%, 97%, 98%, or 99% sequence identity to the amino acid sequence of SEQ ID NO: 352, (ii) LCDR2 comprising at least 85%, 90%, 95%, 96%, 97%, 98%, or 99% sequence identity to the amino acid sequence of SEQ ID NO: 353, and (iii) LCDR3 comprising at least 85%, 90%, 95%, 96%, 97%, 98%, or 99% sequence identity to the amino acid sequence of SEQ ID NO: 355;
(g) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 351, (ii) LCDR2 comprising the amino acid sequence of SEQ ID NO: 353, and (iii) LCDR3 comprising the amino acid sequence of SEQ ID NO: 355;
(h) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 351, (ii) LCDR2 comprising the amino acid sequence of SEQ ID NO: 354, and (iii) LCDR3 comprising the amino acid sequence of SEQ ID NO: 355;
(i) a heavy chain variable domain (VH) comprising (i) HCDR1 comprising the amino acid sequence of SEQ ID NO: 346, (ii) HCDR2 comprising the amino acid sequence of SEQ ID NO: 347, and (iii) HCDR3 comprising the amino acid sequence of SEQ ID NO: 349, and a light chain variable domain (VL) comprising (i) LCDR1 comprising the amino acid sequence of SEQ ID NO: 352, (ii) LCDR2 comprising the amino acid sequence of SEQ ID NO: 353, and (iii) LCDR3 comprising the amino acid sequence of SEQ ID NO: 355;
(j) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 351, (ii) LCDR2 comprising the amino acid sequence of SEQ ID NO: 353, and (iii) LCDR3 comprising the amino acid sequence of SEQ ID NO: 355;
(k) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 351, (ii) LCDR2 comprising the amino acid sequence of SEQ ID NO: 354, and (iii) LCDR3 comprising the amino acid sequence of SEQ ID NO: 355;
(l) a heavy chain variable domain (VH) comprising (i) HCDR1 comprising the amino acid sequence of SEQ ID NO: 346, (ii) HCDR2 comprising the amino acid sequence of SEQ ID NO: 348, and (iii) HCDR3 comprising the amino acid sequence of SEQ ID NO: 350, and a light chain variable domain (VL) comprising (i) LCDR1 comprising the amino acid sequence of SEQ ID NO: 352, (ii) LCDR2 comprising the amino acid sequence of SEQ ID NO: 353, and (iii) LCDR3 comprising the amino acid sequence of SEQ ID NO: 355;
(m) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 190;
(n) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 342;
(o) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 340, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 343;
(p) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 340, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 344;
(q) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 341, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 344;
(r) a heavy chain variable domain (VH) comprising HCDR1, HCDR2, and HCDR3 that is at least 85%, 90%, 95%, 96%, 97%, 98%, or 99% sequence identical to the amino acids of HCDR1, HCDR2, and HCDR3 comprised in SEQ ID NO: 189, and a light chain variable domain (VL) comprising LCDR1, LCDR2, and LCDR3 that is at least 85%, 90%, 95%, 96%, 97%, 98%, or 99% sequence identical to the amino acids of LCDR1, LCDR2, and LCDR3 comprised in SEQ ID NO: 191;
(s) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 190;
(t) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 342;
(u) a heavy chain variable domain (VH) comprising HCDR1, HCDR2, and HCDR3 that is identical to the amino acids of HCDR1, HCDR2, and HCDR3 comprised in SEQ ID NO: 340, and a light chain variable domain (VL) comprising LCDR1, LCDR2, and LCDR3 that is identical to the amino acids of LCDR1, LCDR2, and LCDR3 comprised in SEQ ID NO: 343;
(v) a heavy chain variable domain (VH) comprising HCDR1, HCDR2, and HCDR3 that is identical to the amino acids of HCDR1, HCDR2, and HCDR3 comprised in SEQ ID NO: 340, and a light chain variable domain (VL) comprising LCDR1, LCDR2, and LCDR3 that is identical to the amino acids of LCDR1, LCDR2, and LCDR3 comprised in SEQ ID NO: 344;
(w) a heavy chain variable domain (VH) comprising HCDR1, HCDR2, and HCDR3 that is identical to the amino acids of HCDR1, HCDR2, and HCDR3 comprised in SEQ ID NO: 341, and a light chain variable domain (VL) comprising LCDR1, LCDR2, and LCDR3 that is identical to the amino acids of LCDR1, LCDR2, and LCDR3 comprised in SEQ ID NO: 344;
(x) a heavy chain variable domain (VH) comprising HCDR1, HCDR2, and HCDR3 that is identical to the amino acids of HCDR1, HCDR2, and HCDR3 comprised in SEQ ID NO: 189, and a light chain variable domain (VL) comprising LCDR1, LCDR2, and LCDR3 that is identical to the amino acids of LCDR1, LCDR2, and LCDR3 comprised in SEQ ID NO: 191;
(i) a heavy chain variable domain (VH) comprising an amino acid sequence with at least 85%, 90%, 95%, 96%, 97%, 98%, or 99% sequence identity to the amino acid sequence of SEQ ID NO:189, and a light chain variable domain (VL) comprising an amino acid sequence with at least 85%, 90%, 95%, 96%, 97%, 98%, or 99% sequence identity to the amino acid sequence of SEQ ID NO:190;
(ii) a heavy chain variable domain (VH) comprising an amino acid sequence with at least 85%, 90%, 95%, 96%, 97%, 98%, or 99% sequence identity to the amino acid sequence of SEQ ID NO: 189, and a light chain variable domain (VL) comprising an amino acid sequence with at least 85%, 90%, 95%, 96%, 97%, 98%, or 99% sequence identity to the amino acid sequence of SEQ ID NO: 342;
(iii) a heavy chain variable domain (VH) comprising an amino acid sequence with at least 85%, 90%, 95%, 96%, 97%, 98%, or 99% sequence identity to the amino acid sequence of SEQ ID NO: 340, and a light chain variable domain (VL) comprising an amino acid sequence with at least 85%, 90%, 95%, 96%, 97%, 98%, or 99% sequence identity to the amino acid sequence of SEQ ID NO: 343;
(iv) a heavy chain variable domain (VH) comprising an amino acid sequence with at least 85%, 90%, 95%, 96%, 97%, 98%, or 99% sequence identity to the amino acid sequence of SEQ ID NO: 340, and a light chain variable domain (VL) comprising an amino acid sequence with at least 85%, 90%, 95%, 96%, 97%, 98%, or 99% sequence identity to the amino acid sequence of SEQ ID NO: 344;
(v) a heavy chain variable domain (VH) comprising an amino acid sequence with at least 85%, 90%, 95%, 96%, 97%, 98%, or 99% sequence identity to the amino acid sequence of SEQ ID NO: 341, and a light chain variable domain (VL) comprising an amino acid sequence with at least 85%, 90%, 95%, 96%, 97%, 98%, or 99% sequence identity to the amino acid sequence of SEQ ID NO:344;
(vi) a heavy chain variable domain (VH) comprising an amino acid sequence with at least 85%, 90%, 95%, 96%, 97%, 98%, or 99% sequence identity to the amino acid sequence of SEQ ID NO: 189, and a light chain variable domain (VL) comprising an amino acid sequence with at least 85%, 90%, 95%, 96%, 97%, 98%, or 99% sequence identity to the amino acid sequence of SEQ ID NO: 191;
(vii) a heavy chain variable domain (VH) comprising the amino acid sequence of SEQ ID NO: 189, and a light chain variable domain (VL) comprising the amino acid sequence of SEQ ID NO: 190;
(viii) a heavy chain variable domain (VH) comprising the amino acid sequence of SEQ ID NO: 189, and a light chain variable domain (VL) comprising the amino acid sequence of SEQ ID NO: 342;
(ix) a heavy chain variable domain (VH) comprising the amino acid sequence of SEQ ID NO: 340, and a light chain variable domain (VL) comprising the amino acid sequence of SEQ ID NO: 343;
(x) a heavy chain variable domain (VH) comprising the amino acid sequence of SEQ ID NO: 340, and a light chain variable domain (VL) comprising the amino acid sequence of SEQ ID NO: 344;
(xi) a heavy chain variable domain (VH) comprising the amino acid sequence of SEQ ID NO: 341, and a light chain variable domain (VL) comprising the amino acid sequence of SEQ ID NO: 344; and
(xii) a heavy chain variable domain (VH) comprising the amino acid sequence of SEQ ID NO: 189, and a light chain variable domain (VL) comprising the amino acid sequence of SEQ ID NO: 191.

In some embodiments of the present application, wherein the protein complex comprises or consists of two subunits associated with each other, each subunit comprises a first polypeptide and a second polypeptide, wherein the second polypeptide is selected from the group of: a polypeptide comprising the amino acid sequence of SEQ ID NO: 121, SEQ ID NO: 122; SEQ ID NO: 201; SEQ ID NO: 202; and SEQ ID NO: 209; and wherein the first polypeptide is selected from the group of: a polypeptide comprising the amino acid sequence of SEQ ID NO: 204, SEQ ID NO: 205; SEQ ID NO:207; SEQ ID NO: 208; SEQ ID NO: 210; SEQ ID NO: 244; SEQ ID NO: 245; SEQ ID NO: 249; and SEQ ID NO: 264.

In some embodiments of the present application, the two subunits are bound to each other via one or more disulfide bonds.

In some embodiments of the present application, wherein the protein complex comprises or consists of two subunits associated with each other, each subunit comprises a first polypeptide and a second polypeptide, wherein
(a) the first polypeptide consists of the amino acid sequence of SEQ ID NO: 204, and the second polypeptide consists of the amino acid sequence of SEQ ID NO: 121; or
(b) the first polypeptide consists of the amino acid sequence of SEQ ID NO: 204, and the second polypeptide consists of the amino acid sequence of SEQ ID NO: 201; or
(c) the first polypeptide consists of the amino acid sequence of SEQ ID NO: 205, and the second polypeptide consists of the amino acid sequence of SEQ ID NO: 202; or
(d) the first polypeptide consists of the amino acid sequence of SEQ ID NO: 264, and the second polypeptide consists of the amino acid sequence of SEQ ID NO: 121; or
(e) the first polypeptide consists of the amino acid sequence of SEQ ID NO: 264, and the second polypeptide consists of the amino acid sequence of SEQ ID NO: 122; or
(f) the first polypeptide consists of the amino acid sequence of SEQ ID NO: 207, and the second polypeptide consists of the amino acid sequence of SEQ ID NO: 121; or
(g) the first polypeptide consists of the amino acid sequence of SEQ ID NO: 207, and the second polypeptide consists of the amino acid sequence of SEQ ID NO: 201; or
(h) the first polypeptide consists of the amino acid sequence of SEQ ID NO: 208, and the second polypeptide consists of the amino acid sequence of SEQ ID NO: 202; or
(i) the first polypeptide consists of the amino acid sequence of SEQ ID NO: 208, and the second polypeptide consists of the amino acid sequence of SEQ ID NO: 209; or
(j) the first polypeptide consists of the amino acid sequence of SEQ ID NO: 210, and the second polypeptide consists of the amino acid sequence of SEQ ID NO: 209; or
(k) the first polypeptide consists of the amino acid sequence of SEQ ID NO: 244, and the second polypeptide consists of the amino acid sequence of SEQ ID NO: 121; or
(l) the first polypeptide consists of the amino acid sequence of SEQ ID NO: 245, and the second polypeptide consists of the amino acid sequence of SEQ ID NO: 121; or
(m) the first polypeptide consists of the amino acid sequence of SEQ ID NO: 249, and the second polypeptide consists of the amino acid sequence of SEQ ID NO: 121; or
(n) the first polypeptide consists of the amino acid sequence of SEQ ID NO: 249, and the second polypeptide consists of the amino acid sequence of SEQ ID NO: 122.

In some embodiments of the present application, wherein the protein complex comprises or consists of four polypeptides:
(a) the first and second polypeptide each comprise the amino acid sequence of SEQ ID NO: 204, and the third and fourth polypeptide each comprise the amino acid sequence of SEQ ID NO: 121; or
(b) the first and second polypeptide each comprise the amino acid sequence of SEQ ID NO: 204, and the third and fourth polypeptide each comprise the amino acid sequence of SEQ ID NO: 201; or
(c) the first and second polypeptide each comprise the amino acid sequence of SEQ ID NO: 205, and the third and fourth polypeptide each comprise the amino acid sequence of SEQ ID NO: 202; or
(d) the first and second polypeptide each comprise the amino acid sequence of SEQ ID NO: 264, and the third and fourth polypeptide each comprise the amino acid sequence of SEQ ID NO: 121; or
(e) the first and second polypeptide each comprise the amino acid sequence of SEQ ID NO: 264, and the third and fourth polypeptide each comprise the amino acid sequence of SEQ ID NO: 122; or
(f) the first and second polypeptide each comprise the amino acid sequence of SEQ ID NO: 207, and the third and fourth polypeptide each comprise the amino acid sequence of SEQ ID NO: 121; or
(g) the first and second polypeptide each comprise the amino acid sequence of SEQ ID NO: 207, and the third and fourth polypeptide each comprise the amino acid sequence of SEQ ID NO: 201; or
(h) the first and second polypeptide each comprise the amino acid sequence of SEQ ID NO: 208, and the third and fourth polypeptide each comprise the amino acid sequence of SEQ ID NO: 202; or
(i) the first and second polypeptide each comprise the amino acid sequence of SEQ ID NO: 208, and the third and fourth polypeptide each comprise the amino acid sequence of SEQ ID NO: 209; or
(j) the first and second polypeptide each comprise the amino acid sequence of SEQ ID NO: 210, and the third and fourth polypeptide each comprise the amino acid sequence of SEQ ID NO: 209; or
(k) the first and second polypeptide each comprise the amino acid sequence of SEQ ID NO: 244, and the third and fourth polypeptide each comprise the amino acid sequence of SEQ ID NO: 121; or
(l) the first and second polypeptide each comprise the amino acid sequence of SEQ ID NO: 245, and the third and fourth polypeptide each comprise the amino acid sequence of SEQ ID NO: 121; or
(m) the first and second polypeptide each comprise the amino acid sequence of SEQ ID NO: 249, and the third and fourth polypeptide each comprise the amino acid sequence of SEQ ID NO: 121; or
(n) the first and second polypeptide each comprise the amino acid sequence of SEQ ID NO: 249, and the third and fourth polypeptide each comprise the amino acid sequence of SEQ ID NO: 122.

In some embodiments of the present application, wherein the protein complex consists of four polypeptides:
(a) the first and second polypeptide each consist of the amino acid sequence of SEQ ID NO: 204, and the third and fourth polypeptide each consist of the amino acid sequence of SEQ ID NO: 121; or
(b) the first and second polypeptide each consist of the amino acid sequence of SEQ ID NO: 204, and the third and fourth polypeptide each consist of the amino acid sequence of SEQ ID NO: 201; or
(c) the first and second polypeptide each consist of the amino acid sequence of SEQ ID NO: 205, and the third and fourth polypeptide each consist of the amino acid sequence of SEQ ID NO: 202; or
(d) the first and second polypeptide each consist of the amino acid sequence of SEQ ID NO: 264, and the third and fourth polypeptide each consist of the amino acid sequence of SEQ ID NO: 121; or
(e) the first and second polypeptide each consist of the amino acid sequence of SEQ ID NO: 264, and the third and fourth polypeptide each consist of the amino acid sequence of SEQ ID NO: 122; or
(f) the first and second polypeptide each consist of the amino acid sequence of SEQ ID NO: 207, and the third and fourth polypeptide each consist of the amino acid sequence of SEQ ID NO: 121; or
(g) the first and second polypeptide each consist of the amino acid sequence of SEQ ID NO: 207, and the third and fourth polypeptide each consist of the amino acid sequence of SEQ ID NO: 201; or
(h) the first and second polypeptide each consist of the amino acid sequence of SEQ ID NO: 208, and the third and fourth polypeptide each consist of the amino acid sequence of SEQ ID NO: 202; or
(i) the first and second polypeptide each consist of the amino acid sequence of SEQ ID NO: 208, and the third and fourth polypeptide each consist of the amino acid sequence of SEQ ID NO: 209; or
(j) the first and second polypeptide each consist of the amino acid sequence of SEQ ID NO: 210, and the third and fourth polypeptide each consist of the amino acid sequence of SEQ ID NO: 209; or
(k) the first and second polypeptides, each consists of the amino acid sequence of SEQ ID NO: 244, and the third and fourth polypeptides, each consists of the amino acid sequence of SEQ ID NO: 121; or
(l) the first and second polypeptide each consist of the amino acid sequence of SEQ ID NO: 245, and the third and fourth polypeptide each consist of the amino acid sequence of SEQ ID NO: 121; or
(m) the first and second polypeptide each consist of the amino acid sequence of SEQ ID NO: 249, and the third and fourth polypeptide each consist of the amino acid sequence of SEQ ID NO: 121; or
(n) the first and second polypeptide each consist of the amino acid sequence of SEQ ID NO: 249, and the third and fourth polypeptide each consist of the amino acid sequence of SEQ ID NO: 122.

In some embodiments of the present application, the protein complex is obtainable by a) culturing a mammalian host cell expressing the polypeptides, and b) retrieving the protein complex from the supernatant, preferably wherein the mammalian host cell is a Chinese Hamster Ovary (CHO) cell; and/or the protein complex is glycosylated and/or is produced recombinantly by expression in a mammalian host cell, preferably wherein the mammalian host cell is a Chinese Hamster Ovary (CHO) cell.

In some embodiments of the present application, wherein the protein complex comprises or consists of four polypeptides, the first and second polypeptides are each a heavy chain, and the third and fourth polypeptides are each a light chain, the heavy and light chains are bound to each other via one or more disulfide bonds; and/or the first and second polypeptides are each a heavy chain, and the third and fourth polypeptides are each a light chain, and the heavy chain comprises a heavy chain variable domain (VH), and the light chain comprises a light chain variable domain (VL), wherein the VH is associated with the VL; and/or the first and second polypeptides are each a heavy chain, and the third and fourth polypeptides are each a light chain, wherein the heavy chain comprises a CH1 domain, and wherein the light chain comprises a CL domain, and wherein the CH1 domain is associated with the CL domain.

In another aspect, a heavy chain variable domain (VH) sequence as defined herein can contain substitutions (e.g., conservative substitutions), insertions, or deletions relative to the reference sequence, but the ligand binding domain of the fusion protein comprising that sequence retains the ability to bind to IL-12. In certain embodiments, a total of 1 to 10 amino acids have been substituted, inserted and/or deleted in a heavy chain variable domain (VH) sequence as defined herein. In certain embodiments, substitutions, insertions, or deletions occur in regions outside the HVRs (i.e., in the FRs). Optionally, the fusion protein or protein complex comprising the ligand-binding domain that binds IL-12 comprises a heavy chain variable domain (VH) sequence as defined herein, including post-translational modifications of that sequence. Post-translational modifications include but are not limited to a modification of glutamine or glutamate in N-terminal of heavy chain or light chain to pyroglutamic acid by pyroglutamylation.

In another aspect, a fusion protein or protein complex comprising a ligand-binding domain that binds IL-12 is provided, the light chain variable domain (VL) as defined herein can contain substitutions (e.g., conservative substitutions), insertions, or deletions relative to the reference sequence, but the ligand-binding domain of the fusion protein or the protein complex comprising that sequence retains the ability to bind to IL-12. In certain embodiments, a total of 1 to 10 amino acids have been substituted, inserted and/or deleted in the light chain variable domain (VL) as defined herein. In certain embodiments, the substitutions, insertions, or deletions occur in regions outside the HVRs (i.e., in the FRs). Optionally, the fusion protein or the protein complex comprising the ligand-binding domain that binds IL-12 comprises the light chain variable domain (VL) as defined herein, including post-translational modifications of that sequence. Post-translational modifications include but are not limited to a modification of glutamine or glutamate in N-terminal of heavy chain or light chain to pyroglutamic acid by pyroglutamylation.

In another aspect, a fusion protein or protein complex comprising ligand-binding domain that binds IL-12 is provided, wherein the ligand-binding domain that binds IL-12 comprises a VH as in any of the embodiments provided above, and a VL as in any of the embodiments provided above. In one embodiment, the fusion protein or protein complex comprising ligand-binding domain that binds IL-12 comprises the VH and VL as defined above, including post-translational modifications of those sequences. In a preferred embodiment, the fusion protein or protein complex comprising ligand-binding domain that binds IL-12 comprises the VH and VL sequences as defined above, including post-translational modifications of those sequences. Post-translational modifications include but are not limited to a modification of glutamine or glutamate in N-terminal of heavy chain or light chain to pyroglutamic acid by pyroglutamylation.

In a further aspect, the present invention includes an antibody that binds to human IL-12 and comprises: (a) a HCDR1 of SEQ ID NO: 346; (b) a HCDR2 of SEQ ID NO: 347 or 348; (c) a HCDR3 of SEQ ID NO: 349 or 350; (d) a LCDR1 of SEQ ID NO: 351 or 352; (e) a LCDR2 of SEQ ID NO: 353 or 354; and (f) a LCDR3 of SEQ ID NO: 355, wherein the HCDR1, HCDR2, HCDR3, LCDR1, LCDR2 and LCDR3 does not comprise SEQ ID NO: 346, 348, 350, 352, 353 and 355, respectively. In some embodiments, the HCDR1, HCDR2, HCDR3, LCDR1, LCDR2 and LCDR3 comprises SEQ ID NO: 346, 347, 349, 351, 353 and 355, respectively. In some embodiments, the HCDR1, HCDR2, HCDR3, LCDR1, LCDR2 and LCDR3 comprises SEQ ID NO: 346, 347, 349, 351, 354 and 355, respectively. In some embodiments, the HCDR1, HCDR2, HCDR3, LCDR1, LCDR2 and LCDR3 comprises SEQ ID NO: 346, 348, 350, 351, 353, and 355, respectively. In some embodiments, the HCDR1, HCDR2, HCDR3, LCDR1, LCDR2 and LCDR3 comprises SEQ ID NO: 346, 348, 350, 351, 354, and 355, respectively. In some embodiments, the HCDR1, HCDR2, HCDR3, LCDR1, LCDR2 and LCDR3 comprises SEQ ID NO: 346, 347, 349, 352, 353, and 355, respectively. In some embodiments, the antibody comprises a VH and VL, wherein the VH comprises SEQ ID NO: 189, 340 or 341 and the VL comprises SEQ ID NO: 190, 191, 342, 343 or 344, wherein the VH and VL does not comprise SEQ ID NO: 189 and 190, respectively. In some embodiments, the VH and VL comprises SEQ ID NO: 189 and 342, respectively. In some embodiments, the VH and VL comprises SEQ ID NO: 340 and 343, respectively. In some embodiments, the VH and VL comprises SEQ ID NO: 340 and 344, respectively. In some embodiments, the VH and VL comprises SEQ ID NO: 341 and 344, respectively. In some embodiments, the VH and VL comprises SEQ ID NO: 189 and 191, respectively. In some embodiments, the antibody comprises a heavy chain (HC) and a light chain (LC), wherein the HC and the LC comprises SEQ ID NO: 198 and 121, respectively, or SEQ ID NO: 198 and 201, respectively, or SEQ ID NO: 203 and 202, respectively. In some embodiments, the HC and LC comprises SEQ ID NO: 198 and 121, respectively. In some embodiments, the HC and LC comprises SEQ ID NO: 198 and 201, respectively. In some embodiments, the HC and LC comprises SEQ ID NO: 203 and 202, respectively. In some embodiments, the antibody is an IgG antibody, IgG1 antibody, scFv, single-chain antibody, Fv, scFv2, Fab or F(ab′)2. In a further aspect, the present invention includes a fusion protein or protein complex comprising the antibody and a heterologous polypeptide. In some embodiments, the heterologous polypeptide is IL-12. Polypeptide, e.g., a single chain IL-12. In a further aspect, the present invention includes a polynucleotide encoding the antibody or a plurality of polynucleotides encoding the fusion protein or protein complex. In a further aspect, the present invention includes a method of treating an IL-12 mediated disease or disorder in an individual in need thereof, comprising administering to said individual a therapeutically effective amount of the antibody.

Additional fusion polypeptides or protein complexes, including IL-12 fusion polypeptides or protein complexes are disclosed in International Publication No. WO2023/002952, which is incorporated herein by reference in its entirety for all purposes.

The present disclosure includes a method of producing the protein complex, polypeptide, fusion protein, binding molecule complex, or binding molecule described herein, comprising the steps of:
(a) culturing a host cell comprising a polynucleotide or a plurality of polynucleotides encoding the protein complex, polypeptide, fusion protein, binding molecule complex, or binding molecule described herein, and
(b) recovering the protein complex, polypeptide, fusion protein, binding molecule complex, or binding molecule described herein.

The present disclosure includes a protein complex, polypeptide, fusion protein, binding molecule complex, or binding molecule produced by the aforementioned method.

It should be understood by those skilled in the art that arbitrary combinations of one or more embodiments described in the present specification are also included in the present invention unless there is technical contradiction on the basis of the technical common sense of those skilled in the art. Also, the present invention excluding arbitrary combinations of one or more embodiments described in the present specification is intended in the present specification and should be interpreted as the described invention, unless there is technical contradiction on the basis of the technical common sense of those skilled in the art.

Hereinafter, examples of the method and the composition of the present invention will be described. It shall be understood that various other embodiments can be carried out in light of the general description mentioned above.

### III. EXAMPLES

The following are examples of methods and compositions of the invention. It is understood that various other embodiments may be practiced, given the general description provided above.

### Example 1: IL-12 Fusion Protein (Also Referenced Herein as a Protein Complex) has Long Systemic Half-Life Before Protease Cleavage and Short Systemic Half-Life after Protease Cleavage

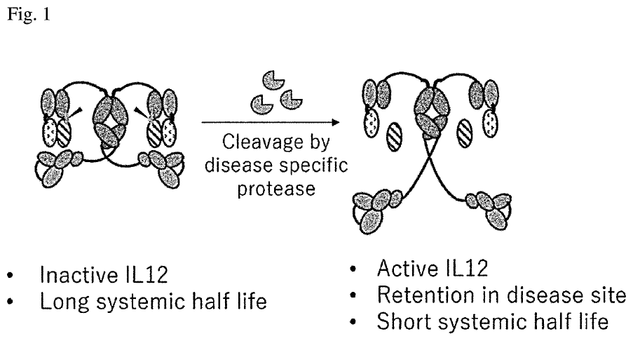

In order to deliver cytokines, such as Interleukin-12 (IL-12), at high dose with low or negligible systemic toxicity, the present inventors have developed an IL-12 fusion protein having protease cleavable linkers. The IL-12 fusion protein remains in an inactivated state until otherwise exposed to an environment with high concentrations of proteases which activates the protein by cleaving the protease cleavage site within the protein. Once cleaved, IL-12 no longer binds to the protein, restoring its physiological activity to bind to its receptor and becomes capable of exerting its biological activity to activate IL-12 receptor signaling (FIG. 1). The presently described IL-12 fusion protein allows for target site specific activation that harness the naturally high concentrations of proteases in certain environments, e.g. tumor or inflammatory tissues. The presently described IL-12 fusion protein in the non-cleaved state (inactive) has a long systemic half-life compared to recombinant IL-12 alone. It has been reported that IL-12 is known to have a very short half-life of approximately 5-10 hours (AACR Journals. 1999 January; 5(1): 9-16).

### 1-1 Preparation of IL-12 Fusion Protein (Also Referenced Herein as a Protein Complex)—Monovalent Release Type

Several IL-12 fusion proteins were constructed by fusing IL-12 molecules, comprising the p40 (SEQ ID NO: 44) and p35 (SEQ ID NO: 45) subunits, with polypeptides that bind IL-12 via protease cleavable linkers, or cleavable peptide linkers comprising a protease cleavage site. IL-12 binding polypeptides employed incorporates Ab1 (WO2010017598), Ab2, (WO2002012500) and Ab3 (WO2000056772). Further, unless otherwise noted, modifications were performed in the Fc region of said protein that abolishes Fc gamma R binding, comprising amino acid mutations L235R/G236R according to the EU numbering.

Three IL-12 fusion proteins, each comprising a monovalent heterodimer of a polypeptide comprising an IL-12 binding domain (anti-IL-12) and a polypeptide comprising a KLH binding domain (anti-KLH), as follows:
a) Monovalent IL-12 release FP1 is a hetero-dimer of a pair of light chain 1 (SEQ ID NO: 49)/heavy chain 1(SEQ ID NO: 54) and light chain 2 (SEQ ID NO: 55)/heavy chain 2 (SEQ ID NO: 56). In heavy chain 1 (SEQ ID NO: 54), cleavable linker (SEQ ID NO: 46) was introduced into elbow hinge region between FP1 VH (SEQ ID NO: 51) and CH1 domains and cleavable linker (SEQ ID: 52) was introduced between Fc and single chain IL-12 (SEQ ID: 67).
b) Monovalent IL12 release FP2 is a hetero dimer of a pair of light chain 1 (SEQ ID NO: 60)/heavy chain 1 (SEQ ID NO: 104) and light chain 2 (SEQ ID NO: 55)/heavy chain 2 (SEQ ID NO: 56). In heavy chain 1 (SEQ ID NO: 104), cleavable linker (SEQ ID NO: 46) was introduced into elbow hinge region between FP2 VH (SEQ ID NO: 62) and CH1 domains and cleavable linker (SEQ ID: 52) was introduced between Fc and single chain IL-12 (SEQ ID: 67).
c) Monovalent IL12 release FP3 is a hetero dimer of a pair of light chain 1 (SEQ ID NO: 63)/heavy chain 1 (SEQ ID NO: 105) and light chain 2 (SEQ ID NO: 55)/heavy chain 2 (SEQ ID NO: 56). In heavy chain 1 (SEQ ID NO: 105), cleavable linker (SEQ ID NO: 46) was introduced into elbow hinge region between FP3 VH (SEQ ID NO: 65) and CH1 domains and cleavable linker (SEQ ID: 156) was introduced between Fc and single chain IL-12 (SEQ ID: 67).

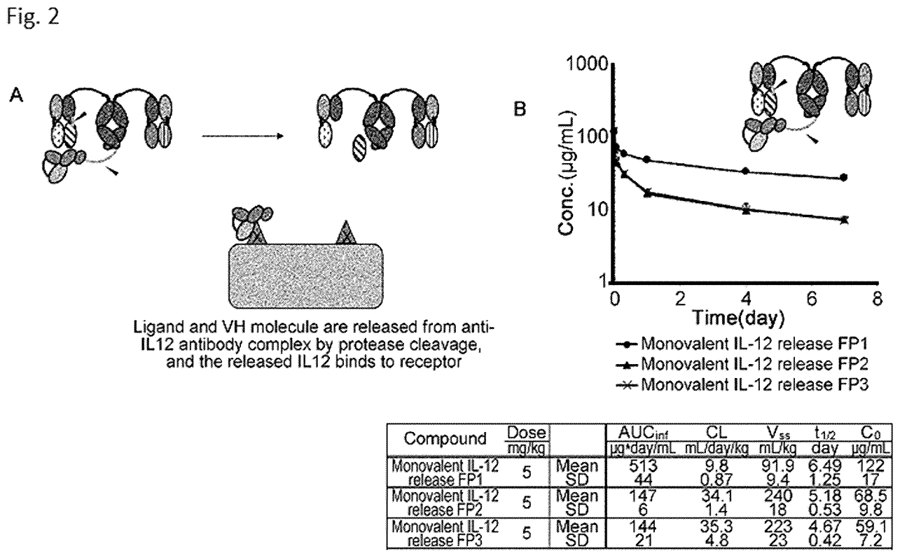

For each of the above, in order to promote hetero-dimerization and precise association of heavy and light chains, knobs-into-holes mutations (Nat. Biotechnol, 1998, 16, 677-681) were introduced in heavy chain CH3 domains and CrossMab technology (PNAS, 2011, 108, 11187-11192) was employed in heavy chain 2 and light chain 2. Heavy chain 1 and heavy chain 2 contain knob mutations (Y349C/T366W) and hole mutations (E356C/T366S/L368A/Y407V), respectively. Light chain 2 was composed of VH domain of anti-KLH with human kappa constant region. Heavy chain 2 was composed of VL domain of anti-KLH and modified IgG1 Fc region. Once the cleavable linker is digested by protease, active IL-12 freely dissociates from the fusion polypeptide and binds to its receptor (FIG. 2A).

Expression vectors of each chain were prepared by a method known to those skilled in the art and expressed using Expi293 (Life Technologies Corp.) by combining each chain as shown in Table 2. Purification of the fusion protein was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 2
Monovalent IL-12 fusion proteins (also referenced herein as protein
complexes) of release format and sequence IDs of each chain

| IL-12 fusion | Light | Heavy | Light | Heavy |
|---|---|---|---|---|
| proteins | chain 1 | chain 1 | chain 2 | chain 2 |
| Monovalent | SEQ ID NO: | SEQ ID NO: | SEQ ID NO: | SEQ ID NO: |
| IL-12 release | 49 | 54 | 55 | 56 |
| FP1 | | | | |
| Monovalent | SEQ ID NO: | SEQ ID NO: | SEQ ID NO: | SEQ ID NO: |
| IL-12 release | 60 | 104 | 55 | 56 |
| FP2 | | | | |
| Monovalent | SEQ ID NO: | SEQ ID NO: | SEQ ID NO: | SEQ ID NO: |
| IL-12 release | 63 | 105 | 55 | 56 |
| FP3 | | | | |

### 1-2 Long Systemic Half-Life of Non-Cleaved IL-12 Fusion Proteins (Also Referenced Herein as Protein Complexes)

In the present invention, the inventors sought to obtain IL-12 fusion proteins that exhibit long systemic half-life in the non-cleaved (inactive) state before protease digestion but exhibit a short systemic half-life in the cleaved (active) state.

5 mg/kg of monovalent IL-12 release FP1 to FP3 as described in Table 2 above were intravenously administered into the tail vein of 6-week-old CB17/Icr-Prkdcscid/CrlCrlj female mice (Charles River Laboratories, Japan) and their plasma was successively collected from the jugular vein at the following timepoints: 5 minutes, 1 hour, 7 hours, 1 day, 4 days, and 7 days after administration. The total concentrations of fusion proteins in mouse plasma were measured by LC/ESI-MS/MS. Calibration standards were prepared by serial dilution in mouse plasma. Calibration standard concentrations were 0.195, 0.391, 0.781, 1.56, 3.13, 6.25, 12.5, 25 and 50 microgram (micro g)/mL. A 3 microliter (micro L) of the calibration standards and plasma samples were mixed with 50 micro L of the magnetic beads coated with an anti-human Fc region antibody (in-house). After washing the beads 3 times with 0.05% Tween-20 containing PBS and 1 time with PBS, the samples were mixed with 25 micro L of mixed reagent (8 mmol/L dithiothreitol, 7.5 mol/L urea and 99 ng/mL lysozyme (chicken egg white) in 50 mmol/L ammonium bicarbonate) and incubated for approximately 45 min at 56 degrees C. Then, 2 micro L of 500 mmol/L iodoacetamide was added and incubated for approximately 30 min at 37 degrees C. in the dark. Next, 160 micro L of 0.621 micro g/mL sequencing grade modified trypsin (Cat. No.: V5117, Promega) in 50 mmol/L ammonium bicarbonate was added and incubated at 37 degrees C. overnight. Finally, 5 micro L of 10% trifluoroacetic acid was added to deactivate any residual trypsin. An aliquot of 80 micro L of digestion samples was subjected to analysis by LC/ESI-MS/MS.

LC/ESI-MS/MS was performed using Xevo TQ-S triple quadrupole instrument (Waters) equipped with Acquity I-class 2D high-performance liquid chromatography systems (Waters). The antibody-derived tryptic peptide, TLTIQVK (SEQ ID NO: 366), was monitored by the selected reaction monitoring (SRM). SRM transition was m/z 401.755>588.372. The calibration curve of the antibody was constructed by the weighted (1/x2) linear regression using the peak area plotted against the concentrations respectively. The concentrations in mouse plasma were calculated from the calibration curve using the analytical software Masslynx Ver. 4.1 (Waters).

Concentration of each antibody was determined by the calibration curve based on the peak area of monitored peptide in calibration samples. Pharmacokinetic parameters were calculated by non-compartmental analysis using Phoenix WinNonlin version 8.0 (Certara USA inc.). The averaged plasma concentrations and parameters of 3 animals are shown in FIG. 2B with their standard deviations.

Monovalent IL-12 release FP1 demonstrated approximately 3-fold lower plasma clearance (9.8 ml/day/kg) than the other 2 fusion proteins, i.e. FP2 (34.1 ml/day/kg) and FP3 (35.3 ml/day/kg) in the same monovalent heterodimer fusion protein format. The above result implies that the fast clearance of IL-12 is blocked in the non-cleaved (inactive) monovalent IL-12 release FP1.

### 1-3 Preparation of Bivalent IL-12 Fusion Protein (Also Referenced Herein as a Protein Complex) of Release and Fusion Formats

Bivalent IL-12 release FP4 is a homodimer of a pair of light chain (SEQ ID NO: 49) and heavy chain (SEQ ID NO: 53). FP1VL-k0 (SEQ ID NO: 49) was employed as light chain without modification. In heavy chain, cleavable linker was introduced into elbow hinge region between FP1 VH (SEQ ID NO: 51) and CH1 domains. GS linker (SEQ ID NO: 106) was inserted in hinge region and single-chain IL-12 (SEQ ID NO: 67) was attached to C-terminal of Fc domain via cleavable linker (SEQ ID NO: 52). Once these cleavable linkers were digested by proteases, active IL-12 molecules are released (FIG. 3A).

Bivalent IL-12 fusion FP5 is a homodimer of a light chain (SEQ ID NO: 49) and heavy chain (SEQ ID NO: 58). FP1VL-k0 (SEQ ID NO: 49) was employed as light chain without modification. In heavy chain, cleavable linker (SEQ ID NO: 46) was introduced into elbow hinge region between FP1VH (SEQ ID NO: 51) and CH1 domains. GS linker (SEQ ID NO: 106) was inserted in hinge region and single-chain IL-12 (SEQ ID NO: 67) was attached to C-terminal of Fc domain via GS linker (SEQ ID NO: 68). Once the cleavable linker was digested by protease, active IL-12 molecules that remain fused to the C-terminal of Fc region dissociate from the ligand-binding domain, i.e. IL-12 binding domain, of the fusion protein and bind to the IL-12 receptors (FIG. 3B).

Expression vectors of each chain were prepared by a method known to those skilled in the art and expressed using Expi293 (Life Technologies Corp.) by combining each chain as shown in Table 3. Purification of fusion proteins was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 3
Bivalent IL-12 fusion proteins (also referenced
herein as protein complexes) of release and fusion
formats and sequence IDs of each chain.

| IL-12 fusion | Light | Heavy | Light | Heavy |
|---|---|---|---|---|
| proteins | chain 1 | chain 1 | chain 2 | chain 2 |
| Bivalent IL-12 | SEQ ID NO: | SEQ ID NO: | — | — |
| release FP4 | 49 | 53 | | |
| Bivalent IL-12 | SEQ ID NO: | SEQ ID NO: | — | — |
| fusion FP5 | 49 | 58 | | |

### 1-4 Evaluation of Activity of Bivalent IL-12 Fusion Proteins (Also Referenced Herein as Protein Complexes) in Release and Fusion Formats

IL-12 was purified by co-expression of vectors expressing p40 (SEQ ID NO: 44) and p35 (SEQ ID NO: 45) with TEV protease recognition site followed by (His)₆ tag fused at the C-terminus. Expression vectors of each chain were prepared by a method known to those skilled in the art and expressed using Expi293 (Life Technologies Corp.) by combining each chain. Purification of proteins was done using affinity purification by Ni Sepharose excel (Cat. No: 17-3712-02, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

To assess IL-12 bioactivity of bivalent IL-12 fusion proteins of the release and fusion formats with or without MT-SP1 protease treatment, IL-12 luciferase assay was conducted. Briefly, 2.5×10⁴ cells/well IL-12 bioassay cell (Promega, Cat #CS2018A02A) which express human IL-12Rb1, IL-12Rb2, and STAT4, were plated in 96-well plate and incubated overnight. As control, IL-12 was utilized. Bivalent IL-12 fusion proteins of both release and fusion formats were added to the culture plate and incubated for 18 hours. For protease-treated samples, IL-12 and bivalent IL-12 fusion proteins of both release and fusion formats were treated with equimolar concentration of MT-SP1 for 4 hours and serial diluents were prepared. Luciferase activity was detected with Bio-Glo luciferase assay system (Promega, G7940) according to manufacturer's instructions. Luminescence was detected using GloMax (registered trademark) Explorer System (Promega #GM3500). Data analysis was done by Microsoft (registered trademark) Excel (registered trademark) 2013 and the analyzed data was plotted using GraphPad Prism 7.

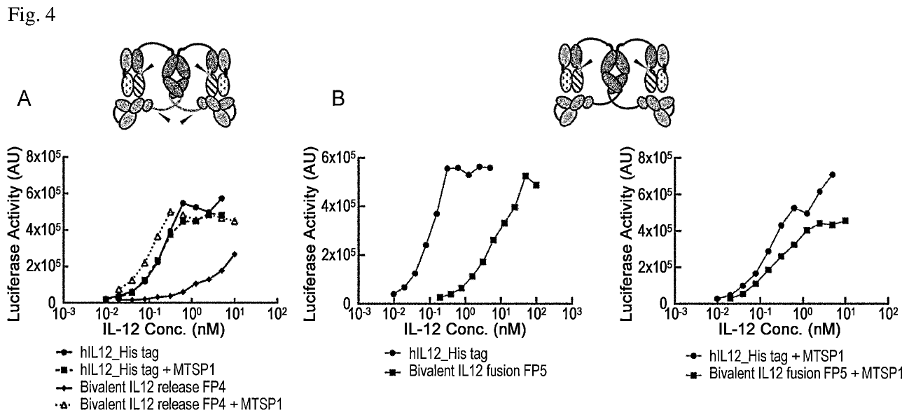

Bivalent IL-12 release FP4, and Bivalent IL-12 fusion FP5 were subjected to the IL-12 luciferase assay. Both variants showed lower IL-12 bioactivity than hIL-12_His tag in the absence of MT-SP1, and the IL-12 bioactivity was recovered to the same level as hIL-12_His tag upon MT-SP1 treatment (FIGS. 4A and 4B).

Based on the above, it has been demonstrated that bivalent IL-12 fusion proteins of both the release and fusion formats lose their ability (i.e., the ability was attenuated) to bind their ligand receptor, i.e. IL-12 receptor, and this binding was inhibited in the non-cleaved (inactive) state. However, once cleaved in the presence of protease (active state), the biological activity of the ligand is restored, i.e. IL-12 was capable of binding to its ligand receptor, i.e. IL-12 receptor.

### Example 2: Fusion Format Accumulates in Higher Concentrations within the Tumor and Exhibits Faster Clearance than Release Format

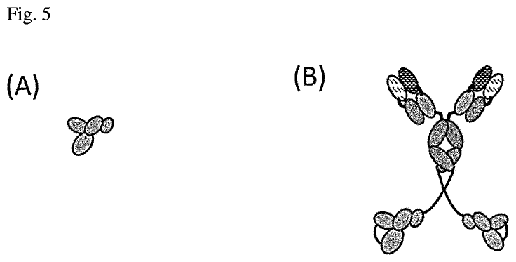

IL-12 fusion proteins (also referenced herein as a protein complex) in the inactivated state binds to IL-12 and inhibits the ability of IL-12 to bind its receptor. When exposed to proteases, for example, tumor specific proteases, the IL-12 fusion protein is activated, the ability of IL-12 to bind its receptor is restored. As described in Example 1, in their separate protease activated forms, the additional cleavage site between Fc region and IL-12 allows for the full release of IL-12 (release format), while without the additional cleavage site, the IL-12 remains fused to the C-terminal of the Fc region (fusion format). Tumor accumulation and clearance studies were performed using recombinant IL-12 and KLH Bivalent IL-12 fusion protein as surrogates of the activated form of the release format and fusion format respectively (FIG. 5).

### 2-1 Preparation of Activated Forms of Release Type and Fusion Type IL-12 Fusion Proteins (Also Referenced Herein as Protein Complexes)

IL-12 protein which is a heterodimer protein comprising p40 subunit (SEQ ID NO: 44) and p35 subunit (SEQ ID NO: 107), each comprising TEV protease recognition site (SEQ ID NO: 108) and FLAG tag (SEQ ID NO: 109) was prepared.

In addition, KLH Bivalent IL-12 fusion protein FP6 is prepared, which comprises a homodimer of a light chain (SEQ ID NO: 91) and heavy chain (SEQ ID NO: 110). SEQ ID NO: 91 was employed as light chain without modifications. In heavy chain (SEQ ID NO: 110), KLH VH (SEQ ID NO: 99) is fused to constant region (SEQ ID NO: 111) followed by single-chain IL-12 (SEQ ID NO: 67) attached to C-terminal of Fc via GS linker (SEQ ID NO: 68). KLH Bivalent IL-12 fusion protein FP7 is prepared, which comprises a homodimer of a light chain (SEQ ID NO: 91) and heavy chain (SEQ ID NO: 112). SEQ ID NO: 91 was employed as light chain without modifications. In heavy chain (SEQ ID NO: 112), KLH VH (SEQ ID NO: 99) is fused to constant region, C6 (SEQ ID NO: 111) followed by single-chain IL-12 (SEQ ID NO: 113) attached to C-terminal of Fc via GS linker (SEQ ID NO: 68).

Expression vectors of each chain were prepared by a method known to those skilled in the art and expressed using Expi293 (Life Technologies Corp.) by combining each chain as shown in Table 4. Purification of IL-12 was done using affinity purification by Anti-FLAG M2 resin followed by size exclusion chromatography using Superdex 200 gel filtration column (Cat. No: 28-9893-35, GE Healthcare). Purification of fusion protein was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 4
Activated bivalent IL-12 fusion proteins (also referenced herein as
protein complexes) of fusion format and sequence IDs of each chain.

| KLH bivalent | | | | |
|---|---|---|---|---|
| IL-12 fusion | Light | Heavy | Light | Heavy |
| proteins | chain 1 | chain 1 | chain 2 | chain 2 |
| KLH Bivalent | SEQ ID NO: | SEQ ID NO: | — | — |
| IL-12 fusion | 91 | 110 | | |
| FP6 | | | | |
| KLH Bivalent | SEQ ID NO: | SEQ ID NO: | | |
| IL-12 fusion | 91 | 112 | | |
| FP7 | | | | |

### 2-2 Evaluation of Tumor Accumulation of Bivalent IL-12 Fusion Proteins (Also Referenced Herein as Protein Complexes) In Vivo Using Tumor Bearing Mice

### 2-2-1 In Vivo Testing Using Human T Cell Injected Tumor Bearing (TB) Mice Model

The LS1034 human colorectal carcinoma cell line was obtained from American Type Culture Collection. Cells were cultured in RPMI-1640 medium (SIGMA) plus 0.45% D-glucose (SIGMA), 10 mM HEPES (SIGMA), 1 mM Sodium Pyruvate (Gibco) with 10% fetal bovine serum (FBS; Nichirei Biosciences). NOD/ShiJic-scidJcl female mice of 6 weeks of age were purchased from CLEA Japan, Inc and were acclimated for 2 weeks before the inoculation. LS1034 cells in log phase growth were harvested and washed with Hank's balanced salt solution (HBSS; SIGMA), resuspended in 50% HBSS and 50% Matrigel (CORNING) at a concentration of 5×10⁷ cells/mL. Mice were subcutaneously inoculated with 1×10⁷ LS1034 cells in 200 micro L of HBSS:Matrigel (1:1). When mean tumor volume reached about 100-300 mm³ (7 days after inoculation), mice were randomized into groups based on tumor volume and body weight. Tumor volume was measured with caliper, and tumor volume was calculated as ½×1×w², l=length, w=width. 1 day after the randomization, mice were inoculated with 3×10⁷ human T cells. After T cell inoculation, 11.3 pmol of IL-12 or 11.3 pmol of KLH-Bivalent IL-12 fusion protein FP6 were intratumorally administered 3 times a week for 2 weeks. Tumor was resected on 12 and 13 days after first treatment.

### 2-2-2 Preparation of Tumor Lysate and Tumor Interstitial Fluid

One-fourth of tumor sections were placed on the tube with mesh and centrifuged at 400×g at 4 degrees C. for 10 minutes. The collected fluid samples were centrifuged again at 10,000×g at 4 degrees C. for 10 minutes, then supernatants were kept as tumor interstitial fluid. Add 9 times the weight of the Lysis buffer (50 mM Tris, 150 mM NaCl, 0.5% Sodium deoxycholate, 2% NP-40 at pH8.0 with protease inhibitor cocktail) into the remaining tumor sections and homogenized by TissueLyser II (Qiagen). The homogenates were centrifuged at 14,000 rpm at 4 degree C. for 10 minutes and the supernatants were kept as 10% tumor lysate.

### 2-2-3 Measurement of IL-12 Concentration in Tumor Lysate and Tumor Interstitial Fluid by Electrochemiluminescence (ECL)

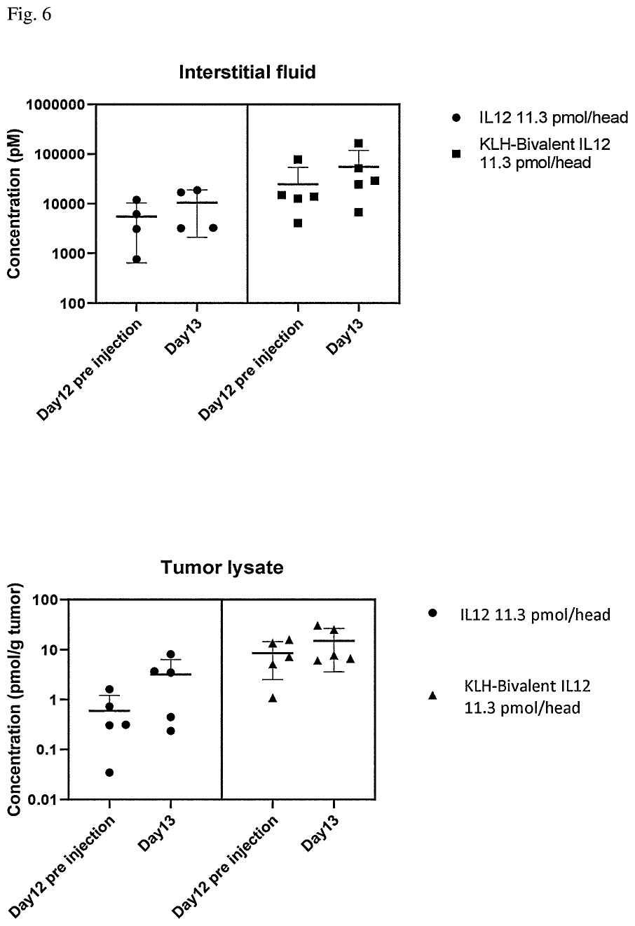

The concentration of IL-12 in tumor lysate and tumor interstitial fluid were measured by electrochemiluminescence (ECL). MSD GOLD 96-well Streptavidin SECTOR Plates (Meso Scale Discovery) were blocked with assay buffer (PBS-T+1% BSA from Roche and Sigma, respectively) for 1 hour at room temperature. Anti-IL-12 immobilized plates were prepared by dispensing biotinylated anti-IL-12 polyclonal antibody (R&D Systems) onto blocked plates and incubating in assay buffer for 1 hour at room temperature. Calibration curve samples of IL-12 and KLH-Bivalent IL-12 fusion protein FP6, tumor lysate samples, and tumor interstitial fluid samples were prepared by serial dilutions, of 100-fold, or more, were prepared. Subsequently, the samples were added onto an anti-IL-12-immobilized plate and allowed to bind for 1 hour at room temperature before washing. Next, SULFO TAG labelled anti-IL-12 antibody (U-CyTech biosciences, SULFO TAG labelled using MSD GOLD SULFO-TAG NHS-Ester) was added and the plate was incubated for 1 hour at room temperature before washing. Read Buffer T (×4) (Meso Scale Discovery) was immediately added to the plate and signal was detected by SECTOR Imager 2400 (Meso Scale Discovery). Recombinant IL-12 or KLH-Bivalent IL-12 fusion protein FP6 concentration was calculated based on the response of the calibration curve using the analytical software SOFTmax PRO (Molecular Devices). The tumor lysate and tumor interstitial fluid concentrations of recombinant IL-12 and KLH-Bivalent IL-12 fusion protein FP6 measured by this method is shown in FIG. 6.

### 2-2-4 Tumor Retention of IL-12 and KLH-Bivalent IL-12 Fusion FP6 in Mice after Intra-Tumor Injection

Tumor retention after intra-tumor injection was evaluated. FIG. 6 illustrates IL-12 concentrations in tumor lysate and tumor interstitial fluid of recombinant IL-12 or KLH-Bivalent IL-12 fusion protein FP6 in human T cell injected LS1034 tumor bearing mice model after intra-tumor injection, repeated for a total of six times. KLH-Bivalent IL-12 fusion protein FP6 showed higher concentration than recombinant IL-12 in both tumor lysate and interstitial fluid at pre-dose and 1 day after last dosing.

### 2-3 Evaluation of Pharmacokinetics of Bivalent IL-12 Fusion Proteins (Also Referenced Herein as Protein Complexes) in Cynomolgus Monkey

### 2-3-1 Measurement of KLH-Bivalent IL-12 Fusion Protein FP6 Concentration in Cynomolgus Monkey Plasma by ELISA

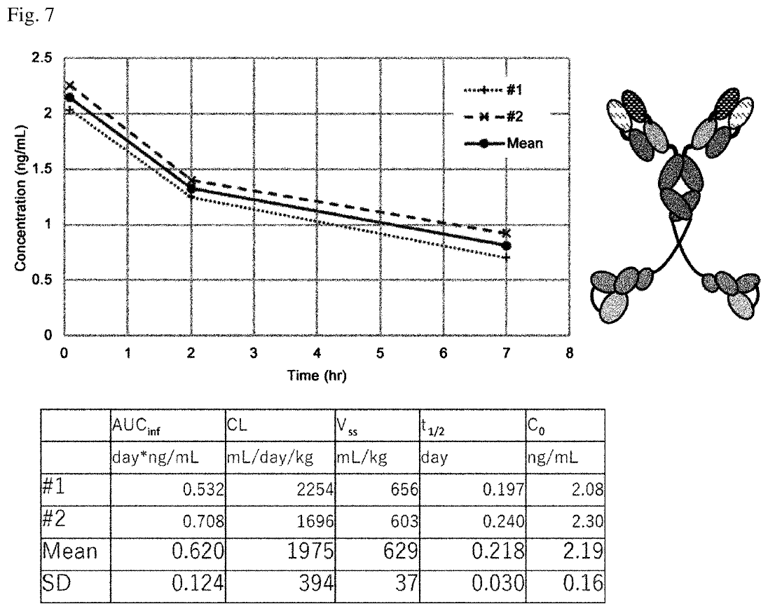

The concentration of KLH-Bivalent IL-12 fusion protein FP6 in plasma derived from cynomolgus monkey was measured by IL-12 High Sensitivity Human ELISA kit (Abcam) according to the manufacturer's instruction. KLH-Bivalent IL-12 fusion protein FP6 concentration was calculated based on the response of the calibration curve using the analytical software SOFTmax PRO (Molecular Devices). The time course of KLH-Bivalent IL-12 fusion protein FP6 concentration in plasma measured by this method is shown in FIG. 7.

### 2-3-2 In Vivo Test Using Cynomolgus Monkey

The pharmacokinetics of KLH-Bivalent IL-12 fusion protein FP6 was assessed in cynomolgus monkey (FIG. 7). KLH-Bivalent IL-12 fusion protein FP6 (0.48 mg/mL) was administrated at a single dose of 2.5 mL/kg into the cephalic vein of the forearm. Blood was collected at 5 minutes, 2 hours and 7 hours after administration. The collected blood was centrifuged immediately at 1700×g at 4 degrees C. for 5 minutes to separate the plasma. The separated plasma was stored at below −70 degrees C. until measurement.

### 2-3-3 Pharmacokinetics of KLH-Bivalent IL-12 Fusion Protein FP6 in Cynomolgus Monkey

Pharmacokinetic profiles of KLH-Bivalent IL-12 fusion protein FP6 in cynomolgus monkey was evaluated. FIG. 7 illustrates the time course of plasma KLH-Bivalent IL-12 fusion protein FP6 concentration after intravenous administration in cynomolgus monkey. KLH-Bivalent IL-12 fusion protein FP6 was eliminated rapidly and the clearance was 1975 mL/day/kg which is 13-times faster than recombinant IL-12 clearance reported in literature which is 6.23 mL/hour/kg (150 mL/day/kg) (Pharmacology 2010; 85:319-327).

As demonstrated in the tumor bearing mice model, higher concentrations of the surrogate active form of the fusion format IL-12 fusion protein were detected when compared to recombinant IL-12 of the release format IL-12 fusion protein (FIG. 6). Despite the higher concentrations accumulated within the tumor microenvironment, the surrogate active form of the fusion format IL-12 fusion protein demonstrated considerably much faster clearance than recombinant IL-12 (FIG. 7). It is therefore envisioned that fusion format IL-12 fusion protein will exhibit lower systemic toxicity compared to the release format IL-12 fusion protein.

### Example 3: Screening of IL-12 Fusion Proteins (Also Referenced Herein as Protein Complexes)—Variable Region

In order to achieve the characteristics of the presently described IL-12 fusion proteins (see Example 1), the variable region of the IL-12 fusion protein needs to bind to IL-12 sufficiently before protease digestion and yet release the IL-12 sufficiently after protease digestion. Several IL-12 binding variable region, i.e. IL-12 binding domains, were screened. The screened variable regions include (l) affinity enhancing modifications that enhance the binding affinity of the IL-12 binding domain to bind IL-12 before protease digestion and (2) VH release modifications that enhance the dissociation of VH from the fusion protein such that IL-12 is released and binds IL-12R upon protease digestion.

### Example 3-1 Selection of Variable Regions with Improved IL-12 Neutralization and Sufficient VH Release after Protease Digestion

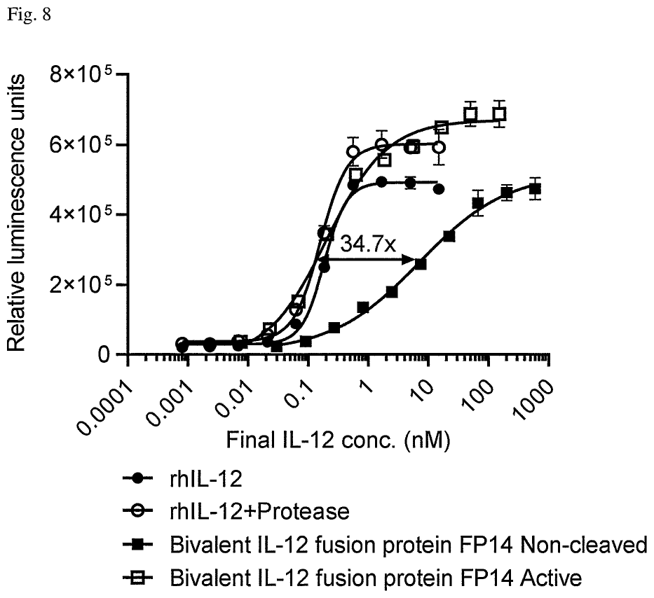

Bivalent IL-12 fusion protein FP14 (Ab101H89-12aa0054-C4-L4-IL12v1.KHKE/Ab102L69-SK1) is a homodimer made of a light chain (SEQ ID: 121) and a heavy chain (SEQ ID: 207), comprising amino acid modifications at the VH/VL interface that promotes VH dissociation, after protease digestion i.e. VH release modifications of variant Ab101H89/Ab102L69 described in Table 27B in Reference Example 1. Said heavy chain is fused to a protease resistant single-chain IL-12 (SEQ ID NO: 195), identified in Reference Example 2, at the C-terminus of constant region by a GS linker (SEQ ID NO: 68). Bivalent IL-12 fusion protein FP14 was subjected to in vitro activity assessment to check the differences in activity between non-cleaved (inactive) form and cleaved (active) form (FIG. 8). To evaluate this difference in activity, the window between non-cleaved form and cleaved form of Bivalent IL-12 fusion protein, IL-12 luciferase assay was conducted. The window, which is calculated by the difference in EC50 value, between the non-cleaved form and cleaved form was found to be ˜34.7× (times) (FIG. 8). To improve the window between the non-cleaved and cleaved form while maintaining the ability to release VH after protease digestion, affinity enhancement was performed using Ab101H89/Ab102L69 variable region as template.

### Example 3-2: Affinity Enhancement

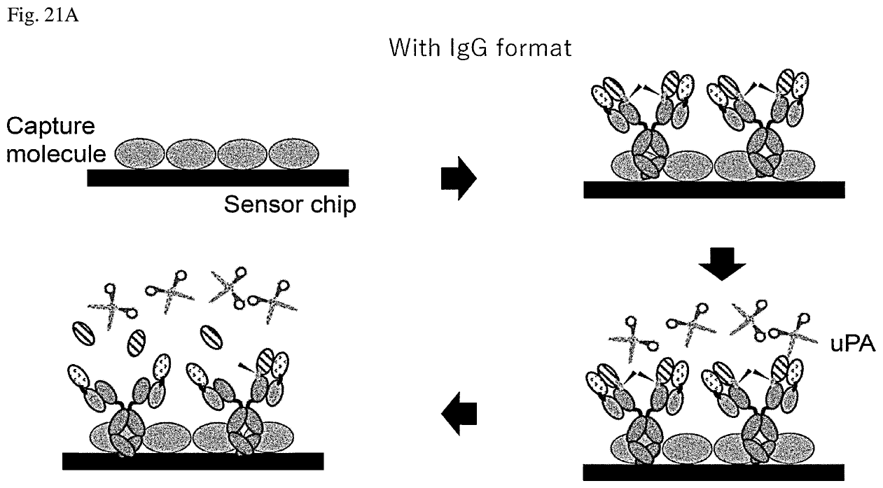

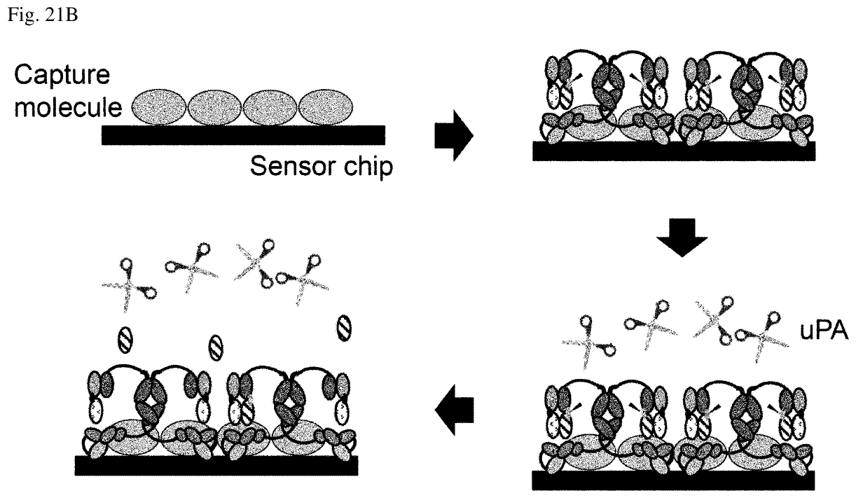

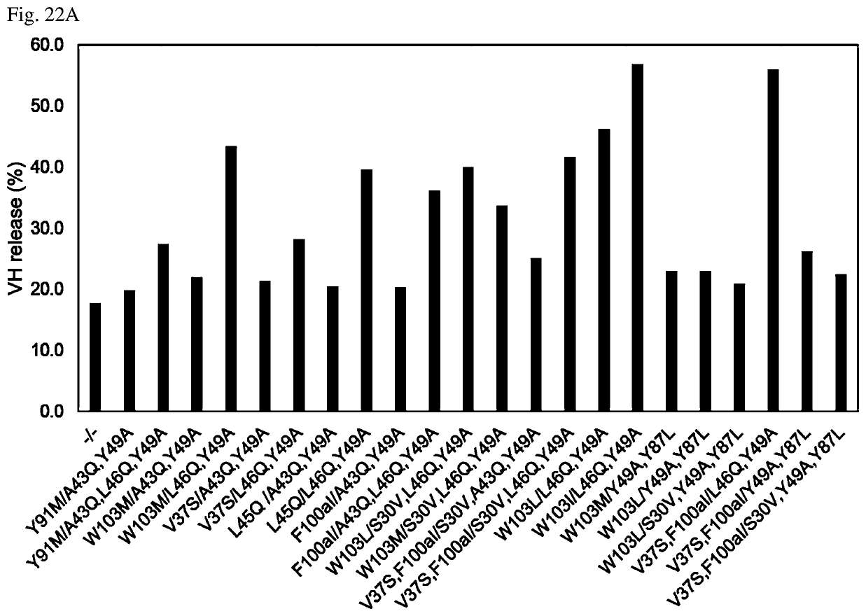

Anti-IL-12 antibody, Ab1, of the format as shown in FIG. 21A, comprising VH release enhancing mutations (Ab101H89-12aa0054-C8/Ab102L69-SK1) is a homodimer made up of a light chain (SEQ ID NO: 121) and a heavy chain (SEQ ID NO: 198). In heavy chain (SEQ ID NO: 198), VH, Ab101H89 (SEQ ID NO: 189) is fused to constant region, C8 (SEQ ID NO: 192) via a cleavable linker, 12aa0054 (SEQ ID NO: 199). Affinity enhancement mutations were performed at one or more CDR regions of VH and VL using Anti-IL-12 antibody, Ab1 with VH release mutations (Ab101H89-12aa0054-C8/Ab102L69-SK1) as a template to generate different variable region variants as shown in Table 5.

Expression vectors of each chain were prepared by a method known to those skilled in the art and expressed using Expi293 (Life Technologies Corp.) by combining each chain as shown in Table 5 and purified by ProA purification method.

TABLE 5
Ab 1 variable region variants and sequence IDs of each chain.

| Variable region (Fab) | Mutations | Light | Heavy |
|---|---|---|---|
| variants | (Heavy/Light) | chain | chain |
| Ab101H/Ab102L | —/— | SEQ ID | SEQ ID |
| | | NO: 114 | NO: 200 |
| Ab101H89/ | V37S, F100aI/L46Q, | SEQ ID | SEQ ID |
| Ab102L69 | Y49A | NO: 121 | NO: 198 |
| Ab101H89/ | V37S, F100aI/L46Q, | SEQ ID | SEQ ID |
| Ab102L994 | Y49A, N92Y | NO: 201 | NO: 198 |
| Ab101H725/ | V37S, G55Q, N95T, | SEQ ID | SEQ ID |
| Ab102L1087 | F100aI/A25S, Q27D, | NO: 202 | NO: 203 |
| | L46Q, Y49A | | |

### 3-2-1 Binding Kinetics of Affinity Enhanced Anti-IL12 Antibody Ab1 with VH Release Mutations

The affinity of anti-IL12 antibodies binding to human IL-12 at pH 7.4 was determined at 37 degrees C. using BIACORE® T200 instrument (GE Healthcare). Anti-human Fe antibody (GE Healthcare) was immobilized onto all flow cells of a CM4 sensor chip using amine coupling kit (GE Healthcare). All antibodies and analytes were prepared in ACES pH 7.4 containing 20 mM ACES, 150 mM NaCl, 0.05% Tween 20, 0.005% NaN₃. Each antibody was captured onto the sensor surface by anti-human Fc antibody. Antibody capture levels were aimed at 50 resonance unit (RU). Human IL-12 was injected at 5 and 20 nM, followed by dissociation. Sensor surface was regenerated each cycle with 3M MgCl₂. Binding affinity was determined by processing and fitting the data to 1:1 binding model using BIACORE® T200 Evaluation software (GE Healthcare). Binding kinetics were shown in Table 6. The expression E used to express the Kon, Koff, and KD values in the table means “10 to the power of” and, for instance, 1.90E+06=1.90×10⁶).

TABLE 6
Binding kinetics of combination mutants
of Ab1 variants towards IL-12

| Variable region | Mutations | | | |
|---|---|---|---|---|
| variants | (Heavy/Light) | Kon | Koff | KD (M) |
| Ab101H/Ab102L | —/— | 1.90E+06 | 2.75E−03 | 1.45E−09 |
| Ab101H89/ | V37S, F100aI/ | 2.79E+06 | 1.82E−02 | 6.52E−09 |
| Ab102L69 | L46Q, Y49A | | | |
| Ab101H89/ | V37S, F100aI/ | 2.36E+06 | 8.28E−03 | 3.51E−09 |
| Ab102L994 | L46Q, Y49A, | | | |
| | N92Y | | | |
| Ab101H725/ | V37S, G55Q, | 1.32E+07 | 1.68E−02 | 1.27E−09 |
| Ab102L1087 | N95T, F100aI/ | | | |
| | A25S, Q27D, | | | |
| | L46Q, Y49A | | | |

### Example 3-3: VH Release Evaluation of IL-12 Fusion Proteins (Also Referenced Herein as Protein Complexes) Comprising IL-12 Affinity Enhanced Variable Region

### 3-3-1 Preparation of IL-12 Fusion Proteins (Also Referenced Herein as Protein Complexes) Comprising IL-12 Affinity Enhanced Variable Region

In Table 7, several fusion protein variants were prepared by incorporating the identified IL-12 affinity enhanced variable regions assessed in Example 3-2. Bivalent IL-12 fusion proteins with IL-12 affinity enhanced variable region are homodimers made up of a light chain and a heavy chain as shown in the Table 7. For each protein, in heavy chain, VH is fused to the N-terminus of constant region, C4 (SEQ ID NO: 75) via a cleavable linker, N0222 (SEQ ID NO: 194) or 12aa0054 (SEQ ID NO: 199) and a protease-resistant single chain IL-12 (SEQ ID NO: 195) is fused to the C-terminus of constant region by a GS linker (SEQ ID NO: 68).

Expression vectors of each chain were prepared by a method known to those skilled in the art and expressed using Expi293 (Life Technologies Corp.) by combining each chain as shown in Table 7. Purification of fusion protein was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 7
Bivalent IL-12 fusion proteins (also referenced herein
as protein complexes) with IL-12 affinity enhanced
variable region and sequence IDs of each chain.

| | | Cleav- | | |
|---|---|---|---|---|
| Bivalent IL-12 | Mutations | able | Light | Heavy |
| fusion proteins | (Heavy/Light) | linker | chain | chain |
| Bivalent IL-12 | —/— | N0222 | SEQ ID | SEQ ID |
| fusion protein | | | NO: 114 | NO: 193 |
| FP13 | | | | |
| Bivalent IL-12 | V37S, F100aI/ | N0222 | SEQ ID | SEQ ID |
| fusion protein | L46Q, Y49A | | NO: 121 | NO: 204 |
| FP15 | | | | |
| Bivalent IL-12 | V37S, F100aI/ | N0222 | SEQ ID | SEQ ID |
| fusion protein | L46Q, Y49A, | | NO: 201 | NO: 204 |
| FP16 | N92Y | | | |
| Bivalent IL-12 | V37S, G55Q, | N0222 | SEQ ID | SEQ ID |
| fusion | N95T, F100aI/ | | NO: 202 | NO: 205 |
| protein FP17 | A25S, Q27D, | | | |
| | L46Q, Y49A | | | |
| Bivalent IL-12 | —/— | 12aa0054 | SEQ ID | SEQ ID |
| fusion protein | | | NO: 114 | NO: 206 |
| FP18 | | | | |
| Bivalent IL-12 | V37S, F100aI/ | 12aa0054 | SEQ ID | SEQ ID |
| fusion protein | L46Q, Y49A | | NO: 121 | NO: 207 |
| FP14 | | | | |
| Bivalent IL-12 | V37S, F100aI/ | 12aa0054 | SEQ ID | SEQ ID |
| fusion protein | L46Q, Y49A, | | NO: 201 | NO: 207 |
| FP19 | N92Y | | | |
| Bivalent IL-12 | V37S, G55Q, | 12aa0054 | SEQ ID | SEQ ID |
| fusion protein | N95T, F100aI/ | | NO: 202 | NO: 208 |
| FP20 | A25S, Q27D, | | | |
| | L46Q, Y49A | | | |
| Bivalent IL-12 | V37S, P52aA, | 12aa0054 | SEQ ID | SEQ ID |
| fusion protein | G55Q, N95T, | | NO: 209 | NO: 210 |
| FP21 | F100aI, V102Q/ | | | |
| | A25S, Q27D, | | | |
| | L46Q, Y49A, S56E | | | |
| Bivalent IL-12 | V37S, G55Q, | 12aa0054 | SEQ ID | SEQ ID |
| fusion protein | N95T, F100aI/ | | NO: 209 | NO: 208 |
| FP22 | A25S, Q27D, | | | |
| | L46Q, Y49A, S56E | | | |

### 3-3-2 VH Release Evaluation of IL-12 Fusion Proteins (Also Referenced Herein as Protein Complexes) Comprising IL-12 Affinity Enhanced Variable Region

The bivalent IL-12 fusion proteins shown in the Table 7 were subjected to VH release evaluation. VH release of IL-12 fusion proteins were determined at 37 degrees C. using BIACORE® T200 instrument (Cytiva). Protein A/G (PIERCE) was immobilized onto all flow cells of a CM4 sensor chip using amine coupling kit (Cytiva). All proteins and analytes were prepared in HBS-EP+. Each protein was captured onto the sensor surface by Protein A/G captured at flow cells (FC)—FC2, FC3, or FC4 to a level of 500 RU, and then followed by 1800 seconds injection of 400 nM of recombinant human uPA or buffer across all FC. Sensor surface was regenerated after each cycle with 10 mM Glycine-HCl, pH 1.5. The RU value at 5 seconds before the sample injection onto flow cells—FC2, FC3, or FC4 ended was adopted as the final response for each protein. The percentage of VH release was calculated using the following formula:

$$\frac{\begin{matrix}Final response of buffer injection - \\ Final response of uPA injection\end{matrix}}{Final response of buffer injection} \times 100$$

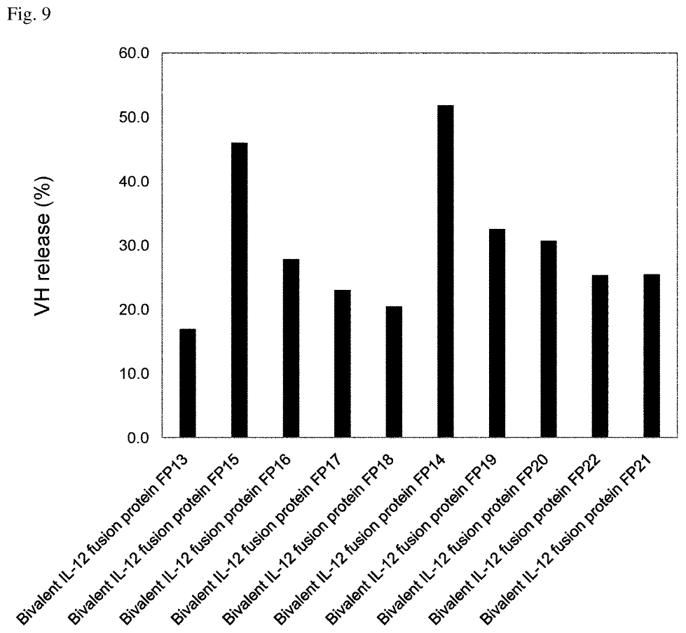

As shown in Table 8 and FIG. 9, all tested proteins demonstrated higher VH release tendency than the ones without VH release mutations.

TABLE 8
Bivalent IL-12 fusion proteins (also referenced
herein as protein complexes) with IL-12 affinity
enhanced variable region and the VH release %

| | | Cleav- | % re- | VH |
|---|---|---|---|---|
| Bivalent IL-12 | Mutations | able | duction | re- |
| fusion proteins | (Heavy/Light) | linker | in RU | lease % |
| Bivalent IL-12 | —/— | N0222 | 1.70 | 17.0 |
| fusion protein | | | | |
| FP13 | | | | |
| Bivalent IL-12 | V37S, F100aI/L46Q, | N0222 | 4.61 | 46.1 |
| fusion protein | Y49A | | | |
| FP15 | | | | |
| Bivalent IL-12 | V37S, F100aI/L46Q, | N0222 | 2.79 | 27.9 |
| fusion protein | Y49A, N92Y | | | |
| FP16 | | | | |
| Bivalent IL-12 | V37S, G55Q, N95T, | N0222 | 2.31 | 23.1 |
| fusion protein | F100aI/A25S, Q27D | | | |
| FP17 | L46Q, Y49A | | | |
| Bivalent IL-12 | —/— | 12aa0054 | 2.05 | 20.5 |
| fusion protein | | | | |
| FP18 | | | | |
| Bivalent IL-12 | V37S, F100aI/L46Q, | 12aa0054 | 5.19 | 51.9 |
| fusion protein | Y49A | | | |
| FP14 | | | | |
| Bivalent IL-12 | V37S, F100aI/L46Q, | 12aa0054 | 3.26 | 32.6 |
| fusion protein | Y49A, N92Y | | | |
| FP19 | | | | |
| Bivalent IL-12 | V37S, G55Q, N95T, | 12aa0054 | 3.08 | 30.8 |
| fusion protein | F100aI/A25S, Q27D | | | |
| FP20 | L46Q, Y49A | | | |
| Bivalent IL-12 | V37S, P52aA, G55Q, | 12aa0054 | 2.54 | 25.4 |
| fusion protein | N95T, F100aI, | | | |
| FP21 | V102Q/A25S, Q27D, | | | |
| | L46Q, Y49A, S56E | | | |
| Bivalent IL-12 | V37S, G55Q, N95T, | 12aa0054 | 2.55 | 25.5 |
| fusion protein | F100aI/A25S, Q27D | | | |
| FP22 | L46Q, Y49A, S56E | | | |

### Example 3-4: Evaluation of the Activity of IL-12 Fusion Proteins (Also Referenced Herein as Protein Complexes) with IL-12 Affinity Enhanced Variable Region Using IL-12 Reporter Assay

To assess if the fusion proteins comprising the affinity enhanced variable region identified in Example 3-2 possess improved neutralization capacity of IL-12, several fusion proteins shown in Table 9 have been subjected to IL-12 luciferase assay. Briefly, 2.5×10⁴ cells/well IL-12 bioassay cell (Promega, Cat #CS2018A02A) which express human IL-12Rb1, IL-12Rb2, and STAT4, were plated in 96-well plate and incubated overnight. Then, IL-12 fusion proteins were added to the culture plate and incubated for 18 hours. Luciferase activity was detected with Bio-Glo luciferase assay system (Promega, G7940) according to manufacturer's instructions. Luminescence was detected using GloMax (registered trademark) Explorer System (Promega #GM3500).

Data analysis was performed using Microsoft (registered trademark) Excel (registered trademark) 2013 and the analyzed data was plotted using GraphPad Prism ver. 9.0.2. EC50 values were derived using non-linear regression analysis and Agonist concentration vs. normalized response—Variable slope (four parameter) equation.

TABLE 9
EC50 values with and without protease digestion (Non-cleaved
vs Cleaved) for Bivalent IL-12 fusion proteins (also referenced
herein as protein complexes) with IL-12 affinity enhanced
variable region in IL-12 Luciferase assay

| Bivalent IL-12 | | EC50 (Non- | EC50 | Fold change |
|---|---|---|---|---|
| fusion protein | Linker | cleaved) | (Cleaved) | (window) |
| Bivalent IL-12 | N0222 | 2.86 | 0.1831 | 15.6x |
| fusion protein FP15 | | | | |
| Bivalent IL-12 | N0222 | 6.412 | 0.2376 | 27.0x |
| fusion protein FP16 | | | | |
| Bivalent IL-12 | N0222 | 8.872 | 0.2896 | 30.6x |
| fusion protein FP17 | | | | |
| Bivalent IL-12 | 12aa0054 | 7.024 | 0.2024 | 34.7x |
| fusion protein FP14 | | | | |
| Bivalent IL-12 | 12aa0054 | 13.13 | 0.2393 | 54.9x |
| fusion protein FP19 | | | | |
| Bivalent IL-12 | 12aa0054 | 17.37 | 0.2294 | 75.7x |
| fusion protein FP20 | | | | |
| Bivalent IL-12 | 12aa0054 | 10.28 | 0.2395 | 42.9x |
| fusion protein FP21 | | | | |
| Bivalent IL-12 | 12aa0054 | 12.69 | 0.2958 | 42.9x |
| fusion protein FP22 | | | | |

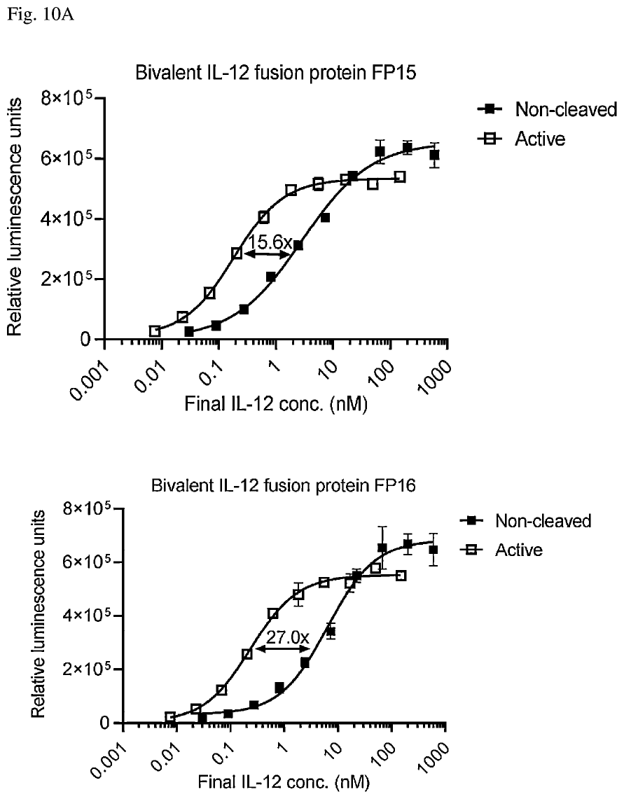

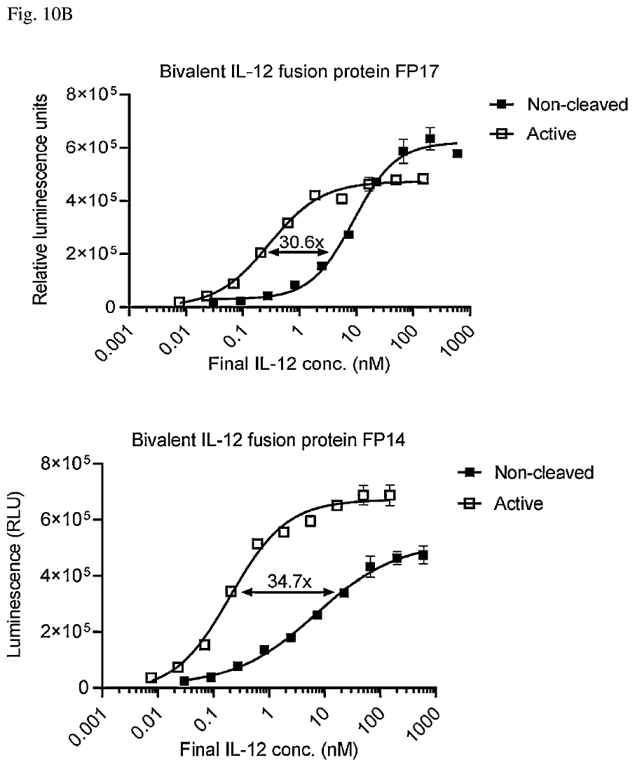

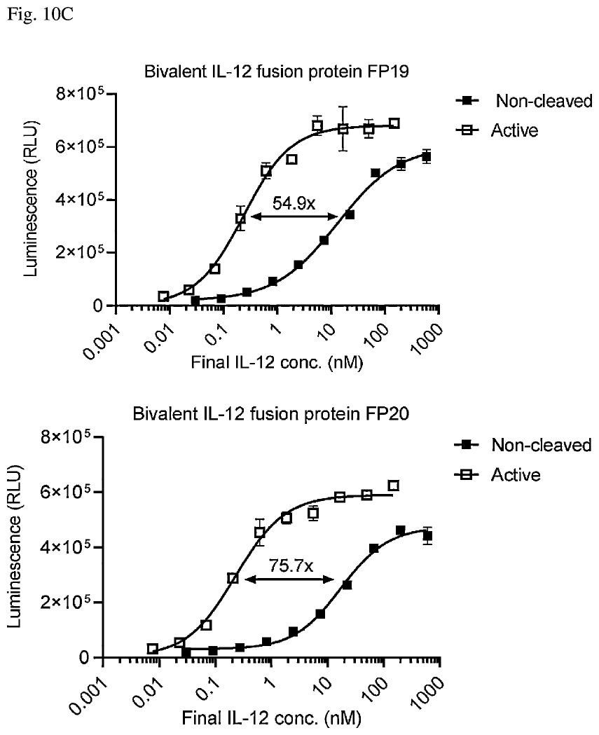

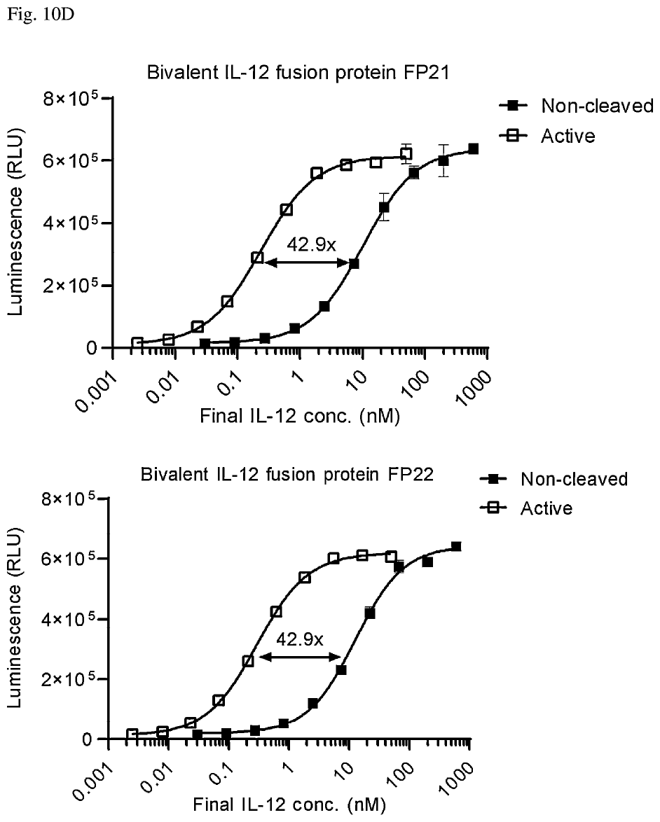

Within molecules of the same cleavable linker, fusion proteins comprising IL-12 affinity enhanced variable region showed improved neutralization in Bivalent IL-12 fusion protein format (FIGS. 10A to 10D and Table 9), as evidenced by the increased EC50 values of non-cleaved (inactive) forms relative to the fusion proteins without the IL-12 affinity enhanced variable region (“control”)—Bivalent IL-12 fusion protein FP14 and Bivalent IL-12 fusion protein FP15. Notably, EC50 values of active proteins were consistent amongst all proteins.

### Example 3-5 Evaluation of the Activity of IL-12 Fusion Proteins (Also Referenced Herein as Protein Complexes) Using Human PBMCs

To check if the Bivalent IL-12 fusion proteins comprising the affinity enhanced variable region identified in Example 3-2 could show neutralization in primary cells, the activity of several fusion proteins shown in Table 10 was assessed in human peripheral blood mononuclear cells (PBMCs). Briefly, PBMCs (Stem cell technologies #70025) were stimulated with 5 micro g/mL of phytohemagglutinin-L (PHA-L) (Thermofisher Scientific #00-4977-03) at a cell concentration of 1×10⁶/mL for 48 h in culture. Stimulated cells were then harvested and seeded at 5×10⁴ cells/well, before addition of non-cleaved (inactive) or cleaved (active), i.e. human uPA-treated, (huPA; R&D Systems; 1310-SE-010) IL-12 fusion proteins. Culture supernatant was harvested 18 h after incubation and IL-12 activity was determined by detecting IFN gamma levels using an ELISA kit (R&D systems #SIF50C) according to manufacturer's instructions. Optical densities were detected using Multiskan GO (Thermofisher Scientific #N10588). Data analysis was performed using Microsoft (registered trademark) Excel (registered trademark) 2013 and GraphPad Prism ver. 9.0.2. EC50 values were derived using nonlinear regression analysis and Agonist concentration vs. normalized response—Variable slope (four parameter) equation.

TABLE 10
EC50 values with and without protease digestion (Non-cleaved
vs Cleaved) for Bivalent IL-12 fusion proteins (also referenced
herein as protein complexes) in PBMC assay

| | Donor 1 | | Donor 2 | | Donor 3 | |
|---|---|---|---|---|---|---|
| | EC50 | | EC50 | | EC50 | |
| Bivalent IL-12 | (Non- | EC50 | (Non- | EC50 | (Non- | EC50 |
| fusion protein | cleaved) | (Cleaved) | cleaved) | (Cleaved) | cleaved) | (Cleaved) |
| rhIL-12 | 4.46 | | 7.22 | | 3.93 | |
| Bivalent IL-12 fusion | 1144 | 7.57 | 1687 | 32.25 | 533.3 | 6.68 |
| protein FP14 | | | | | | |
| Bivalent IL-12 fusion | 299.5 | 17.36 | 1562 | 50.32 | 421.3 | 11.99 |
| protein FP20 | | | | | | |
| Bivalent IL-12 fusion | 865.7 | 21.10 | 1959 | 46.22 | 841.3 | 16.52 |
| protein FP21 | | | | | | |
| Bivalent IL-12 fusion | 1156 | 12.17 | 2635 | 20.47 | 1558 | 9.73 |
| protein FP22 | | | | | | |

Within each PBMC donor, non-cleaved bivalent IL-12 fusion proteins showed IL-12 neutralization to varying degrees (FIGS. 11A to 11D). Following protease digestion, IL-12 activity of each protein was increased between 17-160-fold relative to their respective non-cleaved form. Such trend was observed across all 3 PBMC donors, confirming the reproducibility of IL-12 fusion proteins' activity.

### Example 4: Screening of IL-12 Fusion Proteins (Also Referenced Herein as Protein Complexes)—Cleavable Linker

The cleavable linker in the presently described IL-12 fusion protein is specifically cleaved in the tumor microenvironment. Protease cleavable linkers which are disclosed in WO2019/107384 or WO2020/246567 were selected for cleavage evaluation.

### Example 4-1 Preparation of IL-12-Fusion Proteins (Also Referenced Herein as Protein Complexes) Having Different Cleavable Linkers In Vitro

Bivalent IL-12 fusion protein FP23 is a homodimer of a heavy chain SEQ ID NO: 211 and a light chain SEQ ID NO: 212. There is no cleavable linker between the VH (SEQ ID NO: 51) and CH1 domain in the heavy chain of Bivalent IL-12 fusion protein FP23. The selected cleavable linkers shown in Table 11 were introduced into the elbow hinge region of the heavy chain of Bivalent IL-12 fusion protein FP23 to produce fusion proteins with different cleavable linkers (cleavable variants). Each of these heavy chains were then combined with the light chain to form the cleavable variants as shown in Table 12. The cleavable variants shown in Table 12 were expressed by transient expression using Expi293 cells (Thermo Fisher Scientific) by a method known in the art, and purified with Protein A and gel filtration chromatography by a method known in the art.

TABLE 11
Protease cleavable linkers

| SEQ ID NO | Linker sequence |
|---|---|
| 213 | GGGSGGGSGGGS |
| 214 | TSTLGRSANPRG |
| 215 | TSTSGRSAEFRG |
| 46 | TSTSGRSANPRG |
| 199 | TSGSGRSATPRG |
| 216 | YAYTGRSANPRG |
| 194 | YSATGRSANPRG |
| 217 | TSPTGRSANPRG |
| 218 | TSYTGRSAVYRG |
| 219 | TSYTGRSAVVRG |

TABLE 12
Bivalent IL-12 fusion protein FP23
(also referenced herein as a
protein complex) with different
cleavable linkers
(Cleavable Variants)

| | | Heavy | Light |
|---|---|---|---|
| | | chain | chain |
| Cleavable | | SEQ | SEQ |
| Variant | Linker | ID | ID |
| name | sequence | NO | NO |
| G3Sx3 | GGGSGGGSGGGS | 220 | 212 |
| | (SEQ ID NO: 213) | | |
| 12aaT10. | TSTLGRSANPRG | 221 | 212 |
| 0018 | (SEQ ID NO: 214) | | |
| 12aa0049 | TSTSGRSAEFRG | 222 | 212 |
| | (SEQ ID NO: 215) | | |
| 12aa | TSTSGRSANPRG | 223 | 212 |
| | (SEQ ID NO: 46) | | |
| 12aa0054 | TSGSGRSATPRG | 224 | 212 |
| | (SEQ ID NO: 199) | | |
| N0206 | YAYTGRSANPRG | 225 | 212 |
| | (SEQ ID NO: 216) | | |
| N0222 | YSATGRSANPRG | 226 | 212 |
| | (SEQ ID NO: 194) | | |
| 12aa0069 | TSPTGRSANPRG | 227 | 212 |
| | (SEQ ID NO: 217) | | |
| 12aa0089. | TSYTGRSAVYRG | 228 | 212 |
| 005 | (SEQ ID NO: 218) | | |
| 12aa0089. | TSYTGRSAVVRG | 229 | 212 |
| 006 | (SEQ ID NO: 219) | | |

### Example 4-1-1 Cleavage of IL-12-Fused Antibody Variants Having Several Protease Cleavable Linkers by Recombinant Proteases and Human Plasma

The fusion proteins with different cleavable linkers (cleavable variants) produced in Example 4-1 were treated by recombinant human u-Plasminogen Activator/Urokinase (human uPA, huPA) (R&D Systems; 1310-SE-010) and recombinant human Matriptase/ST14 Catalytic Domain (human MT-SP1, hMT-SP1) (R&D Systems; 3946-SEB-010). The variants were incubated with huPA or hMT-SP1 in PBS for 30 minutes at 37 degrees C. under the conditions of 100 nM huPA or 2 nM hMT-SP1 and 0.65 micro M cleavable variants.

The cleavable variants produced in Example 4-1 were treated by human plasma in sodium citrate. The cleavable variants were incubated with plasma for 21 days at 37 degrees C. under the condition shown in Table 13.

TABLE 13
Cleavage condition using human plasma

| | |
|---|---|
| Human plasma in sodium citrate | 80 μL |
| Cleavable variant (3.3 μM) | 10 μL |
| 1M HEPES | 9 μL |
| 10% sodium azide | 1 μL |

### Example 4-1-2 Evaluation of Cleavage of IL-12 Fusion Proteins (Also Referenced Herein as a Protein Complex) with Different Cleavable Linkers In Vitro by Recombinant Proteases and Human Plasma Using LC/ESI-MS/MS

After protease and plasma treatment in Example 4-1-1, linker cleavage of the cleavable variants was evaluated using LC/ESI-MS/MS. The calibration standards (CS) or samples were pretreated with acid dissociation or urea-induced denaturation (in order to dissociate the non-specific binding) and then purified by immunoprecipitation (IP). The sample volumes, CS matrices, CS ranges, denaturing conditions and IP conditions for each sample are shown in Table 14.

TABLE 14
Sample preparation and linker cleavage evaluation using LC/ESI-MS/MS

| | | | | | | | | IP condition | |
|---|---|---|---|---|---|---|---|---|---|
| | | | sample | | CS | CS range | denaturing | before | after |
| condition | study name | | volume | | matrix | (μg/mL) | condition | denaturing | denaturing |
| 1 | in vivo | plasma | 5 | μL | mouse | 0.8~51.2 | A | C | E |
| | | | | | plasma | | | | |
| 2 | | tumor | 60 | μL | lysis | 0.025~3.2 | A | C | F |
| | | lysate | | | buffer | | | | |
| 3 | plasma | human | 5 | μL | human | 0.8~51.2 | A | D | E |
| | | | | | plasma | | | | |
| 4 | in vitro | | 2.5 | μL | PBS-T | 1.5~96 | A | C | E |
| 5 | ex vivo | breast, | 10 | μL | lysis | 0.2~12.8 | A | C | E |
| | (human) | colon, lung | | | buffer | | | | |
| 6 | | heart | 10 | μL | lysis | 0.2~12.8 | B | — | F |
| | | | | | buffer | | | | |
| 7 | | spleen | 8 | μL | lysis | 0.2 ~12.8 | B | — | D |
| | | | | | buffer | | | | |

A: Samples were mixed with 25 micro L of 8 mmol/L dithiothreitol/7.5 mol/L Urea/50 mmol/L ammonium bicarbonate and incubated for 45 min at 56 degrees C. Then, 2 micro L of 500 mmol/L iodoacetamide was added and incubated for 30 min at 37 degrees C. in the dark.
B: Samples were mixed with 50 micro L of 0.3 mol/L Acetic acid and incubated for 30 min at 25 degrees C. Then, 100 micro L of 1 mol/L Tris-HCl (pH 8) was added and incubated for 1 min at 25 degrees C.
C: Samples were mixed with 50 micro L of 50 micro g/mL biotinylated anti-human IgG antibody (Sigma-Aldrich; I9885) and incubated for one hour at 25 degrees C. Then, 25 micro L of streptavidin magnetic beads (MBL; J-MS-S300S) was added and incubated for one hour at 25 degrees C. The beads were washed with PBS-T.
D: Samples were mixed with 0.6~1.25 micro g biotinylated anti-human IL-12 antibody (R&D Systems; AF-219-NA) and incubated for one hour at 25 degrees C. Then, 25 micro L of streptavidin magnetic beads was added and incubated for one hour at 25 degrees C. The beads were washed with PBS-T.
E: Samples were mixed with 10 micro L Protein A magnetic beads (Protenova; P-052-10) in PBS-T and incubated for two hours at 25 degrees C. The beads were washed with PBS-T.
F: Samples were mixed with 50 micro L of anti-human IgG antibody immobilized magnetic beads (Sigma-Aldrich; I9885, MBL; J-MS-S300C) and incubated for two hours at 25 degrees C. The beads were washed with PBS-T.
For the samples pretreated with Acetic acid, IP was followed by adding 25 micro L of 8 mmol/L dithiothreitol/7.5 mol/L Urea/50 mmol/L ammonium bicarbonate and incubated for 45 min at 56 degrees C., then, 2 micro L of 500 mmol/L iodoacetamide was added and incubated for 30 min at 37 degrees C. in the dark.

Next, 160 micro L of 0.625 micro g/mL sequencing grade modified trypsin (Promega; V5117) in 50 mmol/L ammonium bicarbonate was added and incubated at 37 degrees C. overnight. Finally, 5 micro L of 10% trifluoroacetic acid was added to deactivate any residual trypsin, and 80 micro L of digestion samples were subjected to analysis by LC/ESI-MS/MS.

### LC/ESI-MS/MS Analysis

LC/ESI-MS/MS was performed using Xevo TQ-S triple quadrupole instrument (Waters) equipped with Acquity I-class 2D high-performance liquid chromatography systems (Waters). VH region specific peptide SDDTAVYYCNANK (SEQ ID NO: 356) and IL-12 specific peptides DIIKPDPPK (SEQ ID NO: 357) and QTLEFYPCTSEEIDHEDITK (SEQ ID NO: 358) were monitored by the selected reaction monitoring (SRM). As SRM transitions, m/z 760.82 to m/z 932.39 for SDDTAVYYCNANK (SEQ ID NO: 356), m/z 511.798 to m/z 341.218 for DIIKPDPPK (SEQ ID NO: 357), and m/z 819.037 to m/z 837.32 for QTLEFYPCTSEEIDHEDITK (SEQ ID NO: 358) were selected. The calibration curves were constructed by the weighted (1/x2) linear regression using the peak area plotted against the concentrations respectively. The concentrations in samples were calculated from the calibration curve using the analytical software Masslynx Ver. 4.1 (Waters).

The cleavage ratio (%) of the cleavable variants was calculated with the following formula:

$$100 - ( Calculated conc . of VH ) \times 100 / ( Calculated conc . of IL - 12 )$$

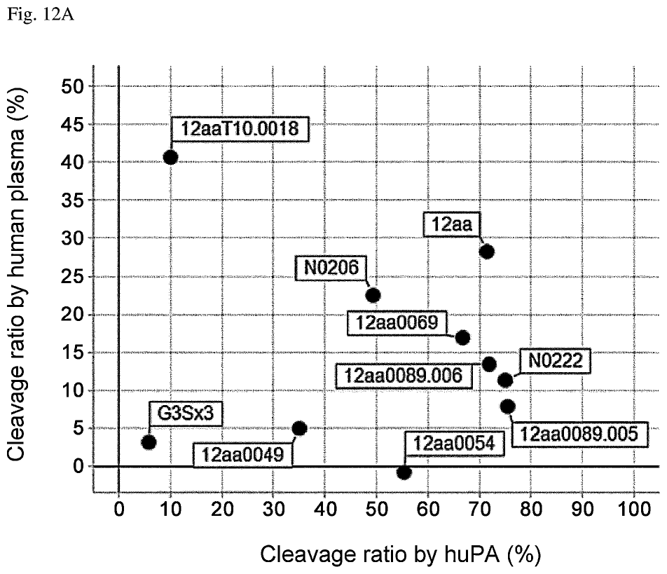

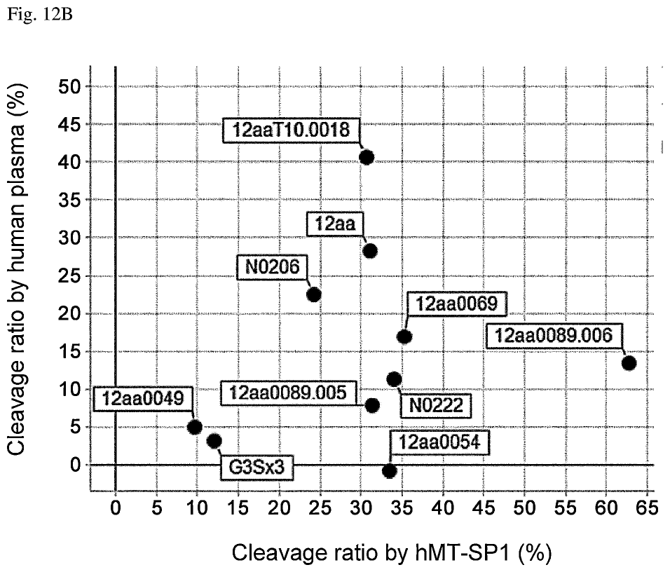

The cleavage ratio (%) of the cleavable variants by huPA, hMT-SP1 and human plasma are shown in Table 15, and FIGS. 12A and 12B. All cleavable variants demonstrated higher cleavage ratio (%) in huPA and hMT-SP1 compared to human plasma.

TABLE 15
Cleavage ratio (%) of IL-12 cleavable variants
by huPA, hMT-SP1 and human plasma

| | Cleavage ratio (%) | | |
|---|---|---|---|
| Cleavable | | | Human |
| Variant name | huPA | hMT-SP1 | plasma |
| G3Sx3 | 5.9 | 12.0 | 3.2 |
| (negative) | | | |
| 12aaT10.0018 | 10.0 | 30.7 | 40.7 |
| (positive) | | | |
| 12aa0049 | 35.0 | 9.7 | 5.0 |
| 12aa | 71.3 | 31.2 | 28.3 |
| 12aa0054 | 55.3 | 33.5 | −0.7 |
| N0206 | 49.3 | 24.2 | 22.5 |
| N0222 | 75.1 | 34.0 | 11.3 |
| 12aa0069 | 66.6 | 35.3 | 16.9 |
| 12aa0089.005 | 75.4 | 31.3 | 7.9 |
| 12aa0089.006 | 71.9 | 62.7 | 13.4 |

### Example 4-2 In Vivo and Ex Vivo Cleavage of IL-12-Fusion Proteins (Also Referenced Herein as Protein Complexes) with Different Cleavable Linkers

### Example 4-2-1 Preparation of IL-12 Fusion Proteins (Also Referenced Herein as Protein Complexes) with Different Cleavable Linkers

Bivalent IL-12 fusion protein FP24 is a homodimer of a heavy chain SEQ ID NO: 230 and a light chain SEQ ID NO: 231. There is no cleavable linker between the VH (SEQ ID NO: 51) and CH1 domain in the heavy chain of Bivalent IL-12 fusion protein FP24. GS linker (SEQ ID NO: 106) was inserted in the hinge region and single-chain IL-12 (SEQ ID NO: 67) was attached to C-terminal of Fc domain via GS linker (SEQ ID NO: 68). The selected cleavable linkers shown in Table 11 were introduced into the elbow hinge region of the heavy chain of Bivalent IL-12 fusion protein FP24 to produce fusion proteins with different cleavable linkers (cleavable variants). Each of these heavy chains were then combined with the light chain to form the cleavable variants as shown in Table 16. The cleavable variants shown in Table 16 were expressed by transient expression using Expi293 cells (Thermo Fisher Scientific) by a method known in the art, and purified with Protein A and gel filtration chromatography by a method known in the art.

TABLE 16
Bivalent IL-12 fusion protein FP24
(also referenced herein as a
protein complex) with different
cleavable linkers
(Cleavable variants)

| | | Heavy | Light |
|---|---|---|---|
| | | chain | chain |
| Cleavable | | SEQ | SEQ |
| Variant | Linker | ID | ID |
| name | sequence | NO | NO |
| G3Sx3 | GGGSGGGSGGGS | 232 | 231 |
| | (SEQ ID NO: 213) | | |
| 12aaT10. | TSTLGRSANPRG | 233 | 231 |
| 0018 | (SEQ ID NO: 214) | | |
| 12aa0049 | TSTSGRSAEFRG | 234 | 231 |
| | (SEQ ID NO: 215) | | |
| 12aa | TSTSGRSANPRG | 235 | 231 |
| | (SEQ ID NO: 46) | | |
| 12aa0054 | TSGSGRSATPRG | 236 | 231 |
| | (SEQ ID NO: 199) | | |
| N0206 | YAYTGRSANPRG | 237 | 231 |
| | (SEQ ID NO: 216) | | |
| N0222 | YSATGRSANPRG | 238 | 231 |
| | (SEQ ID NO: 194) | | |
| 12aa0069 | TSPTGRSANPRG | 239 | 231 |
| | (SEQ ID NO: 217) | | |
| 12aa0089. | TSYTGRSAVYRG | 240 | 231 |
| 005 | (SEQ ID NO: 218) | | |
| 12aa0089. | TSYTGRSAVVRG | 241 | 231 |
| 006 | (SEQ ID NO: 219) | | |

### Example 4-2-2 In Vivo Plasma Digestion of IL-12 Fusion Proteins (Also Referenced Herein as Protein Complexes) with Different Cleavable Linkers in Non-Tumor Bearing (NTB) Normal Mice

To evaluate in vivo stability of the cleavable variants produced in Example 4-2-1, in vivo digestion analysis was conducted. 10-week-old NOD.CB17-Prkdc^{scid}/J female mice (Charles River Laboratories, Japan) were randomized depending on their body weight. After the randomization, the cleavable variants produced in Example 4-2-1 were diluted with 0.05% Tween-20 in phosphate-buffered saline (PBS-T) and intravenously administered into the tail vein at 10 mg/kg. Venous blood was collected 7 days after administration via postcaval vein by using EDTA 2K as an anticoagulant. Collected blood was centrifuged at 15,000×g, at 4 degree C. for 10 minutes, and supernatant plasma was isolated and kept at −30 degree C. for the following LC/ESI-MS/MS analysis in Example 4-2-5.

### Example 4-2-3 In Vivo Tumor Digestion of IL-12 Fusion Proteins (Also Referenced Herein as Protein Complexes) with Different Cleavable Linkers in Tumor Bearing (TB) Mice

To evaluate in vivo digestion of the cleavable variants produced in Example 4-2-1 by proteases expressed in tumors, in vivo digestion analysis in tumor bearing mice was conducted. Human uPA transfected NCI-H446 tumorigenic cells were obtained and cultured as described in Example 4-3-2. 1×10⁷ cells in Hank's balanced salt solution (HBSS; Ca. No: H9269, SIGMA) were subcutaneously injected into right flank of 7-week-old NOD.CB17-Prkdc^{scid}/J female mice (Charles River Laboratories, Japan) with equal volume of Matrigel (Cat. No: 356234, Corning) to establish the xenograft tumor bearing model. Once the tumor volume reached around 150 mm³ (10-week-old), randomization was conducted depending on tumor size and body weight. After the randomization, the cleavable variants produced in Example 4-2-1 were diluted with 0.05% Tween-20 in PBS-T and intravenously administered into the tail vein at 10 mg/kg. Subcutaneous tumors were collected 7 days after administration and were frozen with liquid nitrogen immediately after the collection. Frozen tumors were thawed and homogenized in lysis buffer (50 mM Tris, 150 mM NaCl, 0.5% sodium deoxycholate, 2% NP-40, pH 8.0) with protease inhibitor cocktail cOmplete™ (Cat. No: 04693116001, Roche) by using TissueLyser II (QIAGEN). 400 micro L lysis buffer per 100 mg tumor was used for the lysis. After centrifugation at 16,000×g, at 4 degree C. for 15 minutes, supernatant was collected and further diluted with equal volume of lysis buffer (10% lysate). Resulting tumor lysate were kept at −80 degree C. for the following LC/ESI-MS/MS analysis in Example 4-2-5.

### Example 4-2-4 Ex Vivo Digestion of IL-12 Fusion Proteins (Also Referenced Herein as Protein Complexes) with Different Cleavable Linkers

Stability of the cleavable variants produced in Example 4-1 and 4-2-1 was evaluated in membrane lysates prepared from human tumor/normal tissues. Commercially available, fresh-frozen tissue fragments of human tumors (breast carcinoma, adenocarcinoma of the large intestine (colon) and lung adenocarcinoma) and of human normal tissues (breast, heart and spleen) were purchased from BioIVT (Westbury, NY). Lysates from membrane fraction of these tissues were extracted by using Mem-PER™ Plus membrane protein extraction kit (Thermo Scientific™, 89842). After the brief wash of the frozen tissue fragments, tissues were minced and homogenized in permeabilization buffer. Then, homogenates were centrifuged at 16,000×g, at 4 degree C. for 15 minutes, supernatant cytosol fractions were discarded. Pellets were resuspended in solubilization buffer by rotating at 4 degree C. for 30 minutes. After centrifugation at 16,000×g, at 4 degree C. for 15 minutes, supernatant membrane fractions were collected. In each step of extraction, assuming each tissue is 1 g/mL, 3-fold volume of initial weight of lysis buffer was used for the extraction. Resulting 25% membrane lysates were aliquoted, and each cleavable variant was added at a rate shown in Table 17. As a negative control of cleavage, samples were pre-treated with protease inhibitor cocktail cOmplete (Cat. No: 04693116001, Roche)

TABLE 17
Sample preparation for Ex vivo digestion analysis

| | |
|---|---|
| 25% membrane lysate (final 20%) | 80% |
| Assay buffer/protease inhibitor cocktail (negative ctrl) | 10% |
| ↓pre-treatment: 1 hr at 37 degree C | |
| Cleavable variants (final 10 μg/mL) | 10% |

Cleavable variants were added into these membrane lysates at final concentration of 10 ug/mL, followed by incubation at 37 degree C. for 4 hours for tumor lysate, and 20 hours for normal tissue lysate.

After incubation, protease inhibitor cocktail cOmplete (Cat. No: 04693116001, Roche) was added to the analytes at final concentration of 4× to stop further proteolytic cleavage. Analytes were kept at −30 degree C. For the following LC/ESI-MS/MS analysis in Example 4-2-5.

### Example 4-2-5 Evaluation of In Vivo and Ex Vivo Cleavage of IL-12 Fusion Proteins (Also Referenced Herein as Protein Complexes) with Different Cleavable Linkers Using LC/ESI-MS/MS

After performing in vivo plasma digestion (Example 4-2-2), in vivo tumor digestion (Examples 4-2-3) and ex vivo digestion (Example 4-2-4), linker cleavage of the fusion proteins with different cleavable linkers (cleavable variants) was evaluated using high-performance liquid chromatography-electrospray tandem mass spectrometry (LC/ESI-MS/MS) described in Example 4-1-2. The remaining intact (%) of the cleavable variants was calculated with the following formula:

$$( Calculated conc . of VH ) \times 100 / ( Calculated conc . of IL - 12 )$$

The remaining intact (%) of the IL-12 fusion proteins with different cleavable linkers (cleavable variants) in mice plasma and tumor, on human normal and human tumor tissue lysate are shown in Table 18 and Table 19 respectively.

TABLE 18
In vivo cleavable linker analysis in NTB and TB mice

| | |
|---|---|
| | Remaining intact % |
| Cleavable | In vivo (mice) |

| Variant name | Plasma in NTB | Tumor in TB |
|---|---|---|
| G3Sx3 (negative) | 122% | 117% |
| 12aaT10.0018 (positive) | 46% | 39% |
| 12aa0049 | 101% | 73% |
| 12aa | 91% | 60% |
| 12aa0054 | 102% | 70% |
| N0206 | 94% | 69% |
| N0222 | 90% | 42% |
| 12aa0069 | 91% | 63% |
| 12aa0089.005 | 85% | 57% |
| 12aa0089.006 | 95% | 58% |

TABLE 19
Ex vivo cleavable linker analysis using human tissues

| | Remaining | | | Remaining | | |
|---|---|---|---|---|---|---|
| | intact % (20 h) | | | intact % (4 h) | | |
| | Ex vivo human | | | | | |
| Cleavable | | | | Breast | Colon | Lung |
| Variant name | Breast | Heart | Spleen | tumor | tumor | tumor |
| G3Sx3 | 96% | 104% | 113% | 102% | 95% | 95% |
| 12aaT10.0018 | 87% | 98% | 70% | 79% | 73% | 72% |
| 12aa0049 | 91% | 103% | 74% | 90% | 96% | 87% |
| 12aa | 87% | 104% | 81% | 78% | 69% | 66% |
| 12aa0054 | 89% | 99% | 88% | 72% | 66% | 62% |
| N0206 | 92% | 100% | 90% | 80% | 86% | 78% |
| N0222 | 90% | 101% | 94% | 71% | 70% | 59% |
| 12aa0069 | 80% | 99% | 102% | 67% | 61% | 63% |
| 12aa0089.005 | 83% | 96% | 23% | 68% | 66% | 60% |
| 12aa0089.006 | 90% | 100% | 43% | 69% | 73% | 62% |

As shown in Table 19 above, cleavable variants remain intact in non-tumor tissues after 20 hours, but showed cleavage in tumor tissues in 4 hours.

### Example 4-3 Accumulation of Activated IL-12 Fusion Proteins (Also Referenced Herein as Protein Complexes) in Tumor

### Example 4-3-1 Preparation of IL-12 Fusion Proteins (Also Referenced Herein as Protein Complexes) Comprising Different Cleavable Linkers

Bivalent IL-12 fusion proteins comprising IL-12 affinity enhanced variable region further incorporated with different cleavable linkers were prepared. The resulting Bivalent IL-12 fusion proteins are homodimers made up of a light chain and heavy chain as shown in the Table 20.

Expression vectors of each chain were prepared by a method known to those skilled in the art and expressed using Expi293 (Life Technologies Corp.) by combining each chain as shown in Table 20. Purification of fusion protein was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 20
Bivalent IL-12 fusion protein (also referenced herein as a
protein complex) with IL-12 affinity enhanced variable region
and different linkers and sequence IDs of each chain.

| Bivalent IL-12 | | Light | Heavy |
|---|---|---|---|
| fusion protein | Linker | chain | chain |
| Bivalent IL-12 | N0222 | SEQ ID | SEQ ID |
| fusion protein FP17 | | NO: 202 | NO: 205 |
| Bivalent IL-12 | 12aa0054 | SEQ ID | SEQ ID |
| fusion protein FP20 | | NO: 202 | NO: 208 |
| Bivalent IL-12 | G3Sx3 | SEQ ID | SEQ ID |
| fusion protein FP25 | | NO: 202 | NO: 242 |

### Example 4-3-2 In Vivo Digestion of Bivalent IL-12 Fusion Proteins (Also Referenced Herein as Protein Complexes) Comprising with Different Cleavable Linkers in TB Mice with uPA/MTSP1 Expressing Cell Lines

The NCI-H446 human lung carcinoma cell line and the LS 174T human colorectal carcinoma cell line were obtained from American Type Culture Collection (ATCC). huPA was transfected in the NCI-H446 cells by Lipofectamine (trademark) 2000 (Invitrogen). The huPA transfected NCI-H446 cells were cultured in RPMI-1640 medium (SIGMA) plus 0.2 mg/mL G418 sulfate (Gibco) with 10% fetal bovine serum (FBS; CORNING) to produce huPA-overexpressing NCI-H446 cells. The LS 174T cells were cultured in E-MEM medium (SIGMA) plus 0.1 mM MEM Non-Essential Amino Acids Solution (Gibco), 2 mM L-Glutamine (Gibco), 1.5 g/L sodium bicarbonate (Gibco), 1 mM Sodium Pyruvate (Gibco) with 10% fetal bovine serum (FBS; CORNING). NOD.CB17-Prkdcscid/J female mice of 4 weeks of age were purchased from Charles River Laboratories Japan, Inc and were acclimated for 2 weeks before the inoculation. The huPA transfected NCI-H446 cells and the LS 174T cells in log phase growth were harvested and washed with Hank's balanced salt solution (HBSS; SIGMA), resuspended in 50% HBSS and 50% Matrigel (CORNING) at a concentration of 5×10⁷ cells/mL. Mice were subcutaneously inoculated with 1×10⁷ cells in 200 micro L of HBSS:Matrigel (1:1). When mean tumor volume reached about 100-300 mm³, mice were randomized into groups based on tumor volume. Tumor volume was measured with caliper, and tumor volume was calculated as ½×1×w², l=length, w=width. 1 day after the randomization, 10 mg/kg of bivalent IL-12 fusion proteins shown in Table 20 were intravenously administered. Tumor was resected on 14 days or 7 days after treatment.

One-fourth of tumor sections were placed on the tube with mesh and centrifuged at 420×g at 4 degrees C. for 10 minutes. The collected fluid samples were centrifuged again at 10,000×g at 4 degrees C. for 10 minutes, then supernatants were kept as tumor interstitial fluid. Blood samples were collected 5 minutes, 4 hours, 1 day, 3 days and 7 days after administration by using EDTA 2K as an anticoagulant. Collected blood was centrifuged at 1,900×g, at 4 degree C. for 10 minutes, and supernatant plasma was isolated.

### Example 4-3-3 Evaluation of Total and Active IL-12 Fusion Proteins (Also Referenced Herein as Protein Complexes) in Tumor and Plasma

Whole tumor lysate was prepared as follows according to the tumor weight by assuming the tumor density of 1 g/mL. 5 mm diameter-stainless steel beads (Qiagen) or 3 mL metal corn beads (Yasui Kikai) were added into the low protein adsorption tubes (Sumitomo Bakelite) or homogenization tubes (Yasui Kikai) containing resected tumors and a certain-fold volume of buffer (50 mM Tris, 150 mM NaCl, 0.5% sodium deoxycholate, 2% NP-40, pH 8.0) supplemented with cOmplete Protease Inhibitor Cocktail (Roche) was added into each tube. Tumor samples were then homogenized using TissueLyser II (Qiagen) for 1 minute at 25 Hz or Multi-beads shocker (Yasui Kikai) for 15 seconds at 2000 rpm. Homogenate samples were incubated at 4 degrees C. for 1 hour and then centrifuged at 14000 rpm at 4 degrees C. for 15 minutes. The supernatant was collected for analysis.

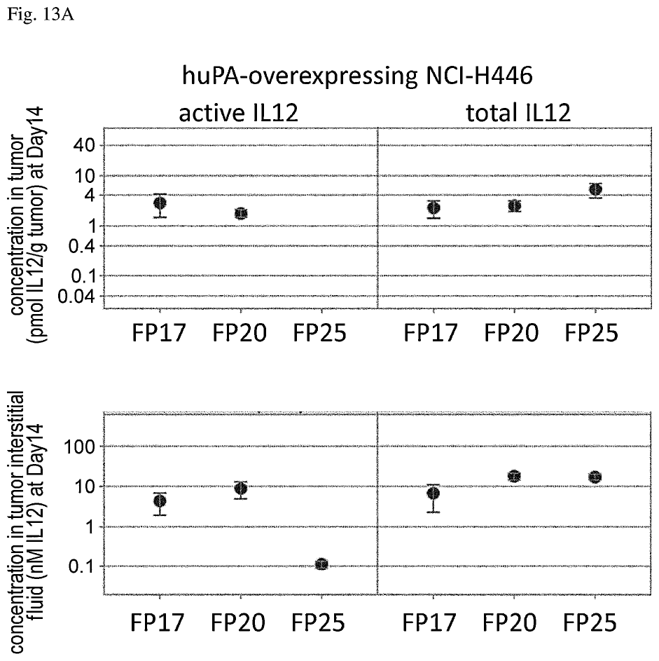

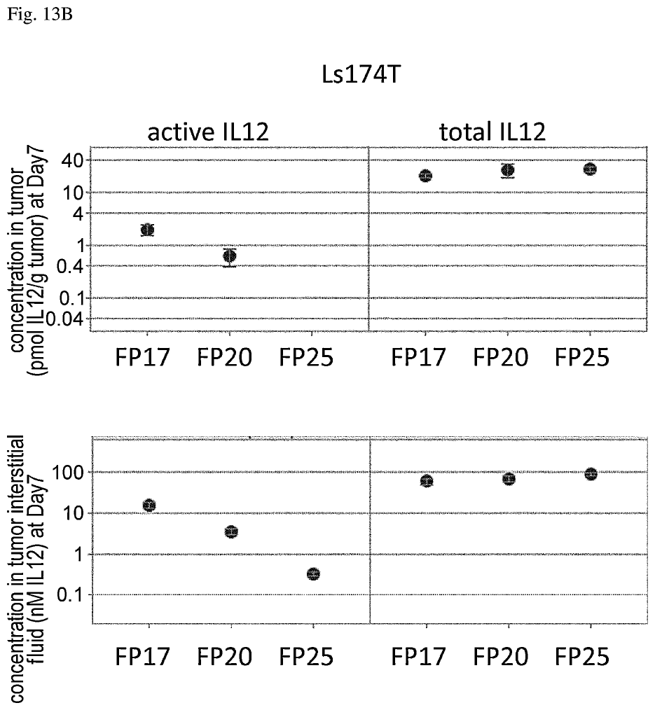

The concentrations of total IL-12 fusion proteins in whole tumor lysate, tumor interstitial fluid and plasma were measured by high-sensitive IL-12 ELISA kit (Abcam). The concentrations of active IL-12 fusion proteins in whole tumor lysate, tumor interstitial fluid and plasma were measured by ELISA assay using the 384-well flat-bottomed plate (Nunc) coated with 1 micro g/mL of Anti-IL12 Antibody (a homodimer made up of light chain (SEQ ID: 121) and heavy chain (SEQ ID: 243), 1 micro g/mL of the biotinylated anti-IL-12 p70 antibody (Abcam), 25 ng/mL of the Poly HRP-labeled streptavidin (Stereospecific Detection Technologies), and the TMB substrate (Surmodics). The absorption intensities were measured at 450 and 650 nm by a CLARIOstar ELISA plate reader (BMG Labtech). Accumulation of active IL-12 in tumor and tumor interstitial fluid was observed for Bivalent IL-12 fusion protein FP17 and Bivalent IL-12 fusion protein FP20, which possess cleavable linker, whereas Bivalent IL-12 fusion protein FP25 which possesses non-cleavable linker did not show detectable active IL-12 accumulation in tumor compared to Bivalent IL-12 fusion protein FP17 and Bivalent IL-12 fusion protein FP20 (FIGS. 13A and 13B).

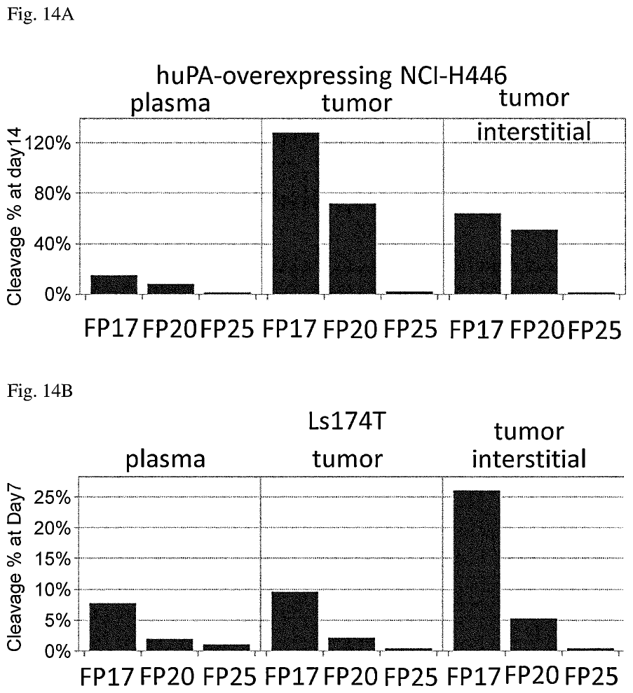

The percentage of cleavage in tumor and plasma was calculated by dividing the concentrations of active IL-12 fusion proteins by the concentrations of total IL-12 fusion proteins (FIGS. 14A and 14B). Bivalent IL-12 fusion protein FP17 and Bivalent IL-12 fusion protein FP20 showed higher percentage of cleavage compared to Bivalent IL-12 fusion protein FP25 which possesses non-cleavable linker. Bivalent IL-12 fusion protein FP17 and Bivalent IL-12 fusion protein FP20 with cleavable linkers showed higher percentage of cleavage in tumor and tumor interstitial fluid compared to plasma.

### Example 5: Screening of IL-12 Fusion Proteins (Also Referenced Herein as Protein Complexes)—IL-12 Variants

The IL-12 in the presently described IL-12 fusion protein binds to the IL-12 binding domain of the fusion protein before protease cleavage and is released to bind IL-12R upon protease cleavage. To prevent non-specific IL-12 digestion by proteases, IL-12 comprising protease resistant modifications are incorporated in the IL-12 fusion proteins. To improve the window between the non-cleaved and cleaved forms of the fusion proteins while maintaining the ability to release VH after protease digestion, various IL-12 variants were screened, including IL-12 with low affinity to IL-12R, i.e. attenuated/low active/low potent IL-12 variants.

### Example 5-1 Preparation of Fusion Proteins (Also Referenced Herein as Protein Complexes) Comprising Low Active IL-12 Variants

Known low active IL-12 variants (IL-12 comprising E59A and F60A mutations reported in Cell, Volume 184, Issue 4, 2021) were introduced in Bivalent IL-12 fusion protein FP14.

Expression vectors of each chain were prepared by a method known to those skilled in the art and expressed using Expi293 (Life Technologies Corp.) by combining each chain as shown in Table 21 and purified by ProA purification method.

TABLE 21
EC50 values with and without protease digestion (Non-cleaved vs Cleaved)
for Bivalent IL-12 fusion proteins (also referenced herein as protein
complexes) with low active IL-12 in IL-12 Luciferase assay

| | | | | EC50 | | Fold |
|---|---|---|---|---|---|---|
| Bivalent IL-12 | Mutations | Light | Heavy | (Non- | EC50 | change |
| fusion protein | on IL12 | chain | chain | cleaved) | (Cleaved) | (window) |
| Bivalent IL-12 | — | SEQ ID | SEQ ID | 12.24 | 0.2839 | 43.11 |
| fusion protein FP14 | | NO: 121 | NO: 207 | | | |
| Bivalent IL-12 | E59A | SEQ ID | SEQ ID | 76.31 | 0.2236 | 341.28 |
| fusion protein FP26 | | NO: 121 | NO: 244 | | | |
| Bivalent IL-12 | F60A | SEQ ID | SEQ ID | 142.0 | 0.3219 | 441.13 |
| fusion protein FP27 | | NO: 121 | NO: 245 | | | |

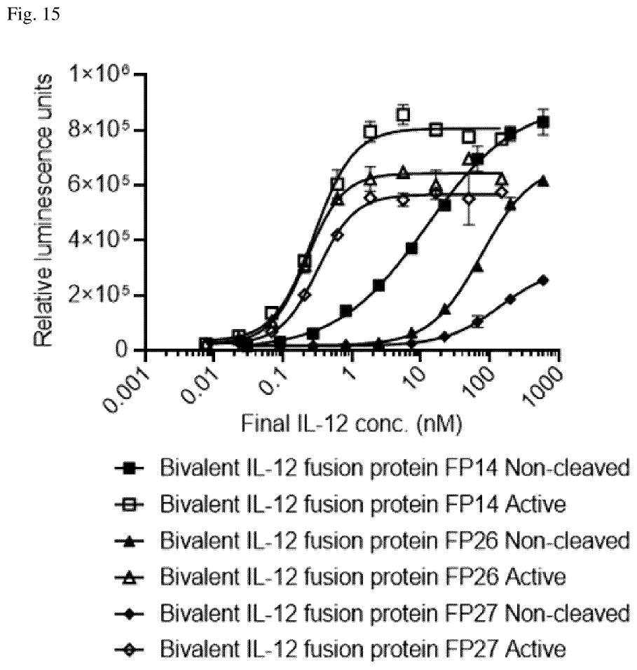

Based on the in vitro activity assessment of Bivalent IL-12 fusion proteins, with and without protease digestion (cleaved and non-cleaved), the proteins comprising IL-12 variants with lower activity (low active IL-12 variants) have improved window between non-cleaved and the cleaved form (FIG. 15 and Table 21).

### Example 5-2 Screening and Preparation of Low Active IL-12 Variants to Improve the Window Between Non-Cleaved and Cleaved (Active) IL-12 Fusion Proteins (Also Referenced Herein as Protein Complexes)

To reduce the activity of IL-12, Y16 position of IL-12 in the Bivalent IL-12 fusion protein was mutated to other possible amino acids. Expression vectors of each chain were prepared by a method known to those skilled in the art and expressed using Expi293 (Life Technologies Corp.) by combining each chain as shown in Table 22. Purification of fusion protein was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 22
Bivalent IL-12 fusion proteins (also referenced herein as
protein complexes) with modifications on Y16 of IL-12.

| Bivalent IL-12 | Mutations | Light | Heavy |
|---|---|---|---|
| fusion protein | on IL12 | chain | chain |
| Bivalent IL-12 | — | SEQ ID | SEQ ID |
| fusion protein FP14 | | NO: 121 | NO: 207 |
| Bivalent IL-12 | Y16F | SEQ ID | SEQ ID |
| fusion protein FP28 | | NO: 121 | NO: 246 |
| Bivalent IL-12 | Y16N | SEQ ID | SEQ ID |
| fusion protein FP29 | | NO: 121 | NO: 247 |
| Bivalent IL-12 | Y16E | SEQ ID | SEQ ID |
| fusion protein FP30 | | NO: 121 | NO: 248 |
| Bivalent IL-12 | Y16P | SEQ ID | SEQ ID |
| fusion protein FP31 | | NO: 121 | NO: 249 |
| Bivalent IL-12 | Y16G | SEQ ID | SEQ ID |
| fusion protein FP32 | | NO: 121 | NO: 250 |
| Bivalent IL-12 | Y16K | SEQ ID | SEQ ID |
| fusion protein FP33 | | NO: 121 | NO: 251 |
| Bivalent IL-12 | Y16A | SEQ ID | SEQ ID |
| fusion protein FP34 | | NO: 121 | NO: 252 |
| Bivalent IL-12 | Y16D | SEQ ID | SEQ ID |
| fusion protein FP35 | | NO: 121 | NO: 253 |
| Bivalent IL-12 | Y16H | SEQ ID | SEQ ID |
| fusion protein FP36 | | NO: 121 | NO: 254 |
| Bivalent IL-12 | Y16I | SEQ ID | SEQ ID |
| fusion protein FP37 | | NO: 121 | NO: 255 |
| Bivalent IL-12 | Y16L | SEQ ID | SEQ ID |
| fusion protein FP38 | | NO: 121 | NO: 256 |
| Bivalent IL-12 | Y16M | SEQ ID | SEQ ID |
| fusion protein FP39 | | NO: 121 | NO: 257 |
| Bivalent IL-12 | Y16Q | SEQ ID | SEQ ID |
| fusion protein FP40 | | NO: 121 | NO: 258 |
| Bivalent IL-12 | Y16R | SEQ ID | SEQ ID |
| fusion protein FP41 | | NO: 121 | NO: 259 |
| Bivalent IL-12 | Y16S | SEQ ID | SEQ ID |
| fusion protein FP42 | | NO: 121 | NO: 260 |
| Bivalent IL-12 | Y16T | SEQ ID | SEQ ID |
| fusion protein FP43 | | NO: 121 | NO: 261 |
| Bivalent IL-12 | Y16V | SEQ ID | SEQ ID |
| fusion protein FP44 | | NO: 121 | NO: 262 |
| Bivalent IL-12 | Y16W | SEQ ID | SEQ ID |
| fusion protein FP45 | | NO: 121 | NO: 263 |
| Bivalent IL-12 | Y16P | SEQ ID | SEQ ID |
| fusion protein FP46 | | NO: 122 | NO: 249 |
| Bivalent IL-12 | Y16P | SEQ ID | SEQ ID |
| fusion protein FP47 | | NO: 121 | NO: 264 |
| Bivalent IL-12 | Y16P | SEQ ID | SEQ ID |
| fusion protein FP48 | | NO: 122 | NO: 264 |

### Example 5-3 Evaluation of the Activity of IL-12 Fusion Proteins (Also Referenced Herein as Protein Complexes) with Low Active IL-12 Variants Using IL-12 Reporter Assay

To assess if the low active IL-12 variants can improve the neutralization of IL-12 in Bivalent IL-12 fusion protein, several low active IL-12 variants shown in Table 22 have been subjected to IL-12 luciferase assay. Briefly, 2.5×10⁴ cells/well IL-12 bioassay cell (Promega, Cat #CS2018A02A) which express human IL-12Rb1, IL-12Rb2, and STAT4, were plated in 96-well plate and incubated overnight. Then, IL-12 fusion proteins were added to the culture plate and incubated for 18 hours. Luciferase activity was detected with Bio-Glo luciferase assay system (Promega, G7940) according to manufacturer's instructions. Luminescence was detected using GloMax (registered trademark) Explorer System (Promega #GM3500). Data analysis was performed using Microsoft (registered trademark) Excel (registered trademark) 2013 and GraphPad Prism ver. 9.0.2. EC50 values were derived using nonlinear regression analysis and Agonist concentration vs. normalized response—Variable slope (four parameter) equation.

TABLE 23
EC50 values with and without protease digestion (Non-
cleaved vs Cleaved) for Bivalent IL-12 fusion proteins
(also referenced herein as protein complexes) with
low active IL-12 variants in IL-12 luciferase assay

| | EC50 | | Fold |
|---|---|---|---|
| Bivalent IL-12 | (Non- | EC50 | change |
| fusion protein | cleaved) | (Cleaved) | (window) |
| Bivalent IL-12 | 7.479 | 0.3252 | 23.0 |
| fusion protein FP14 | | | |
| Bivalent IL-12 | 9.004 | 0.1111 | 81.0 |
| fusion protein FP28 | | | |
| Bivalent IL-12 | 21.81 | 0.1264 | 172.5 |
| fusion protein FP29 | | | |
| Bivalent IL-12 | 17.42 | 0.1016 | 171.5 |
| fusion protein FP30 | | | |
| Bivalent IL-12 | 103.3 | 0.1772 | 583.0 |
| fusion protein FP31 | | | |
| Bivalent IL-12 | 27.71 | 0.1164 | 238.1 |
| fusion protein FP32 | | | |
| Bivalent IL-12 | 10.15 | 0.1268 | 80.0 |
| fusion protein FP33 | | | |
| Bivalent IL-12 | 10.22 | 0.215 | 47.5 |
| fusion protein FP34 | | | |
| Bivalent IL-12 | 21.67 | 0.2415 | 89.7 |
| fusion protein FP35 | | | |
| Bivalent IL-12 | 11.92 | 0.2665 | 44.7 |
| fusion protein FP36 | | | |
| Bivalent IL-12 | 11.69 | 0.2233 | 52.4 |
| fusion protein FP37 | | | |
| Bivalent IL-12 | 12.79 | 0.2132 | 60.0 |
| fusion protein FP38 | | | |
| Bivalent IL-12 | 9.496 | 0.2005 | 47.4 |
| fusion protein FP39 | | | |
| Bivalent IL-12 | 9.768 | 0.2319 | 42.1 |
| fusion protein FP40 | | | |
| Bivalent IL-12 | 7.37 | 0.2877 | 25.6 |
| fusion protein FP41 | | | |
| Bivalent IL-12 | 10.72 | 0.2437 | 44.0 |
| fusion protein FP42 | | | |
| Bivalent IL-12 | 9.126 | 0.2179 | 41.9 |
| fusion protein FP43 | | | |
| Bivalent IL-12 | 5.482 | 0.1619 | 33.9 |
| fusion protein FP44 | | | |
| Bivalent IL-12 | 16.95 | 0.2522 | 67.2 |
| fusion protein FP45 | | | |
| Bivalent IL-12 | 115.8 | 0.232 | 498.3 |
| fusion protein FP46 | | | |
| Bivalent IL-12 | 123.5 | 0.211 | 584.8 |
| fusion protein FP47 | | | |
| Bivalent IL-12 | 123.6 | 0.316 | 390.9 |
| fusion protein FP48 | | | |

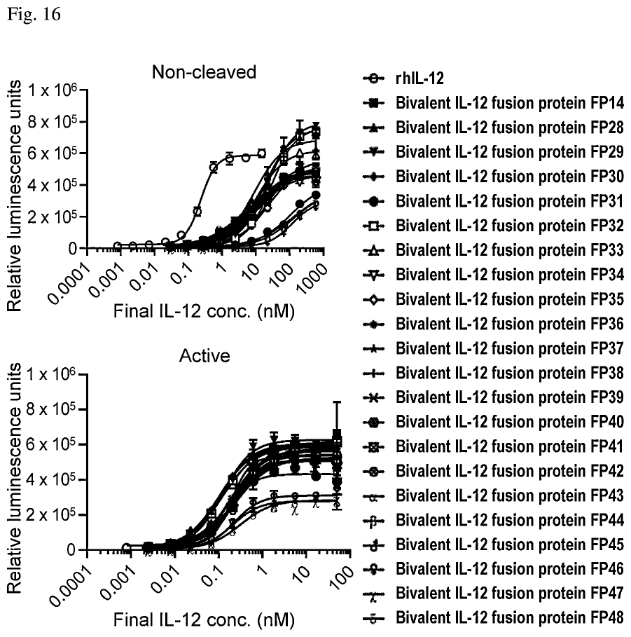

While screening for low active IL-12 variants to be incorporated into the fusion protein, various amino acid positions in IL-12 were selected. Compared to Bivalent IL-12 fusion protein that does not comprise low active IL-12 variant (Bivalent IL-12 fusion protein FP14), all fusion proteins comprising low active IL-12 variants displayed wider fold change in IL-12 activity between non-cleaved and cleaved forms (FIG. 16 and Table 23). In particular, fusion protein FP31 comprising a Y16P mutation in the IL-12 achieved more than 25-fold improvement in window (comparing FP14 vs FP31 in Table 23).

### Example 5-4 Evaluation of Low Active IL-12 Variants as IL-12 Fusion Proteins (Also Referenced Herein as Protein Complexes) Using Human PBMC

To check if the Bivalent IL-12 fusion proteins with low active IL-12 variants identified in Example 5-2 could show improved activity window in primary cells, the activity of several fusion proteins shown in Table 22 was assessed in human peripheral blood mononuclear cells (PBMCs). Briefly, PBMCs (Stem cell technologies #70025) were stimulated with 5 micro g/mL of phytohemagglutinin-L (PHA-L) (Thermofisher Scientific #00-4977-03) at a cell concentration of 1×10⁶/mL for 48 h in culture. Stimulated cells were then harvested and seeded at 5×10⁴ cells/well, before addition of non-cleaved (inactive) or cleaved (active), i.e. human uPA-treated, (huPA; R&D Systems; 1310-SE-010) IL-12 fusion proteins. Culture supernatant was harvested 18 h after incubation and IL-12 activity was determined by detecting IFN gamma levels using an ELISA kit (R&D systems #SIF50C) according to manufacturer's instructions. Optical densities were detected using Multiskan GO (Thermofisher Scientific #N10588). Data analysis was performed using Microsoft (registered trademark) Excel (registered trademark) 2013 and GraphPad Prism ver. 9.0.2. EC50 values were derived using nonlinear regression analysis and Agonist concentration vs. normalized response—Variable slope (four parameter) equation.

TABLE 24
EC50 values with and without protease digestion (Non-cleaved vs Cleaved) for Bivalent
IL-12 fusion proteins (also referenced herein as protein complexes) in PBMC assay

| Bivalent | | Donor 1 | | | Donor 2 | | | Donor 3 | | |
|---|---|---|---|---|---|---|---|---|---|---|
| IL-12 | | EC50 | | Fold | EC50 | | Fold | EC50 | | Fold |
| fusion | | (Non- | EC50 | change | (Non- | EC50 | change | (Non- | EC50 | change |
| protein | Mutation | cleaved) | (Cleaved) | (window) | cleaved) | (Cleaved) | (window) | cleaved) | (Cleaved) | (window) |
| rhIL-12 | — | 6.057 | | | 3.360 | | | 6.206 | | |
| Bivalent | — | 1377 | 14.59 | 94.4 | 1257 | 11.09 | 113.3 | 1866 | 34.00 | 54.9 |
| IL-12 | | | | | | | | | | |
| fusion | | | | | | | | | | |
| protein | | | | | | | | | | |
| FP14 | | | | | | | | | | |
| Bivalent | Y16P | 4753 | 17.56 | 270.7 | 4846 | 20.61 | 235.1 | 8945 | 28.08 | 318.6 |
| IL-12 | | | | | | | | | | |
| fusion | | | | | | | | | | |
| protein | | | | | | | | | | |
| FP31 | | | | | | | | | | |
| Bivalent | E59A | 3209 | 18.50 | 173.5 | 2960 | 24.13 | 122.7 | 5003 | 38.87 | 128.7 |
| IL-12 | | | | | | | | | | |
| fusion | | | | | | | | | | |
| protein | | | | | | | | | | |
| FP26 | | | | | | | | | | |
| Bivalent | F60A | 6954 | 24.62 | 282.5 | 5919 | 34.14 | 173.4 | 7529 | 37.05 | 203.2 |
| IL-12 | | | | | | | | | | |
| fusion | | | | | | | | | | |
| protein | | | | | | | | | | |
| FP27 | | | | | | | | | | |

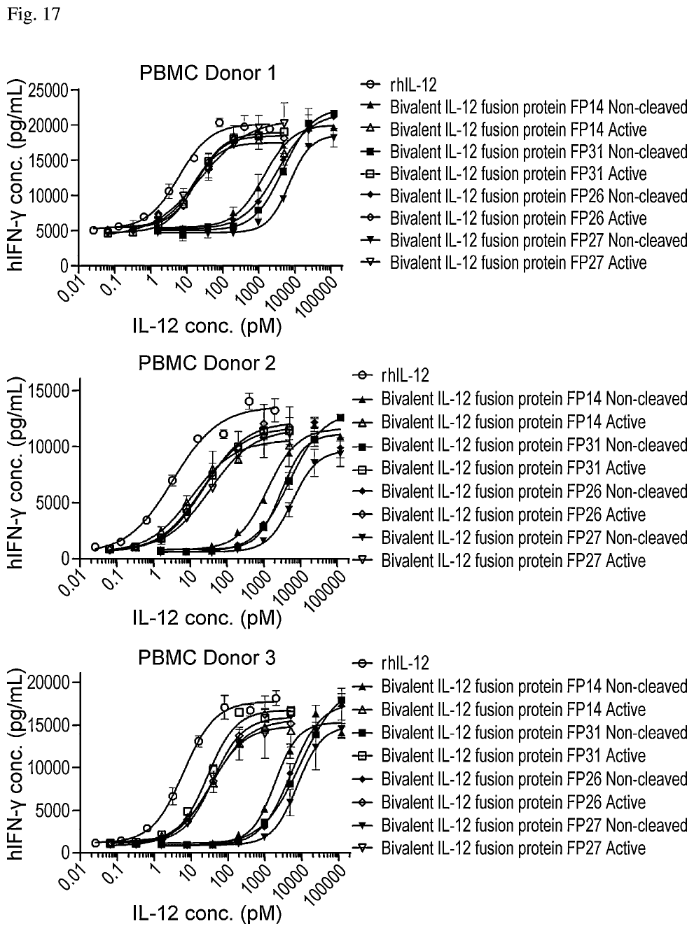

Within each PBMC donor, non-cleaved bivalent IL-12 fusion proteins showed IL-12 neutralization to varying degrees (FIG. 17 and Table 24). The fusion proteins comprising IL-12 variants showed significantly improved fold-change in IL-12 activity window between non-cleaved and cleaved proteins. Such trend was observed across PBMC donors, confirming the reproducibility of JIL-12 fusion type proteins' activity. In particular, fusion protein FP31 comprising a Y16P mutation in the IL-12 achieved up to 5.8-fold improvement in window (comparing FP14 vs FP31 in Table 24).

### Example 6: Evaluation of IL-12 Fusion Proteins (Also Referenced Herein as Protein Complexes) for Accumulation of Active IL-12 in Tumor

### Example 6-1 Preparation of Bivalent IL-12 Fusion Proteins (Also Referenced Herein as Protein Complexes)

Bivalent IL-12 fusion proteins comprising variable region, cleavable linker and IL-12 variants identified in the previous Examples, which are homodimers, each made up of a light chain and a heavy chain as shown in the Table 25 were prepared.

Expression vectors of each chain were prepared by a method known to those skilled in the art and expressed using CHO or Expi293 cells by combining each chain as shown in Table 25. Purification of fusion protein was done using affinity purification by MabSelect SuRe (Cat. No: 17-5438-01, GE Healthcare) followed by different chromatography techniques like ion exchange and/or hydrophobic and/or size exclusion chromatography using Superdex 200 gel filtration column.

TABLE 25
Bivalent IL-12 fusion protein (also referenced herein
as a protein complex) and sequence IDs of each chain.

| Bivalent IL-12 | Light | Heavy |
|---|---|---|
| fusion protein | chain | chain |
| Bivalent IL-12 | SEQ ID | SEQ ID |
| fusion protein FP25 | NO: 202 | NO: 242 |
| Bivalent IL-12 | SEQ ID | SEQ ID |
| fusion protein FP20 | NO: 202 | NO: 208 |
| Bivalent IL-12 | SEQ ID | SEQ ID |
| fusion protein FP22 | NO: 209 | NO: 208 |
| Bivalent IL-12 | SEQ ID | SEQ ID |
| fusion protein FP31 | NO: 121 | NO: 249 |

### Example 6-2 Evaluation of Total and Active IL-12 Fusion Proteins (Also Referenced Herein as Protein Complexes) in Tumor and Plasma

Experimental details are described in Examples 4-3-2 and 4-3-3.

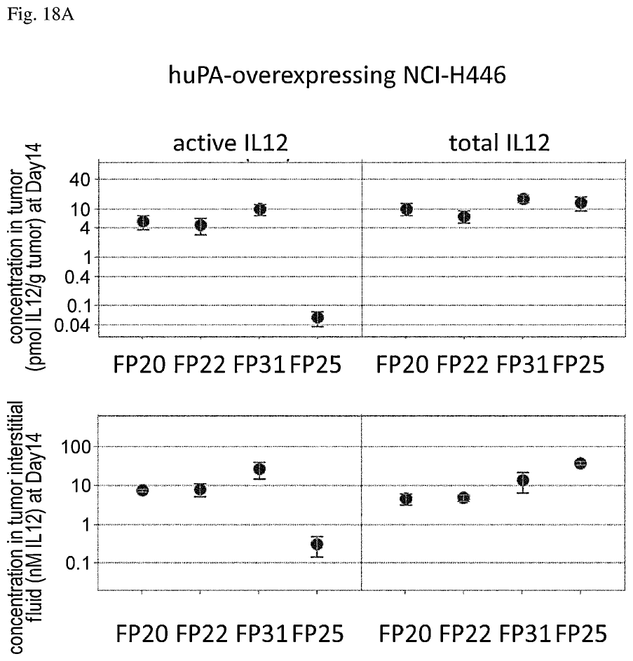

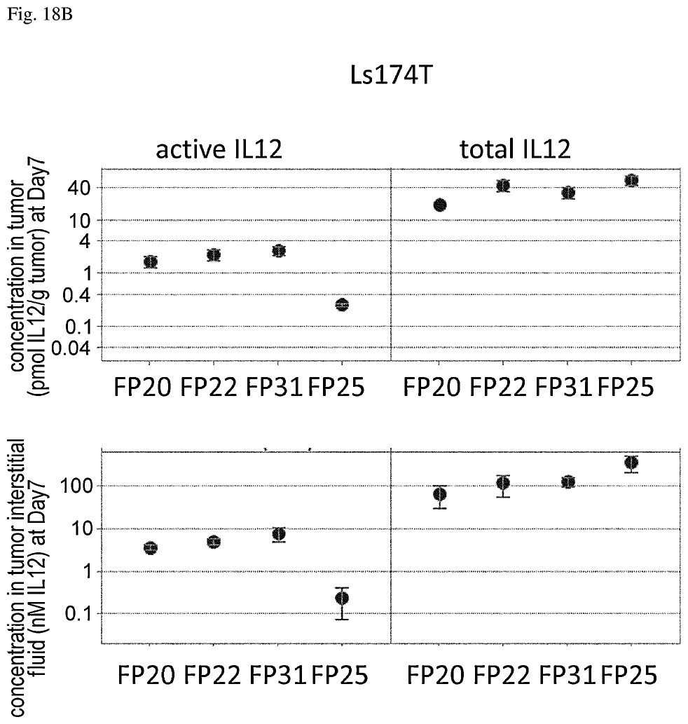

Accumulation of active IL-12 in tumor and tumor interstitial fluid was observed for Bivalent IL-12 fusion protein FP20, Bivalent IL-12 fusion protein FP22 and Bivalent IL-12 fusion protein FP31 which possess cleavable linker, whereas Bivalent IL-12 fusion protein FP25 which possesses non-cleavable linker did not show significant active IL12 accumulation in tumor (FIGS. 18A and 18B).

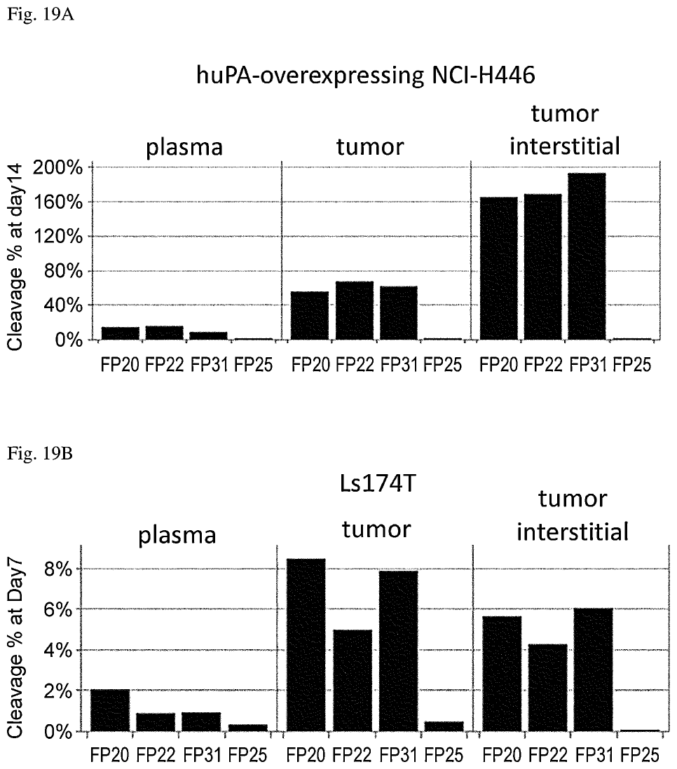

The percentage of cleavage in tumor and plasma was calculated by dividing the concentrations of active IL-12 fusion proteins by the concentrations of total IL-12 fusion proteins (FIGS. 19A and 19B)._Bivalent IL-12 fusion protein FP20, Bivalent IL-12 fusion protein FP22 and Bivalent IL-12 fusion protein FP31 showed higher percentage of cleavage compared to Bivalent IL-12 fusion protein FP25 which possesses non-cleavable linker. Bivalent IL-12 fusion protein FP20, Bivalent IL-12 fusion protein FP22 and Bivalent IL-12 fusion protein FP31 with cleavable linkers showed higher percentage of cleavage in tumor and tumor interstitial fluid compared to plasma.

### Example 7: Evaluation of Systemic Toxicity of IL-12 Fusion Proteins (Also Referenced Herein as Protein Complexes)

### Example 7-1: Preparation of IL-12 Fusion Protein (Also Referenced Herein as a Protein Complex) and IL12 Fc Protein

Bivalent IL-12 fusion protein FP31 comprising variable region, cleavable linker and IL-12 variant identified respectively in each of the previously presented Examples above, which is a homodimer made up of a light chain and a heavy chain as shown in the Table 25, was prepared. IL12 Fc protein FP32 comprising an IL12 fused to an Fc, which is a heterodimer made up of two chains (Chain 1 comprising an Fc fused to IL12 p40 and Chain 2 comprising an Fc fused to IL12 p35) as shown in the Table 35, was prepared.

Expression vectors of each chain were prepared by a method known to those skilled in the art and expressed using CHO or Expi293 cells by combining each chain as shown in Tables 25 and 35. Purification of fusion protein and Fc protein were done using affinity purification by MabSelect SuRe (Cat. No: 17-5438-01, GE Healthcare) followed by different chromatography techniques like ion exchange and/or hydrophobic and/or size exclusion chromatography using Superdex 200 gel filtration column.

TABLE 35
IL-12 Fc protein and sequence IDs of each chain.

| IL-12 | Chain 1 | Chain 2 |
|---|---|---|
| Fc protein | (Fc-IL12 p40) | (Fc-IL12 p35) |
| IL-12 Fc | SEQ ID | SEQ ID |
| protein FP32 | NO: 367 | NO: 368 |

### Example 7-2: Tumor Growth Inhibition In Vivo

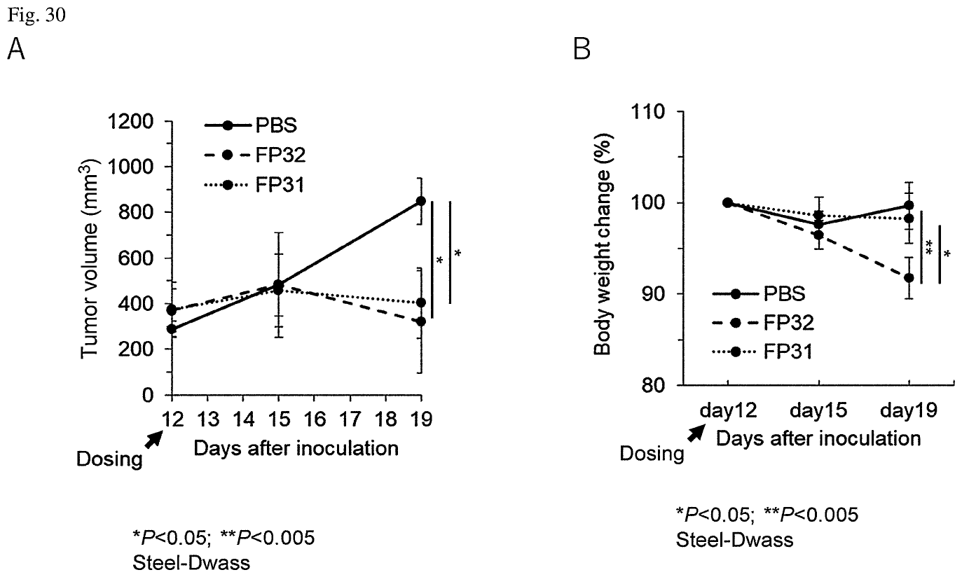

Tumor growth inhibition by treatment with FP31 and FP32 was evaluated. Firstly, human IL12 receptor beta 1 knock-in (human IL12RB1 KI) and human IL12 receptor beta 2 transgenic (IL12RB2 Tg) mice were established to evaluate human IL12 activity in mouse model. The MC38 cell line (Colon38), a colon adenocarcinoma cell line, was used. Colon38 was subcutaneously inoculated on the flank of the genetically engineered mice. Tumor-bearing mice were randomized into three groups, n=5 per group (PBS group, FP31 treatment group, and FP32 treatment group). Mice were treated intravenously with either PBS, FP31, or FP32 on day 12 after tumor inoculation. Tumor growth was assessed over time. FIG. 30A is a graph showing tumor volume (TV (mm³)) vs Days after inoculation. The graph shows group averages of tumor growth+/−standard deviation. Both FP31 and FP32 treatment reduced tumor volume to similar extents in Colon38 tumor-bearing mice, however, as shown in FIG. 30B, while the body weights of the mice in the FP31 treatment group were substantially the same as PBS treatment (control) group, the body weights of the mice in FP32 treatment group were lower than the FP31 and PBS treatment group.

### Example 7-3: Systemic Toxicity In Vivo

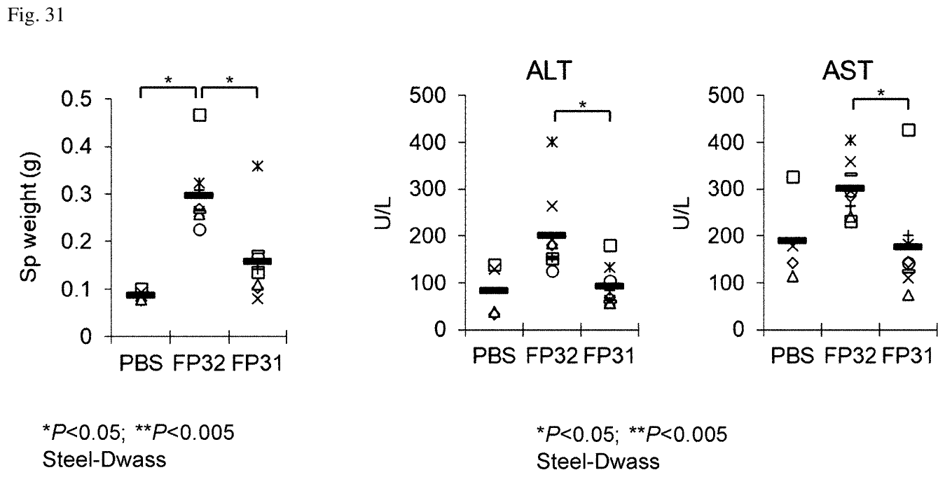

As a measure of systemic toxicity resulting from the administration of FP31 and FP32 treatments, markers indicative of hepatocellular damage, for example, aspartate transaminase (AST) and alanine transaminase (ALT) were assessed in the tumor-bearing mice. Blood sampling was done on day 7 after initiation of FP31 or FP32 treatment. EDTA was used as anti-coagulant. Plasma was prepared by centrifugation and subjected to measurement for AST and ALT. AST and ALT were measured by TBA-120FR; Canon Medical Systems Corporation. Spleen weight was also assessed as another systemic toxicity indicator. As shown in the FIG. 31, while the ALT and AST levels, and spleen weight, were significantly increased in mice of the FP32 treatment group, this ALT and AST elevation, and increase of spleen weight, were not observed in mice of the FP31 treatment group. FP31 clearly demonstrates better therapeutic efficacy (achieving the same reduction in tumor volume without a reduction in body weight) and lower systemic toxicity (no elevation in hepatocellular damage markers) compared to FP32.

### Reference Example 1: VH Release—Engineering VH/VL Interface to Promote Dissociation of VH Upon Protease Digestion

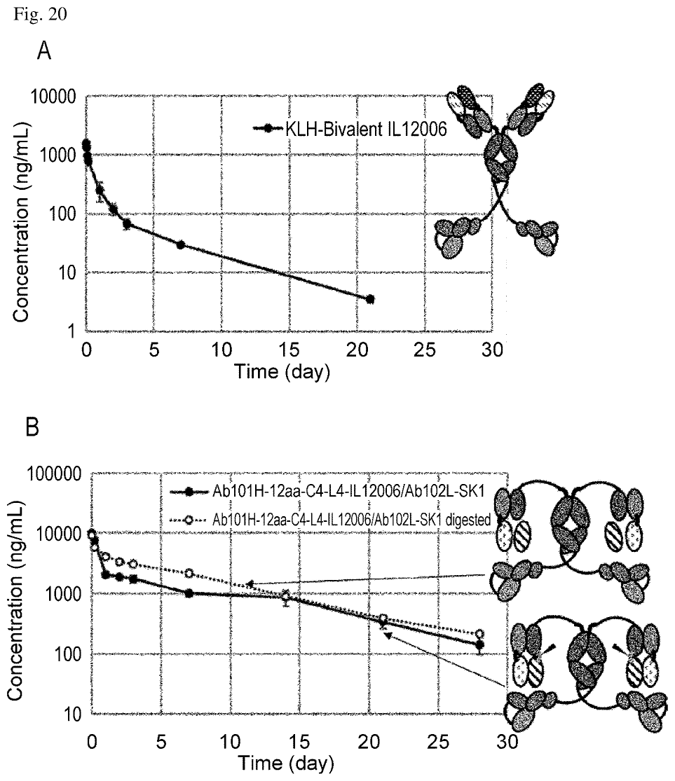

Similar to our observation of rapid elimination in cynomolgus monkey (Example 2), KLH bivalent fusion FP7 showed a clearance of 335 ml/day/kg in SCID mice (FIG. 20A). Meanwhile, in a particular example, it was observed that the activated form of the IL-12 fusion protein (also referenced herein as a protein complex) had a clearance that was comparable to its inactive form (FIG. 20B), and plausibly did not fully dissociate due to remaining avidity between VH domain, VL domain and IL-12. To promote VH release, engineering of the interface between VH and VL, predominantly amino acid modifications in the FR regions, was performed to reduce the association between VH and VL, while not disrupting IL-12 binding to the variable region of the IL-12 fusion protein.

In vitro VH release assay was conducted using BIACORE® T200 instrument (Cytiva) (FIG. 21B). Capture molecule was first immobilized onto all flow cells (FCs) of a sensor chip using amine coupling kit (Cytiva). Next, bivalent fusion proteins were captured onto the flow cells of the sensor surface via the capture protein. This is followed by an injection with recombinant human u-Plasminogen Activator/Urokinase (human uPA, huPA) (R&D Systems; 1310-SE-010) or buffer across the flow cells. Further, the binding affinity of ligand with the ligand binding domain was evaluated to ensure that the amino acid modifications performed to improve VH release did not impact the ability of the ligand binding domain to neutralize the ligand. Finally, the clearance levels of protease activated bivalent fusion protein comprising the amino acid modifications to improve VH release was evaluated.

### 1-1 Preparation of IL-12 Fusion Proteins (Also Referenced Herein as Protein Complexes) Comprising Modifications within the VH/VL Interface

Bivalent IL-12 fusion protein FP8 (Ab101H-12aa-C4-L4-IL12006/Ab102L-SK1) is a homodimer made up of a light chain (SEQ ID NO: 114) and heavy chain (SEQ ID NO: 117). In heavy chain (SEQ ID NO: 117), VH, Ab101H (SEQ ID NO: 116) is fused to the N-terminus of constant region, C4 (SEQ ID NO: 75) via a cleavable linker, 12aa (SEQ ID NO: 46) and single chain IL-12, IL12006 (SEQ ID NO: 113) is fused to the C-terminus of constant region by a GS linker (SEQ ID NO: 68).

Bivalent IL-12 fusion protein FP13 (Ab101H-N0222-C4-L4-IL12v1.KHKE/Ab102L-SK1) is a homodimer made up of a light chain (SEQ ID NO: 114) and heavy chain (SEQ ID NO: 193). In the heavy chain (SEQ ID NO: 193), VH, Ab101H (SEQ ID NO: 116) is fused to the N-terminus of constant region, C4 (SEQ ID NO: 75) via a cleavable linker, N0222 (SEQ ID NO: 194) and single chain IL-12, IL12v1.KHKE (SEQ ID NO: 195) is fused to the C-terminus of constant region by a GS linker (SEQ ID NO: 68). Each of the heavy chains and the light chains were further modified at the VH/VL interface by performing amino acid modification(s) at the VH/VL interface, and optionally additionally with modification(s) in the CDR region. Several fusion proteins of FP8 and FP13 with modifications in the VH/VL interface were examined as shown in Table 27A and Table 27B.

Expression vectors of each chain were prepared by a method known to those skilled in the art and expressed using Expi293 (Life Technologies Corp.) by combining each chain as shown in Table 26. Purification of fusion protein was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 26
Bivalent IL-12 fusion protein (also referenced herein
as a protein complex) and sequence IDs of each chain.

| IL-12 fusion | Light | Heavy | Light | Heavy |
|---|---|---|---|---|
| protein | chain 1 | chain 1 | chain 2 | chain 2 |
| Bivalent IL-12 | SEQ ID | SEQ ID | — | — |
| fusion protein FP8 | NO: 114 | NO: 117 | | |
| Bivalent IL-12 | SEQ ID | SEQ ID | | |
| fusion protein FP13 | NO: 114 | NO: 193 | | |

### 1-2 Screening of VH Release Amino Acid Modifications Using Bivalent IL-12 Fusion Protein (Also Referenced Herein as a Protein Complex) (FP8 and FP13) Variants

VH release for bivalent IL-12 fusion proteins was determined at 37 degrees C. using BIACORE® T200 instrument (Cytiva). Protein A/G (PIERCE) was immobilized onto all flow cells (FCs) of a CM4 sensor chip using amine coupling kit (Cytiva). All proteins and analytes were prepared in HBS-EP+buffer. Each protein was captured onto the sensor surface by Protein A/G captured at flow cells—FC2, FC3, or FC4 to a level of 500 RU, and then followed by 1800 seconds injection of 400 nM of recombinant human uPA or buffer across all FCs. Sensor surface was regenerated after each cycle with 10 mM Glycine-HCl, pH 1.5. The RU value at 10 seconds before the sample injection onto flow cells—FC2, FC3, or FC4 ended was adopted as the final response for each protein. The percentage reduction in RU was calculated using the following formula:

$$\frac{\begin{matrix}Final response of buffer injection - \\ Final response of uPA injection\end{matrix}}{Final response of buffer injection} \times 100$$

As VH corresponds to 10% of the molecular weight of bivalent IL-12 fusion proteins, 10% reduction in response implies that 100% of VH has been released.

$$VH release \% = \% reduction in RU \times 100 / 10$$

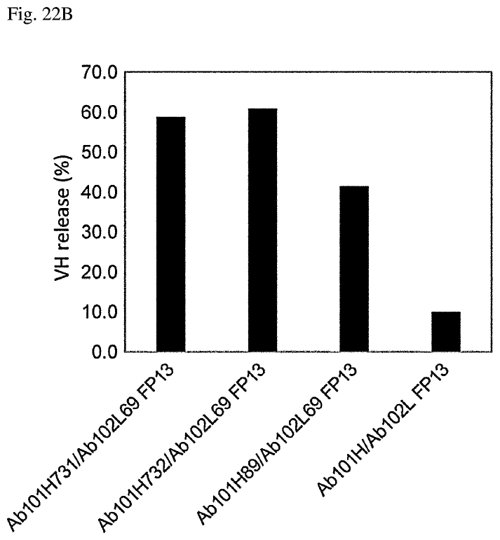

As shown in Tables 27A and 27B and FIGS. 22A and 22B, all tested variants demonstrated improved VH release tendency.

TABLE 27A
Bivalent IL-12 fusion proteins (also referenced herein
as protein complexes) with VH release mutations,
sequence IDs of each chain and the VH release %

| | | % | |
|---|---|---|---|
| | VH/VL | reduction | VH |
| Variant name | mutation | in RU | release % |
| Ab101H/Ab102L | —/— | 1.77 | 17.7 |
| Ab101H10/Ab102L67 | Y91M/A43Q, Y49A | 1.98 | 19.8 |
| Ab101H10/Ab102L80 | Y91M/A43Q, | 2.74 | 27.4 |
| | L46Q, Y49A | | |
| Ab101H15/Ab102L67 | W103M/A43Q, Y49A | 2.19 | 21.9 |
| Ab101H15/Ab102L69 | W103M/L46Q, Y49A | 4.35 | 43.5 |
| Ab101H17/Ab102L67 | V37S/A43Q, Y49A | 2.14 | 21.4 |
| Ab101H17/Ab102L69 | V37S/L46Q, Y49A | 2.81 | 28.1 |
| Ab101H24/Ab102L67 | L45Q/A43Q, Y49A | 2.05 | 20.5 |
| Ab101H24/Ab102L69 | L45Q/L46Q, Y49A | 3.96 | 39.6 |
| Ab101H26/Ab102L67 | F100aI/A43Q, Y49A | 2.02 | 20.2 |
| Ab101H26/Ab102L80 | F100aI/A43Q, | 3.61 | 36.1 |
| | L46Q, Y49A | | |
| Ab101H12/Ab102L103 | W103L/S30V, | 4.00 | 40.0 |
| | L46Q, Y49A | | |
| Ab101H15/Ab102L103 | W103M/S30V, | 3.37 | 33.7 |
| | L46Q, Y49A | | |
| Ab101H89/Ab102L101 | V37S, F100aI/S30V, | 2.51 | 25.1 |
| | A43Q, Y49A | | |
| Ab101H89/Ab102L103 | V37S, F100aI/S30V, | 4.16 | 41.6 |
| | L46Q, Y49A | | |
| Ab101H12/Ab102L69 | W103L/L46Q, Y49A | 4.62 | 46.2 |
| Ab101H13/Ab102L69 | W103I/L46Q, Y49A | 5.69 | 56.9 |
| Ab101H15/Ab102L70 | W103M/Y49A, Y87L | 2.29 | 22.9 |
| Ab101H12/Ab102L70 | W103L/Y49A, Y87L | 2.29 | 22.9 |
| Ab101H12/Ab102L130 | W103L/S30V, | 2.09 | 20.9 |
| | Y49A, Y87L | | |
| Ab101H89/Ab102L69 | V37S, F100aI/ | 5.60 | 56.0 |
| | L46Q, Y49A | | |
| Ab101H89/Ab102L70 | V37S, F100aI/ | 2.62 | 26.2 |
| | Y49A, Y87L | | |
| Ab101H89/Ab102L130 | V37S, F100aI/S30V, | 2.24 | 22.4 |
| | Y49A, Y87L | | |

TABLE 27B
Bivalent IL-12 fusion proteins FP13 (also referenced herein as protein
complexes) with VH release mutations and the VH release %

| | | % | |
|---|---|---|---|
| | VH/VL | reduction | VH |
| Variant | mutation | in RU | release % |
| Ab101H/Ab102L | —/— | 1.00% | 10.0% |
| Ab101H89/Ab102L69 | V37S, F100aI/ | 4.15% | 41.5% |
| | L46Q, Y49A | | |
| Ab101H731/Ab102L69 | V37S, F100aI, | 5.88% | 58.8% |
| | W103M/L46Q, Y49A | | |
| Ab101H732/Ab102L69 | V37S, F100aI, | 6.09% | 60.9% |
| | W103L/L46Q, Y49A | | |

### 1-3 Preparation of Activated Bivalent IL-12 Fusion Proteins (Also Referenced Herein as Protein Complexes) Comprising Amino Acid Modifications at VH/VL Interface

Bivalent IL-12 fusion proteins comprising of amino acid modifications at the VH/VL interface that promotes VH dissociation as described in Table 27A were prepared and additionally subjected to protease treatment. Recombinant Human u-Plasminogen Activator/Urokinase (uPA) (R&D Systems, Inc., 1310-SE-010) was used as the protease. Protease and each bivalent IL-12 fusion protein were reacted in PBS under a condition of 37 degrees C. for 4 hours at 1:1 ratio. The protease was removed from the digestion mixture by pulldown using Ni Sepharose excel resin (Cat. No: 17371201, Cytiva). Table 28 shows the list of variants (VH release variants), VH and VL mutations and sequence IDs.

TABLE 28
Bivalent IL-12 fusion proteins (also referenced herein as
protein complexes) with VH release mutations, sequence
IDs of each chain that were subjected protease digestion

| | VH/VL | Heavy | Light |
|---|---|---|---|
| Variant Name | mutation | chain | Chain |
| Ab101H/Ab102L | —/— | SEQ ID | SEQ ID |
| | | NO: 117 | NO: 114 |
| Ab101H15/Ab102L69 | W103M/L46Q, Y49A | SEQ ID | SEQ ID |
| | | NO: 118 | NO: 121 |
| Ab101H15/Ab102L103 | W103M/S30V, L46Q, | SEQ ID | SEQ ID |
| | Y49A | NO: 118 | NO: 122 |
| Ab101H15/Ab102L70 | W103M/Y49A, Y87L | SEQ ID | SEQ ID |
| | | NO: 118 | NO: 123 |
| Ab101H12/Ab102L69 | W103L/L46Q, Y49A | SEQ ID | SEQ ID |
| | | NO: 119 | NO: 121 |
| Ab101H12/Ab102L103 | W103L/S30V, L46Q, | SEQ ID | SEQ ID |
| | Y49A | NO: 119 | NO: 122 |
| Ab101H15/Ab102L70 | W103L/Y49A, Y87L | SEQ ID | SEQ ID |
| | | NO: 119 | NO: 123 |
| Ab101H15/Ab102L130 | W103L/S30V, Y49A, | SEQ ID | SEQ ID |
| | Y87L | NO: 119 | NO: 124 |
| Ab101H89/Ab102L69 | V37S, F100aI/L46Q, | SEQ ID | SEQ ID |
| | Y49A | NO: 120 | NO: 121 |
| Ab101H89/Ab102L103 | V37S, F100aI/S30V, | SEQ ID | SEQ ID |
| | L46Q, Y49A | NO: 120 | NO: 122 |

### 1-4 Pharmacokinetics of Activated Bivalent IL-12 Fusion Proteins (Also Referenced Herein as Protein Complexes) Comprising Amino Acid Modifications at the VH/VL Interface

### 1-4-1 In Vivo Test Using SCID Mice

The pharmacokinetics of VH release variants (Table 28) were assessed in SCID mice. VH release variants (0.04 mg/mL) were administrated at a single intravenous administration of 10 mL/kg. Blood was collected at 5 minutes, 4 hours, 1 day, 2 days, 3 days, 7 days, 14 days, 21 days, and 28 days after administration. The collected blood was centrifuged immediately at 14000 rpm at 4 degrees C. for 10 minutes to separate the plasma. The separated plasma was stored at below −20 degrees C. until measurement.

### 1-4-2 Measurement of VH Release Variants in SCID Mice Plasma by ELISA

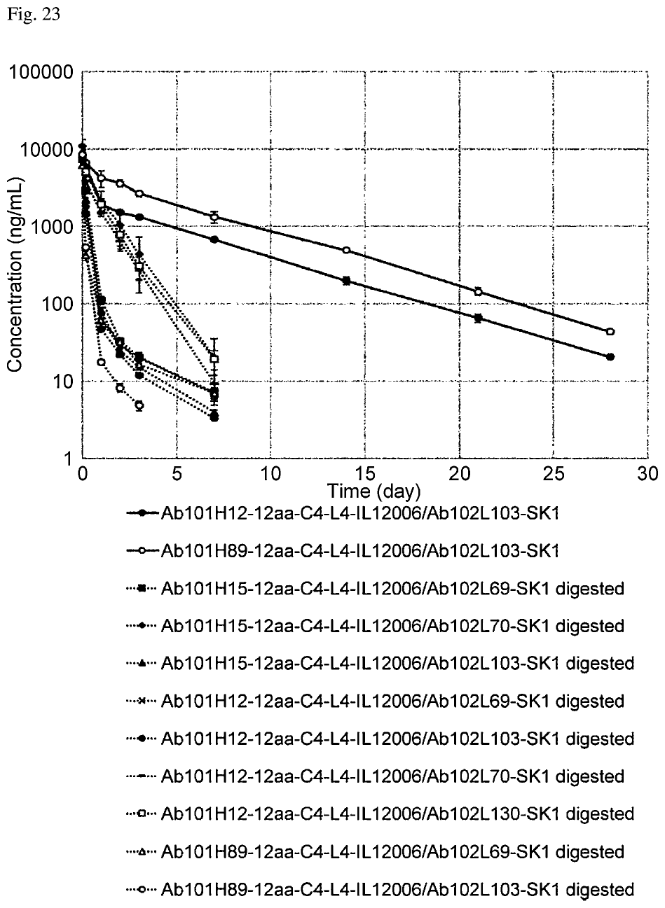

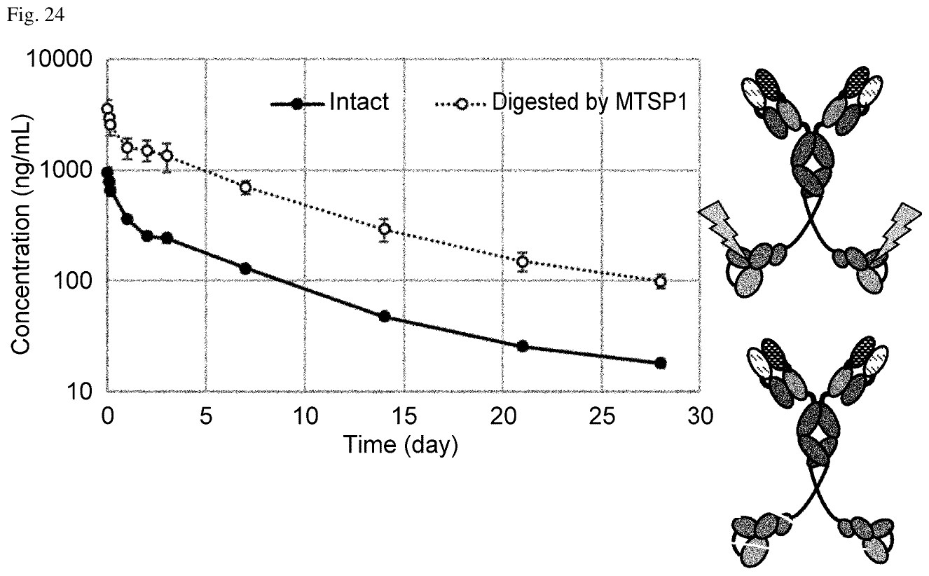

The concentrations of VH release variants in SCID mice plasma were measured by IL-12 High Sensitivity Human ELISA kit (Abcam) according to the manufacturer's instruction. Concentrations of VH release variants were calculated based on the response of the calibration curve using the analytical software SOFTmax PRO (Molecular Devices). The time course of plasma VH release variants concentrations measured by this method is shown in FIG. 23.

### 1-4-3 Pharmacokinetics VH Release Variants in SCID Mice

Pharmacokinetic profile of VH release variants in SCID mice was evaluated. FIG. 23 illustrates the time course of plasma concentrations of VH release variants after intravenous administration in SCID mice. The clearance of non-cleaved bivalent IL-12 fusion proteins, Ab101H12/Ab102L103 and Ab101H89/Ab102L103 was 26.6 and 13.9 mL/day/kg, respectively. Compared with non-cleaved fusion proteins, all VH release variants had higher clearance levels, particularly Ab101H89/Ab102L69 digested and Ab101H89/Ab102L103 digested had the highest clearance among these variants, with 599 and 615 mL/day/kg, respectively (Table 29, FIG. 23). These results indicate that the VH release variants promoted VH dissociation from the fusion protein activated via protease digestion of its cleavable linker. This exposes the two heparin binding sites on the IL-12 which were otherwise masked when it is bound by the ligand-binding domain of the fusion protein. As a result of the amino acid modifications performed to promote VH dissociation, there is quick uptake to extracellular matrix resulting in rapid elimination.

TABLE 29
Pharmacokinetic parameters (ml/day/kg) of activated bivalent IL-12 fusion proteins (also referenced
herein as protein complexes) comprising amino acid modifications at VH/VL interface

| | VH/VL | | AUC_{inf} | CL | Vₛₛ | t_{1/2} | C₀ |
|---|---|---|---|---|---|---|---|
| Variant Name | mutations | | day*ng/mL | mL/day/kg | mL/kg | day | ng/mL |
| Ab101H/Ab102L | —/— | Mean | 27368 | 14.8 | 132 | 5.37 | 10215 |
| | | SD | 4083 | 2.2 | 9 | 0.25 | 893 |
| Ab101H12/Ab102L103 | W103L/S30V, | Mean | 15051 | 26.6 | 137 | 4.31 | 8137 |
| | L46Q, Y49A | SD | 383 | 0.7 | 1 | 0.18 | 139 |
| Ab101H89/Ab102L103 | V37S, F100aI/ | Mean | 28892 | 13.9 | 80.1 | 4.03 | 8324 |
| | S30V, L46Q, | SD | 1993 | 1 | 7.9 | 0.2 | 332 |
| | Y49A | | | | | | |
| Ab101H15/Ab102L69 | W103M/L46Q, | Mean | 1696 | 236 | 137 | 2.38 | 7698 |
| digested | Y49A | SD | 31 | 4 | 12 | 0.07 | 445 |
| Ab101H15/Ab102L103 | W103M/S30V, | Mean | 1481 | 271 | 116 | 1.93 | 9671 |
| digested | L46Q, Y49A | SD | 72 | 13 | 5 | 0.06 | 392 |
| Ab101H15/Ab102L70 | W103M/Y49A, | Mean | 7134 | 59.8 | 61.6 | 0.86 | 11049 |
| digested | Y87L | SD | 2368 | 17 | 8.4 | 0.05 | 2588 |
| Ab101H12/Ab102L69 | W103L/L46Q, | Mean | 1313 | 305 | 189 | 2.44 | 8199 |
| digested | Y49A | SD | 75 | 17 | 18 | 0.23 | 325 |
| Ab101H12/Ab102L103 | W103L/S30V, | Mean | 1039 | 385 | 175 | 1.93 | 7412 |
| digested | L46Q, Y49A | SD | 36 | 13 | 11 | 0.08 | 102 |
| Ab101H12/Ab102L70 | W103L/Y49A, | Mean | 4677 | 85.6 | 90.1 | 0.817 | 6840 |
| digested | Y87L | SD | 194 | 3.6 | 7.6 | 0.029 | 296 |
| Ab101H12/Ab102L130 | W103L/S30V, | Mean | 5946 | 68 | 72.2 | 0.932 | 8176 |
| digested | Y49A, Y87L | SD | 771 | 8.6 | 2.6 | 0.038 | 270 |
| Ab101H89/Ab102L69 | V37S, F100aI/ | Mean | 671 | 599 | 764 | 3.17 | 6469 |
| digested | L46Q, Y49A | SD | 53 | 50 | 316 | 1.68 | 152 |
| Ab101H89/Ab102L103 | V37S, F100aI/ | Mean | 654 | 615 | 132 | 1.07 | 9019 |
| digested | S30V, L46Q, | SD | 54 | 52 | 11 | 0.08 | 1119 |
| | Y49A | | | | | | |
| Ab101H/Ab102L | —/— | Mean | 29102 | 13.8 | 67 | 3.44 | 9871 |
| digested | | SD | 2656 | 1.3 | 5 | 0.25 | 246 |

Engineering performed at the VH/VL interface via modification of the amino acids residing at the interface between VH and VL reduced the association between VH and VL and promoted the dissociation of VH from the bivalent IL12 fusion proteins of the present invention.

### Reference Example 2: Protease Resistant IL-12

Single-chain IL-12 (SEQ ID NO: 67) is made up of p40 (SEQ ID NO: 44) fused to p135 (SEQ ID NO: 45) by GS linker (SEQ ID: 134). It is known that the p40 subunit of IL-12 comprises a heparin binding site which is prone to cleavage by tumor specific proteases, especially Human Matriptase/ST14 Catalytic Domain (MT-SP1). Cleavage can occur in the heparin binding region (SEQ ID NO: 135), between K260 and R261 of p40. Additionally, MT-SP1 cleavage could occur at position Arginine (R) of the N-terminus of p35 (SEQ ID NO: 136) followed by GS linker (SEQ ID: 134) within the single chain IL-12 (SEQ ID NO: 67). In the particular case of Bivalent IL-12 fusion protein FP8, the heparin binding site of IL-12 is in close proximity to the epitope of the variable region of IL-12 fusion protein (SEQ ID NO: 117). Protease cleavage at the heparin binding site affects the fast clearance of activated IL-12 fusion protein of the fusion format (FIG. 24). Selective modifications were performed that prevented unintentional cleavage of IL-12 at the heparin binding site but preserved the epitope of IL-12 fusion protein, maintaining its ability to bind IL-12 in the inactive state of the IL-12 fusion protein.

### 2-1 Preparation of KLH Bivalent IL-12 Fusion Proteins (Also Referenced Herein as Protein Complexes)

KLH Bivalent IL-12 fusion protein FP10 (KLH-Bivalent IL12006v1) is a homodimer made up of a light chain (SEQ ID NO: 91) and a heavy chain (SEQ ID NO: 137). SEQ ID NO: 91 was employed as light chain without modifications. In heavy chain (SEQ ID NO: 137), KLH VH (SEQ ID NO: 99) is fused to constant region (SEQ ID NO: 111) followed by single-chain IL-12 (SEQ ID NO: 138) attached to C-terminal of Fc via GS linker (SEQ ID NO: 68). Several other fusion proteins comprising of FP10 (KLH-Bivalent IL12006v1) and IL-12 variants (“IL-12 variants”) were examined as shown in Table 31.

Expression vectors of each chain were prepared by a method known to those skilled in the art and expressed using Expi293 (Life Technologies Corp.) by combining each chain as shown in Table 30 and Table 31. Purification of proteins was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 30
Bivalent KLH fusion protein (also referenced herein
as a protein complex) and sequence IDs of each chain.

| | Light | Heavy | Light | Heavy |
|---|---|---|---|---|
| KLH fusion protein | chain 1 | chain 1 | chain 2 | chain 2 |
| Bivalent KLH | SEQ ID | SEQ ID | — | — |
| fusion protein FP10 | NO: 91 | NO: 137 | | |

TABLE 31
KLH-Bivalent IL-12 fusion proteins (also referenced herein as
protein complexes) with protease resistant IL-12
mutations, IL-12 variants

| Variant name | Heavy chain | Light Chain | IL-12 mutation |
|---|---|---|---|
| KLH-Bivalent | SEQ ID NO: 137 | SEQ ID NO: 91 | — |
| IL12006v1 | | | |
| KLH-Bivalent | SEQ ID NO: 139 | SEQ ID NO: 91 | KSHRE |
| IL12006v1.KSHRE | | | |
| KLH-Bivalent | SEQ ID NO: 140 | SEQ ID NO: 91 | KSHHE |
| IL12006v1.KSHHE | | | |
| KLH-Bivalent | SEQ ID NO: 141 | SEQ ID NO: 91 | KSHKE |
| IL12006v1.KSHKE | | | |
| KLH-Bivalent | SEQ ID NO: 142 | SEQ ID NO: 91 | KSHSE |
| IL12006v1.KSHSE | | | |
| KLH-Bivalent | SEQ ID NO: 143 | SEQ ID NO: 91 | KSKHRE |
| IL12006v1.KSKHRE | | | |
| KLH-Bivalent | SEQ ID NO: 144 | SEQ ID NO: 91 | KSKQRE |
| IL12006v1.KSKQRE | | | |
| KLH-Bivalent | SEQ ID NO: 145 | SEQ ID NO: 91 | KSKERE |
| IL12006v1.KSKERE | | | |
| KLH-Bivalent | SEQ ID NO: 146 | SEQ ID NO: 91 | KSKPRE |
| IL12006v1.KSKPRE | | | |
| KLH-Bivalent | SEQ ID NO: 147 | SEQ ID NO: 91 | KHKE |
| IL12006v1.KHKE | | | |
| KLH-Bivalent | SEQ ID NO: 148 | SEQ ID NO: 91 | KHHE |
| IL12006v1.KHHE | | | |
| KLH-Bivalent | SEQ ID NO: 149 | SEQ ID NO: 91 | KHRE |
| IL12006v1.KHRE | | | |
| KLH-Bivalent | SEQ ID NO: 150 | SEQ ID NO: 91 | KKHE |
| IL12006v1.KKHE | | | |
| KLH-Bivalent | SEQ ID NO: 151 | SEQ ID NO: 91 | KRHE |
| IL12006v1.KRHE | | | |
| KLH-Bivalent | SEQ ID NO: 152 | SEQ ID NO: 91 | KRE |
| IL12006v1.KRE | | | |
| KLH-Bivalent | SEQ ID NO: 153 | SEQ ID NO: 91 | KHE |
| IL12006v1.KHE | | | |
| KLH-Bivalent | SEQ ID NO: 154 | SEQ ID NO: 91 | KKE |
| IL12006v1.KKE | | | |

### 2-2 Protease Digestion of KLH-Bivalent IL-12 Fusion Proteins (Also Referenced Herein as Protein Complexes)

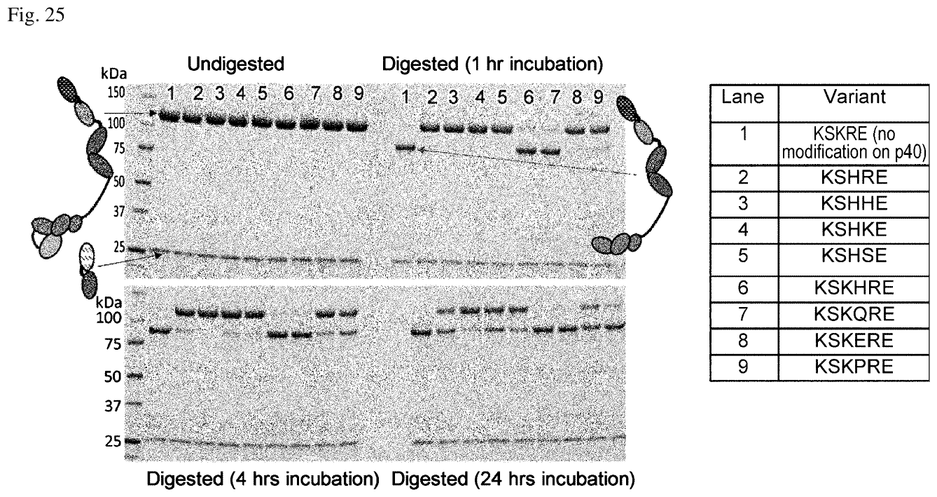

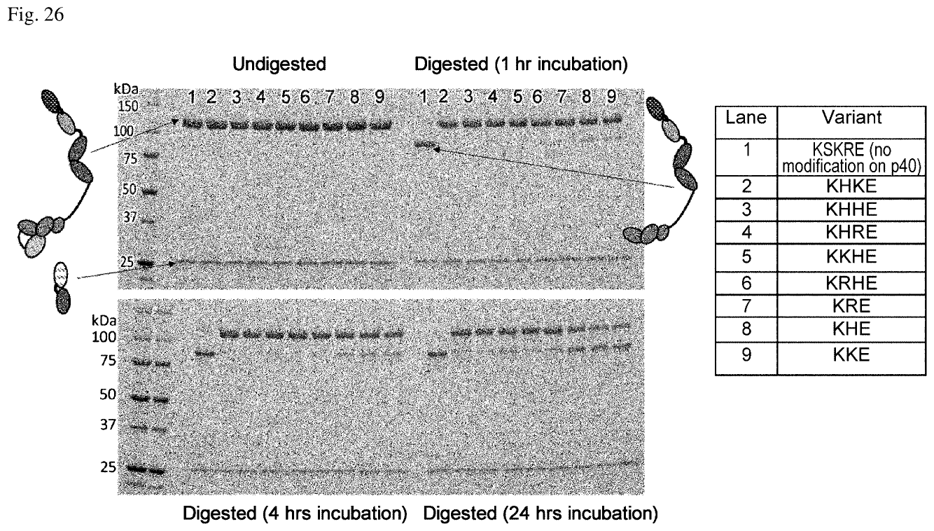

Recombinant Human Matriptase/ST14 Catalytic Domain (MT-SP1) (R&D Systems, Inc., 3946-SE-010) was used as the protease. 75 nM protease and 750 nM of each fusion protein were reacted in PBS under a condition of 37 degrees C. for 1, 4 and 24 hours. Then, cleavage by the protease was evaluated by reducing SDS-PAGE. The results are shown in FIGS. 25 and 26. It is noted that after protease digestion of 1 h and 4 h, there is no change in molecular weight of the heavy chain of most of the fusion proteins comprising the mutations listed in Table 31. This means that most IL-12 variants above were stable and resistant to protease digestion unlike the control (Lane 1). While the results after 1 h and 4 h digestion is considered representative for the expected half-life of the activated form of the bivalent IL-12 fusion protein of the present invention, as a worst case, protease digestion of the IL-12 variants up to 24 h was also performed. As shown, several of these variants (“digested 24 hrs”) still exhibited molecular weights comparable to the start of the incubation (“undigested”) and remain stable despite the long incubation with protease.

### 2-3 Evaluation of In Vitro Activity of IL-12 Variants with Improved Protease Resistance

To assess if the protease resistant modifications affect the activity of IL-12 variants with and without protease treatment, IL-12 luciferase assay was conducted. Briefly, 2.5×10⁴ cells/well IL-12 bioassay cell (Promega, Cat #CS2018A02A) which express human IL-12Rb1, IL-12Rb2, and STAT4, were plated in 96-well plate and incubated overnight. Then, IL-12 or KLH-Bivalent IL-12 fusion proteins (also referenced herein as a protein complex) comprising protease resistant IL-12 (“IL-12 variants”) that were selected from Reference Example 2-2, were added to the culture plate and incubated for 18 hours. The list of fusion proteins are listed in Table 32 below. For protease-treated samples, IL-12 or IL-12 variants were treated with equimolar concentration of MT-SP1 for 4 hours and serial diluents were prepared. Luciferase activity was detected with Bio-Glo luciferase assay system (Promega, G7940) according to manufacturer's instructions. Luminescence was detected using GloMax (registered trademark) Explorer System (Promega #GM3500). Data analysis was done by Microsoft (registered trademark) Excel (registered trademark) 2013 and the analyzed data was plotted using GraphPad Prism 8.4.3.

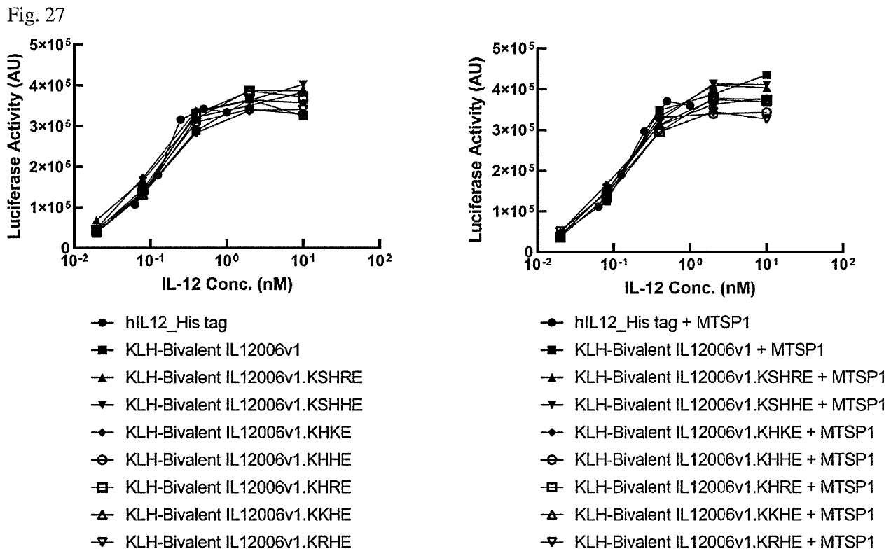

IL-12 activity of protease resistant IL-12 variants was evaluated by the luciferase assay. All the protease resistant IL-12 variants indicated similar activity to hIL12_His tag regardless of protease treatment (FIG. 27).

TABLE 32
Selected IL-12 variants for
Pharmacokinetics analysis

| Variant | Heavy | Light | IL12 |
|---|---|---|---|
| name | chain | Chain | mutation |
| KLH-Bivalent | SEQ ID | SEQ ID | — |
| IL12006v1 | NO: 137 | NO: 91 | |
| KLH-Bivalent | SEQ ID | SEQ ID | KSHRE |
| IL12006v1.KSHRE | NO: 139 | NO: 91 | |
| KLH-Bivalent | SEQ ID | SEQ ID | KSHHE |
| IL12006v1.KSHHE | NO: 140 | NO: 91 | |
| KLH-Bivalent | SEQ ID | SEQ ID | KHKE |
| IL12006v1.KHKE | NO: 147 | NO: 91 | |
| KLH-Bivalent | SEQ ID | SEQ ID | KHHE |
| IL12006v1.KHHE | NO: 148 | NO: 91 | |
| KLH-Bivalent | SEQ ID | SEQ ID | KHRE |
| IL12006v1.KHRE | NO: 149 | NO: 91 | |
| KLH-Bivalent | SEQ ID | SEQ ID | KKHE |
| IL12006v1.KKHE | NO: 150 | NO: 91 | |
| KLH-Bivalent | SEQ ID | SEQ ID | KRHE |
| IL12006v1.KRHE | NO: 151 | NO: 91 | |

### 2-4 Evaluation of Pharmacokinetics of IL-12 Variants with Improved Protease Resistance

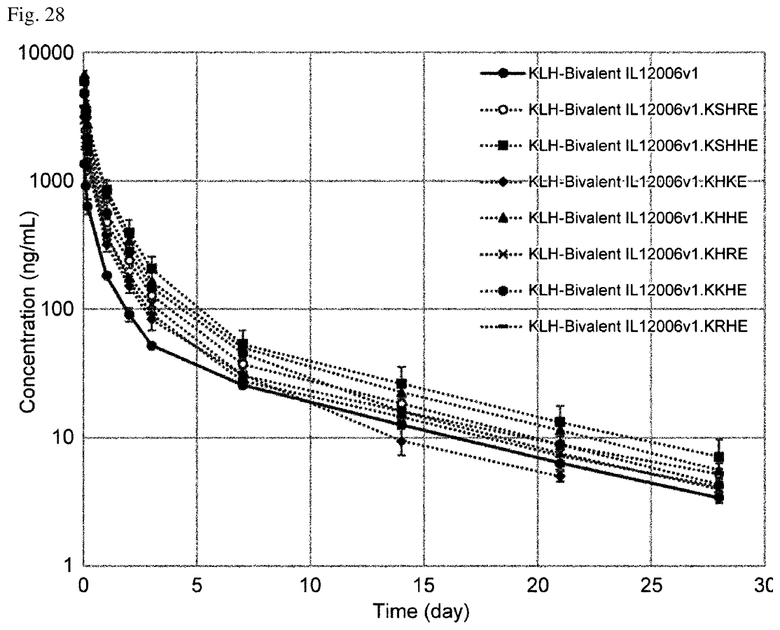

The pharmacokinetics of protease resistant variants were assessed in SCID mice (FIG. 28) Protease resistant variants (0.04 mg/mL) were administrated at a single intravenous administration of 10 mL/kg. Blood was collected at 5 minutes, 2 hours, 4 hours, 1 day, 2 days, 3 days, 7 days, 14 days, 21 days, and 28 days after administration. The collected blood was centrifuged immediately at 14000 rpm at 4 degrees C. for 10 minutes to separate the plasma. The separated plasma was stored at below −20 degrees C. until measurement. The following protease resistant variants shown in Table 32 were tested.

### 2-4-1 Measurement of Protease Resistant Variants in SCID Mice Plasma by ELISA

The concentrations of protease resistant variants in SCID mice plasma were measured by IL-12 High Sensitivity Human ELISA kit (Abcam) according to the instruction. Protease resistant variants concentrations were calculated based on the response of the calibration curve using the analytical software SOFTmax PRO (Molecular Devices). The time course of plasma protease resistant variants concentrations measured by this method is shown in FIG. 28.

### 2-4-2 Pharmacokinetics Protease Resistant Variants in SCID Mice

Pharmacokinetic profile of protease resistant variants in SCID mice was evaluated. FIG. 28 illustrates the time course of plasma concentrations of protease resistant IL-12 variants after intravenous administration in SCID mice. With reference to FIG. 28 and Table 33, all protease resistant variants demonstrated slower elimination than control (KLH-Bivalent IL12006v1) having a clearance level of 376 mL/day/kg. Amongst these, fusion proteins variants KLH-Bivalent IL12006v1.KHKE and KLH-Bivalent IL12006v1.KRHE showed the highest clearance levels of 241 and 206 mL/day/kg respectively. These variants exemplified a pharmacokinetic profile that demonstrated both protease resistance while retaining high clearance levels.

TABLE 33
Pharmacokinetic parameters (ml/day/kg) of activated bivalent
KLH fusion proteins (also referenced herein as protein
complexes) comprising protease resistant mutations

| | | AUC_{inf} | CL | Vₛₛ | t_{1/2} | C₀ |
|---|---|---|---|---|---|---|
| Variant Name | | day*ng/mL | mL/day/kg | mL/kg | day | ng/mL |
| KLH-Bivalent | Mean | 1065 | 376 | 2006 | 7.13 | 1391 |
| IL12006v1 | SD | 43 | 15 | 235 | 0.53 | 231 |
| KLH-Bivalent | Mean | 2511 | 160 | 612 | 7.73 | 4896 |
| IL12006v1.KSHRE | SD | 159 | 10 | 58 | 0.32 | 553 |
| KLH-Bivalent | Mean | 3617 | 112 | 419 | 7.44 | 6158 |
| IL12006v1.KSHHE | SD | 563 | 17 | 61 | 0.86 | 1223 |
| KLH-Bivalent | Mean | 1672 | 241 | 705 | 4.71 | 3228 |
| IL12006v1.KHKE | SD | 176 | 24 | 58 | 0.12 | 215 |
| KLH-Bivalent | Mean | 3646 | 110 | 350 | 6.63 | 6817 |
| IL12006v1.KHHE | SD | 195 | 6 | 53 | 0.21 | 614 |
| KLH-Bivalent | Mean | 1944 | 206 | 806 | 6.87 | 3061 |
| IL12006v1.KHRE | SD | 92 | 10 | 96 | 0.17 | 356 |
| KLH-Bivalent | Mean | 2865 | 140 | 464 | 7.06 | 4940 |
| IL12006v1.KKHE | SD | 216 | 10 | 88 | 0.94 | 583 |
| KLH-Bivalent | Mean | 2003 | 200 | 735 | 7.47 | 3885 |
| IL12006v1.KRHE | SD | 49 | 5 | 25 | 0.43 | 306 |

### Reference Example 3: Biological Activity of IL-12 Fusion Proteins (Also Referenced Herein as Protein Complexes) Restored Upon Protease Activation

### 3-1 Preparation of IL-12 Fusion Proteins (Also Referenced Herein as Protein Complexes) with Selected Amino Acid Modifications at VH/VL Interface and Protease Resistant IL-12 Modifications

The ability of IL-12 obtained in Reference Example 2 to bind the ligand-binding domain obtained in Reference Example 1 of the fusion protein of the present invention was evaluated. Bivalent IL-12 fusion protein FP8 (Ab101H-12aa-C4-L4-IL12006/Ab102L-SK1) is a homodimer made up of a light chain (SEQ ID NO: 114) and heavy chain (SEQ ID NO: 117). SEQ ID NO: 114 was employed as light chain with modification(s) in the VH/VL interface. In heavy chain (SEQ ID NO: 117), VH, Ab101H (SEQ ID NO: 116) is fused to the N-terminus of constant region, C4 (SEQ ID NO: 75) via a cleavable linker, 12aa (SEQ ID NO: 46) and single chain IL-12, IL12006 (SEQ ID NO: 113) is fused to the C-terminus of constant region by a GS linker (SEQ ID NO: 68). Ligand-binding domains (Ab101H89/Ab102L69 and Ab101H89/Ab102L103) which have shown improved VH dissociation from the fusion protein after protease cleavage in Reference Example 2 were selected. Single-chain IL-12 variant obtained in Reference Example 3 was attached to C-terminal of Fc domain via GS linker (L4, SEQ ID NO: 68). Several fusion proteins comprising of FP8, FP11 and FP12 with/without protease resistant IL-12 variants were produced (Table 34).

Expression vectors of each chain were prepared by a method known to those skilled in the art and expressed using Expi293 (Life Technologies Corp.) by combining each chain as shown in Table 34. Purification of proteins was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 34
IL-12 fusion proteins (also referenced
herein as protein complexes) with
protease resistant IL-12 variants

| Bivalent IL-12 | Heavy | Light | IL 12 |
|---|---|---|---|
| fusion protein | chain | chain | mutation |
| Bivalent IL-12 | SEQ ID | SEQ ID | - |
| fusion protein | NO: 117 | NO: 114 | |
| FP8 | | | |
| Bivalent IL-12 | SEQ ID | SEQ ID | KHKE |
| fusion protein | NO: 155 | NO: 121 | |
| FP11 | | | |
| Bivalent IL-12 | SEQ ID | SEQ ID | KHKE |
| fusion protein | NO: 155 | NO: 122 | |
| FP12 | | | |

### 3-2 Evaluation of In Vitro Activity of IL-12 Fusion Type Fusion Proteins (Also Referenced Herein as Protein Complexes) with Protease Resistant IL-12

To assess if the protease resistant modifications affect the binding of IL-12 to the IL-12 fusion proteins of the present invention, IL-12 luciferase assay was conducted. Briefly, 2.5×10⁴ cells/well IL-12 bioassay cell (Promega, Cat #CS2018A02A) which express human IL-12Rb1, IL-12Rb2, and STAT4, were plated in 96-well plate (#Corning, #3917). Then, the IL-12 fusion proteins were added to the culture plate and incubated for 18 hours. For protease-treated samples, fusion proteins were treated with equimolar concentration of MT-SP1 for 4 hours and serial diluents were prepared. Luciferase activity was detected with Bio-Glo luciferase assay system (Promega, G7940) according to manufacturer's instructions. Luminescence was detected using GloMax (registered trademark) Explorer System (Promega #GM3500). Data analysis was done by Microsoft (registered trademark) Excel (registered trademark) 2013 and the analyzed data was plotted using GraphPad Prism ver. 9.0.2.

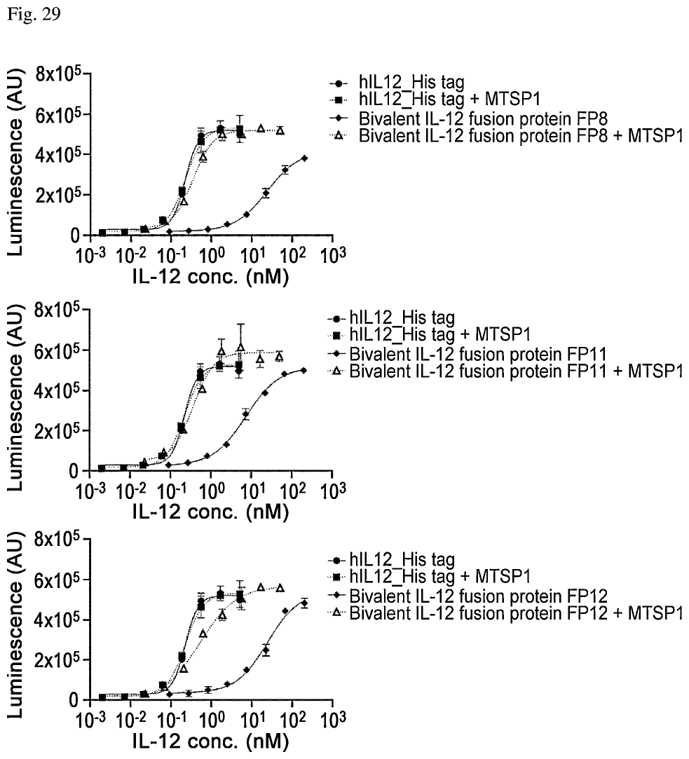

Bivalent IL-12 fusion proteins FP8, FP11 and FP12 were subjected to the IL-12 luciferase assay. All three fusion proteins showed lower IL-12 bioactivity than hIL-12_His tag in the absence of MT-SP1, and the IL-12 bioactivity was restored to the same level as hIL-12_His tag upon MT-SP1 treatment (FIG. 29).

### Reference Example 4: Biological Activity of Bivalent IL-12 Fusion Proteins (Also Referenced Herein as Protein Complexes) Restored Upon Protease Activation

### 4-1 Preparation of Bivalent IL-12 Fusion Proteins (Also Referenced Herein as Protein Complexes)

Bivalent IL-12 fusion protein FP11 (Ab101H89-12aa-C4-L4-IL12006v1.KHKE//Ab102L69-SK1) is a homodimer made of a light chain (SEQ ID NO: 121) and a heavy chain (SEQ ID NO: 155), comprising a de-glycosylated protease resistant single-chain IL-12, IL12006v1.KHKE (SEQ ID NO: 181).
Bivalent IL-12 fusion protein FP14 (Ab101H89-12aa0054-C4-L4-IL12v1.KHKE/Ab102L69-SK1) is a homodimer made up of a light chain (SEQ ID NO: 121) and a heavy chain (SEQ ID NO: 207), comprising a wild type (WT) protease resistant single-chain IL-12, IL12v1.KHKE (SEQ ID NO: 317).

Expression vectors of each chain were prepared by a method known to those skilled in the art and expressed using Expi293 (Life Technologies Corp.) by combining each chain as shown in Table 36. Purification of the fusion protein was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 36
IL-12 fusion proteins with Wild Type
IL-12 (WT IL-12) or De-glycosylated
IL-12 (IL12006v1)

| Bivalent IL-12 | Heavy | Light | |
|---|---|---|---|
| fusion protein | chain | chain | IL12 |
| Bivalent IL-12 | SEQ ID | SEQ ID | IL12006v1.KHKE |
| fusion protein | NO: 155 | NO: 121 | (SEQ ID |
| FP11 | | | NO: 181) |
| Bivalent IL-12 | SEQ ID | SEQ ID | IL12v1.KHKE |
| fusion protein | NO: 207 | NO: 121 | (SEQ ID |
| FP14 | | | NO: 317) |

### 4-2 Evaluation of In Vitro Activity of Bivalent IL-12 Fusion Proteins (Also Referenced Herein as Protein Complexes)

To determine if the glycosylation status of IL-12 affects the activity of IL-12 fusion proteins in the present invention, IL-12 luciferase assay was conducted. Briefly, 2.5×10⁴ cells/well IL-12 bioassay cell (Promega, Cat #CS2018A02A) which express human IL-12Rb1, IL-12Rb2, and STAT4, were plated in 96-well plate (#Corning, #3917). Then, the IL-12 fusion proteins were added to the culture plate and incubated for 18 hours. For protease-treated samples, fusion proteins were treated with equimolar concentration of uPA for 4 hours and serial diluents were prepared. Luciferase activity was detected with Bio-Glo luciferase assay system (Promega, G7940) according to manufacturer's instructions. Luminescence was detected using GloMax (registered trademark) Explorer System (Promega #GM3500). Data analysis was done by Microsoft (registered trademark) Excel (registered trademark) 2013 and the analyzed data was plotted using GraphPad Prism ver. 9.0.2.

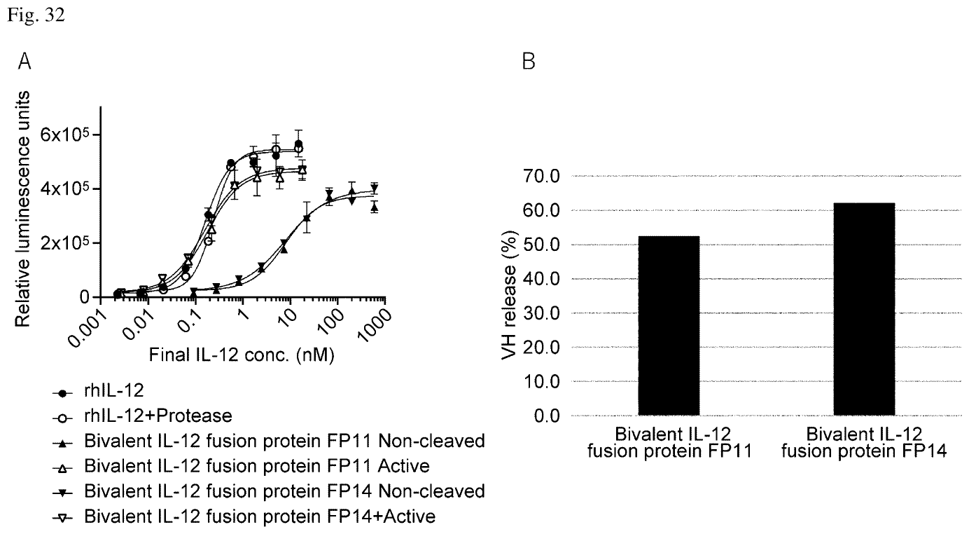

As shown in FIG. 32A, the results showed that there was no significant difference in activity between fusion proteins with de-glycosylated IL-12 and fusion proteins with WT IL-12. Similar activities were also observed in both non-cleaved and protease treated active conditions, suggesting both neutralization and activity of IL-12 were unaffected by IL-12 glycosylation status.

### 4-3 Evaluation of VH Release of Bivalent IL-12 Fusion Proteins (Also Referenced Herein as Protein Complexes)

VH release for bivalent IL-12 fusion proteins was determined at 37 degrees C. using BIACORE® T200 instrument (Cytiva). Protein A/G (PIERCE) was immobilized onto all flow cells (FCs) of a CM4 sensor chip using amine coupling kit (Cytiva). All proteins and analytes were prepared in HBS-EP+buffer. Each protein was captured onto the sensor surface by Protein A/G captured at flow cells—FC2, FC3, or FC4 to a level of 500 RU, and then followed by 1800 seconds injection of 400 nM of recombinant human uPA or buffer across all FCs. Sensor surface was regenerated after each cycle with 10 mM Glycine-HCl, pH 1.5. The RU value at 10 seconds before the sample injection onto flow cells—FC2, FC3, or FC4 ended was adopted as the final response for each protein. The percentage reduction in RU was calculated using the following formula:

$$\frac{\begin{matrix}Final response of buffer injection - \\ Final response of uPA injection\end{matrix}}{Final response of buffer injection} \times 100$$

As VH corresponds to 10% of the molecular weight of bivalent IL-12 fusion proteins, 10% reduction in response implies that 100% of VH has been released.

$$VH release \% = \% reduction in RU \times 100 / 10$$

As shown in FIG. 32B, both fusion proteins with de-glycosylated IL-12 and fusion protein with WT IL-12 showed more than 50% VH release, demonstrating that IL-12 glycosylation status does not affect the release of VH and thereby activation of the fusion protein upon protease treatment.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

What is claimed is:

1. An attenuated IL-12 polypeptide comprising a p40 subunit and a p35 subunit, wherein the p40 subunit comprises an asparagine (N), glutamic acid (E), glycine (G) or proline (P) residue at the amino acid position corresponding to residue 16 of SEQ ID NO: 44.

2. The attenuated IL-12 polypeptide of claim 1 characterized by one or more of
(a) wherein the p40 subunit does not comprise the amino acid sequence of KSKREK (SEQ ID NO: 197), or KSKRE (SEQ ID NO: 361);
(b) wherein the attenuated IL-12 polypeptide is a single chain IL-12 polypeptide; and
(c) wherein the attenuated IL-12 polypeptide comprises the amino acid sequence of SEQ ID NO: 325 or 335.

3. A fusion protein or protein complex comprising the attenuated IL-12 polypeptide of claim 1.

4. A polynucleotide encoding the attenuated IL-12 polypeptide of claim 1.

5. A recombinant host cell comprising a polynucleotide encoding the attenuated IL-12 polypeptide of claim 1.

6. A method of treating a disease or disorder in an individual in need thereof, comprising administering to said individual a therapeutically effective amount of the attenuated IL-12 polypeptide of claim 1.

7. A protein complex comprising two subunits associated with each other, wherein each subunit comprises a first polypeptide and a second polypeptide, wherein the first polypeptide comprises a light chain variable domain (VL) and a light chain constant domain (CL), and the second polypeptide is a fusion protein represented by the general formula (I), from the N- to the C-terminus: 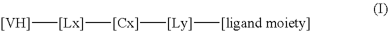
wherein:
VH represents a heavy chain variable domain (VH), wherein the VH is associated with the VL of the first polypeptide to form a ligand-binding domain that binds to the ligand moiety,
Lx represents a cleavable peptide linker comprising a protease cleavage site,
Cx represents a constant domain associated with the CL of the first polypeptide,
wherein Cx comprises, from the N- to the C-terminus, a CH1 domain, a second peptide linker, a CH2 domain, and a CH3 domain,
Ly represents a third peptide linker, and
the ligand moiety is a protease resistant attenuated single-chain IL-12 fusion protein comprising a p40 subunit and a p35 subunit, wherein the p40 subunit comprises an asparagine (N), glutamic acid (E), glycine (G) or proline (P) residue at the amino acid position corresponding to residue 16 of SEQ ID NO: 44, and does not comprise the amino acid sequence of KSKREK (SEQ ID NO: 197), or KSKRE (SEQ ID NO: 361).

8. The protein complex of claim 7, wherein the protease resistant single-chain IL-12 fusion protein comprises the amino acid sequence of SEQ ID NO: 325 or 335.

9. The protein complex of claim 7, wherein
(a) the VH and VL comprises HCDR1, HCDR2, HCDR3, LCDR1, LCDR2, and LCDR3 comprising SEQ ID NO: 346, 348, 350, 352, 353, and 355, respectively, or SEQ ID NO: 346, 347, 349, 351, 353, and 355, respectively, or SEQ ID NO: 346, 347, 349, 351, 354, and 355, respectively; or SEQ ID NO: 346, 348, 350, 351, 353, and 355, respectively, or SEQ ID NO: 346, 348, 350, 351, 354, and 355, respectively, or SEQ ID NO: 346, 347, 349, 352, 353, and 355, respectively; or
(b) the VH and VL comprises SEQ ID NO: 189 and 190, respectively, or SEQ ID NO: 340 and 343, respectively, or SEQ ID NO: 340 and 344, respectively, or SEQ ID NO: 341 and 344, respectively, or SEQ ID NO: 189 and 342, respectively, or SEQ ID NO: 189 and 191, respectively.

10. The protein complex of claim 7 characterized by one or more of
(a) wherein Lx comprises the amino acid sequence of SEQ ID NO: 194 or 199;
(b) wherein Cx comprises the amino acid sequence of SEQ ID NO: 75; and
(c) wherein Ly comprises the amino acid sequence of SEQ ID NO: 68.

11. The protein complex of claim 7 comprising two first polypeptides and two second polypeptides, wherein the first and second polypeptide comprises the amino acid sequence of SEQ ID NO: 249 and 121, respectively; or SEQ ID NO: 249 and 122, respectively; or SEQ ID NO: 264 and 121, respectively; or SEQ ID NO: 264 and 122, respectively; or SEQ ID NO: 247 and 121, respectively; or SEQ ID NO: 248 and 121, respectively; or SEQ ID NO: 250 and 121, respectively.

12. A polynucleotide or a plurality of polynucleotides encoding the protein complex of claim 7.

13. A method of treating a disease or disorder in an individual in need thereof, comprising administering to said individual a therapeutically effective amount of the protein complex of claim 7.

14. An antibody that binds to human IL-12 and comprises:
(a) a HCDR1 of SEQ ID NO: 346;
(b) a HCDR2 of SEQ ID NO: 347 or 348;
(c) a HCDR3 of SEQ ID NO: 349 or 350;
(d) a LCDR1 of SEQ ID NO: 351 or 352;
(e) a LCDR2 of SEQ ID NO: 353 or 354; and
(f) a LCDR3 of SEQ ID NO: 355,
wherein the HCDR1, HCDR2, HCDR3, LCDR1, LCDR2 and LCDR3 does not comprise SEQ ID NO: 346, 348, 350, 352, 353 and 355, respectively.

15. The antibody of claim 14, wherein the HCDR1, HCDR2, HCDR3, LCDR1, LCDR2 and LCDR3 comprises SEQ ID NO: 346, 347, 349, 351, 353 and 355, respectively, or SEQ ID NO: 346, 347, 349, 351, 354 and 355, respectively.

16. The antibody of claim 14, wherein
(a) the VH comprises SEQ ID NO: 189, 340 or 341 and the VL comprises SEQ ID NO: 190, 191, 342, 343 or 344, wherein the VH and VL does not comprise SEQ ID NO: 189 and 190, respectively; or
(b) wherein the VH and VL comprises SEQ ID NO: 189 and 342, respectively, SEQ ID NO: 340 and 343, respectively, SEQ ID NO: 340 and 344, respectively, SEQ ID NO: 341 and 344, respectively, or SEQ ID NO: 189 and 191, respectively.

17. The antibody of claim 14 which comprises an IgG antibody, IgG1 antibody, scFv, single-chain antibody, Fv, scFv2, Fab or F(ab′)2.

18. A fusion protein or protein complex comprising the antibody of claim 14 and an IL-12 polypeptide.

19. A polynucleotide encoding the antibody of claim 14.

20. A method of treating a disease or disorder in an individual in need thereof, comprising administering to said individual a therapeutically effective amount of the antibody of claim 14.

21. A protein complex comprising two subunits associated with each other, wherein each subunit comprises a first polypeptide and a second polypeptide, wherein the first polypeptide comprises a light chain variable domain (VL) and a light chain constant domain (CL), and the second polypeptide is a fusion protein represented by the general formula (I), from the N- to the C-terminus: 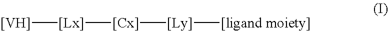
wherein:
VH represents a heavy chain variable domain (VH), wherein the VH is associated with the VL of the first polypeptide to form an affinity matured ligand-binding domain that binds to the ligand moiety, and wherein the VH and VL comprises the VH and VL of the antibody according to claim 16,
Lx represents a cleavable peptide linker comprising a protease cleavage site,
Cx represents a constant domain associated with the CL of the first polypeptide,
wherein Cx comprises, from the N- to the C-terminus, a CH1 domain, a second peptide linker, a CH2 domain, and a CH3 domain,
Ly represents a third peptide linker, and
the ligand moiety is a protease resistant single-chain IL-12 fusion protein.

22. The protein complex of claim 21 characterized by one or more of
(a) wherein Lx comprises the amino acid sequence of SEQ ID NO: 194 or 199;
(b) wherein Cx comprises the amino acid sequence of SEQ ID NO: 75; and
(c) wherein Ly comprises the amino acid sequence of SEQ ID NO: 68.

23. The protein complex of claim 21, wherein the protease resistant single-chain IL-12 fusion protein comprises:
(i) the amino acid sequence of SEQ ID NO: 317;
(ii) an amino acid sequence of SEQ ID NO: 317, wherein the tyrosine at position 16 of SEQ ID NO: 317 is substituted with an amino acid selected from the group consisting of asparagine (N), glutamic acid (E), glycine (G) and proline (P), preferably wherein the tyrosine at position 16 of SEQ ID NO: 317 is substituted with P;
(iii) the amino acid sequence of SEQ ID NO: 285;
(iv) an amino acid sequence of SEQ ID NO: 285, wherein the tyrosine at position 16 of SEQ ID NO: 285 is substituted with an amino acid selected from the group consisting of asparagine (N), glutamic acid (E), glycine (G) and proline (P), preferably wherein the tyrosine at position 16 of SEQ ID NO: 285 is substituted with P;
(v) the amino acid sequence of SEQ ID NO: 325; or
(vi) the amino acid sequence of SEQ ID NO: 335.

24. The protein complex of claim 21 comprising two first polypeptides and two second polypeptides, wherein the first and second polypeptide comprises the amino acid sequence of SEQ ID NO: 204 and 201, respectively; or SEQ ID NO: 205 and 202, respectively; or SEQ ID NO: 264 and 122, respectively; or SEQ ID NO: 207 and 201, respectively; or SEQ ID NO: 208 and 202, respectively; or SEQ ID NO: 208 and 209, respectively; or SEQ ID NO: 210 and 209, respectively; or SEQ ID NO: 249 and 122, respectively.

25. A polynucleotide or a plurality of polynucleotides encoding the protein complex of claim 21.

26. A method of treating a disease or disorder in an individual in need thereof, comprising administering to said individual a therapeutically effective amount of the protein complex of claim 21.

27. A protein complex comprising two subunits associated with each other, wherein each subunit comprises a first polypeptide and a second polypeptide, wherein the first polypeptide comprises a light chain variable domain (VL) and a light chain constant domain (CL), and the second polypeptide is a fusion protein represented by the general formula (I), from the N- to the C-terminus: 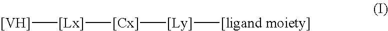
wherein:
VH represents a heavy chain variable domain (VH), wherein the VH is associated with the VL of the first polypeptide to form a ligand-binding domain that binds to the ligand moiety,
Lx represents a cleavable peptide linker comprising a protease cleavage site,
Cx represents a constant domain associated with the CL of the first polypeptide,
wherein Cx comprises, from the N- to the C-terminus, a CH1 domain, a second peptide linker, a CH2 domain, and a CH3 domain,
Ly represents a third peptide linker, and
the ligand moiety comprises a cytokine or chemokine, and
wherein (a) in a first state, the ligand moiety is bound by the ligand-binding domain and the biological activity of the ligand moiety is attenuated, and in a second state, the biological activity of the ligand moiety is restored, and (b) the protein complex in the first state has a longer half-life in blood than in the second state, and (c) switching from the first state to the second state is mediated by the presence of a protease that cleaves the protease cleavage site.

28. The protein complex of claim 27, characterized by one or more of
(a) the protease cleavage site is cleavable by a protease selected from the group consisting of matriptase (MT-SP1), urokinase-type plasminogen activator (uPA) and matrix metalloprotease (MMP);
(b) Lx represents a peptide linker comprising an amino acid sequence selected from SEQ ID NO: 194, 199, and 214 to 219;
(c) the ligand moiety is selected from the group consisting of IL-12, IL-22, IL-2, IL-7, IL-15, IL-18, IL-21, CXCL9, CXCL10, CXCL11, IFN-alpha, IFN-beta, IFN-gamma, MIG, I-TAC, RANTES, MIP-1a, MIP-1b, IL-1R1, IL-1R2, IL-1RAcP and IL-1Ra; and
(d) the VH comprises a substitution at the amino acid position corresponding to a position selected from residue 37, 45, 91, 100a, or 103 of SEQ ID NO: 116 (according to Kabat numbering), and/or the VL comprises a substitution at the amino acid position corresponding to a position selected from residue 30, 43, 46, 49 or 87 of SEQ ID NO: 114 (according to Kabat numbering).

29. A polynucleotide or a plurality of polynucleotides encoding the protein complex of claim 27.

30. A method of treating a disease or disorder in an individual in need thereof, comprising administering to said individual a therapeutically effective amount of the protein complex of claim 27.


SEQUENCE LISTING (1202 sequences)

SEQ ID NO: 1 - protein, 4 aa, synthetic construct
GGGS

SEQ ID NO: 2 - protein, 4 aa, synthetic construct
GGSG

SEQ ID NO: 3 - protein, 4 aa, synthetic construct
GSGG

SEQ ID NO: 4 - protein, 4 aa, synthetic construct
SGGG

SEQ ID NO: 5 - protein, 4 aa, synthetic construct
GSSG

SEQ ID NO: 6 - protein, 5 aa, synthetic construct
GGGGS

SEQ ID NO: 7 - protein, 5 aa, synthetic construct
GGGSG

SEQ ID NO: 8 - protein, 5 aa, synthetic construct
GGSGG

SEQ ID NO: 9 - protein, 5 aa, synthetic construct
GSGGG

SEQ ID NO: 10 - protein, 5 aa, synthetic construct
GSGGS

SEQ ID NO: 11 - protein, 5 aa, synthetic construct
SGGGG

SEQ ID NO: 12 - protein, 5 aa, synthetic construct
GSSGG

SEQ ID NO: 13 - protein, 5 aa, synthetic construct
GSGSG

SEQ ID NO: 14 - protein, 5 aa, synthetic construct
SGGSG

SEQ ID NO: 15 - protein, 5 aa, synthetic construct
GSSSG

SEQ ID NO: 16 - protein, 6 aa, synthetic construct
GGGGGS

SEQ ID NO: 17 - protein, 6 aa, synthetic construct
SGGGGG

SEQ ID NO: 18 - protein, 7 aa, synthetic construct
GGGGGGS

SEQ ID NO: 19 - protein, 7 aa, synthetic construct
SGGGGGG

SEQ ID NO: 20 - protein, 214 aa, synthetic construct
EIVMTQSPATLSVSPGERATLSCRASQSISINLHWYQQKPGQAPRLLIYFASQSISGIPARFSGSGSGTEFTLTISSLQSEDFAVYYCQQSNSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 18 aa, synthetic construct
GGSTSTSGRSANPRGGGS

SEQ ID NO: 22 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 23 - protein, 5 aa, synthetic construct
GGGGA

SEQ ID NO: 24 - protein, 5 aa, synthetic construct
GGGGE

SEQ ID NO: 25 - protein, 4 aa, synthetic construct
GGGA

SEQ ID NO: 26 - protein, 4 aa, synthetic construct
GGGE

SEQ ID NO: 27 - protein, 4 aa, synthetic construct
QQQG

SEQ ID NO: 28 - protein, 5 aa, synthetic construct
QQQQG

SEQ ID NO: 29 - protein, 4 aa, synthetic construct
SSSG

SEQ ID NO: 30 - protein, 5 aa, synthetic construct
SSSSG

SEQ ID NO: 31 - protein, 338 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 32 - protein, 18 aa, synthetic construct
GGSGGSGGSGGSGGSGGS

SEQ ID NO: 33 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 34 - protein, 338 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 35 - protein, 214 aa, synthetic construct
EIVMTQSPATLSVSPGERATLSCRASQSISINLHWYQQKPGQAPRLLIYFASQSISGIPARFSGSGSGTEFTLTISSLQSEDFAVYYCQQSNSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 36 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 37 - protein, 338 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 38 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 39 - protein, 338 aa, synthetic construct
ASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 40 - protein, 25 aa, synthetic construct
GGGGSGGGGSGGGGSGGGGSGGGGS

SEQ ID NO: 41 - protein, 338 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 42 - protein, 25 aa, synthetic construct
GGGGSGGGGSEPKSCDKTHTCPPCP

SEQ ID NO: 43 - protein, 25 aa, synthetic construct
EPKSCDKTHTGGGGSGGGGSCPPCP

SEQ ID NO: 44 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 45 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 46 - protein, 12 aa, synthetic construct
TSTSGRSANPRG

SEQ ID NO: 47 - protein, 550 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSGGSGGSTSTSGRSANPRGGGSGGSGGSEIVMTQSPATLSVSPGERATLSCRASQSISINLHWYQQKPGQAPRLLIYFASQSISGIPARFSGSGSGTEFTLTISSLQSEDFAVYYCQQSNSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 683 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 49 - protein, 214 aa, synthetic construct; identical to SEQ ID NO: 20

SEQ ID NO: 50 - protein, 30 aa, synthetic construct
GGSGGSGGSTSTSGRSANPRGGGSGGSGGS

SEQ ID NO: 51 - protein, 118 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSS

SEQ ID NO: 52 - protein, 18 aa, synthetic construct
GGSTSTSGRSANPRGGGS

SEQ ID NO: 53 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 54 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGSTSTSGRSANPRGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 55 - protein, 223 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 447 aa, synthetic construct
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 57 - protein, 683 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 58 - protein, 1009 aa, synthetic construct; identical to SEQ ID NO: 22

SEQ ID NO: 59 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 60 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 1010 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 62 - protein, 119 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 63 - protein, 219 aa, synthetic construct
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 1006 aa, synthetic construct
QVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 65 - protein, 115 aa, synthetic construct [sequence omitted]

SEQ ID NO: 66 - protein, 338 aa, synthetic construct; identical to SEQ ID NO: 31

SEQ ID NO: 67 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 68 - protein, 18 aa, synthetic construct
GGSGGSGGSGGSGGSGGS

SEQ ID NO: 69 - protein, 1009 aa, synthetic construct; identical to SEQ ID NO: 33

SEQ ID NO: 70 - protein, 338 aa, synthetic construct; identical to SEQ ID NO: 34

SEQ ID NO: 71 - protein, 214 aa, synthetic construct; identical to SEQ ID NO: 35

SEQ ID NO: 72 - protein, 1009 aa, synthetic construct; identical to SEQ ID NO: 36

SEQ ID NO: 73 - protein, 338 aa, synthetic construct; identical to SEQ ID NO: 37

SEQ ID NO: 74 - protein, 1009 aa, synthetic construct; identical to SEQ ID NO: 38

SEQ ID NO: 75 - protein, 338 aa, synthetic construct; identical to SEQ ID NO: 39

SEQ ID NO: 76 - protein, 146 aa, synthetic construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 77 - protein, 216 aa, synthetic construct
QAALTQPASVSGSPGQSITISCTGASGDVGAYNFVSWYQQHPGKAPKLIIYDVNKRPSGVSNRFSGSKSGNTASLTISGLQAEDEADYYCSSYTSTFSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 78 - protein, 635 aa, synthetic construct
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 79 - protein, 121 aa, synthetic construct
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSS

SEQ ID NO: 80 - protein, 216 aa, synthetic construct
QAALTQPASVSGSPGQSITISCTGASGDVGAYNFVSWYQQHPGKAPKLIIYDVNKRPSGVSNRFSGSKSGNTASLTISGLQAEDEADYYCSSYTSTFSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTESS

SEQ ID NO: 81 - protein, 635 aa, synthetic construct
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 82 - protein, 623 aa, synthetic construct
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 83 - protein, 141 aa, synthetic construct
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 84 - protein, 637 aa, synthetic construct
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSPGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 85 - protein, 218 aa, synthetic construct
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 637 aa, synthetic construct
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSGGGSGGGSGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSPGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 87 - protein, 635 aa, synthetic construct
QVQLVQSGGGVVQPGRSLRLSCAASGFTFSNYAMNWVRQAPGKGLEWVTLISYDGSQKYYADSVKGRFTTSRDNSKNTLYLQMNSLRAEDTAVYYCARDSTTLGAFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSPGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 88 - protein, 214 aa, synthetic construct
SYELTQPPSLSVSPGQTATISCSGDAFPRKFAYWYQQKSGQAPVLVIYEDTRRPSGIPERFSGSSSGTMATLTISGAQVEDEADYFCYSTDTTGTHRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 89 - protein, 635 aa, synthetic construct
QVQLVQSGGGVVQPGRSLRLSCAASGFTFSNYAMNWVRQAPGKGLEWVTLISYDGSQKYYADSVKGRFTTSRDNSKNTLYLQMNSLRAEDTAVYYCARDSTTLGAFDVWGQGTMVTVSSGGGSGGGSGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSPGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 90 - protein, 632 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSGGGSGGGSGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSPGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 91 - protein, 214 aa, synthetic construct
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 25 aa, synthetic construct
GGGGSGGGGSGGGGSGGGGSGGGGS

SEQ ID NO: 93 - protein, 12 aa, synthetic construct
GGGSGGGSGGGS

SEQ ID NO: 94 - protein, 635 aa, synthetic construct
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 95 - protein, 635 aa, synthetic construct
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 96 - protein, 121 aa, synthetic construct [sequence omitted]

SEQ ID NO: 97 - protein, 338 aa, synthetic construct; identical to SEQ ID NO: 41

SEQ ID NO: 98 - protein, 119 aa, synthetic construct
QVQLVQSGGGVVQPGRSLRLSCAASGFTFSNYAMNWVRQAPGKGLEWVTLISYDGSQKYYADSVKGRFTTSRDNSKNTLYLQMNSLRAEDTAVYYCARDSTTLGAFDVWGQGTMVTVSS

SEQ ID NO: 99 - protein, 116 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSS

SEQ ID NO: 100 - protein, 25 aa, synthetic construct
EPKSCGGGGSGGGGSDKTHTCPPCP

SEQ ID NO: 101 - protein, 5 aa, synthetic construct
EPKSC

SEQ ID NO: 102 - protein, 25 aa, synthetic construct
GGGGSGGGGSEPKSCDKTHTCPPCP

SEQ ID NO: 103 - protein, 25 aa, synthetic construct
EPKSCDKTHTGGGGSGGGGSCPPCP

SEQ ID NO: 104 - protein, 1010 aa, synthetic construct
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGSTSTSGRSANPRGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 105 - protein, 1010 aa, synthetic construct
QVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGGGSTSTSGRSANPRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 106 - protein, 10 aa, synthetic construct
GGGGSGGGGS

SEQ ID NO: 107 - protein, 212 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASENLYFQGDYKDDDDK

SEQ ID NO: 108 - protein, 7 aa, synthetic construct
ENLYFQG

SEQ ID NO: 109 - protein, 8 aa, synthetic construct
DYKDDDDK

SEQ ID NO: 110 - protein, 985 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 111 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 112 - protein, 985 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 113 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 114 - protein, 214 aa, synthetic construct
DIVMTQSPLSLPVTPGEPASISCRASQSISINLHWYQQKPGQAPRLLIYFASQSISGVPSRFSGSGSGTDFTLTISSLQPEDFATYYCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 458 aa, synthetic construct
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVWGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 116 - protein, 118 aa, synthetic construct
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVWGQGTLVTVSS

SEQ ID NO: 117 - protein, 1009 aa, synthetic construct
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVWGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 118 - protein, 1009 aa, synthetic construct
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVMGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 119 - protein, 1009 aa, synthetic construct
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVLGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 120 - protein, 1009 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 121 - protein, 214 aa, synthetic construct
DIVMTQSPLSLPVTPGEPASISCRASQSISINLHWYQQKPGQAPRQLIAFASQSISGVPSRFSGSGSGTDFTLTISSLQPEDFATYYCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 214 aa, synthetic construct
DIVMTQSPLSLPVTPGEPASISCRASQSIVINLHWYQQKPGQAPRQLIAFASQSISGVPSRFSGSGSGTDFTLTISSLQPEDFATYYCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 214 aa, synthetic construct
DIVMTQSPLSLPVTPGEPASISCRASQSISINLHWYQQKPGQAPRLLIAFASQSISGVPSRFSGSGSGTDFTLTISSLQPEDFATYLCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 214 aa, synthetic construct
DIVMTQSPLSLPVTPGEPASISCRASQSIVINLHWYQQKPGQAPRLLIAFASQSISGVPSRFSGSGSGTDFTLTISSLQPEDFATYLCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 78 aa, synthetic construct
VFPLSRTVRCTCISISNQPVNPRSLEKLEIIPASQFCPRVEIIATMKKKGEKRCLNPESKAIKNLLKAVSKERSKASP

SEQ ID NO: 126 - protein, 216 aa, synthetic construct
QSVLTQPPSASGTPGQRVTISCSGSSSNIGSNTVNWYQRLPGAAPQLLIYNNDQRPSGIPDRFSGSKSGTSGSLVISGLQSEDEADYYCASWDDSLNGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 127 - protein, 561 aa, synthetic construct
VFPLSRTVRCTCISISNQPVNPRSLEKLEIIPASQFCPRVEIIATMKKKGEKRCLNPESKAIKNLLKAVSKERSKASPGGGGSGGGGQVQLVESGGGVVQPGRSLRLSCAASGFTFSSFSITWIRQPPGKGLEWIGEITPMFGIANYAQKFQGQVTISADKSISTAYLQWSSLKASDTAMYYCARDGRFDVSDLLTDKPKVTINYNGMDVWGQGTLVTVSSTSGSGRSATPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 128 - protein, 216 aa, synthetic construct
QSVLTQPPSASGTPGQRVTISCSGSSSNIGSNTVNWYQELPGAAPQLLIYNNDQRPSGIPDRFSGSKSGTSGSLVISGLQSEDEADYYCASWDDSLNGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 129 - protein, 134 aa, synthetic construct
QVQLVESGGGVVQPGRSLRLSCAASGFTFSSFSITWIRQPPGKGLEWIGEITPMFGIANYAQKFQGQVTISADKSISTAYLQWSSLKASDTAMYYCARDGRFDVSDLLTDKPKVTINYNGMDVWGQGTLVTVSS

SEQ ID NO: 130 - protein, 9 aa, synthetic construct
GGGGSGGGG

SEQ ID NO: 131 - protein, 328 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 132 - protein, 12 aa, synthetic construct
TSGSGRSATPRG

SEQ ID NO: 133 - protein, 233 aa, synthetic construct
VFPLSRTVRCTCISISNQPVNPRSLEKLEIIPASQFCPRVEIIATMKKKGEKRCLNPESKAIKNLLKAVSKERSKASPGGGGSGGGGQVQLVESGGGVVQPGRSLRLSCAASGFTFSSFSITWIRDPPGKGLEWIGEITPMFGIANYAQKFQGQVTISADKSISTAYLQWSSLKASDTAMYYCARDGRFDVSDLLTDKPKVTINYNGMDVWGQGTLVTVSSTSGSGRSATPRG

SEQ ID NO: 134 - protein, 20 aa, synthetic construct
GGGGSGGGGSGGGGSGGGGS

SEQ ID NO: 135 - protein, 9 aa, synthetic construct
KSKREKKDR

SEQ ID NO: 136 - protein, 24 aa, synthetic construct
GGGGSGGGGSGGGGSGGGGSRNLP

SEQ ID NO: 137 - protein, 984 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 138 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 139 - protein, 984 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 140 - protein, 984 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 141 - protein, 984 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 142 - protein, 984 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHSEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 143 - protein, 985 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 144 - protein, 985 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKQREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 145 - protein, 985 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKEREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 146 - protein, 985 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKPREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 147 - protein, 983 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 148 - protein, 983 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 149 - protein, 983 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 150 - protein, 983 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKKHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 151 - protein, 983 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKRHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 152 - protein, 982 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 153 - protein, 982 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 154 - protein, 982 aa, synthetic construct
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 155 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 156 - protein, 22 aa, synthetic construct
GGGGSTSTSGRSANPRGGGGGS

SEQ ID NO: 157 - protein, 5 aa, synthetic construct
KSHRE

SEQ ID NO: 158 - protein, 5 aa, synthetic construct
KSHHE

SEQ ID NO: 159 - protein, 5 aa, synthetic construct
KSHKE

SEQ ID NO: 160 - protein, 5 aa, synthetic construct
KSHSE

SEQ ID NO: 161 - protein, 6 aa, synthetic construct
KSKHRE

SEQ ID NO: 162 - protein, 6 aa, synthetic construct
KSKQRE

SEQ ID NO: 163 - protein, 6 aa, synthetic construct
KSKERE

SEQ ID NO: 164 - protein, 6 aa, synthetic construct
KSKPRE

SEQ ID NO: 165 - protein, 4 aa, synthetic construct
KHKE

SEQ ID NO: 166 - protein, 4 aa, synthetic construct
KHHE

SEQ ID NO: 167 - protein, 4 aa, synthetic construct
KHRE

SEQ ID NO: 168 - protein, 4 aa, synthetic construct
KKHE

SEQ ID NO: 169 - protein, 4 aa, synthetic construct
KRHE

SEQ ID NO: 170-172 - skipped (3 entries)

SEQ ID NO: 173 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 174 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 175 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 176 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHSEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 177 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 178 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKQREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 179 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKEREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 180 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKPREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 181 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 182 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 183 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 184 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKKHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 185 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKRHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 186 - protein, 520 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 187 - protein, 520 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 188 - protein, 520 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 189 - protein, 118 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSS

SEQ ID NO: 190 - protein, 107 aa, synthetic construct
DIVMTQSPLSLPVTPGEPASISCRASQSISINLHWYQQKPGQAPRQLIAFASQSISGVPSRFSGSGSGTDFTLTISSLQPEDFATYYCQQSNSFPLTFGQGTKVEIK

SEQ ID NO: 191 - protein, 107 aa, synthetic construct
DIVMTQSPLSLPVTPGEPASISCRASQSIVINLHWYQQKPGQAPRQLIAFASQSISGVPSRFSGSGSGTDFTLTISSLQPEDFATYYCQQSNSFPLTFGQGTKVEIK

SEQ ID NO: 192 - protein, 328 aa, synthetic construct
ASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 193 - protein, 1007 aa, synthetic construct
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVWGQGTLVTVSSYSATGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 194 - protein, 12 aa, synthetic construct
YSATGRSANPRG

SEQ ID NO: 195 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 196 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 197 - protein, 6 aa, synthetic construct
KSKREK

SEQ ID NO: 198 - protein, 458 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 199 - protein, 12 aa, synthetic construct
TSGSGRSATPRG

SEQ ID NO: 200 - protein, 458 aa, synthetic construct
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 201 - protein, 214 aa, synthetic construct
DIVMTQSPLSLPVTPGEPASISCRASQSISINLHWYQQKPGQAPRQLIAFASQSISGVPSRFSGSGSGTDFTLTISSLQPEDFATYYCQQSYSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 202 - protein, 214 aa, synthetic construct
DIVMTQSPLSLPVTPGEPASISCRSSDSISINLHWYQQKPGQAPRQLIAFASQSISGVPSRFSGSGSGTDFTLTISSLQPEDFATYYCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 203 - protein, 458 aa, synthetic construct
QVQLVQSGAEVKKPGSSVKVSCKASGGTFSDYYLHWSRQAPGQGLEWMGWIDPENQDTEYAPKFQGRVTITADESTSTAYMELSSLRSEDTAVYYCARTKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 204 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSYSATGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 205 - protein, 1007 aa, synthetic construct
QVQLVQSGAEVKKPGSSVKVSCKASGGTFSDYYLHWSRQAPGQGLEWMGWIDPENQDTEYAPKFQGRVTITADESTSTAYMELSSLRSEDTAVYYCARTKELRYIDVWGQGTLVTVSSYSATGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 206 - protein, 1007 aa, synthetic construct
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 207 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 208 - protein, 1007 aa, synthetic construct
QVQLVQSGAEVKKPGSSVKVSCKASGGTFSDYYLHWSRQAPGQGLEWMGWIDPENQDTEYAPKFQGRVTITADESTSTAYMELSSLRSEDTAVYYCARTKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 209 - protein, 214 aa, synthetic construct
DIVMTQSPLSLPVTPGEPASISCRSSDSISINLHWYQQKPGQAPRQLIAFASQSIEGVPSRFSGSGSGTDFTLTISSLQPEDFATYYCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 1007 aa, synthetic construct
QVQLVQSGAEVKKPGSSVKVSCKASGGTFSDYYLHWSRQAPGQGLEWMGWIDAENQDTEYAPKFQGRVTITADESTSTAYMELSSLRSEDTAVYYCARTKELRYIDQWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 211 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGVAMPGAEDDVV

SEQ ID NO: 212 - protein, 226 aa, synthetic construct
EIVMTQSPATLSVSPGERATLSCRASQSISINLHWYQQKPGQAPRLLIYFASQSISGIPARFSGSGSGTEFTLTISSLQSEDFAVYYCQQSNSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGVAMPGAEDDVV

SEQ ID NO: 213 - protein, 12 aa, synthetic construct
GGGSGGGSGGGS

SEQ ID NO: 214 - protein, 12 aa, synthetic construct
TSTLGRSANPRG

SEQ ID NO: 215 - protein, 12 aa, synthetic construct
TSTSGRSAEFRG

SEQ ID NO: 216 - protein, 12 aa, synthetic construct
YAYTGRSANPRG

SEQ ID NO: 217 - protein, 12 aa, synthetic construct
TSPTGRSANPRG

SEQ ID NO: 218 - protein, 12 aa, synthetic construct
TSYTGRSAVYRG

SEQ ID NO: 219 - protein, 12 aa, synthetic construct
TSYTGRSAVVRG

SEQ ID NO: 220 - protein, 1021 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSGGGSGGGSGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGVAMPGAEDDVV

SEQ ID NO: 221 - protein, 1021 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTLGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGVAMPGAEDDVV

SEQ ID NO: 222 - protein, 1021 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSAEFRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGVAMPGAEDDVV

SEQ ID NO: 223 - protein, 1021 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGVAMPGAEDDVV

SEQ ID NO: 224 - protein, 1021 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSGSGRSATPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGVAMPGAEDDVV

SEQ ID NO: 225 - protein, 1021 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSYAYTGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGVAMPGAEDDVV

SEQ ID NO: 226 - protein, 1021 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSYSATGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGVAMPGAEDDVV

SEQ ID NO: 227 - protein, 1021 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSPTGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGVAMPGAEDDVV

SEQ ID NO: 228 - protein, 1021 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSYTGRSAVYRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGVAMPGAEDDVV

SEQ ID NO: 229 - protein, 1021 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSYTGRSAVVRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGVAMPGAEDDVV

SEQ ID NO: 230 - protein, 997 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 231 - protein, 214 aa, synthetic construct; identical to SEQ ID NO: 20

SEQ ID NO: 232 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSGGGSGGGSGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 233 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTLGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 234 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSAEFRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 235 - protein, 1009 aa, synthetic construct; identical to SEQ ID NO: 22

SEQ ID NO: 236 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSGSGRSATPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 237 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSYAYTGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 238 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSYSATGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 239 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSPTGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 240 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSYTGRSAVYRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 241 - protein, 1009 aa, synthetic construct
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSYTGRSAVVRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 242 - protein, 1007 aa, synthetic construct
QVQLVQSGAEVKKPGSSVKVSCKASGGTFSDYYLHWSRQAPGQGLEWMGWIDPENQDTEYAPKFQGRVTITADESTSTAYMELSSLRSEDTAVYYCARTKELRYIDVWGQGTLVTVSSGGGSGGGSGGGSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 243 - protein, 446 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 244 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKAFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 245 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 246 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWFPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 247 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWNPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 248 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWEPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 249 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWPPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 250 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWGPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 251 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWKPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 252 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWAPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 253 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWDPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 254 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWHPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 255 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWIPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 256 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWLPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 257 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWMPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 258 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWQPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 259 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWRPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 260 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWSPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 261 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWTPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 262 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWVPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 263 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSGSGRSATPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWWPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 264 - protein, 1007 aa, synthetic construct
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSYSATGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWPPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 265 - protein, 306 aa, synthetic construct
IWELKKDVYVVELDWPPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 266 - protein, 306 aa, synthetic construct
IWELKKDVYVVELDWYPDAPGEMVVLTCDTPEEDGITWTLDQSSEVLGSGKTLTIQVKAFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 267 - protein, 306 aa, synthetic construct
IWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 268 - protein, 306 aa, synthetic construct
IWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 269 - protein, 523 aa, synthetic construct
IWELKKDVYVVELDWPPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 270 - protein, 523 aa, synthetic construct
IWELKKDVYVVELDWYPDAPGEMVVLTCDTPEEDGITWTLDQSSEVLGSGKTLTIQVKAFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 271 - protein, 523 aa, synthetic construct
IWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 272 - protein, 523 aa, synthetic construct
IWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 273 - protein, 522 aa, synthetic construct
IWELKKDVYVVELDWPPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 274 - protein, 522 aa, synthetic construct
IWELKKDVYVVELDWYPDAPGEMVVLTCDTPEEDGITWTLDQSSEVLGSGKTLTIQVKAFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 275 - protein, 522 aa, synthetic construct
IWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 276 - protein, 522 aa, synthetic construct
IWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 277 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHREKKDRVFTDKTSATVICRKNASISVRAQDRYYSSSWSEWASVPCS

SEQ ID NO: 278 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHHEKKDRVFTDKTSATVICRKNASISVRAQDRYYSSSWSEWASVPCS

SEQ ID NO: 279 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHKEKKDRVFTDKTSATVICRKNASISVRAQDRYYSSSWSEWASVPCS

SEQ ID NO: 280 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHSEKKDRVFTDKTSATVICRKNASISVRAQDRYYSSSWSEWASVPCS

SEQ ID NO: 281 - protein, 307 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKHREKKDRVFTDKTSATVICRKNASISVRAQDRYYSSSWSEWASVPCS

SEQ ID NO: 282 - protein, 307 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKQREKKDRVFTDKTSATVICRKNASISVRAQDRYYSSSWSEWASVPCS

SEQ ID NO: 283 - protein, 307 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKEREKKDRVFTDKTSATVICRKNASISVRAQDRYYSSSWSEWASVPCS

SEQ ID NO: 284 - protein, 307 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKPREKKDRVFTDKTSATVICRKNASISVRAQDRYYSSSWSEWASVPCS

SEQ ID NO: 285 - protein, 305 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCS

SEQ ID NO: 286 - protein, 305 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHHEKKDRVFTDKTSATVICRKNASISVRAQDRYYSSSWSEWASVPCS

SEQ ID NO: 287 - protein, 305 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHREKKDRVFTDKTSATVICRKNASISVRAQDRYYSSSWSEWASVPCS

SEQ ID NO: 288 - protein, 305 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKKHEKKDRVFTDKTSATVICRKNASISVRAQDRYYSSSWSEWASVPCS

SEQ ID NO: 289 - protein, 305 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKRHEKKDRVFTDKTSATVICRKNASISVRAQDRYYSSSWSEWASVPCS

SEQ ID NO: 290 - protein, 304 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKREKKDRVFTDKTSATVICRKNASISVRAQDRYYSSSWSEWASVPCS

SEQ ID NO: 291 - protein, 304 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHEKKDRVFTDKTSATVICRKNASISVRAQDRYYSSSWSEWASVPCS

SEQ ID NO: 292 - protein, 304 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKKEKKDRVFTDKTSATVICRKNASISVRAQDRYYSSSWSEWASVPCS

SEQ ID NO: 293 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 294 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHHEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 295 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHKEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 296 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHSEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 297 - protein, 524 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKHREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 298 - protein, 524 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKQREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 299 - protein, 524 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKEREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 300 - protein, 524 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKPREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 301 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 302 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHHEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 303 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 304 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKKHEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 305 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKRHEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 306 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 307 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 308 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKKEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 309 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHREKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 310 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHHEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 311 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 312 - protein, 522 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSHSEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 313 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKHREKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 314 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKQREKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 315 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKEREKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 316 - protein, 523 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKPREKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 317 - protein, 521 aa, synthetic construct; identical to SEQ ID NO: 195

SEQ ID NO: 318 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHHEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 319 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHREKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 320 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKKHEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 321 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKRHEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 322 - protein, 520 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKREKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 323 - protein, 520 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 324 - protein, 520 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 325 - protein, 305 aa, synthetic construct
IWELKKDVYVVELDWPPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCS

SEQ ID NO: 326 - protein, 305 aa, synthetic construct
IWELKKDVYVVELDWYPDAPGEMVVLTCDTPEEDGITWTLDQSSEVLGSGKTLTIQVKAFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCS

SEQ ID NO: 327 - protein, 305 aa, synthetic construct
IWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCS

SEQ ID NO: 328 - protein, 305 aa, synthetic construct
IWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCS

SEQ ID NO: 329 - protein, 305 aa, synthetic construct
IWELKKDVYVVELDWPPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCS

SEQ ID NO: 330 - protein, 522 aa, synthetic construct
IWELKKDVYVVELDWPPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 331 - protein, 522 aa, synthetic construct
IWELKKDVYVVELDWYPDAPGEMVVLTCDTPEEDGITWTLDQSSEVLGSGKTLTIQVKAFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 332 - protein, 522 aa, synthetic construct
IWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 333 - protein, 522 aa, synthetic construct
IWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 334 - protein, 522 aa, synthetic construct
IWELKKDVYVVELDWPPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 335 - protein, 521 aa, synthetic construct
IWELKKDVYVVELDWPPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 336 - protein, 521 aa, synthetic construct
IWELKKDVYVVELDWYPDAPGEMVVLTCDTPEEDGITWTLDQSSEVLGSGKTLTIQVKAFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 337 - protein, 521 aa, synthetic construct
IWELKKDVYVVELDWYPDAPGEMVVLTCDTPEEDGITWTLDQSSEVLGSGKTLTIQVKEAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 338 - protein, 521 aa, synthetic construct
IWELKKDVYVVELDWYPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 339 - protein, 521 aa, synthetic construct
IWELKKDVYVVELDWPPDAPGEMVVLTCDTPEEDGITWTLDQSSEVLGSGKTLTIQVKAAGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 340 - protein, 118 aa, synthetic construct
QVQLVQSGAEVKKPGSSVKVSCKASGGTFSDYYLHWSRQAPGQGLEWMGWIDPENQDTEYAPKFQGRVTITADESTSTAYMELSSLRSEDTAVYYCARTKELRYIDVWGQGTLVTVSS

SEQ ID NO: 341 - protein, 118 aa, synthetic construct
QVQLVQSGAEVKKPGSSVKVSCKASGGTFSDYYLHWSRQAPGQGLEWMGWIDAENQDTEYAPKFQGRVTITADESTSTAYMELSSLRSEDTAVYYCARTKELRYIDQWGQGTLVTVSS

SEQ ID NO: 342 - protein, 107 aa, synthetic construct
DIVMTQSPLSLPVTPGEPASISCRASQSISINLHWYQQKPGQAPRQLIAFASQSISGVPSRFSGSGSGTDFTLTISSLQPEDFATYYCQQSYSFPLTFGQGTKVEIK

SEQ ID NO: 343 - protein, 107 aa, synthetic construct
DIVMTQSPLSLPVTPGEPASISCRSSDSISINLHWYQQKPGQAPRQLIAFASQSISGVPSRFSGSGSGTDFTLTISSLQPEDFATYYCQQSNSFPLTFGQGTKVEIK

SEQ ID NO: 344 - protein, 107 aa, synthetic construct
DIVMTQSPLSLPVTPGEPASISCRSSDSISINLHWYQQKPGQAPRQLIAFASQSIEGVPSRFSGSGSGTDFTLTISSLQPEDFATYYCQQSNSFPLTFGQGTKVEIK

SEQ ID NO: 345 - protein, 196 aa, synthetic construct
NLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 346 - protein, 5 aa, synthetic construct
DYYLH

SEQ ID NO: 347 - protein, 17 aa, synthetic construct
WIDPENQDTEYAPKFQG

SEQ ID NO: 348 - protein, 17 aa, synthetic construct
WIDPENGDTEYAPKFQG

SEQ ID NO: 349 - protein, 9 aa, synthetic construct
TKELRYIDV

SEQ ID NO: 350 - protein, 9 aa, synthetic construct
NKELRYIDV

SEQ ID NO: 351 - protein, 11 aa, synthetic construct
RSSDSISINLH

SEQ ID NO: 352 - protein, 11 aa, synthetic construct
RASQSISINLH

SEQ ID NO: 353 - protein, 7 aa, synthetic construct
FASQSIS

SEQ ID NO: 354 - protein, 7 aa, synthetic construct
FASQSIE

SEQ ID NO: 355 - protein, 9 aa, synthetic construct
QQSNSFPLT

SEQ ID NO: 356 - protein, 13 aa, synthetic construct
SDDTAVYYCNANK

SEQ ID NO: 357 - protein, 9 aa, synthetic construct
DIIKPDPPK

SEQ ID NO: 358 - protein, 20 aa, synthetic construct
QTLEFYPCTSEEIDHEDITK

SEQ ID NO: 359 - protein, 25 aa, synthetic construct
EPKSCGGGGSGGGGSDKTHTCPPCP

SEQ ID NO: 360 - protein, 5 aa, synthetic construct
EPKSC

SEQ ID NO: 361 - protein, 5 aa, synthetic construct
KSKRE

SEQ ID NO: 362 - protein, 330 aa, synthetic construct [sequence omitted]

SEQ ID NO: 363 - protein, 326 aa, synthetic construct
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 364 - protein, 377 aa, synthetic construct
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 365 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 366 - protein, 7 aa, synthetic construct
TLTIQVK

SEQ ID NO: 367 - protein, 536 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 368 - protein, 443 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGGGGSGGGGSGGGGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 369 - protein, 8 aa, synthetic construct
LSGRSDNH

SEQ ID NO: 370 - protein, 6 aa, synthetic construct
PLGLAG

SEQ ID NO: 371 - protein, 8 aa, synthetic construct
VPLSLTMG

SEQ ID NO: 372 - protein, 13 aa, synthetic construct
ISSGLLSGRSDNH

SEQ ID NO: 373 - protein, 18 aa, synthetic construct
AVGLLAPPGGLSGRSDNH

SEQ ID NO: 374 - protein, 13 aa, synthetic construct
GAGVPMSMRGGAG

SEQ ID NO: 375 - protein, 13 aa, synthetic construct
GAGIPVSLRSGAG

SEQ ID NO: 376 - protein, 8 aa, synthetic construct
GPLGIAGQ

SEQ ID NO: 377 - protein, 10 aa, synthetic construct
GGPLGMLSQS

SEQ ID NO: 378 - protein, 6 aa, synthetic construct
PLGLWA

SEQ ID NO: 379 - protein, 13 aa, synthetic construct
GAGRPFSMIMGAG

SEQ ID NO: 380 - protein, 13 aa, synthetic construct
GAGVPLSLTMGAG

SEQ ID NO: 381 - protein, 13 aa, synthetic construct
GAGVPLSLYSGAG

SEQ ID NO: 382 - protein, 6 aa, synthetic construct
AANLRN

SEQ ID NO: 383 - protein, 6 aa, synthetic construct
AQAYVK

SEQ ID NO: 384 - protein, 6 aa, synthetic construct
AANYMR

SEQ ID NO: 385 - protein, 6 aa, synthetic construct
AAALTR

SEQ ID NO: 386 - protein, 6 aa, synthetic construct
AQNLMR

SEQ ID NO: 387 - protein, 6 aa, synthetic construct
AANYTK

SEQ ID NO: 388 - protein, 16 aa, synthetic construct
GAGPQGLAGQRGIVAG

SEQ ID NO: 389 - protein, 8 aa, synthetic construct
PRFKIIGG

SEQ ID NO: 390 - protein, 8 aa, synthetic construct
PRFRIIGG

SEQ ID NO: 391 - protein, 9 aa, synthetic construct
GAGSGRSAG

SEQ ID NO: 392 - protein, 5 aa, synthetic construct
SGRSA

SEQ ID NO: 393 - protein, 6 aa, synthetic construct
GSGRSA

SEQ ID NO: 394 - protein, 5 aa, synthetic construct
SGKSA

SEQ ID NO: 395 - protein, 5 aa, synthetic construct
SGRSS

SEQ ID NO: 396 - protein, 5 aa, synthetic construct
SGRRA

SEQ ID NO: 397 - protein, 5 aa, synthetic construct
SGRNA

SEQ ID NO: 398 - protein, 5 aa, synthetic construct
SGRKA

SEQ ID NO: 399 - protein, 6 aa, synthetic construct
QRGRSA

SEQ ID NO: 400 - protein, 17 aa, synthetic construct
GAGSLLKSRMVPNFNAG

SEQ ID NO: 401 - protein, 6 aa, synthetic construct
TQGAAA

SEQ ID NO: 402 - protein, 6 aa, synthetic construct
GAAAAA

SEQ ID NO: 403 - protein, 6 aa, synthetic construct
GAGAAG

SEQ ID NO: 404 - protein, 6 aa, synthetic construct
AAAAAG

SEQ ID NO: 405 - protein, 6 aa, synthetic construct
LCGAAI

SEQ ID NO: 406 - protein, 6 aa, synthetic construct
FAQALG

SEQ ID NO: 407 - protein, 6 aa, synthetic construct
LLQANP

SEQ ID NO: 408 - protein, 6 aa, synthetic construct
LAAANP

SEQ ID NO: 409 - protein, 6 aa, synthetic construct
LYGAQF

SEQ ID NO: 410 - protein, 6 aa, synthetic construct
LSQAQG

SEQ ID NO: 411 - protein, 6 aa, synthetic construct
ASAASG

SEQ ID NO: 412 - protein, 6 aa, synthetic construct
FLGASL

SEQ ID NO: 413 - protein, 6 aa, synthetic construct
AYGATG

SEQ ID NO: 414 - protein, 6 aa, synthetic construct
LAQATG

SEQ ID NO: 415 - protein, 17 aa, synthetic construct
GAGSGVVIATVIVITAG

SEQ ID NO: 416 - protein, 8 aa, synthetic construct
APMAEGGG

SEQ ID NO: 417 - protein, 8 aa, synthetic construct
EAQGDKII

SEQ ID NO: 418 - protein, 8 aa, synthetic construct
LAFSDAGP

SEQ ID NO: 419 - protein, 7 aa, synthetic construct
YVADAPK

SEQ ID NO: 420 - protein, 5 aa, synthetic construct
RRRRR

SEQ ID NO: 421 - protein, 6 aa, synthetic construct
RRRRRR

SEQ ID NO: 422 - protein, 10 aa, synthetic construct
GQSSRHRRAL

SEQ ID NO: 423 - protein, 9 aa, synthetic construct
SSRHRRALD

SEQ ID NO: 424 - protein, 14 aa, synthetic construct
RKSSIIIRMRDVVL

SEQ ID NO: 425 - protein, 15 aa, synthetic construct
SSSFDKGKYKKGDDA

SEQ ID NO: 426 - protein, 15 aa, synthetic construct
SSSFDKGKYKRGDDA

SEQ ID NO: 427 - protein, 4 aa, synthetic construct
IEGR

SEQ ID NO: 428 - protein, 4 aa, synthetic construct
IDGR

SEQ ID NO: 429 - protein, 7 aa, synthetic construct
GGSIDGR

SEQ ID NO: 430 - protein, 8 aa, synthetic construct
GPQGIAGQ

SEQ ID NO: 431 - protein, 8 aa, synthetic construct
GPQGLLGA

SEQ ID NO: 432 - protein, 5 aa, synthetic construct
GIAGQ

SEQ ID NO: 433 - protein, 7 aa, synthetic construct
GPLGIAG

SEQ ID NO: 434 - protein, 8 aa, synthetic construct
GPEGLRVG

SEQ ID NO: 435 - protein, 8 aa, synthetic construct
YGAGLGVV

SEQ ID NO: 436 - protein, 8 aa, synthetic construct
AGLGVVER

SEQ ID NO: 437 - protein, 8 aa, synthetic construct
AGLGISST

SEQ ID NO: 438 - protein, 8 aa, synthetic construct
EPQALAMS

SEQ ID NO: 439 - protein, 8 aa, synthetic construct
QALAMSAI

SEQ ID NO: 440 - protein, 8 aa, synthetic construct
AAYHLVSQ

SEQ ID NO: 441 - protein, 8 aa, synthetic construct
MDAFLESS

SEQ ID NO: 442 - protein, 8 aa, synthetic construct
ESLPVVAV

SEQ ID NO: 443 - protein, 8 aa, synthetic construct
SAPAVESE

SEQ ID NO: 444 - protein, 8 aa, synthetic construct
DVAQFVLT

SEQ ID NO: 445 - protein, 8 aa, synthetic construct
VAQFVLTE

SEQ ID NO: 446 - protein, 8 aa, synthetic construct
AQFVLTEG

SEQ ID NO: 447 - protein, 8 aa, synthetic construct
PVQPIGPQ

SEQ ID NO: 448 - protein, 6 aa, synthetic construct
LVPRGS

SEQ ID NO: 449 - protein, 12 aa, synthetic construct
TSGSGRSANARG

SEQ ID NO: 450 - protein, 12 aa, synthetic construct
TSQSGRSANQRG

SEQ ID NO: 451 - protein, 12 aa, synthetic construct
TSPSGRSAYPRG

SEQ ID NO: 452 - protein, 12 aa, synthetic construct
TSQSGRSATPRG

SEQ ID NO: 453 - protein, 12 aa, synthetic construct
TSASGRSATPRG

SEQ ID NO: 454 - protein, 12 aa, synthetic construct
TSYSGRSAVPRG

SEQ ID NO: 455 - protein, 12 aa, synthetic construct
TSYSGRSANFRG

SEQ ID NO: 456 - protein, 12 aa, synthetic construct
TSSSGRSATPRG

SEQ ID NO: 457 - protein, 12 aa, synthetic construct
TSTTGRSASPRG

SEQ ID NO: 458 - protein, 12 aa, synthetic construct
TSASGRSANPRG

SEQ ID NO: 459 - protein, 12 aa, synthetic construct
TSESGRSANPRG

SEQ ID NO: 460 - protein, 12 aa, synthetic construct
TSFSGRSANPRG

SEQ ID NO: 461 - protein, 12 aa, synthetic construct
TSGSGRSANPRG

SEQ ID NO: 462 - protein, 12 aa, synthetic construct
TSHSGRSANPRG

SEQ ID NO: 463 - protein, 12 aa, synthetic construct
TSKSGRSANPRG

SEQ ID NO: 464 - protein, 12 aa, synthetic construct
TSMSGRSANPRG

SEQ ID NO: 465 - protein, 12 aa, synthetic construct
TSNSGRSANPRG

SEQ ID NO: 466 - protein, 12 aa, synthetic construct
TSPSGRSANPRG

SEQ ID NO: 467 - protein, 12 aa, synthetic construct
TSQSGRSANPRG

SEQ ID NO: 468 - protein, 12 aa, synthetic construct
TSWSGRSANPRG

SEQ ID NO: 469 - protein, 12 aa, synthetic construct
TSYSGRSANPRG

SEQ ID NO: 470 - protein, 12 aa, synthetic construct
TSTAGRSANPRG

SEQ ID NO: 471 - protein, 12 aa, synthetic construct
TSTDGRSANPRG

SEQ ID NO: 472 - protein, 12 aa, synthetic construct
TSTEGRSANPRG

SEQ ID NO: 473 - protein, 12 aa, synthetic construct
TSTFGRSANPRG

SEQ ID NO: 474 - protein, 12 aa, synthetic construct
TSTMGRSANPRG

SEQ ID NO: 475 - protein, 12 aa, synthetic construct
TSTPGRSANPRG

SEQ ID NO: 476 - protein, 12 aa, synthetic construct
TSTQGRSANPRG

SEQ ID NO: 477 - protein, 12 aa, synthetic construct
TSTVGRSANPRG

SEQ ID NO: 478 - protein, 12 aa, synthetic construct
TSTWGRSANPRG

SEQ ID NO: 479 - protein, 12 aa, synthetic construct
TSTSARSANPRG

SEQ ID NO: 480 - protein, 12 aa, synthetic construct
TSTSERSANPRG

SEQ ID NO: 481 - protein, 12 aa, synthetic construct
TSTSFRSANPRG

SEQ ID NO: 482 - protein, 12 aa, synthetic construct
TSTSHRSANPRG

SEQ ID NO: 483 - protein, 12 aa, synthetic construct
TSTSIRSANPRG

SEQ ID NO: 484 - protein, 12 aa, synthetic construct
TSTSKRSANPRG

SEQ ID NO: 485 - protein, 12 aa, synthetic construct
TSTSLRSANPRG

SEQ ID NO: 486 - protein, 12 aa, synthetic construct
TSTSMRSANPRG

SEQ ID NO: 487 - protein, 12 aa, synthetic construct
TSTSNRSANPRG

SEQ ID NO: 488 - protein, 12 aa, synthetic construct
TSTSPRSANPRG

SEQ ID NO: 489 - protein, 12 aa, synthetic construct
TSTSQRSANPRG

SEQ ID NO: 490 - protein, 12 aa, synthetic construct
TSTSRRSANPRG

SEQ ID NO: 491 - protein, 12 aa, synthetic construct
TSTSTRSANPRG

SEQ ID NO: 492 - protein, 12 aa, synthetic construct
TSTSVRSANPRG

SEQ ID NO: 493 - protein, 12 aa, synthetic construct
TSTSWRSANPRG

SEQ ID NO: 494 - protein, 12 aa, synthetic construct
TSTSYRSANPRG

SEQ ID NO: 495 - protein, 12 aa, synthetic construct
TSTSGRAANPRG

SEQ ID NO: 496 - protein, 12 aa, synthetic construct
TSTSGRDANPRG

SEQ ID NO: 497 - protein, 12 aa, synthetic construct
TSTSGREANPRG

SEQ ID NO: 498 - protein, 12 aa, synthetic construct
TSTSGRGANPRG

SEQ ID NO: 499 - protein, 12 aa, synthetic construct
TSTSGRHANPRG

SEQ ID NO: 500 - protein, 12 aa, synthetic construct
TSTSGRIANPRG

SEQ ID NO: 501 - protein, 12 aa, synthetic construct
TSTSGRKANPRG

SEQ ID NO: 502 - protein, 12 aa, synthetic construct
TSTSGRLANPRG

SEQ ID NO: 503 - protein, 12 aa, synthetic construct
TSTSGRMANPRG

SEQ ID NO: 504 - protein, 12 aa, synthetic construct
TSTSGRNANPRG

SEQ ID NO: 505 - protein, 12 aa, synthetic construct
TSTSGRPANPRG

SEQ ID NO: 506 - protein, 12 aa, synthetic construct
TSTSGRQANPRG

SEQ ID NO: 507 - protein, 12 aa, synthetic construct
TSTSGRRANPRG

SEQ ID NO: 508 - protein, 12 aa, synthetic construct
TSTSGRTANPRG

SEQ ID NO: 509 - protein, 12 aa, synthetic construct
TSTSGRVANPRG

SEQ ID NO: 510 - protein, 12 aa, synthetic construct
TSTSGRWANPRG

SEQ ID NO: 511 - protein, 12 aa, synthetic construct
TSTSGRYANPRG

SEQ ID NO: 512 - protein, 12 aa, synthetic construct
TSTSGRSENPRG

SEQ ID NO: 513 - protein, 12 aa, synthetic construct
TSTSGRSFNPRG

SEQ ID NO: 514 - protein, 12 aa, synthetic construct
TSTSGRSKNPRG

SEQ ID NO: 515 - protein, 12 aa, synthetic construct
TSTSGRSMNPRG

SEQ ID NO: 516 - protein, 12 aa, synthetic construct
TSTSGRSNNPRG

SEQ ID NO: 517 - protein, 12 aa, synthetic construct
TSTSGRSPNPRG

SEQ ID NO: 518 - protein, 12 aa, synthetic construct
TSTSGRSQNPRG

SEQ ID NO: 519 - protein, 12 aa, synthetic construct
TSTSGRSRNPRG

SEQ ID NO: 520 - protein, 12 aa, synthetic construct
TSTSGRSSNPRG

SEQ ID NO: 521 - protein, 12 aa, synthetic construct
TSTSGRSWNPRG

SEQ ID NO: 522 - protein, 12 aa, synthetic construct
TSTSGRSYNPRG

SEQ ID NO: 523 - protein, 12 aa, synthetic construct
TSTSGRSAAPRG

SEQ ID NO: 524 - protein, 12 aa, synthetic construct
TSTSGRSADPRG

SEQ ID NO: 525 - protein, 12 aa, synthetic construct
TSTSGRSAEPRG

SEQ ID NO: 526 - protein, 12 aa, synthetic construct
TSTSGRSAFPRG

SEQ ID NO: 527 - protein, 12 aa, synthetic construct
TSTSGRSAGPRG

SEQ ID NO: 528 - protein, 12 aa, synthetic construct
TSTSGRSAKPRG

SEQ ID NO: 529 - protein, 12 aa, synthetic construct
TSTSGRSALPRG

SEQ ID NO: 530 - protein, 12 aa, synthetic construct
TSTSGRSAMPRG

SEQ ID NO: 531 - protein, 12 aa, synthetic construct
TSTSGRSAPPRG

SEQ ID NO: 532 - protein, 12 aa, synthetic construct
TSTSGRSAQPRG

SEQ ID NO: 533 - protein, 12 aa, synthetic construct
TSTSGRSAVPRG

SEQ ID NO: 534 - protein, 12 aa, synthetic construct
TSTSGRSAWPRG

SEQ ID NO: 535 - protein, 12 aa, synthetic construct
TSTSGRSAYPRG

SEQ ID NO: 536 - protein, 12 aa, synthetic construct
TSTSGRSANARG

SEQ ID NO: 537 - protein, 12 aa, synthetic construct
TSTSGRSANDRG

SEQ ID NO: 538 - protein, 12 aa, synthetic construct
TSTSGRSANERG

SEQ ID NO: 539 - protein, 12 aa, synthetic construct
TSTSGRSANFRG

SEQ ID NO: 540 - protein, 12 aa, synthetic construct
TSTSGRSANGRG

SEQ ID NO: 541 - protein, 12 aa, synthetic construct
TSTSGRSANIRG

SEQ ID NO: 542 - protein, 12 aa, synthetic construct
TSTSGRSANKRG

SEQ ID NO: 543 - protein, 12 aa, synthetic construct
TSTSGRSANNRG

SEQ ID NO: 544 - protein, 12 aa, synthetic construct
TSTSGRSANQRG

SEQ ID NO: 545 - protein, 12 aa, synthetic construct
TSTSGRSANSRG

SEQ ID NO: 546 - protein, 12 aa, synthetic construct
TSTSGRSANTRG

SEQ ID NO: 547 - protein, 12 aa, synthetic construct
TSTSGRSANWRG

SEQ ID NO: 548 - protein, 12 aa, synthetic construct
TSDSGRSANPRG

SEQ ID NO: 549 - protein, 12 aa, synthetic construct
TSISGRSANPRG

SEQ ID NO: 550 - protein, 12 aa, synthetic construct
TSSSGRSANPRG

SEQ ID NO: 551 - protein, 12 aa, synthetic construct
TSTHGRSANPRG

SEQ ID NO: 552 - protein, 12 aa, synthetic construct
TSTKGRSANPRG

SEQ ID NO: 553 - protein, 12 aa, synthetic construct
TSTTGRSANPRG

SEQ ID NO: 554 - protein, 12 aa, synthetic construct
TSTYGRSANPRG

SEQ ID NO: 555 - protein, 12 aa, synthetic construct
TSTSDRSANPRG

SEQ ID NO: 556 - protein, 12 aa, synthetic construct
TSTSSRSANPRG

SEQ ID NO: 557 - protein, 12 aa, synthetic construct
TSTSGRFANPRG

SEQ ID NO: 558 - protein, 12 aa, synthetic construct
TSTSGRSDNPRG

SEQ ID NO: 559 - protein, 12 aa, synthetic construct
TSTSGRSHNPRG

SEQ ID NO: 560 - protein, 12 aa, synthetic construct
TSTSGRSINPRG

SEQ ID NO: 561 - protein, 12 aa, synthetic construct
TSTSGRSLNPRG

SEQ ID NO: 562 - protein, 12 aa, synthetic construct
TSTSGRSTNPRG

SEQ ID NO: 563 - protein, 12 aa, synthetic construct
TSTSGRSVNPRG

SEQ ID NO: 564 - protein, 12 aa, synthetic construct
TSTSGRSAHPRG

SEQ ID NO: 565 - protein, 12 aa, synthetic construct
TSTSGRSAIPRG

SEQ ID NO: 566 - protein, 12 aa, synthetic construct
TSTSGRSARPRG

SEQ ID NO: 567 - protein, 12 aa, synthetic construct
TSTSGRSASPRG

SEQ ID NO: 568 - protein, 12 aa, synthetic construct
TSTSGRSATPRG

SEQ ID NO: 569 - protein, 12 aa, synthetic construct
TSTSGRSANHRG

SEQ ID NO: 570 - protein, 12 aa, synthetic construct
TSTSGRSANLRG

SEQ ID NO: 571 - protein, 12 aa, synthetic construct
TSTSGRSANMRG

SEQ ID NO: 572 - protein, 12 aa, synthetic construct
TSTSGRSANRRG

SEQ ID NO: 573 - protein, 12 aa, synthetic construct
TSTSGRSANVRG

SEQ ID NO: 574 - protein, 12 aa, synthetic construct
TSTSGRSANYRG

SEQ ID NO: 575 - protein, 12 aa, synthetic construct
TSGSGRSAVPRG

SEQ ID NO: 576 - protein, 12 aa, synthetic construct
TSGSGRSAYPRG

SEQ ID NO: 577 - protein, 12 aa, synthetic construct
TSGSGRSANQRG

SEQ ID NO: 578 - protein, 12 aa, synthetic construct
TSGSGRSANIRG

SEQ ID NO: 579 - protein, 12 aa, synthetic construct
TSGSGRSANFRG

SEQ ID NO: 580 - protein, 12 aa, synthetic construct
TSGSGRSANSRG

SEQ ID NO: 581 - protein, 12 aa, synthetic construct
TSQSGRSAVPRG

SEQ ID NO: 582 - protein, 12 aa, synthetic construct
TSQSGRSAYPRG

SEQ ID NO: 583 - protein, 12 aa, synthetic construct
TSQSGRSANARG

SEQ ID NO: 584 - protein, 12 aa, synthetic construct
TSQSGRSANIRG

SEQ ID NO: 585 - protein, 12 aa, synthetic construct
TSQSGRSANFRG

SEQ ID NO: 586 - protein, 12 aa, synthetic construct
TSQSGRSANSRG

SEQ ID NO: 587 - protein, 12 aa, synthetic construct
TSPSGRSAVPRG

SEQ ID NO: 588 - protein, 12 aa, synthetic construct
TSPSGRSANQRG

SEQ ID NO: 589 - protein, 12 aa, synthetic construct
TSPSGRSANARG

SEQ ID NO: 590 - protein, 12 aa, synthetic construct
TSPSGRSANIRG

SEQ ID NO: 591 - protein, 12 aa, synthetic construct
TSPSGRSANFRG

SEQ ID NO: 592 - protein, 12 aa, synthetic construct
TSPSGRSANSRG

SEQ ID NO: 593 - protein, 12 aa, synthetic construct
TSASGRSAVPRG

SEQ ID NO: 594 - protein, 12 aa, synthetic construct
TSASGRSAYPRG

SEQ ID NO: 595 - protein, 12 aa, synthetic construct
TSASGRSANQRG

SEQ ID NO: 596 - protein, 12 aa, synthetic construct
TSASGRSANARG

SEQ ID NO: 597 - protein, 12 aa, synthetic construct
TSASGRSANIRG

SEQ ID NO: 598 - protein, 12 aa, synthetic construct
TSASGRSANFRG

SEQ ID NO: 599 - protein, 12 aa, synthetic construct
TSASGRSANSRG

SEQ ID NO: 600 - protein, 12 aa, synthetic construct
TSYSGRSENPRG

SEQ ID NO: 601 - protein, 12 aa, synthetic construct
TSGSGRSENPRG

SEQ ID NO: 602 - protein, 8 aa, synthetic construct
ASGRSANP

SEQ ID NO: 603 - protein, 8 aa, synthetic construct
ESGRSANP

SEQ ID NO: 604 - protein, 8 aa, synthetic construct
FSGRSANP

SEQ ID NO: 605 - protein, 8 aa, synthetic construct
GSGRSANP

SEQ ID NO: 606 - protein, 8 aa, synthetic construct
HSGRSANP

SEQ ID NO: 607 - protein, 8 aa, synthetic construct
KSGRSANP

SEQ ID NO: 608 - protein, 8 aa, synthetic construct
MSGRSANP

SEQ ID NO: 609 - protein, 8 aa, synthetic construct
NSGRSANP

SEQ ID NO: 610 - protein, 8 aa, synthetic construct
PSGRSANP

SEQ ID NO: 611 - protein, 8 aa, synthetic construct
QSGRSANP

SEQ ID NO: 612 - protein, 8 aa, synthetic construct
WSGRSANP

SEQ ID NO: 613 - protein, 8 aa, synthetic construct
YSGRSANP

SEQ ID NO: 614 - protein, 8 aa, synthetic construct
TAGRSANP

SEQ ID NO: 615 - protein, 8 aa, synthetic construct
TDGRSANP

SEQ ID NO: 616 - protein, 8 aa, synthetic construct
TEGRSANP

SEQ ID NO: 617 - protein, 8 aa, synthetic construct
TFGRSANP

SEQ ID NO: 618 - protein, 8 aa, synthetic construct
TLGRSANP

SEQ ID NO: 619 - protein, 8 aa, synthetic construct
TMGRSANP

SEQ ID NO: 620 - protein, 8 aa, synthetic construct
TPGRSANP

SEQ ID NO: 621 - protein, 8 aa, synthetic construct
TQGRSANP

SEQ ID NO: 622 - protein, 8 aa, synthetic construct
TVGRSANP

SEQ ID NO: 623 - protein, 8 aa, synthetic construct
TWGRSANP

SEQ ID NO: 624 - protein, 8 aa, synthetic construct
TSARSANP

SEQ ID NO: 625 - protein, 8 aa, synthetic construct
TSERSANP

SEQ ID NO: 626 - protein, 8 aa, synthetic construct
TSFRSANP

SEQ ID NO: 627 - protein, 8 aa, synthetic construct
TSHRSANP

SEQ ID NO: 628 - protein, 8 aa, synthetic construct
TSIRSANP

SEQ ID NO: 629 - protein, 8 aa, synthetic construct
TSKRSANP

SEQ ID NO: 630 - protein, 8 aa, synthetic construct
TSLRSANP

SEQ ID NO: 631 - protein, 8 aa, synthetic construct
TSMRSANP

SEQ ID NO: 632 - protein, 8 aa, synthetic construct
TSNRSANP

SEQ ID NO: 633 - protein, 8 aa, synthetic construct
TSPRSANP

SEQ ID NO: 634 - protein, 8 aa, synthetic construct
TSQRSANP

SEQ ID NO: 635 - protein, 8 aa, synthetic construct
TSRRSANP

SEQ ID NO: 636 - protein, 8 aa, synthetic construct
TSTRSANP

SEQ ID NO: 637 - protein, 8 aa, synthetic construct
TSVRSANP

SEQ ID NO: 638 - protein, 8 aa, synthetic construct
TSWRSANP

SEQ ID NO: 639 - protein, 8 aa, synthetic construct
TSYRSANP

SEQ ID NO: 640 - protein, 8 aa, synthetic construct
TSGRAANP

SEQ ID NO: 641 - protein, 8 aa, synthetic construct
TSGRDANP

SEQ ID NO: 642 - protein, 8 aa, synthetic construct
TSGREANP

SEQ ID NO: 643 - protein, 8 aa, synthetic construct
TSGRGANP

SEQ ID NO: 644 - protein, 8 aa, synthetic construct
TSGRHANP

SEQ ID NO: 645 - protein, 8 aa, synthetic construct
TSGRIANP

SEQ ID NO: 646 - protein, 8 aa, synthetic construct
TSGRKANP

SEQ ID NO: 647 - protein, 8 aa, synthetic construct
TSGRLANP

SEQ ID NO: 648 - protein, 8 aa, synthetic construct
TSGRMANP

SEQ ID NO: 649 - protein, 8 aa, synthetic construct
TSGRNANP

SEQ ID NO: 650 - protein, 8 aa, synthetic construct
TSGRPANP

SEQ ID NO: 651 - protein, 8 aa, synthetic construct
TSGRQANP

SEQ ID NO: 652 - protein, 8 aa, synthetic construct
TSGRRANP

SEQ ID NO: 653 - protein, 8 aa, synthetic construct
TSGRTANP

SEQ ID NO: 654 - protein, 8 aa, synthetic construct
TSGRVANP

SEQ ID NO: 655 - protein, 8 aa, synthetic construct
TSGRWANP

SEQ ID NO: 656 - protein, 8 aa, synthetic construct
TSGRYANP

SEQ ID NO: 657 - protein, 8 aa, synthetic construct
TSGRSENP

SEQ ID NO: 658 - protein, 8 aa, synthetic construct
TSGRSFNP

SEQ ID NO: 659 - protein, 8 aa, synthetic construct
TSGRSKNP

SEQ ID NO: 660 - protein, 8 aa, synthetic construct
TSGRSMNP

SEQ ID NO: 661 - protein, 8 aa, synthetic construct
TSGRSNNP

SEQ ID NO: 662 - protein, 8 aa, synthetic construct
TSGRSPNP

SEQ ID NO: 663 - protein, 8 aa, synthetic construct
TSGRSQNP

SEQ ID NO: 664 - protein, 8 aa, synthetic construct
TSGRSRNP

SEQ ID NO: 665 - protein, 8 aa, synthetic construct
TSGRSSNP

SEQ ID NO: 666 - protein, 8 aa, synthetic construct
TSGRSWNP

SEQ ID NO: 667 - protein, 8 aa, synthetic construct
TSGRSYNP

SEQ ID NO: 668 - protein, 8 aa, synthetic construct
TSGRSAAP

SEQ ID NO: 669 - protein, 8 aa, synthetic construct
TSGRSADP

SEQ ID NO: 670 - protein, 8 aa, synthetic construct
TSGRSAEP

SEQ ID NO: 671 - protein, 8 aa, synthetic construct
TSGRSAFP

SEQ ID NO: 672 - protein, 8 aa, synthetic construct
TSGRSAGP

SEQ ID NO: 673 - protein, 8 aa, synthetic construct
TSGRSAKP

SEQ ID NO: 674 - protein, 8 aa, synthetic construct
TSGRSALP

SEQ ID NO: 675 - protein, 8 aa, synthetic construct
TSGRSAMP

SEQ ID NO: 676 - protein, 8 aa, synthetic construct
TSGRSAPP

SEQ ID NO: 677 - protein, 8 aa, synthetic construct
TSGRSAQP

SEQ ID NO: 678 - protein, 8 aa, synthetic construct
TSGRSAVP

SEQ ID NO: 679 - protein, 8 aa, synthetic construct
TSGRSAWP

SEQ ID NO: 680 - protein, 8 aa, synthetic construct
TSGRSAYP

SEQ ID NO: 681 - protein, 8 aa, synthetic construct
TSGRSANA

SEQ ID NO: 682 - protein, 8 aa, synthetic construct
TSGRSAND

SEQ ID NO: 683 - protein, 8 aa, synthetic construct
TSGRSANE

SEQ ID NO: 684 - protein, 8 aa, synthetic construct
TSGRSANF

SEQ ID NO: 685 - protein, 8 aa, synthetic construct
TSGRSANG

SEQ ID NO: 686 - protein, 8 aa, synthetic construct
TSGRSANI

SEQ ID NO: 687 - protein, 8 aa, synthetic construct
TSGRSANK

SEQ ID NO: 688 - protein, 8 aa, synthetic construct
TSGRSANN

SEQ ID NO: 689 - protein, 8 aa, synthetic construct
TSGRSANQ

SEQ ID NO: 690 - protein, 8 aa, synthetic construct
TSGRSANS

SEQ ID NO: 691 - protein, 8 aa, synthetic construct
TSGRSANT

SEQ ID NO: 692 - protein, 8 aa, synthetic construct
TSGRSANW

SEQ ID NO: 693 - protein, 8 aa, synthetic construct
DSGRSANP

SEQ ID NO: 694 - protein, 8 aa, synthetic construct
ISGRSANP

SEQ ID NO: 695 - protein, 8 aa, synthetic construct
SSGRSANP

SEQ ID NO: 696 - protein, 8 aa, synthetic construct
THGRSANP

SEQ ID NO: 697 - protein, 8 aa, synthetic construct
TKGRSANP

SEQ ID NO: 698 - protein, 8 aa, synthetic construct
TTGRSANP

SEQ ID NO: 699 - protein, 8 aa, synthetic construct
TYGRSANP

SEQ ID NO: 700 - protein, 8 aa, synthetic construct
TSDRSANP

SEQ ID NO: 701 - protein, 8 aa, synthetic construct
TSSRSANP

SEQ ID NO: 702 - protein, 8 aa, synthetic construct
TSGRFANP

SEQ ID NO: 703 - protein, 8 aa, synthetic construct
TSGRSDNP

SEQ ID NO: 704 - protein, 8 aa, synthetic construct
TSGRSHNP

SEQ ID NO: 705 - protein, 8 aa, synthetic construct
TSGRSINP

SEQ ID NO: 706 - protein, 8 aa, synthetic construct
TSGRSLNP

SEQ ID NO: 707 - protein, 8 aa, synthetic construct
TSGRSTNP

SEQ ID NO: 708 - protein, 8 aa, synthetic construct
TSGRSVNP

SEQ ID NO: 709 - protein, 8 aa, synthetic construct
TSGRSAHP

SEQ ID NO: 710 - protein, 8 aa, synthetic construct
TSGRSAIP

SEQ ID NO: 711 - protein, 8 aa, synthetic construct
TSGRSARP

SEQ ID NO: 712 - protein, 8 aa, synthetic construct
TSGRSASP

SEQ ID NO: 713 - protein, 8 aa, synthetic construct
TSGRSATP

SEQ ID NO: 714 - protein, 8 aa, synthetic construct
TSGRSANH

SEQ ID NO: 715 - protein, 8 aa, synthetic construct
TSGRSANL

SEQ ID NO: 716 - protein, 8 aa, synthetic construct
TSGRSANM

SEQ ID NO: 717 - protein, 8 aa, synthetic construct
TSGRSANR

SEQ ID NO: 718 - protein, 8 aa, synthetic construct
TSGRSANV

SEQ ID NO: 719 - protein, 8 aa, synthetic construct
TSGRSANY

SEQ ID NO: 720 - protein, 8 aa, synthetic construct
GSGRSAVP

SEQ ID NO: 721 - protein, 8 aa, synthetic construct
GSGRSAYP

SEQ ID NO: 722 - protein, 8 aa, synthetic construct
GSGRSANQ

SEQ ID NO: 723 - protein, 8 aa, synthetic construct
GSGRSANA

SEQ ID NO: 724 - protein, 8 aa, synthetic construct
GSGRSANI

SEQ ID NO: 725 - protein, 8 aa, synthetic construct
GSGRSANF

SEQ ID NO: 726 - protein, 8 aa, synthetic construct
GSGRSANS

SEQ ID NO: 727 - protein, 8 aa, synthetic construct
QSGRSAVP

SEQ ID NO: 728 - protein, 8 aa, synthetic construct
QSGRSAYP

SEQ ID NO: 729 - protein, 8 aa, synthetic construct
QSGRSANQ

SEQ ID NO: 730 - protein, 8 aa, synthetic construct
QSGRSANA

SEQ ID NO: 731 - protein, 8 aa, synthetic construct
QSGRSANI

SEQ ID NO: 732 - protein, 8 aa, synthetic construct
QSGRSANF

SEQ ID NO: 733 - protein, 8 aa, synthetic construct
QSGRSANS

SEQ ID NO: 734 - protein, 8 aa, synthetic construct
PSGRSAVP

SEQ ID NO: 735 - protein, 8 aa, synthetic construct
PSGRSAYP

SEQ ID NO: 736 - protein, 8 aa, synthetic construct
PSGRSANQ

SEQ ID NO: 737 - protein, 8 aa, synthetic construct
PSGRSANA

SEQ ID NO: 738 - protein, 8 aa, synthetic construct
PSGRSANI

SEQ ID NO: 739 - protein, 8 aa, synthetic construct
PSGRSANF

SEQ ID NO: 740 - protein, 8 aa, synthetic construct
PSGRSANS

SEQ ID NO: 741 - protein, 8 aa, synthetic construct
ASGRSAVP

SEQ ID NO: 742 - protein, 8 aa, synthetic construct
ASGRSAYP

SEQ ID NO: 743 - protein, 8 aa, synthetic construct
ASGRSANQ

SEQ ID NO: 744 - protein, 8 aa, synthetic construct
ASGRSANA

SEQ ID NO: 745 - protein, 8 aa, synthetic construct
ASGRSANI

SEQ ID NO: 746 - protein, 8 aa, synthetic construct
ASGRSANF

SEQ ID NO: 747 - protein, 8 aa, synthetic construct
ASGRSANS

SEQ ID NO: 748 - protein, 8 aa, synthetic construct
YSGRSENP

SEQ ID NO: 749 - protein, 8 aa, synthetic construct
GSGRSENP

SEQ ID NO: 750 - protein, 8 aa, synthetic construct
QSGRSENP

SEQ ID NO: 751 - protein, 8 aa, synthetic construct
PSGRSENP

SEQ ID NO: 752 - protein, 8 aa, synthetic construct
ASGRSENP

SEQ ID NO: 753 - protein, 8 aa, synthetic construct
HSGRSENP

SEQ ID NO: 754 - protein, 8 aa, synthetic construct
TSGRSENQ

SEQ ID NO: 755 - protein, 8 aa, synthetic construct
TSGRSENA

SEQ ID NO: 756 - protein, 12 aa, synthetic construct
TSTSGRSANPAG

SEQ ID NO: 757 - protein, 12 aa, synthetic construct
TSTSGRSANPHG

SEQ ID NO: 758 - protein, 12 aa, synthetic construct
TSTSGRSANPIG

SEQ ID NO: 759 - protein, 12 aa, synthetic construct
TSTSGRSANPLG

SEQ ID NO: 760 - protein, 12 aa, synthetic construct
TSTSGRSANPSG

SEQ ID NO: 761 - protein, 12 aa, synthetic construct
ISTSGRSANPIG

SEQ ID NO: 762 - protein, 12 aa, synthetic construct
YSTSGRSANPIG

SEQ ID NO: 763 - protein, 12 aa, synthetic construct
TSYSGRSAVPAG

SEQ ID NO: 764 - protein, 12 aa, synthetic construct
TSPSGRSANIAG

SEQ ID NO: 765 - protein, 12 aa, synthetic construct
TSPSGRSANFAG

SEQ ID NO: 766 - protein, 12 aa, synthetic construct
TSPTGRSANPAG

SEQ ID NO: 767 - protein, 12 aa, synthetic construct
TSPSGRSAIPAG

SEQ ID NO: 768 - protein, 12 aa, synthetic construct
TSYTGRSANPAG

SEQ ID NO: 769 - protein, 12 aa, synthetic construct
TSYSGRSAIPAG

SEQ ID NO: 770 - protein, 12 aa, synthetic construct
TSISGRSANYAG

SEQ ID NO: 771 - protein, 12 aa, synthetic construct
TSPSGRSAGPAG

SEQ ID NO: 772 - protein, 12 aa, synthetic construct
TSYTGRSAVPAG

SEQ ID NO: 773 - protein, 12 aa, synthetic construct
TSYTGRSAVYAG

SEQ ID NO: 774 - protein, 12 aa, synthetic construct
TSYTGRSAVVAG

SEQ ID NO: 775 - protein, 12 aa, synthetic construct
TSYTGRSAVHAG

SEQ ID NO: 776 - protein, 12 aa, synthetic construct
TSYSGRSAVPHG

SEQ ID NO: 777 - protein, 12 aa, synthetic construct
TSPSGRSANIHG

SEQ ID NO: 778 - protein, 12 aa, synthetic construct
TSPSGRSANFHG

SEQ ID NO: 779 - protein, 12 aa, synthetic construct
TSPTGRSANPHG

SEQ ID NO: 780 - protein, 12 aa, synthetic construct
TSPSGRSAIPHG

SEQ ID NO: 781 - protein, 12 aa, synthetic construct
TSYTGRSANPHG

SEQ ID NO: 782 - protein, 12 aa, synthetic construct
TSYSGRSAIPHG

SEQ ID NO: 783 - protein, 12 aa, synthetic construct
TSISGRSANYHG

SEQ ID NO: 784 - protein, 12 aa, synthetic construct
TSPSGRSAGPHG

SEQ ID NO: 785 - protein, 12 aa, synthetic construct
TSYTGRSAVPHG

SEQ ID NO: 786 - protein, 12 aa, synthetic construct
TSYTGRSAVYHG

SEQ ID NO: 787 - protein, 12 aa, synthetic construct
TSYTGRSAVVHG

SEQ ID NO: 788 - protein, 12 aa, synthetic construct
TSYTGRSAVHHG

SEQ ID NO: 789 - protein, 12 aa, synthetic construct
TSYSGRSAVPIG

SEQ ID NO: 790 - protein, 12 aa, synthetic construct
TSPSGRSANIIG

SEQ ID NO: 791 - protein, 12 aa, synthetic construct
TSPSGRSANFIG

SEQ ID NO: 792 - protein, 12 aa, synthetic construct
TSPTGRSANPIG

SEQ ID NO: 793 - protein, 12 aa, synthetic construct
TSPSGRSAIPIG

SEQ ID NO: 794 - protein, 12 aa, synthetic construct
TSYTGRSANPIG

SEQ ID NO: 795 - protein, 12 aa, synthetic construct
TSYSGRSAIPIG

SEQ ID NO: 796 - protein, 12 aa, synthetic construct
TSISGRSANYIG

SEQ ID NO: 797 - protein, 12 aa, synthetic construct
TSPSGRSAGPIG

SEQ ID NO: 798 - protein, 12 aa, synthetic construct
TSYTGRSAVPIG

SEQ ID NO: 799 - protein, 12 aa, synthetic construct
TSYTGRSAVYIG

SEQ ID NO: 800 - protein, 12 aa, synthetic construct
TSYTGRSAVVIG

SEQ ID NO: 801 - protein, 12 aa, synthetic construct
TSYTGRSAVHIG

SEQ ID NO: 802 - protein, 12 aa, synthetic construct
TSYSGRSAVPLG

SEQ ID NO: 803 - protein, 12 aa, synthetic construct
TSPSGRSANILG

SEQ ID NO: 804 - protein, 12 aa, synthetic construct
TSPSGRSANFLG

SEQ ID NO: 805 - protein, 12 aa, synthetic construct
TSPTGRSANPLG

SEQ ID NO: 806 - protein, 12 aa, synthetic construct
TSPSGRSAIPLG

SEQ ID NO: 807 - protein, 12 aa, synthetic construct
TSYTGRSANPLG

SEQ ID NO: 808 - protein, 12 aa, synthetic construct
TSQSGRSENPRG

SEQ ID NO: 809 - protein, 12 aa, synthetic construct
TSPSGRSENPRG

SEQ ID NO: 810 - protein, 12 aa, synthetic construct
TSASGRSENPRG

SEQ ID NO: 811 - protein, 12 aa, synthetic construct
TSHSGRSENPRG

SEQ ID NO: 812 - protein, 12 aa, synthetic construct
TSTSGRSENQRG

SEQ ID NO: 813 - protein, 12 aa, synthetic construct
TSTSGRSENARG

SEQ ID NO: 814 - protein, 12 aa, synthetic construct
TSTSGRSENIRG

SEQ ID NO: 815 - protein, 12 aa, synthetic construct
TSTSGRSENFRG

SEQ ID NO: 816 - protein, 12 aa, synthetic construct
TSTSGRSENSRG

SEQ ID NO: 817 - protein, 12 aa, synthetic construct
TSYSGRSAEPRG

SEQ ID NO: 818 - protein, 12 aa, synthetic construct
TSGSGRSAEPRG

SEQ ID NO: 819 - protein, 12 aa, synthetic construct
TSQSGRSAEPRG

SEQ ID NO: 820 - protein, 12 aa, synthetic construct
TSPSGRSAEPRG

SEQ ID NO: 821 - protein, 12 aa, synthetic construct
TSASGRSAEPRG

SEQ ID NO: 822 - protein, 12 aa, synthetic construct
TSHSGRSAEPRG

SEQ ID NO: 823 - protein, 12 aa, synthetic construct
TSTSGRSAEQRG

SEQ ID NO: 824 - protein, 12 aa, synthetic construct
TSTSGRSAEARG

SEQ ID NO: 825 - protein, 12 aa, synthetic construct
TSTSGRSAEIRG

SEQ ID NO: 826 - protein, 12 aa, synthetic construct
TSTSGRSAESRG

SEQ ID NO: 827 - protein, 12 aa, synthetic construct
TSGTGRSANPRG

SEQ ID NO: 828 - protein, 12 aa, synthetic construct
TSGKGRSANPRG

SEQ ID NO: 829 - protein, 12 aa, synthetic construct
TSGSGRSAIPRG

SEQ ID NO: 830 - protein, 12 aa, synthetic construct
TSGSGRSASPRG

SEQ ID NO: 831 - protein, 12 aa, synthetic construct
TSGSGRSAHPRG

SEQ ID NO: 832 - protein, 12 aa, synthetic construct
TSGSGRSANYRG

SEQ ID NO: 833 - protein, 12 aa, synthetic construct
TSGSGRSANVRG

SEQ ID NO: 834 - protein, 12 aa, synthetic construct
TSGSGRSANHRG

SEQ ID NO: 835 - protein, 12 aa, synthetic construct
TSQTGRSANPRG

SEQ ID NO: 836 - protein, 12 aa, synthetic construct
TSQKGRSANPRG

SEQ ID NO: 837 - protein, 12 aa, synthetic construct
TSQSGRSAIPRG

SEQ ID NO: 838 - protein, 12 aa, synthetic construct
TSQSGRSASPRG

SEQ ID NO: 839 - protein, 12 aa, synthetic construct
TSQSGRSAHPRG

SEQ ID NO: 840 - protein, 12 aa, synthetic construct
TSQSGRSANYRG

SEQ ID NO: 841 - protein, 12 aa, synthetic construct
TSQSGRSANVRG

SEQ ID NO: 842 - protein, 12 aa, synthetic construct
TSQSGRSANHRG

SEQ ID NO: 843 - protein, 12 aa, synthetic construct
TSPKGRSANPRG

SEQ ID NO: 844 - protein, 12 aa, synthetic construct
TSPSGRSAIPRG

SEQ ID NO: 845 - protein, 12 aa, synthetic construct
TSPSGRSATPRG

SEQ ID NO: 846 - protein, 12 aa, synthetic construct
TSPSGRSASPRG

SEQ ID NO: 847 - protein, 12 aa, synthetic construct
TSPSGRSAHPRG

SEQ ID NO: 848 - protein, 12 aa, synthetic construct
TSPSGRSANYRG

SEQ ID NO: 849 - protein, 12 aa, synthetic construct
TSPSGRSANVRG

SEQ ID NO: 850 - protein, 12 aa, synthetic construct
TSPSGRSANHRG

SEQ ID NO: 851 - protein, 12 aa, synthetic construct
TSATGRSANPRG

SEQ ID NO: 852 - protein, 12 aa, synthetic construct
TSAKGRSANPRG

SEQ ID NO: 853 - protein, 12 aa, synthetic construct
TSASGRSAIPRG

SEQ ID NO: 854 - protein, 12 aa, synthetic construct
TSASGRSASPRG

SEQ ID NO: 855 - protein, 12 aa, synthetic construct
TSASGRSAHPRG

SEQ ID NO: 856 - protein, 12 aa, synthetic construct
TSASGRSANYRG

SEQ ID NO: 857 - protein, 12 aa, synthetic construct
TSASGRSANVRG

SEQ ID NO: 858 - protein, 12 aa, synthetic construct
TSASGRSANHRG

SEQ ID NO: 859 - protein, 12 aa, synthetic construct
TSYTGRSANPRG

SEQ ID NO: 860 - protein, 12 aa, synthetic construct
TSYKGRSANPRG

SEQ ID NO: 861 - protein, 12 aa, synthetic construct
TSYSGRSAIPRG

SEQ ID NO: 862 - protein, 12 aa, synthetic construct
TSYSGRSATPRG

SEQ ID NO: 863 - protein, 12 aa, synthetic construct
TSYSGRSASPRG

SEQ ID NO: 864 - protein, 12 aa, synthetic construct
TSYSGRSAHPRG

SEQ ID NO: 865 - protein, 12 aa, synthetic construct
TSYSGRSANARG

SEQ ID NO: 866 - protein, 12 aa, synthetic construct
TSYSGRSANYRG

SEQ ID NO: 867 - protein, 12 aa, synthetic construct
TSYSGRSANVRG

SEQ ID NO: 868 - protein, 12 aa, synthetic construct
TSYSGRSANHRG

SEQ ID NO: 869 - protein, 12 aa, synthetic construct
TSSTGRSANPRG

SEQ ID NO: 870 - protein, 12 aa, synthetic construct
TSSKGRSANPRG

SEQ ID NO: 871 - protein, 12 aa, synthetic construct
TSSSGRSAVPRG

SEQ ID NO: 872 - protein, 12 aa, synthetic construct
TSSSGRSAIPRG

SEQ ID NO: 873 - protein, 12 aa, synthetic construct
TSSSGRSASPRG

SEQ ID NO: 874 - protein, 12 aa, synthetic construct
TSSSGRSAHPRG

SEQ ID NO: 875 - protein, 12 aa, synthetic construct
TSSSGRSANARG

SEQ ID NO: 876 - protein, 12 aa, synthetic construct
TSSSGRSANFRG

SEQ ID NO: 877 - protein, 12 aa, synthetic construct
TSSSGRSANYRG

SEQ ID NO: 878 - protein, 12 aa, synthetic construct
TSSSGRSANVRG

SEQ ID NO: 879 - protein, 12 aa, synthetic construct
TSSSGRSANHRG

SEQ ID NO: 880 - protein, 12 aa, synthetic construct
TSITGRSANPRG

SEQ ID NO: 881 - protein, 12 aa, synthetic construct
TSIKGRSANPRG

SEQ ID NO: 882 - protein, 12 aa, synthetic construct
TSISGRSAVPRG

SEQ ID NO: 883 - protein, 12 aa, synthetic construct
TSISGRSAIPRG

SEQ ID NO: 884 - protein, 12 aa, synthetic construct
TSISGRSATPRG

SEQ ID NO: 885 - protein, 12 aa, synthetic construct
TSISGRSASPRG

SEQ ID NO: 886 - protein, 12 aa, synthetic construct
TSISGRSAHPRG

SEQ ID NO: 887 - protein, 12 aa, synthetic construct
TSISGRSANARG

SEQ ID NO: 888 - protein, 12 aa, synthetic construct
TSISGRSANFRG

SEQ ID NO: 889 - protein, 12 aa, synthetic construct
TSISGRSANYRG

SEQ ID NO: 890 - protein, 12 aa, synthetic construct
TSISGRSANVRG

SEQ ID NO: 891 - protein, 12 aa, synthetic construct
TSISGRSANHRG

SEQ ID NO: 892 - protein, 12 aa, synthetic construct
TSTTGRSAVPRG

SEQ ID NO: 893 - protein, 12 aa, synthetic construct
TSTTGRSAIPRG

SEQ ID NO: 894 - protein, 12 aa, synthetic construct
TSTTGRSATPRG

SEQ ID NO: 895 - protein, 12 aa, synthetic construct
TSTTGRSAHPRG

SEQ ID NO: 896 - protein, 12 aa, synthetic construct
TSTTGRSANARG

SEQ ID NO: 897 - protein, 12 aa, synthetic construct
TSTTGRSANFRG

SEQ ID NO: 898 - protein, 12 aa, synthetic construct
TSTTGRSANYRG

SEQ ID NO: 899 - protein, 12 aa, synthetic construct
TSTTGRSANVRG

SEQ ID NO: 900 - protein, 12 aa, synthetic construct
TSTTGRSANHRG

SEQ ID NO: 901 - protein, 12 aa, synthetic construct
TSTKGRSAVPRG

SEQ ID NO: 902 - protein, 12 aa, synthetic construct
TSTKGRSAIPRG

SEQ ID NO: 903 - protein, 12 aa, synthetic construct
TSTKGRSATPRG

SEQ ID NO: 904 - protein, 12 aa, synthetic construct
TSTKGRSASPRG

SEQ ID NO: 905 - protein, 12 aa, synthetic construct
TSTKGRSAHPRG

SEQ ID NO: 906 - protein, 12 aa, synthetic construct
TSTKGRSANARG

SEQ ID NO: 907 - protein, 12 aa, synthetic construct
TSTKGRSANFRG

SEQ ID NO: 908 - protein, 12 aa, synthetic construct
TSTKGRSANYRG

SEQ ID NO: 909 - protein, 12 aa, synthetic construct
TSTKGRSANVRG

SEQ ID NO: 910 - protein, 12 aa, synthetic construct
TSTKGRSANHRG

SEQ ID NO: 911 - protein, 12 aa, synthetic construct
TSTSGRSAVYRG

SEQ ID NO: 912 - protein, 12 aa, synthetic construct
TSTSGRSAVVRG

SEQ ID NO: 913 - protein, 12 aa, synthetic construct
TSTSGRSAVHRG

SEQ ID NO: 914 - protein, 12 aa, synthetic construct
TSTSGRSAIYRG

SEQ ID NO: 915 - protein, 12 aa, synthetic construct
TSTSGRSAIVRG

SEQ ID NO: 916 - protein, 12 aa, synthetic construct
TSTSGRSAIHRG

SEQ ID NO: 917 - protein, 12 aa, synthetic construct
TSTSGRSASYRG

SEQ ID NO: 918 - protein, 12 aa, synthetic construct
TSTSGRSASVRG

SEQ ID NO: 919 - protein, 12 aa, synthetic construct
TSTSGRSASHRG

SEQ ID NO: 920 - protein, 12 aa, synthetic construct
TSTSGRSAHYRG

SEQ ID NO: 921 - protein, 12 aa, synthetic construct
TSTSGRSAHVRG

SEQ ID NO: 922 - protein, 12 aa, synthetic construct
TSTSGRSAHHRG

SEQ ID NO: 923 - protein, 12 aa, synthetic construct
TSPSGRSEVPRG

SEQ ID NO: 924 - protein, 12 aa, synthetic construct
TSPSGRSAEPRG

SEQ ID NO: 925 - protein, 12 aa, synthetic construct
TSPSGRSAGPRG

SEQ ID NO: 926 - protein, 12 aa, synthetic construct
TSASGRSENARG

SEQ ID NO: 927 - protein, 12 aa, synthetic construct
TSASGRSAEARG

SEQ ID NO: 928 - protein, 12 aa, synthetic construct
TSASGRSAGARG

SEQ ID NO: 929 - protein, 12 aa, synthetic construct
TSGTGRSATPRG

SEQ ID NO: 930 - protein, 12 aa, synthetic construct
TSGSGRSATYRG

SEQ ID NO: 931 - protein, 12 aa, synthetic construct
TSGSGRSATVRG

SEQ ID NO: 932 - protein, 12 aa, synthetic construct
TSGSGRSATHRG

SEQ ID NO: 933 - protein, 12 aa, synthetic construct
TSGTGRSATYRG

SEQ ID NO: 934 - protein, 12 aa, synthetic construct
TSGTGRSATVRG

SEQ ID NO: 935 - protein, 12 aa, synthetic construct
TSGTGRSATHRG

SEQ ID NO: 936 - protein, 12 aa, synthetic construct
TSGSGRSETPRG

SEQ ID NO: 937 - protein, 12 aa, synthetic construct
TSGTGRSETPRG

SEQ ID NO: 938 - protein, 12 aa, synthetic construct
TSGSGRSETYRG

SEQ ID NO: 939 - protein, 12 aa, synthetic construct
TSGSGRSETVRG

SEQ ID NO: 940 - protein, 12 aa, synthetic construct
TSGSGRSETHRG

SEQ ID NO: 941 - protein, 12 aa, synthetic construct
TSYTGRSAVPRG

SEQ ID NO: 942 - protein, 12 aa, synthetic construct
TSYSGRSAVYRG

SEQ ID NO: 943 - protein, 12 aa, synthetic construct
TSYSGRSAVVRG

SEQ ID NO: 944 - protein, 12 aa, synthetic construct
TSYSGRSAVHRG

SEQ ID NO: 945 - protein, 12 aa, synthetic construct
TSYTGRSAVHRG

SEQ ID NO: 946 - protein, 12 aa, synthetic construct
TSYSGRSEVPRG

SEQ ID NO: 947 - protein, 12 aa, synthetic construct
TSYTGRSEVPRG

SEQ ID NO: 948 - protein, 12 aa, synthetic construct
TSYSGRSEVYRG

SEQ ID NO: 949 - protein, 12 aa, synthetic construct
TSYSGRSEVVRG

SEQ ID NO: 950 - protein, 12 aa, synthetic construct
TSYSGRSEVHRG

SEQ ID NO: 951 - protein, 12 aa, synthetic construct
TSYTGRSAVPGG

SEQ ID NO: 952 - protein, 12 aa, synthetic construct
TSYSGRSAVYGG

SEQ ID NO: 953 - protein, 12 aa, synthetic construct
TSYSGRSAVVGG

SEQ ID NO: 954 - protein, 12 aa, synthetic construct
TSYSGRSAVHGG

SEQ ID NO: 955 - protein, 12 aa, synthetic construct
TSYTGRSAVYGG

SEQ ID NO: 956 - protein, 12 aa, synthetic construct
TSYTGRSAVVGG

SEQ ID NO: 957 - protein, 12 aa, synthetic construct
TSYTGRSAVHGG

SEQ ID NO: 958 - protein, 8 aa, synthetic construct
TSGRSENI

SEQ ID NO: 959 - protein, 8 aa, synthetic construct
TSGRSENF

SEQ ID NO: 960 - protein, 8 aa, synthetic construct
TSGRSENS

SEQ ID NO: 961 - protein, 8 aa, synthetic construct
YSGRSAEP

SEQ ID NO: 962 - protein, 8 aa, synthetic construct
GSGRSAEP

SEQ ID NO: 963 - protein, 8 aa, synthetic construct
QSGRSAEP

SEQ ID NO: 964 - protein, 8 aa, synthetic construct
PSGRSAEP

SEQ ID NO: 965 - protein, 8 aa, synthetic construct
ASGRSAEP

SEQ ID NO: 966 - protein, 8 aa, synthetic construct
HSGRSAEP

SEQ ID NO: 967 - protein, 8 aa, synthetic construct
TSGRSAEQ

SEQ ID NO: 968 - protein, 8 aa, synthetic construct
TSGRSAEA

SEQ ID NO: 969 - protein, 8 aa, synthetic construct
TSGRSAEI

SEQ ID NO: 970 - protein, 8 aa, synthetic construct
TSGRSAEF

SEQ ID NO: 971 - protein, 8 aa, synthetic construct
TSGRSAES

SEQ ID NO: 972 - protein, 8 aa, synthetic construct
GTGRSANP

SEQ ID NO: 973 - protein, 8 aa, synthetic construct
GKGRSANP

SEQ ID NO: 974 - protein, 8 aa, synthetic construct
GSGRSAIP

SEQ ID NO: 975 - protein, 8 aa, synthetic construct
GSGRSATP

SEQ ID NO: 976 - protein, 8 aa, synthetic construct
GSGRSASP

SEQ ID NO: 977 - protein, 8 aa, synthetic construct
GSGRSAHP

SEQ ID NO: 978 - protein, 8 aa, synthetic construct
GSGRSANY

SEQ ID NO: 979 - protein, 8 aa, synthetic construct
GSGRSANV

SEQ ID NO: 980 - protein, 8 aa, synthetic construct
GSGRSANH

SEQ ID NO: 981 - protein, 8 aa, synthetic construct
QTGRSANP

SEQ ID NO: 982 - protein, 8 aa, synthetic construct
QKGRSANP

SEQ ID NO: 983 - protein, 8 aa, synthetic construct
QSGRSAIP

SEQ ID NO: 984 - protein, 8 aa, synthetic construct
QSGRSATP

SEQ ID NO: 985 - protein, 8 aa, synthetic construct
QSGRSASP

SEQ ID NO: 986 - protein, 8 aa, synthetic construct
QSGRSAHP

SEQ ID NO: 987 - protein, 8 aa, synthetic construct
QSGRSANY

SEQ ID NO: 988 - protein, 8 aa, synthetic construct
QSGRSANV

SEQ ID NO: 989 - protein, 8 aa, synthetic construct
QSGRSANH

SEQ ID NO: 990 - protein, 8 aa, synthetic construct
PTGRSANP

SEQ ID NO: 991 - protein, 8 aa, synthetic construct
PKGRSANP

SEQ ID NO: 992 - protein, 8 aa, synthetic construct
PSGRSAIP

SEQ ID NO: 993 - protein, 8 aa, synthetic construct
PSGRSATP

SEQ ID NO: 994 - protein, 8 aa, synthetic construct
PSGRSASP

SEQ ID NO: 995 - protein, 8 aa, synthetic construct
PSGRSAHP

SEQ ID NO: 996 - protein, 8 aa, synthetic construct
PSGRSANY

SEQ ID NO: 997 - protein, 8 aa, synthetic construct
PSGRSANV

SEQ ID NO: 998 - protein, 8 aa, synthetic construct
PSGRSANH

SEQ ID NO: 999 - protein, 8 aa, synthetic construct
ATGRSANP

SEQ ID NO: 1000 - protein, 8 aa, synthetic construct
AKGRSANP

SEQ ID NO: 1001 - protein, 8 aa, synthetic construct
ASGRSAIP

SEQ ID NO: 1002 - protein, 8 aa, synthetic construct
ASGRSATP

SEQ ID NO: 1003 - protein, 8 aa, synthetic construct
ASGRSASP

SEQ ID NO: 1004 - protein, 8 aa, synthetic construct
ASGRSAHP

SEQ ID NO: 1005 - protein, 8 aa, synthetic construct
ASGRSANY

SEQ ID NO: 1006 - protein, 8 aa, synthetic construct
ASGRSANV

SEQ ID NO: 1007 - protein, 8 aa, synthetic construct
ASGRSANH

SEQ ID NO: 1008 - protein, 8 aa, synthetic construct
YTGRSANP

SEQ ID NO: 1009 - protein, 8 aa, synthetic construct
YKGRSANP

SEQ ID NO: 1010 - protein, 8 aa, synthetic construct
YSGRSAVP

SEQ ID NO: 1011 - protein, 8 aa, synthetic construct
YSGRSAIP

SEQ ID NO: 1012 - protein, 8 aa, synthetic construct
YSGRSATP

SEQ ID NO: 1013 - protein, 8 aa, synthetic construct
YSGRSASP

SEQ ID NO: 1014 - protein, 8 aa, synthetic construct
YSGRSAHP

SEQ ID NO: 1015 - protein, 8 aa, synthetic construct
YSGRSANA

SEQ ID NO: 1016 - protein, 8 aa, synthetic construct
YSGRSANF

SEQ ID NO: 1017 - protein, 8 aa, synthetic construct
YSGRSANY

SEQ ID NO: 1018 - protein, 8 aa, synthetic construct
YSGRSANV

SEQ ID NO: 1019 - protein, 8 aa, synthetic construct
YSGRSANH

SEQ ID NO: 1020 - protein, 8 aa, synthetic construct
STGRSANP

SEQ ID NO: 1021 - protein, 8 aa, synthetic construct
SKGRSANP

SEQ ID NO: 1022 - protein, 8 aa, synthetic construct
SSGRSAVP

SEQ ID NO: 1023 - protein, 8 aa, synthetic construct
SSGRSAIP

SEQ ID NO: 1024 - protein, 8 aa, synthetic construct
SSGRSATP

SEQ ID NO: 1025 - protein, 8 aa, synthetic construct
SSGRSASP

SEQ ID NO: 1026 - protein, 8 aa, synthetic construct
SSGRSAHP

SEQ ID NO: 1027 - protein, 8 aa, synthetic construct
SSGRSANA

SEQ ID NO: 1028 - protein, 8 aa, synthetic construct
SSGRSANF

SEQ ID NO: 1029 - protein, 8 aa, synthetic construct
SSGRSANY

SEQ ID NO: 1030 - protein, 8 aa, synthetic construct
SSGRSANV

SEQ ID NO: 1031 - protein, 8 aa, synthetic construct
SSGRSANH

SEQ ID NO: 1032 - protein, 8 aa, synthetic construct
ITGRSANP

SEQ ID NO: 1033 - protein, 8 aa, synthetic construct
IKGRSANP

SEQ ID NO: 1034 - protein, 8 aa, synthetic construct
ISGRSAVP

SEQ ID NO: 1035 - protein, 8 aa, synthetic construct
ISGRSAIP

SEQ ID NO: 1036 - protein, 8 aa, synthetic construct
ISGRSATP

SEQ ID NO: 1037 - protein, 8 aa, synthetic construct
ISGRSASP

SEQ ID NO: 1038 - protein, 8 aa, synthetic construct
ISGRSAHP

SEQ ID NO: 1039 - protein, 8 aa, synthetic construct
ISGRSANA

SEQ ID NO: 1040 - protein, 8 aa, synthetic construct
ISGRSANF

SEQ ID NO: 1041 - protein, 8 aa, synthetic construct
ISGRSANY

SEQ ID NO: 1042 - protein, 8 aa, synthetic construct
ISGRSANV

SEQ ID NO: 1043 - protein, 8 aa, synthetic construct
ISGRSANH

SEQ ID NO: 1044 - protein, 8 aa, synthetic construct
TTGRSAVP

SEQ ID NO: 1045 - protein, 8 aa, synthetic construct
TTGRSAIP

SEQ ID NO: 1046 - protein, 8 aa, synthetic construct
TTGRSATP

SEQ ID NO: 1047 - protein, 8 aa, synthetic construct
TTGRSASP

SEQ ID NO: 1048 - protein, 8 aa, synthetic construct
TTGRSAHP

SEQ ID NO: 1049 - protein, 8 aa, synthetic construct
TTGRSANA

SEQ ID NO: 1050 - protein, 8 aa, synthetic construct
TTGRSANF

SEQ ID NO: 1051 - protein, 8 aa, synthetic construct
TTGRSANY

SEQ ID NO: 1052 - protein, 8 aa, synthetic construct
TTGRSANV

SEQ ID NO: 1053 - protein, 8 aa, synthetic construct
TTGRSANH

SEQ ID NO: 1054 - protein, 8 aa, synthetic construct
TKGRSAVP

SEQ ID NO: 1055 - protein, 8 aa, synthetic construct
TKGRSAIP

SEQ ID NO: 1056 - protein, 8 aa, synthetic construct
TKGRSATP

SEQ ID NO: 1057 - protein, 8 aa, synthetic construct
TKGRSASP

SEQ ID NO: 1058 - protein, 8 aa, synthetic construct
TKGRSAHP

SEQ ID NO: 1059 - protein, 8 aa, synthetic construct
TKGRSANA

SEQ ID NO: 1060 - protein, 8 aa, synthetic construct
TKGRSANF

SEQ ID NO: 1061 - protein, 8 aa, synthetic construct
TKGRSANY

SEQ ID NO: 1062 - protein, 8 aa, synthetic construct
TKGRSANV

SEQ ID NO: 1063 - protein, 8 aa, synthetic construct
TKGRSANH

SEQ ID NO: 1064 - protein, 8 aa, synthetic construct
TSGRSAVY

SEQ ID NO: 1065 - protein, 8 aa, synthetic construct
TSGRSAVV

SEQ ID NO: 1066 - protein, 8 aa, synthetic construct
TSGRSAVH

SEQ ID NO: 1067 - protein, 8 aa, synthetic construct
TSGRSAIY

SEQ ID NO: 1068 - protein, 8 aa, synthetic construct
TSGRSAIV

SEQ ID NO: 1069 - protein, 8 aa, synthetic construct
TSGRSAIH

SEQ ID NO: 1070 - protein, 8 aa, synthetic construct
TSGRSASY

SEQ ID NO: 1071 - protein, 8 aa, synthetic construct
TSGRSASV

SEQ ID NO: 1072 - protein, 8 aa, synthetic construct
TSGRSASH

SEQ ID NO: 1073 - protein, 8 aa, synthetic construct
TSGRSAHY

SEQ ID NO: 1074 - protein, 8 aa, synthetic construct
TSGRSAHV

SEQ ID NO: 1075 - protein, 8 aa, synthetic construct
TSGRSAHH

SEQ ID NO: 1076 - protein, 8 aa, synthetic construct
PSGRSEVP

SEQ ID NO: 1077 - protein, 8 aa, synthetic construct
PSGRSAEP

SEQ ID NO: 1078 - protein, 8 aa, synthetic construct
PSGRSAGP

SEQ ID NO: 1079 - protein, 8 aa, synthetic construct
ASGRSENA

SEQ ID NO: 1080 - protein, 8 aa, synthetic construct
ASGRSAEA

SEQ ID NO: 1081 - protein, 8 aa, synthetic construct
ASGRSAGA

SEQ ID NO: 1082 - protein, 8 aa, synthetic construct
GTGRSATP

SEQ ID NO: 1083 - protein, 8 aa, synthetic construct
GSGRSATY

SEQ ID NO: 1084 - protein, 8 aa, synthetic construct
GSGRSATV

SEQ ID NO: 1085 - protein, 8 aa, synthetic construct
GSGRSATH

SEQ ID NO: 1086 - protein, 8 aa, synthetic construct
GTGRSATY

SEQ ID NO: 1087 - protein, 8 aa, synthetic construct
GTGRSATV

SEQ ID NO: 1088 - protein, 8 aa, synthetic construct
GTGRSATH

SEQ ID NO: 1089 - protein, 8 aa, synthetic construct
GSGRSETP

SEQ ID NO: 1090 - protein, 8 aa, synthetic construct
GTGRSETP

SEQ ID NO: 1091 - protein, 8 aa, synthetic construct
GSGRSETY

SEQ ID NO: 1092 - protein, 8 aa, synthetic construct
GSGRSETV

SEQ ID NO: 1093 - protein, 8 aa, synthetic construct
GSGRSETH

SEQ ID NO: 1094 - protein, 8 aa, synthetic construct
YTGRSAVP

SEQ ID NO: 1095 - protein, 8 aa, synthetic construct
YSGRSAVY

SEQ ID NO: 1096 - protein, 8 aa, synthetic construct
YSGRSAVV

SEQ ID NO: 1097 - protein, 8 aa, synthetic construct
YSGRSAVH

SEQ ID NO: 1098 - protein, 8 aa, synthetic construct
YTGRSAVY

SEQ ID NO: 1099 - protein, 8 aa, synthetic construct
YTGRSAVV

SEQ ID NO: 1100 - protein, 8 aa, synthetic construct
YTGRSAVH

SEQ ID NO: 1101 - protein, 8 aa, synthetic construct
YSGRSEVP

SEQ ID NO: 1102 - protein, 8 aa, synthetic construct
YTGRSEVP

SEQ ID NO: 1103 - protein, 8 aa, synthetic construct
YSGRSEVY

SEQ ID NO: 1104 - protein, 8 aa, synthetic construct
YSGRSEVV

SEQ ID NO: 1105 - protein, 8 aa, synthetic construct
YSGRSEVH

SEQ ID NO: 1106 - protein, 8 aa, synthetic construct
YTGRSAVP

SEQ ID NO: 1107 - protein, 8 aa, synthetic construct
YSGRSAVY

SEQ ID NO: 1108 - protein, 8 aa, synthetic construct
YSGRSAVV

SEQ ID NO: 1109 - protein, 8 aa, synthetic construct
YSGRSAVH

SEQ ID NO: 1110 - protein, 8 aa, synthetic construct
YTGRSAVY

SEQ ID NO: 1111 - protein, 8 aa, synthetic construct
YTGRSAVV

SEQ ID NO: 1112 - protein, 8 aa, synthetic construct
YTGRSAVH

SEQ ID NO: 1113 - protein, 12 aa, synthetic construct
TSYSGRSAIPLG

SEQ ID NO: 1114 - protein, 12 aa, synthetic construct
TSISGRSANYLG

SEQ ID NO: 1115 - protein, 12 aa, synthetic construct
TSPSGRSAGPLG

SEQ ID NO: 1116 - protein, 12 aa, synthetic construct
TSYTGRSAVPLG

SEQ ID NO: 1117 - protein, 12 aa, synthetic construct
TSYTGRSAVYLG

SEQ ID NO: 1118 - protein, 12 aa, synthetic construct
TSYTGRSAVVLG

SEQ ID NO: 1119 - protein, 12 aa, synthetic construct
TSYTGRSAVHLG

SEQ ID NO: 1120 - protein, 12 aa, synthetic construct
TSYSGRSAVPSG

SEQ ID NO: 1121 - protein, 12 aa, synthetic construct
TSPSGRSANISG

SEQ ID NO: 1122 - protein, 12 aa, synthetic construct
TSPSGRSANFSG

SEQ ID NO: 1123 - protein, 12 aa, synthetic construct
TSPTGRSANPSG

SEQ ID NO: 1124 - protein, 12 aa, synthetic construct
TSPSGRSAIPSG

SEQ ID NO: 1125 - protein, 12 aa, synthetic construct
TSYTGRSANPSG

SEQ ID NO: 1126 - protein, 12 aa, synthetic construct
TSYSGRSAIPSG

SEQ ID NO: 1127 - protein, 12 aa, synthetic construct
TSISGRSANYSG

SEQ ID NO: 1128 - protein, 12 aa, synthetic construct
TSPSGRSAGPSG

SEQ ID NO: 1129 - protein, 12 aa, synthetic construct
TSYTGRSAVPSG

SEQ ID NO: 1130 - protein, 12 aa, synthetic construct
TSYTGRSAVYSG

SEQ ID NO: 1131 - protein, 12 aa, synthetic construct
TSYTGRSAVVSG

SEQ ID NO: 1132 - protein, 12 aa, synthetic construct
TSYTGRSAVHSG

SEQ ID NO: 1133 - protein, 12 aa, synthetic construct
ISYSGRSAVPIG

SEQ ID NO: 1134 - protein, 12 aa, synthetic construct
ISPSGRSANIIG

SEQ ID NO: 1135 - protein, 12 aa, synthetic construct
ISPSGRSANFIG

SEQ ID NO: 1136 - protein, 12 aa, synthetic construct
ISPTGRSANPIG

SEQ ID NO: 1137 - protein, 12 aa, synthetic construct
ISPSGRSAIPIG

SEQ ID NO: 1138 - protein, 12 aa, synthetic construct
ISYTGRSANPIG

SEQ ID NO: 1139 - protein, 12 aa, synthetic construct
ISYSGRSAIPIG

SEQ ID NO: 1140 - protein, 12 aa, synthetic construct
ISISGRSANYIG

SEQ ID NO: 1141 - protein, 12 aa, synthetic construct
ISPSGRSAGPIG

SEQ ID NO: 1142 - protein, 12 aa, synthetic construct
ISYTGRSAVPIG

SEQ ID NO: 1143 - protein, 12 aa, synthetic construct
ISYTGRSAVYIG

SEQ ID NO: 1144 - protein, 12 aa, synthetic construct
ISYTGRSAVVIG

SEQ ID NO: 1145 - protein, 12 aa, synthetic construct
ISYTGRSAVHIG

SEQ ID NO: 1146 - protein, 12 aa, synthetic construct
YSYSGRSAVPIG

SEQ ID NO: 1147 - protein, 12 aa, synthetic construct
YSPSGRSANIIG

SEQ ID NO: 1148 - protein, 12 aa, synthetic construct
YSPSGRSANFIG

SEQ ID NO: 1149 - protein, 12 aa, synthetic construct
YSPTGRSANPIG

SEQ ID NO: 1150 - protein, 12 aa, synthetic construct
YSPSGRSAIPIG

SEQ ID NO: 1151 - protein, 12 aa, synthetic construct
YSYTGRSANPIG

SEQ ID NO: 1152 - protein, 12 aa, synthetic construct
YSYSGRSAIPIG

SEQ ID NO: 1153 - protein, 12 aa, synthetic construct
YSISGRSANYIG

SEQ ID NO: 1154 - protein, 12 aa, synthetic construct
YSPSGRSAGPIG

SEQ ID NO: 1155 - protein, 12 aa, synthetic construct
YSYTGRSAVPIG

SEQ ID NO: 1156 - protein, 12 aa, synthetic construct
YSYTGRSAVYIG

SEQ ID NO: 1157 - protein, 12 aa, synthetic construct
YSYTGRSAVVIG

SEQ ID NO: 1158 - protein, 12 aa, synthetic construct
YSATGRSANPRG

SEQ ID NO: 1159 - protein, 12 aa, synthetic construct
TSYTGRSAVPRG

SEQ ID NO: 1160 - protein, 12 aa, synthetic construct
TSYSGRSAVVRG

SEQ ID NO: 1161 - protein, 12 aa, synthetic construct
TSYTGRSAVYRG

SEQ ID NO: 1162 - protein, 12 aa, synthetic construct
TSYTGRSAVHRG

SEQ ID NO: 1163-1170 - skipped (8 entries)

SEQ ID NO: 1171 - protein, 8 aa, synthetic construct
  VARIANT 1: note = X1 is an amino acid selected from A, G, I, P, Q, S and Y
  VARIANT 7: note = X7 is an amino acid selected from H, I and V
  VARIANT 2: note = X2 is an amino acid selected from K or T
  VARIANT 8: note = X8 is an amino acid selected from H, V and Y
XXGRSAXX

SEQ ID NO: 1172 - protein, 8 aa, synthetic construct
  VARIANT 2: note = X2 is an amino acid selected from S and T
  VARIANT 6: note = X6 is an amino acid selected from A and E
  VARIANT 7: note = X7 is an amino acid selected from N and V
  VARIANT 8: note = X8 is an amino acid selected from H, P, V and Y
YXGRSXXX

SEQ ID NO: 1173-1180 - skipped (8 entries)

SEQ ID NO: 1181 - protein, 9 aa, synthetic construct
  VARIANT 1: note = X1 is an amino acid selected from A, G, I, P, Q, S and Y
  VARIANT 2: note = X2 is an amino acid selected from K or T
  VARIANT 7: note = X7 is an amino acid selected from H, I and V
  VARIANT 8: note = X8 is an amino acid selected from H, V and Y
  VARIANT 9: note = X9 is an amino acid selected from A, G, H, I, L and R
XXGRSAXXX

SEQ ID NO: 1182 - protein, 9 aa, synthetic construct
  VARIANT 2: note = X2 is an amino acid selected from S and T
  VARIANT 6: note = X6 is an amino acid selected from A and E
  VARIANT 7: note = X7 is an amino acid selected from N and V
  VARIANT 8: note = X8 is an amino acid selected from H, P, V and Y
  VARIANT 9: note = X9 is an amino acid selected from A, G, H, I, L and R
YXGRSXXXX

SEQ ID NO: 1183-1190 - skipped (8 entries)

SEQ ID NO: 1191 - protein, 10 aa, synthetic construct
  VARIANT 1: note = X1 is an amino acid selected from I, T and Y
  VARIANT 3: note = X3 is an amino acid selected from A, G, I, P, Q, S and Y
  VARIANT 4: note = X4 is an amino acid selected from K or T
  VARIANT 9: note = X9 is an amino acid selected from H, I and V
  VARIANT 10: note = X10 is an amino acid selected from H, V and Y
XSXXGRSAXX

SEQ ID NO: 1192 - protein, 10 aa, synthetic construct
  VARIANT 1: note = X1 is an amino acid selected from I, T and Y
  VARIANT 4: note = X4 is an amino acid selected from S and T
  VARIANT 8: note = X8 is an amino acid selected from A and E
  VARIANT 9: note = X9 is an amino acid selected from N and V
  VARIANT 10: note = X10 is an amino acid selected from H, P, V and Y
XSYXGRSXXX

SEQ ID NO: 1193-1200 - skipped (8 entries)

SEQ ID NO: 1201 - protein, 11 aa, synthetic construct
  VARIANT 1: note = X1 is an amino acid selected from I, T and Y
  VARIANT 3: note = X3 is an amino acid selected from A, G, I, P, Q, S and Y
  VARIANT 4: note = X4 is an amino acid selected from K or T
  VARIANT 9: note = X9 is an amino acid selected from H, I and V
  VARIANT 10: note = X10 is an amino acid selected from H, V and Y
  VARIANT 11: note = X11 is an amino acid selected from A, G, H, I, L and R
XSXXGRSAXXX

SEQ ID NO: 1202 - protein, 11 aa, synthetic construct
  VARIANT 1: note = X1 is an amino acid selected from I, T and Y
  VARIANT 4: note = X4 is an amino acid selected from S and T
  VARIANT 8: note = X8 is an amino acid selected from A and E
  VARIANT 9: note = X9 is an amino acid selected from N and V
  VARIANT 10: note = X10 is an amino acid selected from H, P, V and Y
  VARIANT 11: note = X11 is an amino acid selected from A, G, H, I, L and R
XSYXGRSXXXX